(19) United States
(12) Patent Application Publication
(10) Pub. No.: US 2016/0318856 A1
(43) Pub. Date: Nov. 3, 2016

(54) Substituted Urea eIF2alpha Kinase Activators

(71) Applicant: THE BRIGHAM AND WOMEN'S HOSPITAL, INC., Boston, MA (US)

(72) Inventors: Bertal Aktas, Newton, MA (US); Michael Chorev, Chestnut Hill, MA (US)

(21) Appl. No.: 14/917,718

(22) PCT Filed: Sep. 11, 2014
(86) PCT No.: PCT/US14/55204

§ 371 Date: Mar. 9, 2016

Related U.S. Application Data
(60) Provisional application No. 61/876,576, filed on Sep. 11, 2013.

(51) Int. Cl. C07C 275/30; C07D 213/65; C07C 275/40; C07C 275/34; C07C 335/16

(52) U.S. Cl. CPC C07C 275/30; C07C 275/34; C07C 335/16; C07C 275/40; C07D 213/65; C07C 2101/14

(57) ABSTRACT

This disclosure relates to substituted urea eIF2α kinase activators including methods of making and using the same. For example, such activators can include cycloalkyl aryl ureas, which activate at least one eIF2α kinase. These compounds may be useful for treatment of diseases such as, for example, cancer, hemolytic anemia not caused by infectious agents, Wolcott-Rallison syndrome, neurodegenerative disease, tuberous sclerosis complex, fragile-X syndrome, autism spectrum disorder, and ribosomal defect disease.

### CROSS-REFERENCE TO RELATED APPLICATIONS

This application is claims priority to U.S. Provisional Application Ser. No. 61/876,576, filed on Sep. 11, 2013, which is incorporated by reference in its entirety herein.

### FEDERALLY SPONSORED RESEARCH OR DEVELOPMENT

This invention was made with Government support under Grants No. R21AG032546, 1RO1CA152312, ES02710, and P42 ES04699 awarded by the National Institutes of Health. The Government has certain rights in the invention.

### TECHNICAL FIELD

This invention relates to substituted urea eIF2α kinase activators including methods of making and using the same.

### BACKGROUND

The eukaryotic translation initiation factor 2 (eIF2) forms a complex with initiator methionine transfer RNA (Met-tRNAi) and GTP to form a ternary complex, which is necessary for recognition of mRNA start codon and translation initiation. Hydrolysis of GTP in the eIF2•GTP•Met-tRNAi ternary complex and release of inorganic phosphate (Pi) are thought to be important for translation initiation and start-site selection. The eIF2.GDP binary complex, released concomitantly with initiation of translation, is converted to eIF2.GTP by eIF2B, a guanine nucleotide exchange factor. This GDP-GTP exchange is the rate-limiting step for the formation of the ternary complex and initiation of a new round of translation. Phosphorylation of the alpha subunit of eIF2 (eIF2α) on S51 by eIF2α kinases, HRI, RNA dependent-protein-kinase/protein kinase R (PKR), pancreatic eIF2α kinase/PKR-like endoplasmic reticulum kinase (PERK), and general control non-derepressible-2 (GCN2), is a mechanism that regulates the GDP-GTP exchange. More specifically, S51 phosphorylation on eIF2α can concomitantly increase its affinity for eIF2B and inhibit guanine nucleotide exchange activity of this enzyme. Because the eIF2 is present in excess over eIF2B (low eIF2B/eIF2 stoichiometry), even partial phosphorylation of eIF2α can result in sequestration of eIF2B thereby reducing the amount of the eIF2•GTP•Met-tRNAi ternary complex, and inhibiting translation initiation.

Formation of the eIF2•GTP•Met-tRNAi ternary complex is coupled to cell physiology and plays many roles in normal and patho-biology. Proliferating cells synthesize proteins at a higher rate than quiescent cells of similar types. The lower rate of translation in quiescent cells is achieved in part by higher rates of eIF2α phosphorylation compared to proliferating cells. Phosphorylation of eIF2α is important for coupling protein synthesis to heme availability in red blood cells progenitors to the folding capacity of ER-golgi network in the secretory cells, and to the nutrient and oxygen availability in all cells. eIF2α phosphorylation also plays a role in resisting infection by intracellular invaders.

New compositions and methods for preparing and formulating eIF2α kinase activator(s) would be useful.

### SUMMARY

Deregulation of eIF2α phosphorylation is implicated in the patho-biology of various human disorders. For example, inactivating mutations of the eIF2α kinase PERK has been linked with Wolcott-Rallison syndrome, a rare autosomal recessive disease characterized by neonatal/early-onset non-autoimmune insulin-requiring diabetes associated with skeletal dysplasia and growth retardation syndrome. Insufficiency of eIF2α phosphorylation that occurs in red blood cell progenitors deficient in heme-regulated inhibitor (HRI) can increase the severity of hemolytic anemia, such as β-thalassemia. Deregulation of eIF2α phosphorylation has also been implicated in the pathogenesis of neurodegenerative disorders such as Alzheimer's disease and proliferative disorders including cancer. Forced expression of eIF2α-S51A, a non-phosphorylatable mutant, increases the amount of the ternary complex, renders the translation initiation unrestricted, and can cause transformation of normal cells. Similarly, overexpression of Met-tRNAi causes cellular transformation. In contrast, induction of eIF2α phosphorylation pharmacologically or by over-expressing eIF2α kinases can inhibit proliferation of cancer cells in vitro and tumor growth in vivo.

Provided herein is a compound of Formula (III):

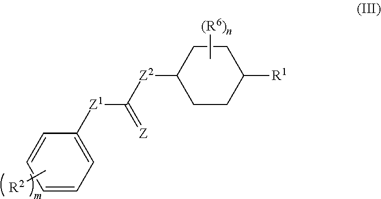

or a pharmaceutically acceptable salt thereof.

Non-limiting examples of a compound of Formula (III) include:

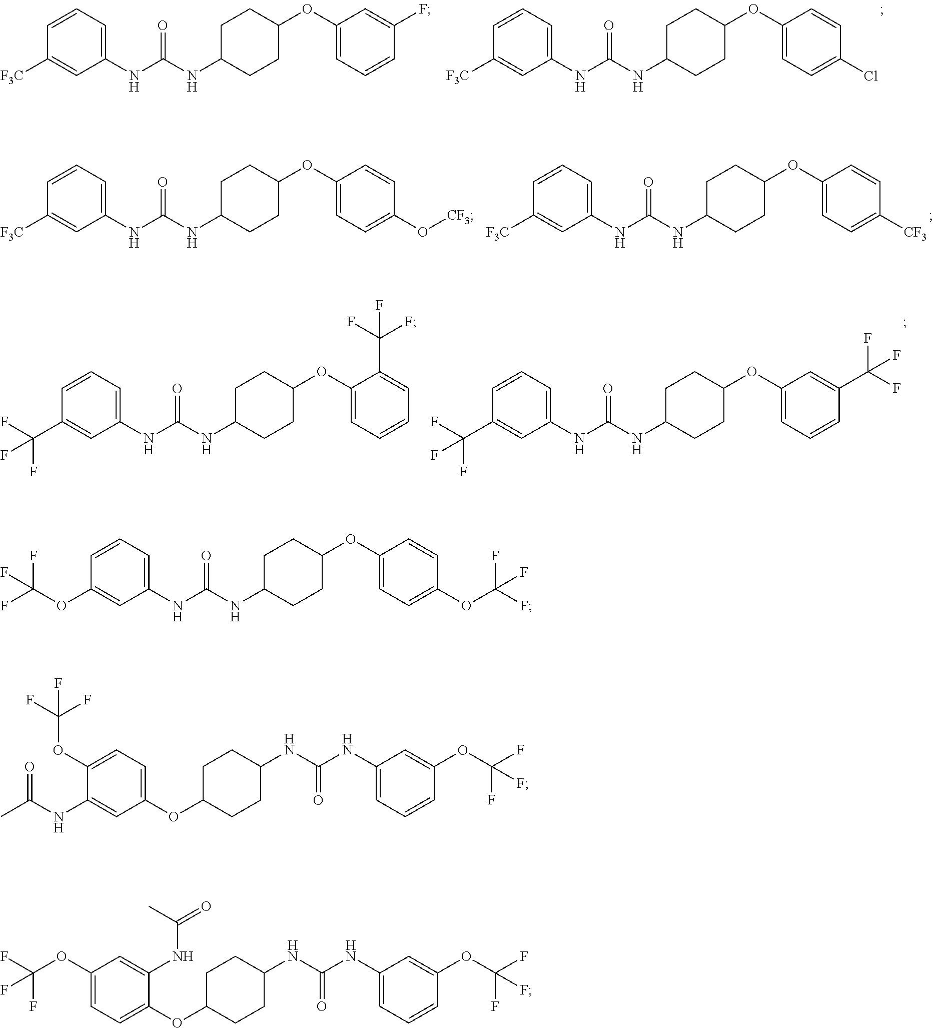

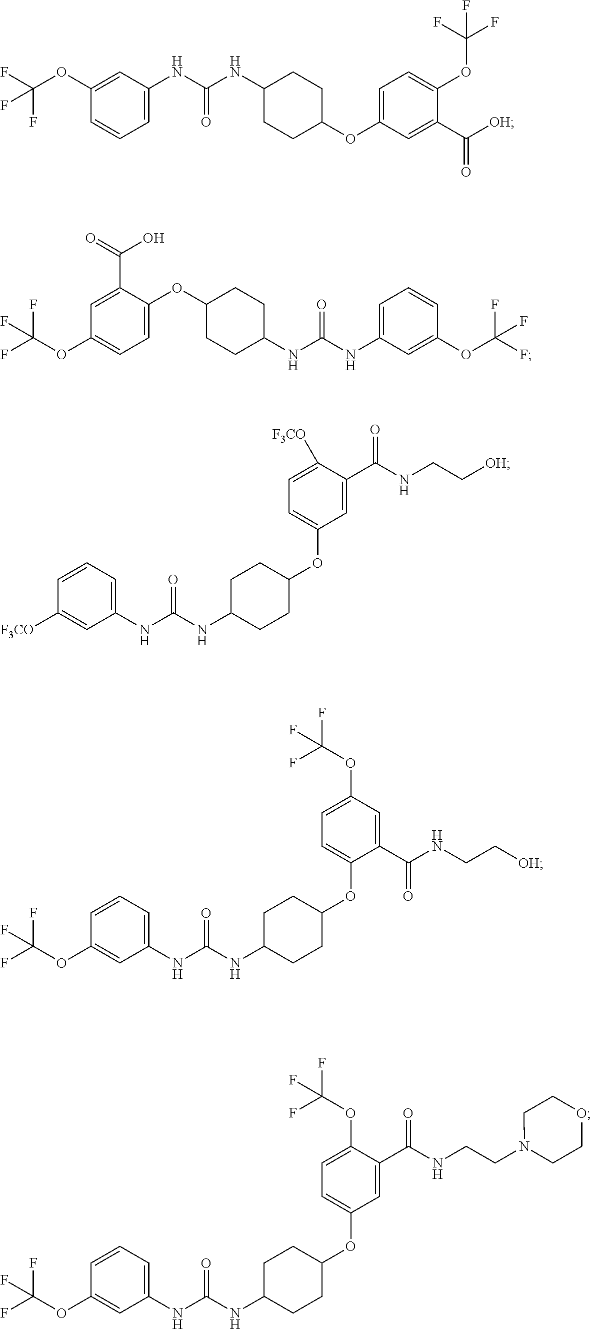 [SMILES: O=C(Nc1cccc(OC(F)(F)F)c1)NC1CCC(Oc2ccc(OC(F)(F)F)c(C(=O)O)c2)CC1 ; O=C(Nc1cccc(OC(F)(F)F)c1)NC1CCC(Oc2ccc(OC(F)(F)F)cc2C(=O)O)CC1 ; O=C(Nc1cccc(OC(F)(F)F)c1)NC1CCC(Oc2ccc(OC(F)(F)F)c(C(=O)NCCO)c2)CC1 ; O=C(Nc1cccc(OC(F)(F)F)c1)NC1CCC(Oc2ccc(OC(F)(F)F)cc2C(=O)NCCO)CC1 ; O=C(Nc1cccc(OC(F)(F)F)c1)NC1CCC(Oc2ccc(OC(F)(F)F)c(C(=O)NCCN3CCOCC3)c2)CC1]

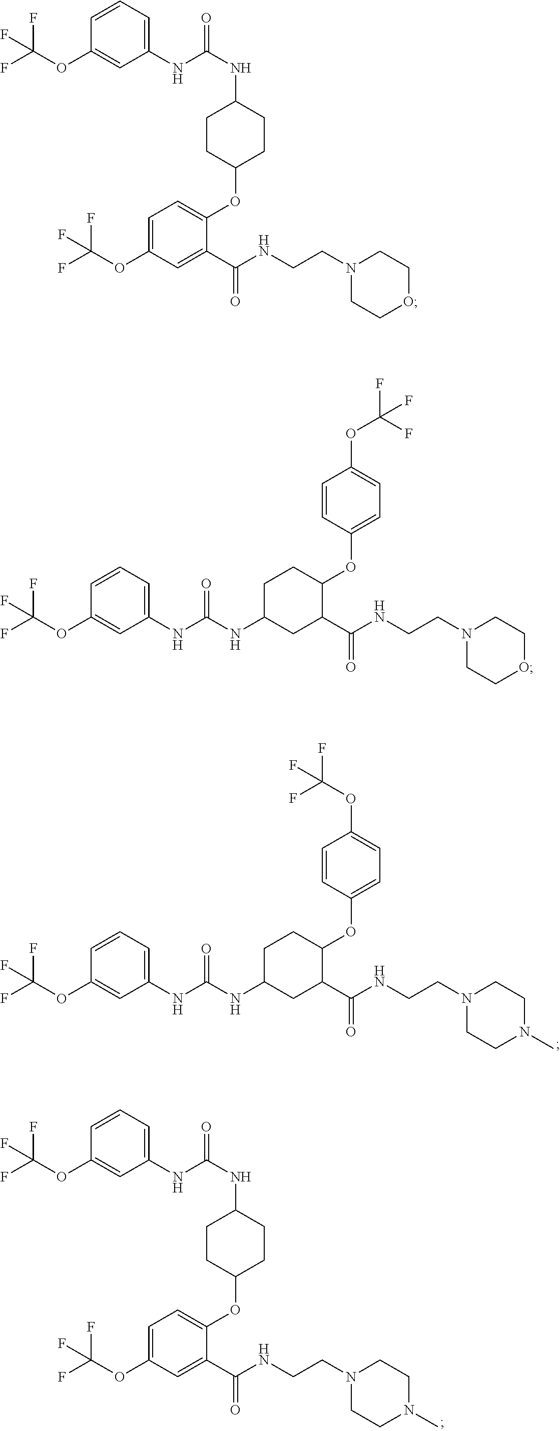 [SMILES: O=C(Nc1cccc(OC(F)(F)F)c1)NC1CCC(Oc2ccc(OC(F)(F)F)cc2C(=O)NCCN2CCOCC2)CC1 ; O=C(Nc1cccc(OC(F)(F)F)c1)NC1CCC(Oc2ccc(OC(F)(F)F)cc2)C(C(=O)NCCN2CCOCC2)C1 ; CN1CCN(CCNC(=O)C2CC(NC(=O)Nc3cccc(OC(F)(F)F)c3)CCC2Oc2ccc(OC(F)(F)F)cc2)CC1 ; CN1CCN(CCNC(=O)c2cc(OC(F)(F)F)ccc2OC2CCC(NC(=O)Nc3cccc(OC(F)(F)F)c3)CC2)CC1]

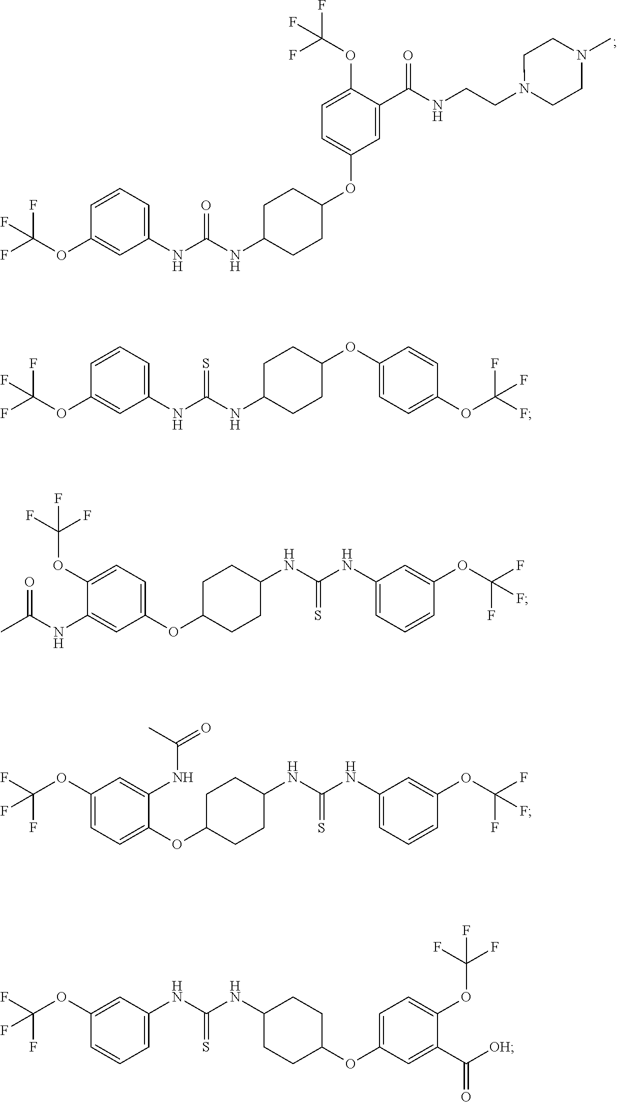

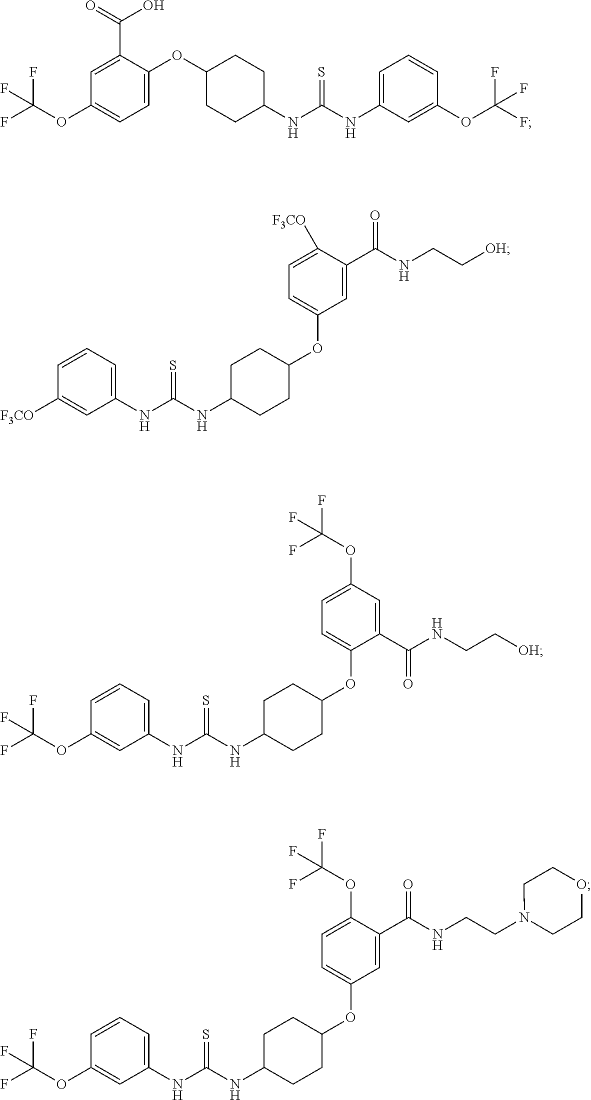 [SMILES: O=C(O)c1cc(OC(F)(F)F)ccc1OC1CCC(NC(=S)Nc2cccc(OC(F)(F)F)c2)CC1 ; O=C(NCCO)c1cc(OC2CCC(NC(=S)Nc3cccc(OC(F)(F)F)c3)CC2)ccc1OC(F)(F)F ; O=C(NCCO)c1cc(OC(F)(F)F)ccc1OC1CCC(NC(=S)Nc2cccc(OC(F)(F)F)c2)CC1 ; O=C(NCCN1CCOCC1)c1cc(OC2CCC(NC(=S)Nc3cccc(OC(F)(F)F)c3)CC2)ccc1OC(F)(F)F]

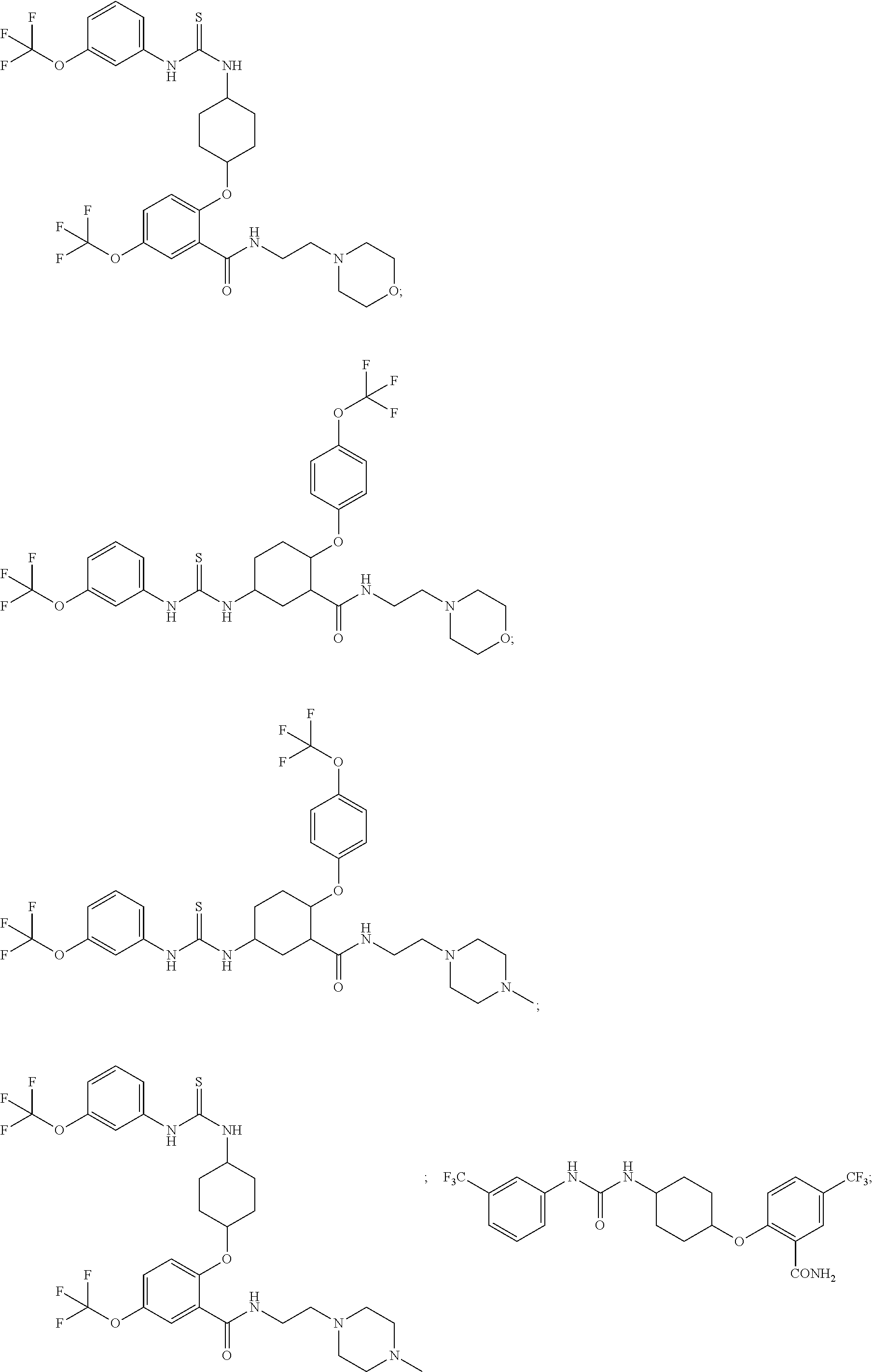 [SMILES: O=C(NCCN1CCOCC1)c1cc(OC(F)(F)F)ccc1OC1CCC(NC(=S)Nc2cccc(OC(F)(F)F)c2)CC1 ; O=C(NCCN1CCOCC1)C1CC(NC(=S)Nc2cccc(OC(F)(F)F)c2)CCC1Oc1ccc(OC(F)(F)F)cc1 ; CN1CCN(CCNC(=O)C2CC(NC(=S)Nc3cccc(OC(F)(F)F)c3)CCC2Oc2ccc(OC(F)(F)F)cc2)CC1 ; CN1CCN(CCNC(=O)c2cc(OC(F)(F)F)ccc2OC2CCC(NC(=S)Nc3cccc(OC(F)(F)F)c3)CC2)CC1 ; NC(=O)c1cc(C(F)(F)F)ccc1OC1CCC(NC(=O)Nc2cccc(C(F)(F)F)c2)CC1]

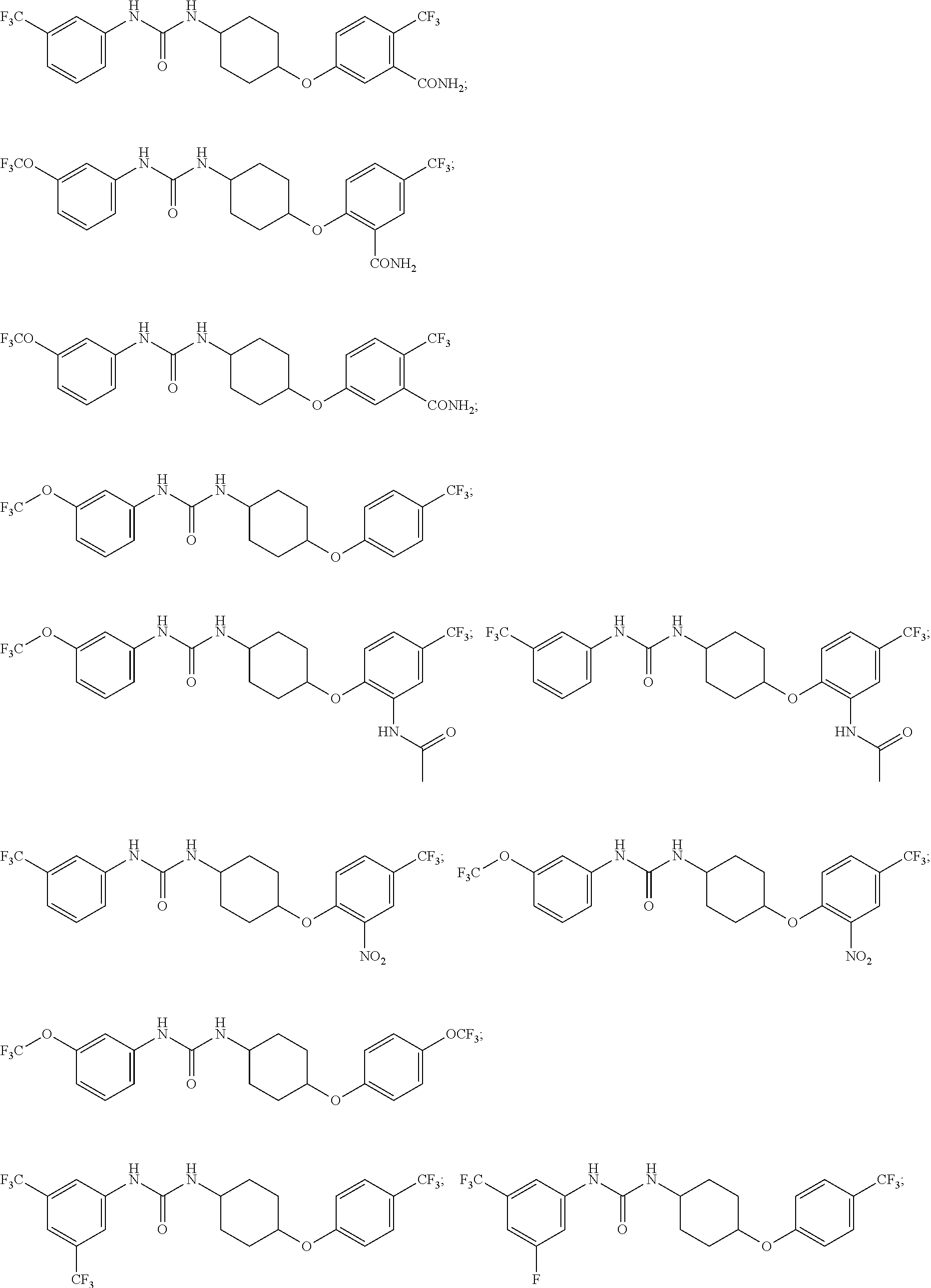

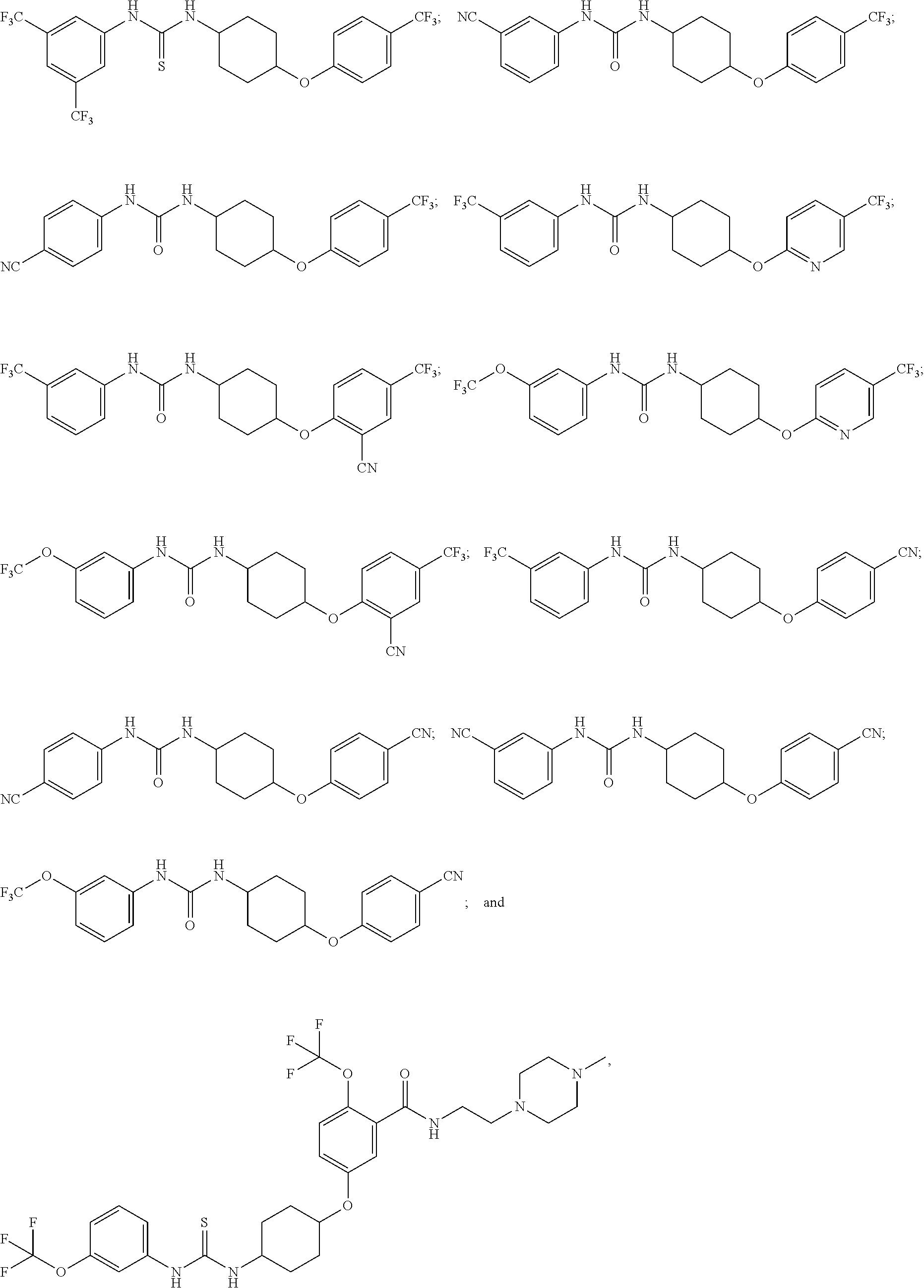

or a pharmaceutically acceptable salt thereof.

Also provided herein is a compound of Formula (I):

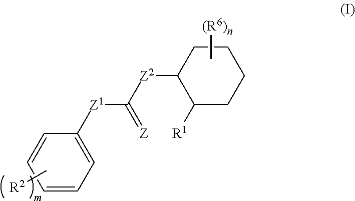

or a pharmaceutically acceptable salt thereof.

A non-limiting example of a compound of Formula (I) includes:

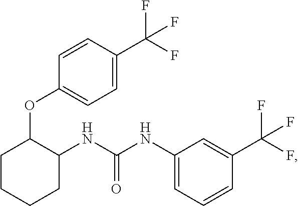 [SMILES: O=C(Nc1cccc(C(F)(F)F)c1)NC1CCCCC1Oc1ccc(C(F)(F)F)cc1]

or a pharmaceutically acceptable salt thereof.

Further provided herein is a compound of Formula (II):

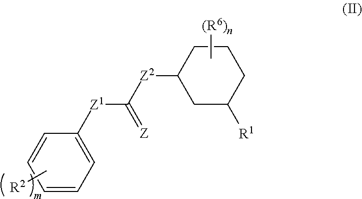

or a pharmaceutically acceptable salt thereof.

A non-limiting example of a compound of Formula (II) includes:

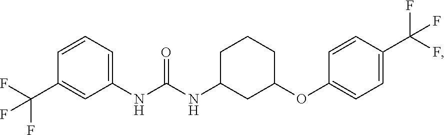 [SMILES: O=C(Nc1cccc(C(F)(F)F)c1)NC1CCCC(Oc2ccc(C(F)(F)F)cc2)C1]

or a pharmaceutically acceptable salt thereof.

This disclosure also provides a compound of Formula (IV):

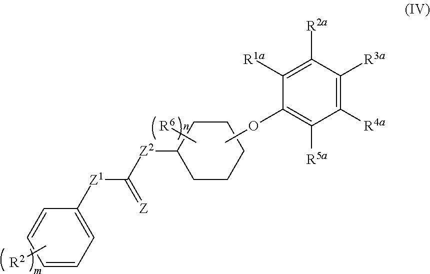

or a pharmaceutically acceptable salt thereof.

Non-limiting examples of a compound of Formula (IV) include:

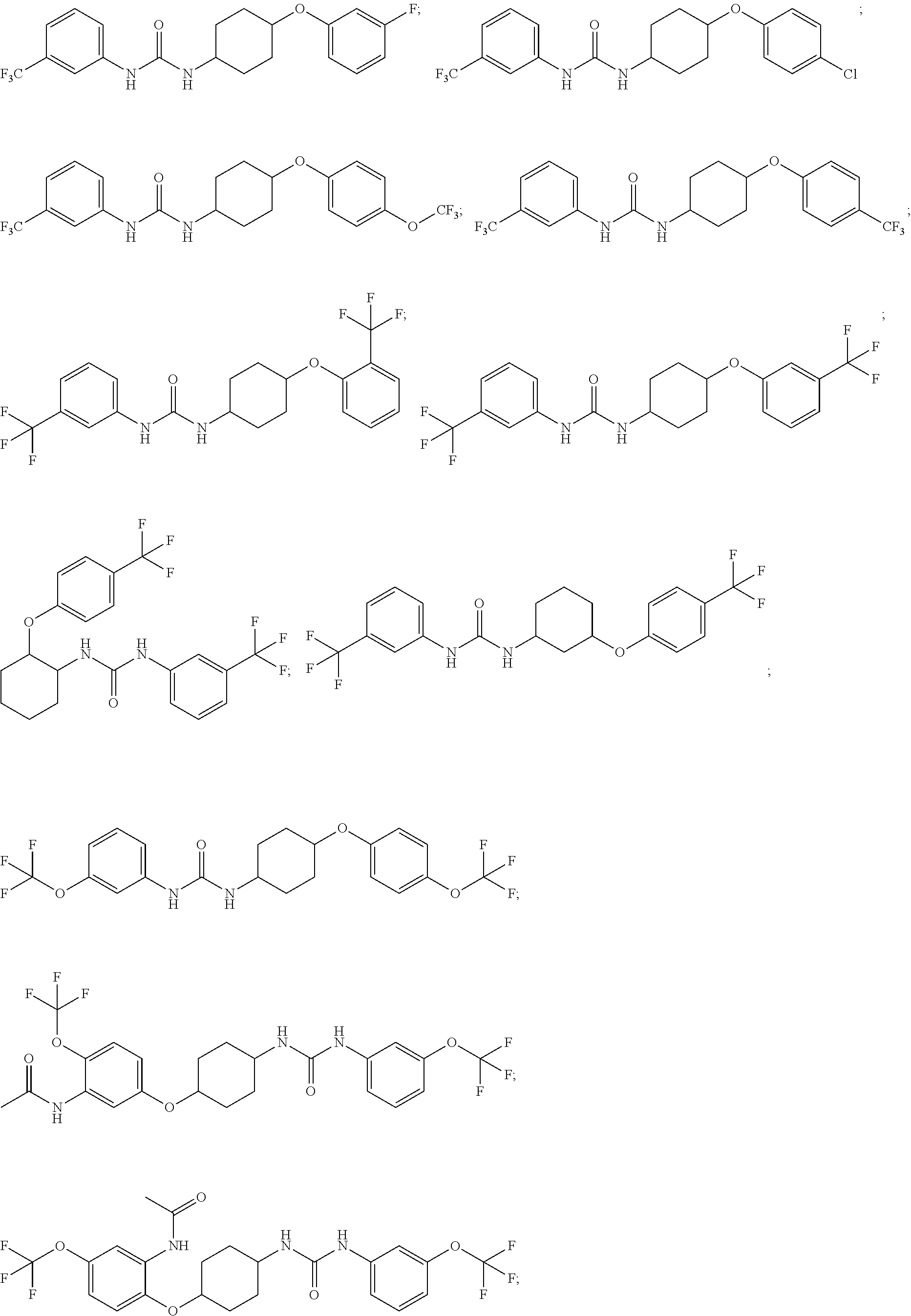

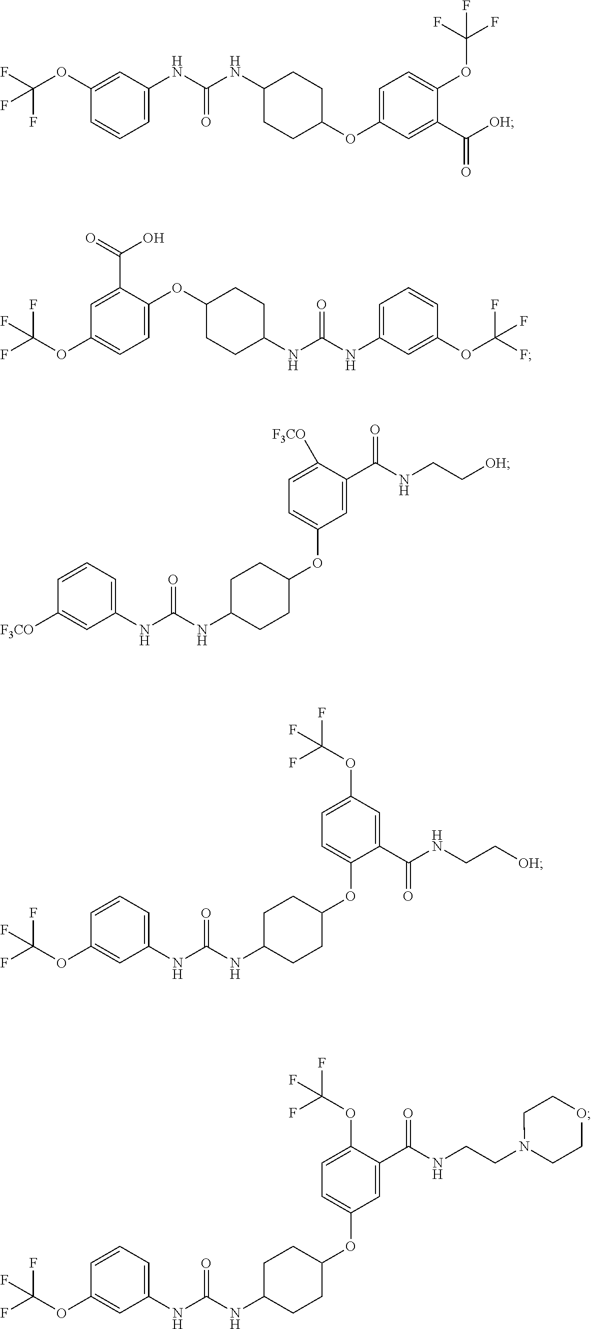 [SMILES: O=C(Nc1cccc(OC(F)(F)F)c1)NC1CCC(Oc2ccc(OC(F)(F)F)c(C(=O)O)c2)CC1 ; O=C(Nc1cccc(OC(F)(F)F)c1)NC1CCC(Oc2ccc(OC(F)(F)F)cc2C(=O)O)CC1 ; O=C(Nc1cccc(OC(F)(F)F)c1)NC1CCC(Oc2ccc(OC(F)(F)F)c(C(=O)NCCO)c2)CC1 ; O=C(Nc1cccc(OC(F)(F)F)c1)NC1CCC(Oc2ccc(OC(F)(F)F)cc2C(=O)NCCO)CC1 ; O=C(Nc1cccc(OC(F)(F)F)c1)NC1CCC(Oc2ccc(OC(F)(F)F)c(C(=O)NCCN3CCOCC3)c2)CC1]

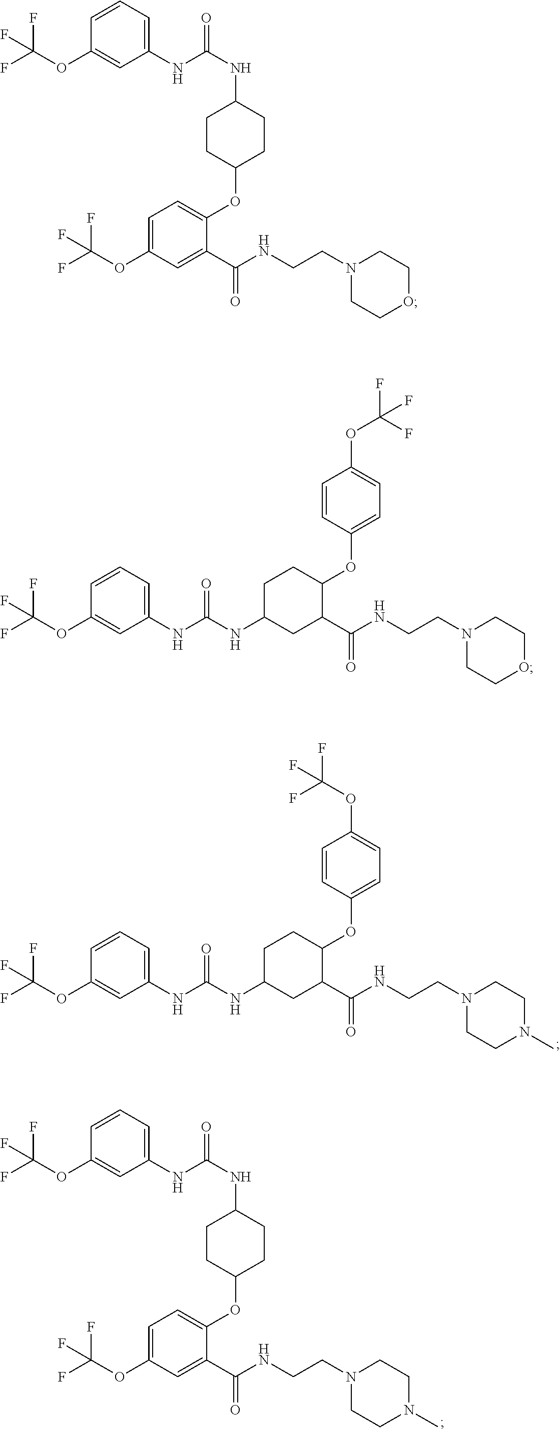 [SMILES: O=C(Nc1cccc(OC(F)(F)F)c1)NC1CCC(Oc2ccc(OC(F)(F)F)cc2C(=O)NCCN2CCOCC2)CC1 ; O=C(Nc1cccc(OC(F)(F)F)c1)NC1CCC(Oc2ccc(OC(F)(F)F)cc2)C(C(=O)NCCN2CCOCC2)C1 ; CN1CCN(CCNC(=O)C2CC(NC(=O)Nc3cccc(OC(F)(F)F)c3)CCC2Oc2ccc(OC(F)(F)F)cc2)CC1 ; CN1CCN(CCNC(=O)c2cc(OC(F)(F)F)ccc2OC2CCC(NC(=O)Nc3cccc(OC(F)(F)F)c3)CC2)CC1]

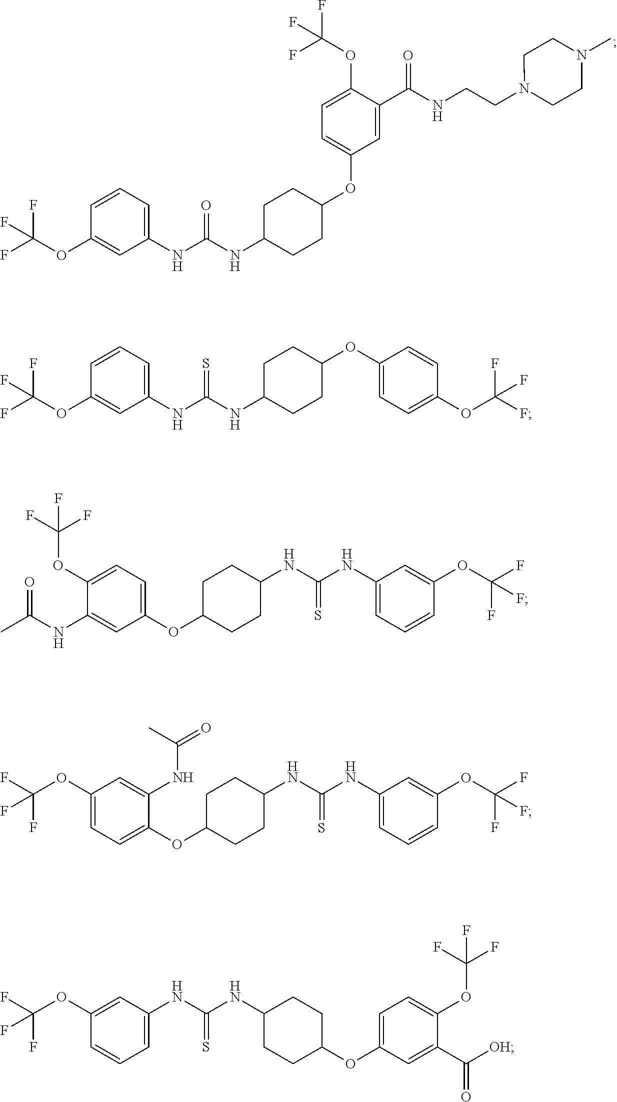

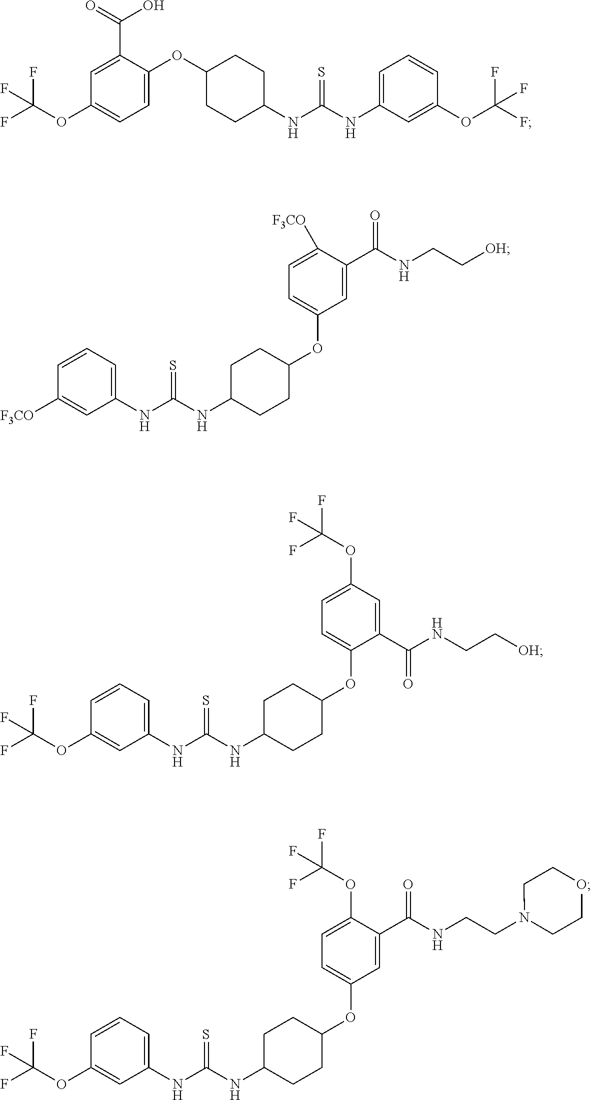 [SMILES: O=C(O)c1cc(OC(F)(F)F)ccc1OC1CCC(NC(=S)Nc2cccc(OC(F)(F)F)c2)CC1 ; O=C(NCCO)c1cc(OC2CCC(NC(=S)Nc3cccc(OC(F)(F)F)c3)CC2)ccc1OC(F)(F)F ; O=C(NCCO)c1cc(OC(F)(F)F)ccc1OC1CCC(NC(=S)Nc2cccc(OC(F)(F)F)c2)CC1 ; O=C(NCCN1CCOCC1)c1cc(OC2CCC(NC(=S)Nc3cccc(OC(F)(F)F)c3)CC2)ccc1OC(F)(F)F]

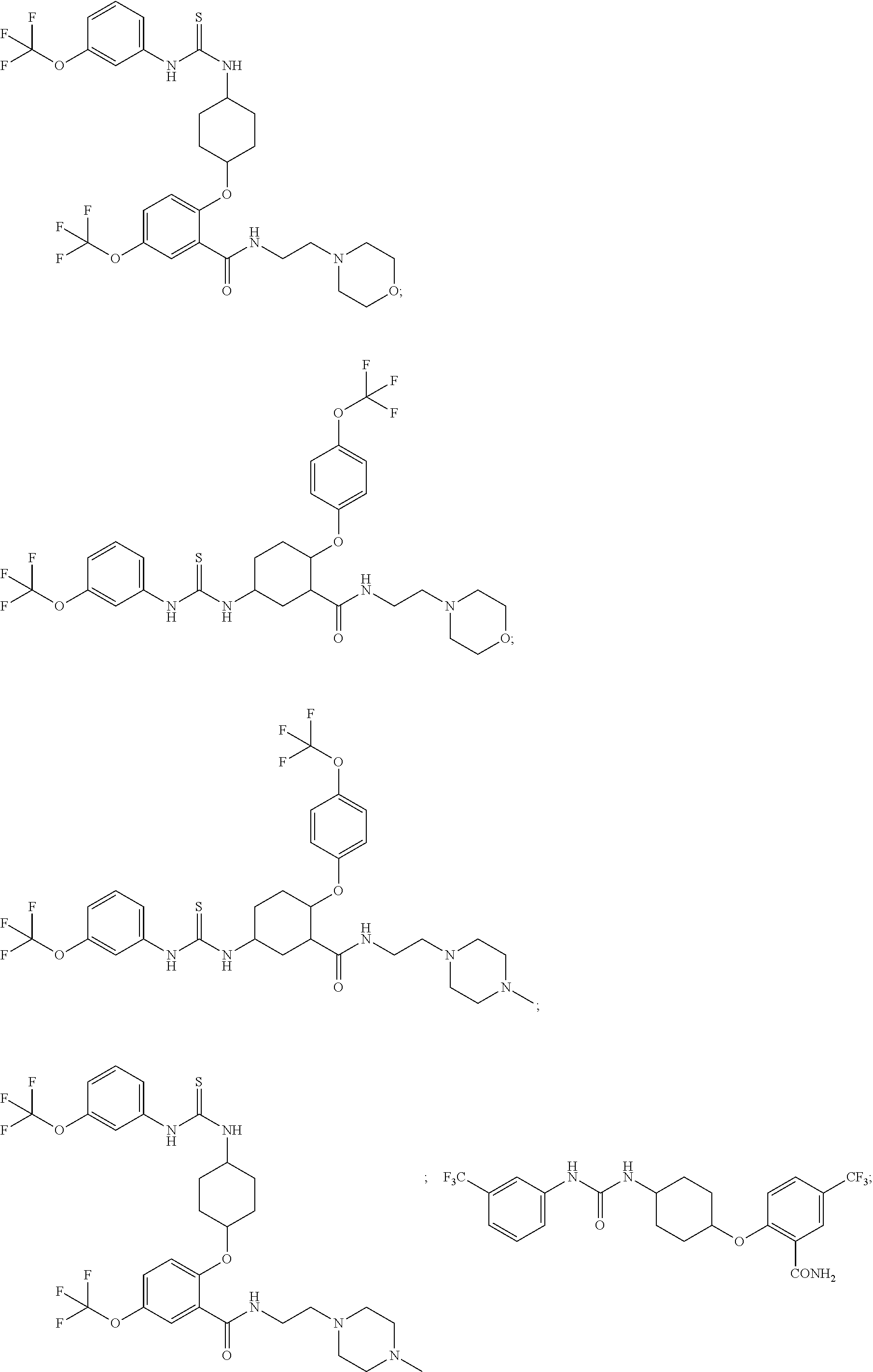 [SMILES: O=C(NCCN1CCOCC1)c1cc(OC(F)(F)F)ccc1OC1CCC(NC(=S)Nc2cccc(OC(F)(F)F)c2)CC1 ; O=C(NCCN1CCOCC1)C1CC(NC(=S)Nc2cccc(OC(F)(F)F)c2)CCC1Oc1ccc(OC(F)(F)F)cc1 ; CN1CCN(CCNC(=O)C2CC(NC(=S)Nc3cccc(OC(F)(F)F)c3)CCC2Oc2ccc(OC(F)(F)F)cc2)CC1 ; CN1CCN(CCNC(=O)c2cc(OC(F)(F)F)ccc2OC2CCC(NC(=S)Nc3cccc(OC(F)(F)F)c3)CC2)CC1 ; NC(=O)c1cc(C(F)(F)F)ccc1OC1CCC(NC(=O)Nc2cccc(C(F)(F)F)c2)CC1]

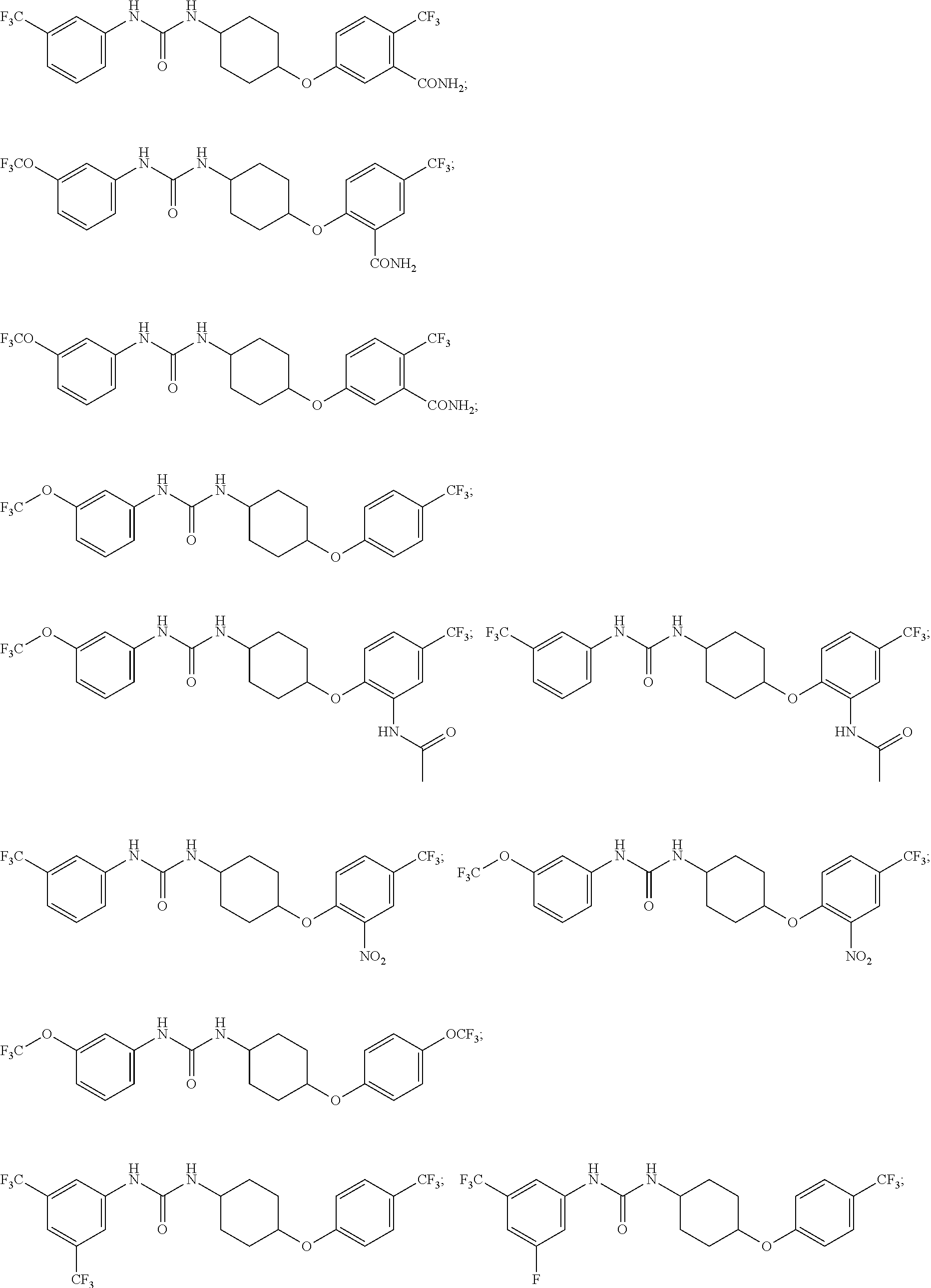

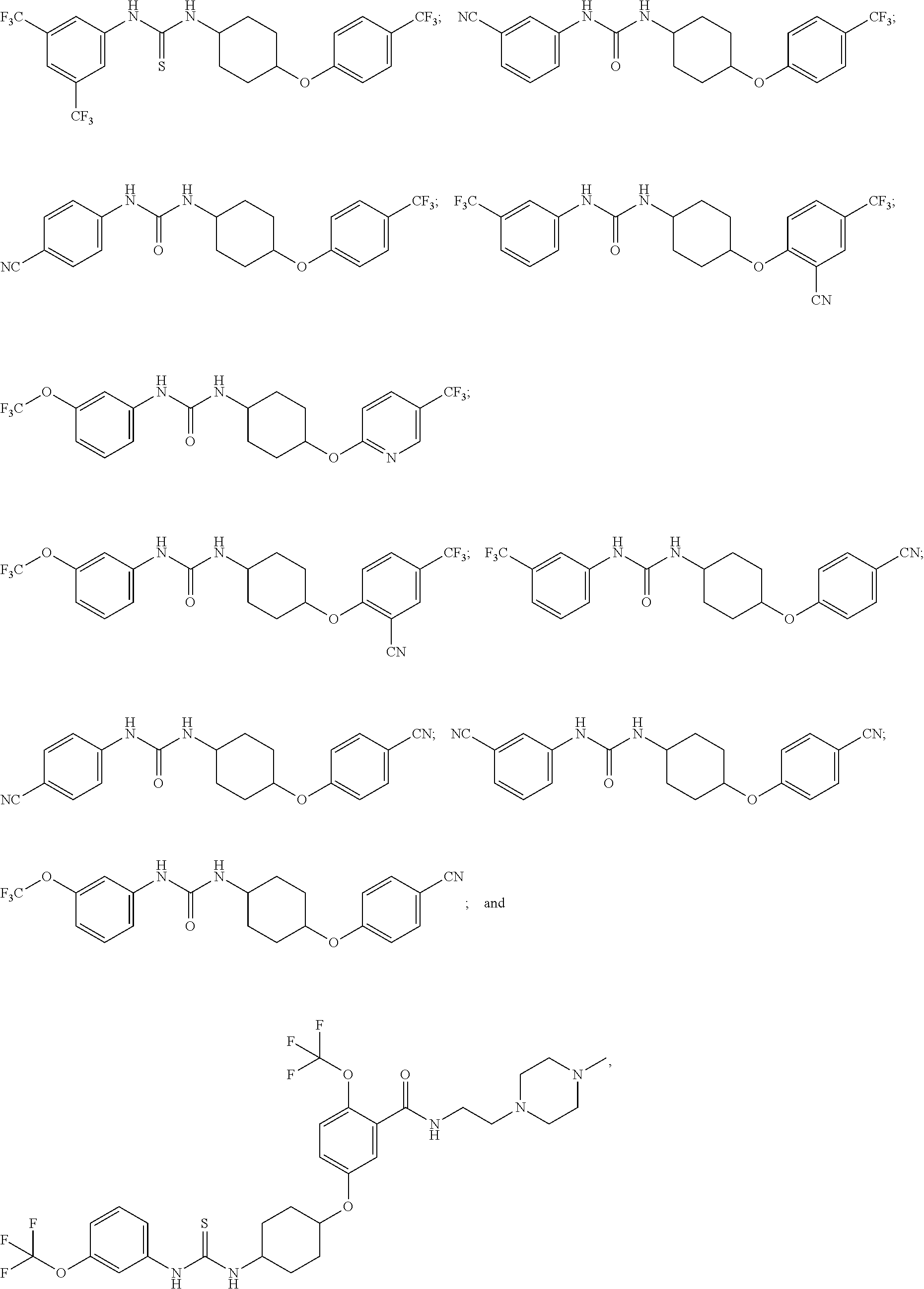

or a pharmaceutically acceptable salt thereof.

In some embodiments, a compound provided herein is a compound of Formula (V):

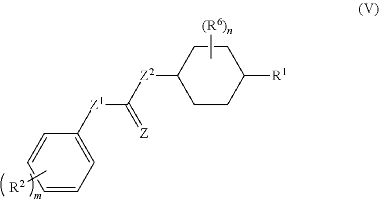

or a pharmaceutically acceptable salt thereof.

A non-limiting example of a compound of Formula (V) includes:

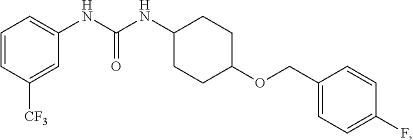 [SMILES: O=C(Nc1cccc(C(F)(F)F)c1)NC1CCC(OCc2ccc(F)cc2)CC1]

or a pharmaceutically acceptable salt thereof.

Provided herein is a compound of Formula (VI):

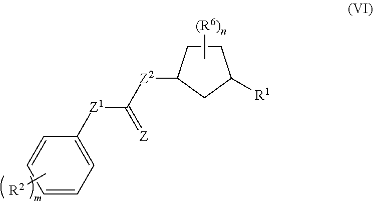

or a pharmaceutically acceptable salt thereof.

Non-limiting examples of a compound of Formula (VI) include:

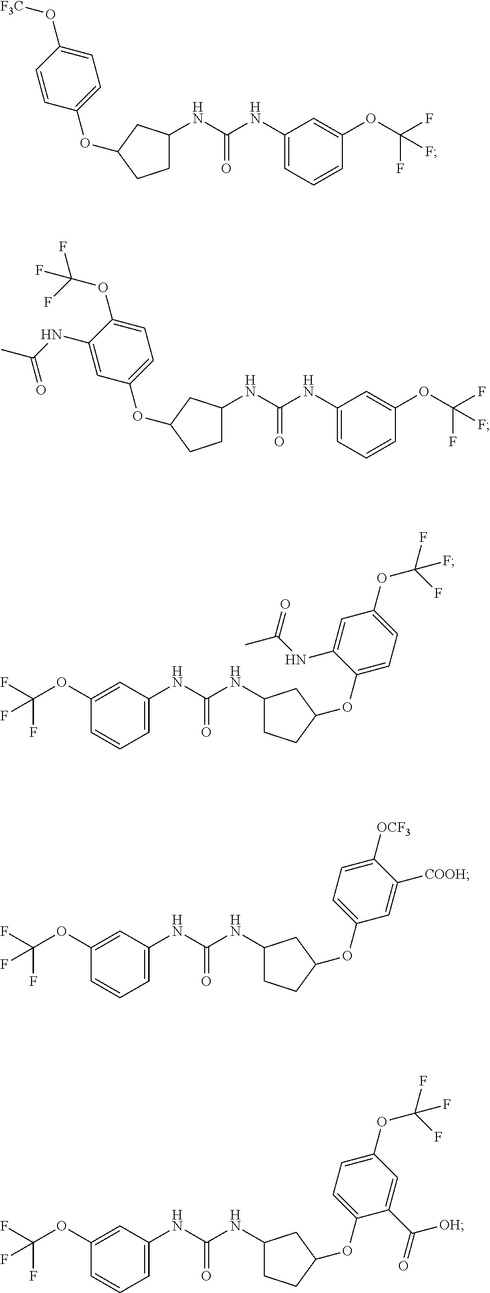 [SMILES: O=C(Nc1cccc(OC(F)(F)F)c1)NC1CCC(Oc2ccc(OC(F)(F)F)cc2)C1 ; CC(=O)Nc1cc(OC2CCC(NC(=O)Nc3cccc(OC(F)(F)F)c3)C2)ccc1OC(F)(F)F ; CC(=O)Nc1cc(OC(F)(F)F)ccc1OC1CCC(NC(=O)Nc2cccc(OC(F)(F)F)c2)C1 ; O=C(Nc1cccc(OC(F)(F)F)c1)NC1CCC(Oc2ccc(OC(F)(F)F)c(C(=O)O)c2)C1 ; O=C(Nc1cccc(OC(F)(F)F)c1)NC1CCC(Oc2ccc(OC(F)(F)F)cc2C(=O)O)C1]

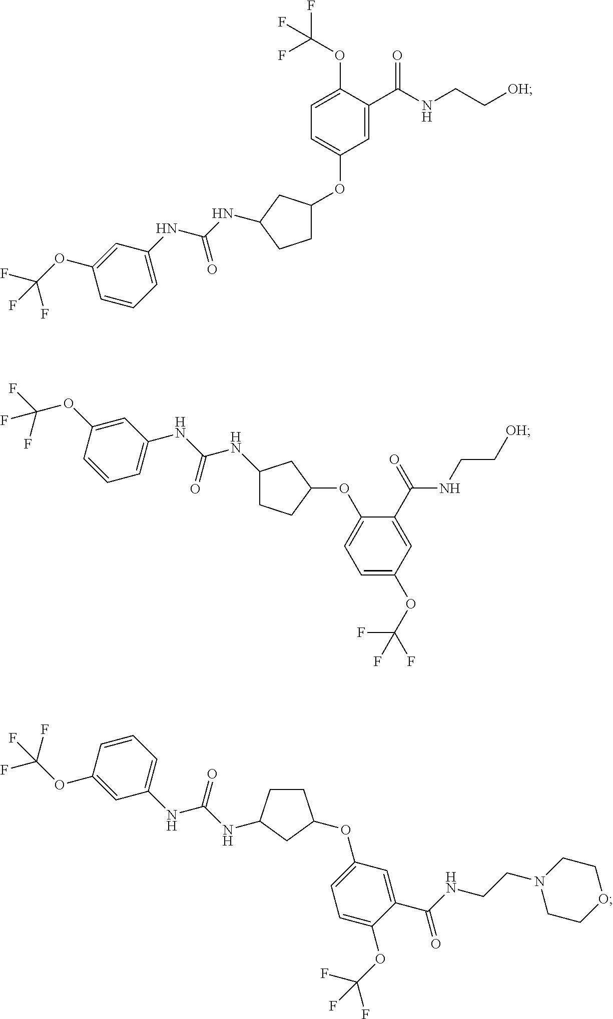 [SMILES: O=C(Nc1cccc(OC(F)(F)F)c1)NC1CCC(Oc2ccc(OC(F)(F)F)c(C(=O)NCCO)c2)C1 ; O=C(Nc1cccc(OC(F)(F)F)c1)NC1CCC(Oc2ccc(OC(F)(F)F)cc2C(=O)NCCO)C1 ; O=C(Nc1cccc(OC(F)(F)F)c1)NC1CCC(Oc2ccc(OC(F)(F)F)c(C(=O)NCCN3CCOCC3)c2)C1]

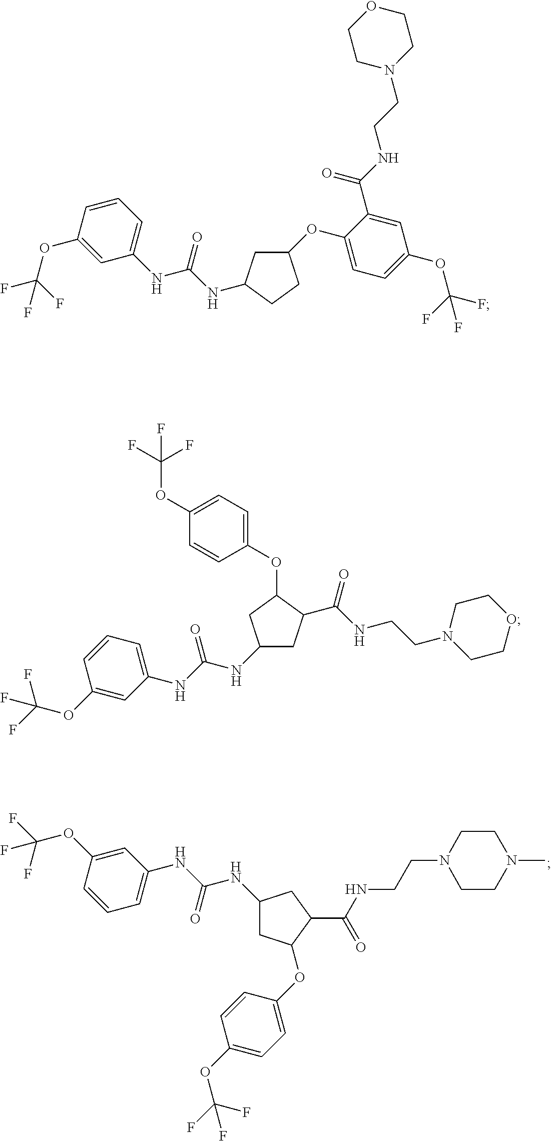 [SMILES: O=C(Nc1cccc(OC(F)(F)F)c1)NC1CCC(Oc2ccc(OC(F)(F)F)cc2C(=O)NCCN2CCOCC2)C1 ; O=C(Nc1cccc(OC(F)(F)F)c1)NC1CC(Oc2ccc(OC(F)(F)F)cc2)C(C(=O)NCCN2CCOCC2)C1 ; CN1CCN(CCNC(=O)C2CC(NC(=O)Nc3cccc(OC(F)(F)F)c3)CC2Oc2ccc(OC(F)(F)F)cc2)CC1]

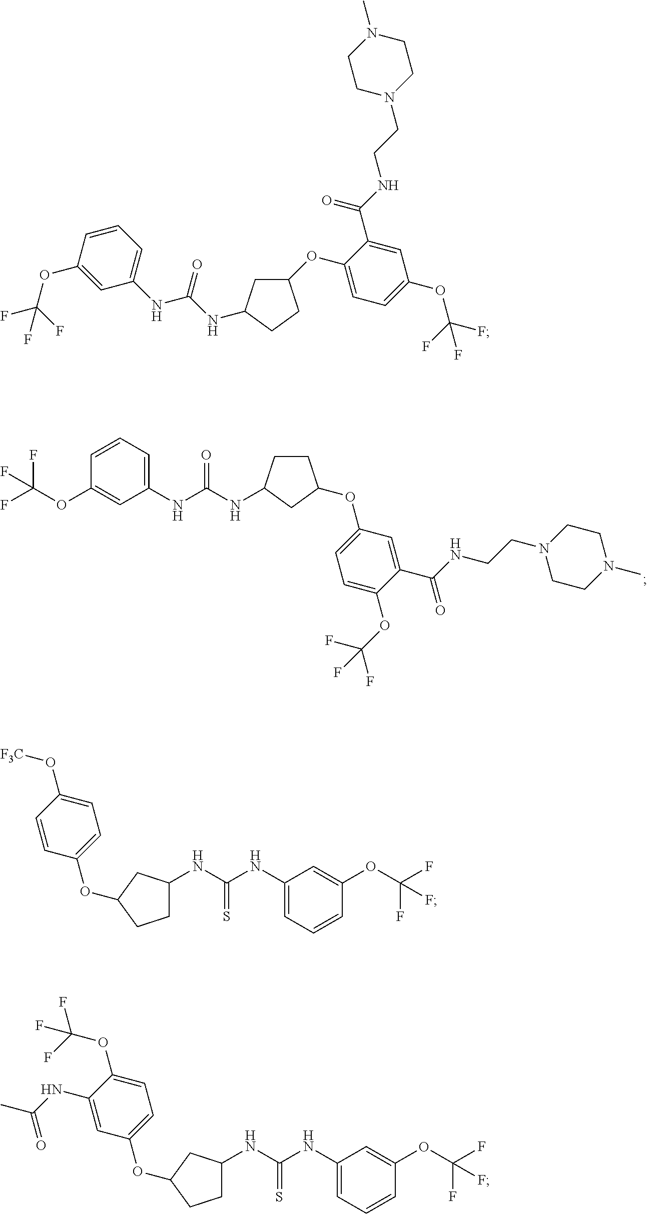

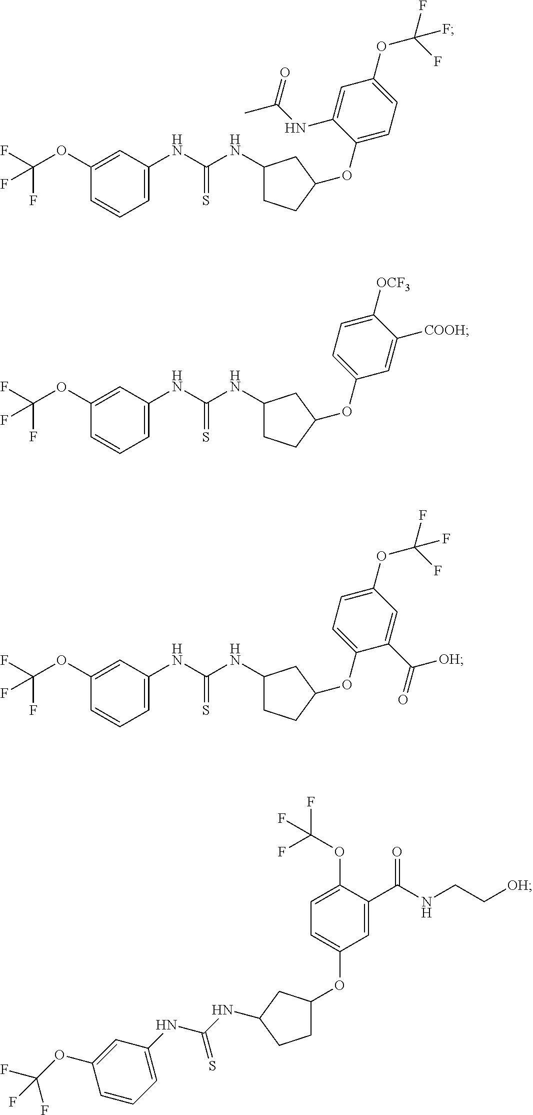 [SMILES: CC(=O)Nc1cc(OC(F)(F)F)ccc1OC1CCC(NC(=S)Nc2cccc(OC(F)(F)F)c2)C1 ; O=C(O)c1cc(OC2CCC(NC(=S)Nc3cccc(OC(F)(F)F)c3)C2)ccc1OC(F)(F)F ; O=C(O)c1cc(OC(F)(F)F)ccc1OC1CCC(NC(=S)Nc2cccc(OC(F)(F)F)c2)C1 ; O=C(NCCO)c1cc(OC2CCC(NC(=S)Nc3cccc(OC(F)(F)F)c3)C2)ccc1OC(F)(F)F]

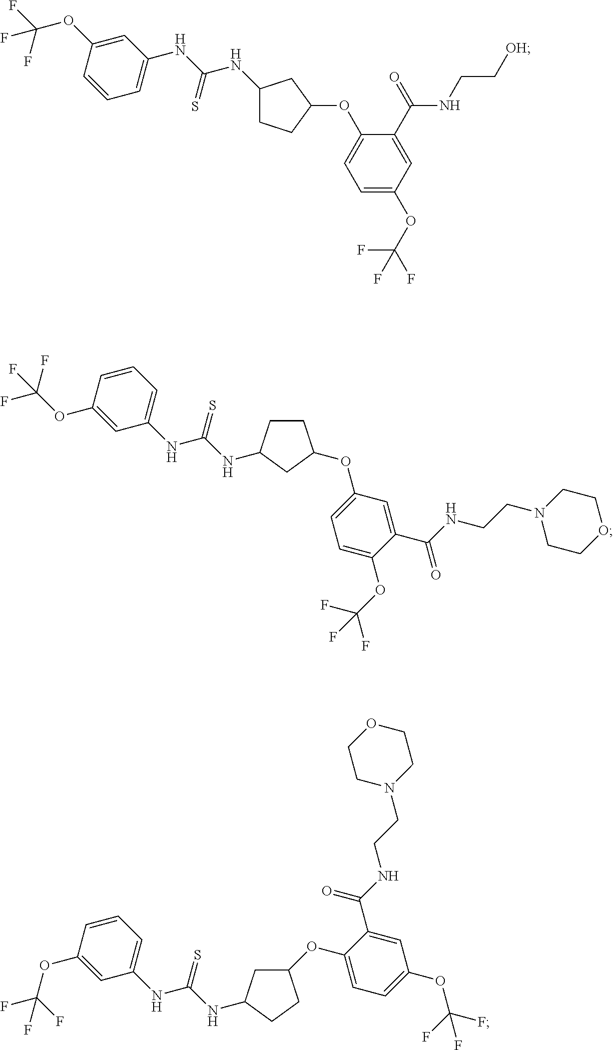 [SMILES: O=C(NCCO)c1cc(OC(F)(F)F)ccc1OC1CCC(NC(=S)Nc2cccc(OC(F)(F)F)c2)C1 ; O=C(NCCN1CCOCC1)c1cc(OC2CCC(NC(=S)Nc3cccc(OC(F)(F)F)c3)C2)ccc1OC(F)(F)F ; O=C(NCCN1CCOCC1)c1cc(OC(F)(F)F)ccc1OC1CCC(NC(=S)Nc2cccc(OC(F)(F)F)c2)C1]

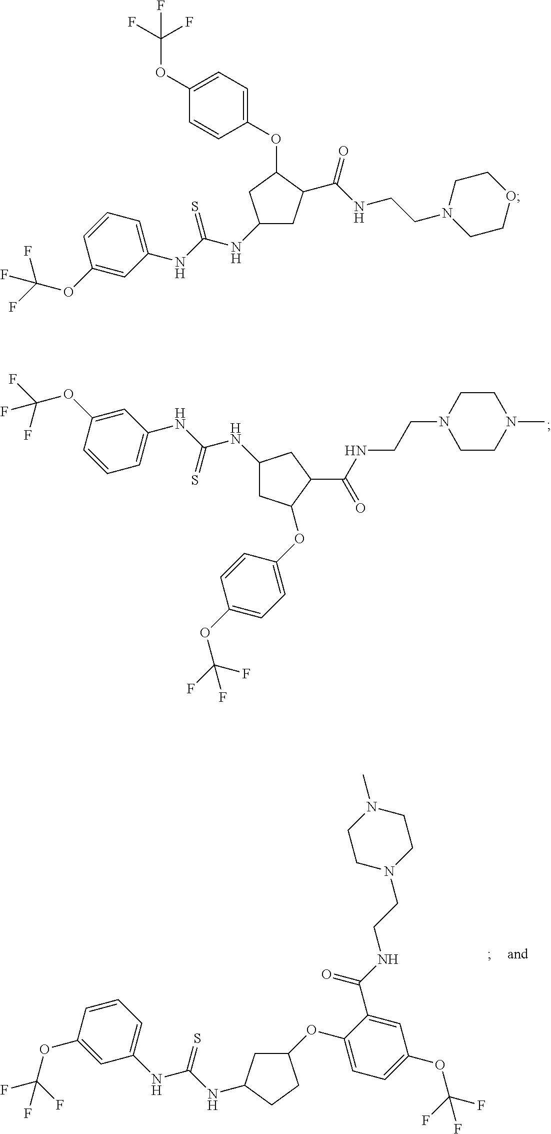 [SMILES: O=C(NCCN1CCOCC1)C1CC(NC(=S)Nc2cccc(OC(F)(F)F)c2)CC1Oc1ccc(OC(F)(F)F)cc1 ; CN1CCN(CCNC(=O)C2CC(NC(=S)Nc3cccc(OC(F)(F)F)c3)CC2Oc2ccc(OC(F)(F)F)cc2)CC1 ; CN1CCN(CCNC(=O)c2cc(OC(F)(F)F)ccc2OC2CCC(NC(=S)Nc3cccc(OC(F)(F)F)c3)C2)CC1]

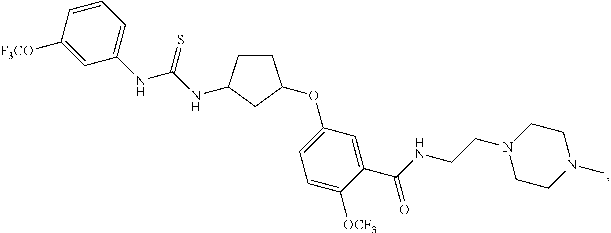 [SMILES: CN1CCN(CCNC(=O)c2cc(OC3CCC(NC(=S)Nc4cccc(OC(F)(F)F)c4)C3)ccc2OC(F)(F)F)CC1]

or a pharmaceutically acceptable salt thereof.

Also provided herein is a compound of Formula (VII):

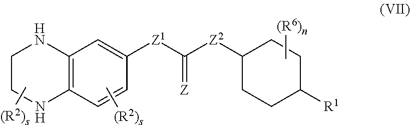

or a pharmaceutically acceptable salt thereof.

Further provided herein is a compound of Formula (VIII):

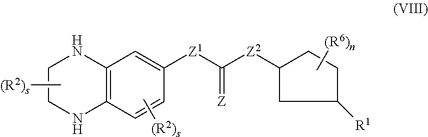

or a pharmaceutically acceptable salt thereof.

Further provided herein is a compound of Formula (IX):

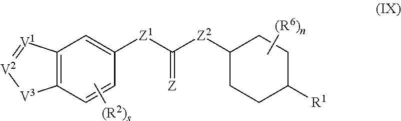

or a pharmaceutically acceptable salt thereof.

This disclosure also provides a compound of Formula (X):

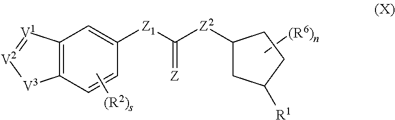

or a pharmaceutically acceptable salt thereof.

The compounds provided herein may also be present in a pharmaceutical composition including a pharmaceutically acceptable carrier or diluent and a compound provided herein, or a pharmaceutically acceptable salt thereof.

Also provided herein are methods of treatment using the compounds provided herein. For example, the compounds may be used in the treatment of cancer, hemolytic anemia, Wolcott-Rallison syndrome, a neurodegenerative disease, motor-neuron disease, tuberous sclerosis complex, an autism spectrum disorder, a ribosomal defect disease, or a mental retardation disorder. Such methods include administration to a patient in need thereof of a therapeutically effective amount of a compound provided herein, or a pharmaceutically acceptable salt thereof, or a pharmaceutical composition comprising the same.

In some embodiments, the compounds provided herein may also be useful for activating one or more eIF2α kinases in a cell. For example, a cell can be contacted with an effective amount of a compound provided herein, or a pharmaceutically acceptable salt thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a series of graphs showing the activity of compounds through 1-13 from Table 1 in an eIF2α assay at concentrations of 30 μM (clear bars), 15 μM (checkered bars), 7.5 μM (solid bars), and 3.75 μM (lined bars). FIG. 1a shows the activity of thirteen ureas from Table 1 with halogen substituents. FIG. 1b shows the activity of seven ureas from Table 1 with electron-donating groups. FIG. 1c shows the activity of eighteen ureas from Table 1 with electron-withdrawing groups.

FIG. 2 shows a Western blot analysis of the effects on phosphorylated eIF2α (p-eIF2α) and total eIF2α (T-eIF2α) of compounds I-5o, I-1m, I-5m, I-5p, I-6p, and I-9p in CRL-2813 human melanoma cells.

FIGS. 3A-C shows the effects of compounds I-5o, I-5m, I-5m, I-5p, I-6p, and I-9p at concentrations of 15 μM (clear bars) and 7.5 μM (diagonal lined bars) on protein and mRNA expressions of CHOP and Cyclin D1.

FIG. 4 shows the effects of compounds I-5m, I-5p, I-6p, and I-9p levels of the ternary complex in CRL-2813-pBISA-DL (ATF-4) cells stably transfected with non-target (clear bars) or HRI RNAi (diagonal shaded bars).

FIG. 5 shows the effects of compounds I-5m, I-5p, I-6p, and I-9p on cancer cells in vitro. FIG. 5A shows the effects of compound I-6p on CRL-2813 human melanoma cells. FIG. 5B shows the effects of compound I-6p on MCF-7 human breast cancer cells. FIG. 5C shows the inhibitory effects of compounds I-5m, I-5p, I-6p, and I-9p on CRL-2813 human melanoma cells. FIG. 5D shows the inhibitory effects of the compounds on MCF-7 human breast cancer cells.

FIG. 6 shows the dose response studies for the phenoxy substituted 1-(4-phenoxycyclohexyl)-3-(3-(trifluoromethyl)phenyl)ureas, 1-14 through 1-20, at concentrations of 30 μM (clear bars), 15 μM (checkered bars), 7.5 μM (solid bars), 3.75 μM (vertical lined bars), 1.86 μM (right-to-left downward diagonal bars), and 0.93 μM (left-to-right downward diagonal bars) in the surrogate eIF2α phosphorylation assays.

FIG. 7 shows the effects of compounds I-18, and III-1 through III-6 at concentrations of 30 μM (clear bars), 15 μM (checkered bars), 7.5 μM (solid bars), 3.75 μM (vertical lined bars), 1.86 μM (right-to-left downward diagonal bars), and 0.93 μM (left-to-right downward diagonal bars) in the surrogate eIF2α phosphorylation assays.

FIG. 8 shows the effects of compounds on the proliferation of CRL-2813 human melanoma cancer cells transfected with siRNA to remove HRI or a non-target siRNA. FIG. 8A shows the effects of compound I-14. FIG. 8B shows the effects of compound I-15. FIG. 8C shows the effects of compound III-4. FIG. 8D shows the effects of compound III-5. FIG. 8E shows the calculated IC₅₀ for compounds tested in CRL-2813 human melanoma cancer cells transfected with non-target siRNA (control, NTC) or siRNA targeting HRI.

FIG. 9 shows time response studies of the selected N-aryl,N′-cyclohexylarylureas in the surrogate eIF2α phosphorylation assays. Reporter cells were incubated with (A) I-14, (B) I-15, (C) III-4 and (D) III-5 for 8, 16, or 32 hours and the F/R was determined by DLR assay. The experiment was conducted in triplicate and each experiment was independently performed three times; data are shown as Mean±S.E.M.

FIG. 10 illustrates the higher HRI dependence for inhibition of cell proliferation of certain compounds. CRL-2813 human melanoma cancer cells were transfected with HRI targeting or non-targeting siRNA, treated with the indicated concentrations of (A) I-14, (B) I-15, (C) I-17 and (D) I-18 and cell proliferation was measured by SRB assay. Calculated IC₅₀ values for library compound I-5m and I-14, I-15, I-17, I-18, and I-20 in CRL-2813 human melanoma cancer cells transfected with non-targeting siRNA or HRI-targeting siRNA are shown in (E). The experiment was conducted in triplicate and each experiment was independently performed three times. Data are shown as Mean±S.E.M. *NTC=non-targeting control.

### DETAILED DESCRIPTION

Deregulation of eIF2α phosphorylation is implicated in the patho-biology of various human disorders. For example, inactivating mutations of PERK has been linked with Wolcott-Rallison syndrome, a rare autosomal recessive disease characterized by neonatal/early-onset non-autoimmune insulin-requiring diabetes associated with skeletal dysplasia and growth retardation syndrome. Insufficiency of eIF2α phosphorylation that occurs in red blood cell progenitors deficient in heme-regulated inhibitor (HRI) can increase the severity of hemolytic anemia such as β-thalassemia. Deregulation of eIF2α phosphorylation has also been implicated in the pathogenesis of neurodegenerative disorders such as Alzheimer's disease and proliferative disorders including cancer. Induction of eIF2α phosphorylation appears to protect motor neurons that die due to Amytrophic Lateral Sclerosis (ALS). Forced expression of eIF2α-S51A, a non-phosphorylatable mutant, increases the amount of the ternary complex, renders the translation initiation unrestricted, and can cause transformation of normal cells. Similarly, overexpression of Met-tRNAi causes cellular transformation. In contrast, induction of eIF2α phosphorylation pharmacologically or by over-expressing eIF2α kinases can inhibit proliferation of cancer cells in vitro and tumor growth in vivo.

See, for example, Chen, T. et al. “Explorations of Substituted Urea Functionality for Discovery of New Activators of the Heme Regulated Inhibitor Kinase.” Journal of Medicinal Chemistry, 2013, 56, 9457-9470, which is herein incorporated by reference in its entirety.

### DEFINITIONS

For the terms “for example” and “such as” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

Resolution of racemic mixtures of compounds can be carried out by any of numerous methods known in the art. An exemplary method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid, or the various optically active camphorsulfonic acids such as camphorsulfonic acid. Other resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent compositions can be determined by one skilled in the art.

Compounds provided herein can also include all isotopes of atoms occurring in the intermediates or final compounds. Isotopes include those atoms having the same atomic number but different mass numbers. For example, isotopes of hydrogen include hydrogen, tritium, and deuterium.

The term, “compound”, as used herein is meant to include all stereoisomers, geometric isomers, tautomers, and isotopes of the structures depicted. Compounds herein identified by name or structure as one particular tautomeric form are intended to include other tautomeric forms unless otherwise specified.

All compounds, and pharmaceutically acceptable salts thereof, can be found together with other substances such as water and solvents (e.g., hydrates and solvates).

The term “C_{x-y}alkyl” refers to substituted or unsubstituted saturated hydrocarbon groups, including straight-chain alkyl and branched-chain alkyl groups that contain from x to y carbons in the chain. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond, respectively.

The term “C₁₋₆alkoxy” refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “C₁₋₆alkoxyalkyl” refers to a C₁₋₆alkyl group substituted with an alkoxy group, thereby forming an ether.

The terms “amine” and “amino” are art-recognized and refer to both unsubstituted and substituted amines and salts thereof, e.g., a moiety that can be represented by the general formulae:

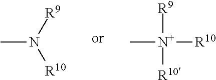

where R⁹, R¹⁰ and R^{10′} each independently represent a hydrogen, an alkyl, an alkenyl, —(CH₂)ₘ—R⁸, or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocyclyl or a polycyclyl; and m is zero or an integer from 1 to 8. In some embodiments, only one of R⁹ or R¹⁰ is a carbonyl, e.g., R⁹, R¹⁰, and the nitrogen together do not form an imide. In some embodiments, R⁹ and R10 (and optionally R^{10′}) each independently represent a hydrogen, an alkyl, an alkenyl, or —(CH₂)ₘ—R⁸. In certain embodiments, an amino group is basic, meaning its protonated form has a pKa above 7.00.

The terms “amide” and “amido” are art-recognized as an amino-substituted carbonyl and include a moiety that can be represented by the general formula:

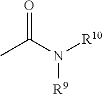

wherein R⁹ and R¹⁰ are as defined above. In some embodiments, the amide will not include imides, which may be unstable.

The term “C₁₋₆alkylamino” refers to a C₁₋₆alkyl group substituted with an amine group.

The term “carbonyl” is art-recognized and includes moieties such as those represented by the general formulae:

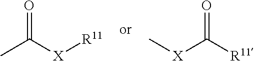

wherein X is a bond or represents an oxygen or a sulfur, and R¹¹ represents a hydrogen, an alkyl, an alkenyl, —(CH₂)ₘ—R⁸ or a pharmaceutically acceptable salt, R^{11′} represents a hydrogen, an alkyl, an alkenyl or —(CH₂)ₘ—R⁸, where m and R⁸ are as defined above. Where X is an oxygen and R¹¹ or R^{11′} is not hydrogen, the formula represents an “ester”. Where X is an oxygen and R¹¹ is a hydrogen, the formula represents a “carboxylic acid”.

The term “aryl” as used herein includes 5-, 6-, and 7-membered substituted or unsubstituted single-ring aromatic groups in which each atom of the ring is carbon. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups can include moieties containing six to fourteen carbons. Aryl groups include benzene, naphthalene, phenanthrene, phenol, aniline, and the like.

The term “C₁₋₆aralkyl”, as used herein, refers to a C₁₋₆alkyl group substituted with an aryl group.

The terms “carbocycle”, “carbocyclyl”, and “cycloalkyl” as used herein, refer to a 3- to 7-membered non-aromatic substituted or unsubstituted ring in which each atom of the ring is carbon. The terms “carbocycle”, “carbocyclyl”, and “cycloalkyl” also include polycyclic ring systems having two or more cyclic rings in which two or more carbons are common to two adjoining rings wherein at least one of the rings is carbocyclic, e.g., the other cyclic rings can be cycloalkyls, cycloalkenyls, cycloalkynyls, aryls, heteroaryls, and/or heterocyclyls. Carbocyclyl groups include moieties containing three to fourteen carbons. Carbocyclyls include cyclopropyl, cyclopentyl, cyclohexyl, cyclohexylmethyl, and 4-methylcyclohexyl. Examples of polycyclic carbocyclyls include bicyclo[2.2.1]heptanyl, norbornyl, and adamantyl.

The term “heteroaryl” includes substituted or unsubstituted aromatic 5- to 7-membered ring structures, for example, 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include moieties containing one to thirteen carbons. Heteroaryl groups include, for example, pyrrole, furan, thiophene, imidazole, oxazole, thiazole, triazole, pyrazole, pyridine, pyrazine, pyridazine, and pyrimidine, and the like. Heteroaryl groups containing two fused aromatic rings can be one of the following moieties: quinoline, isoquinoline, naphthyridine, cinnoline, quinazoline, quinoxaline, benzimidazole, indole, azaindole, indazole, azaindazole, pyrrolopyridazine, and pyrrolopyrazine.

The term “C₁₋₆heteroaralkyl”, as used herein, refers to a C₁₋₆alkyl group substituted with a heteroaryl group.

The term “heteroatom” as used herein means an atom of any element other than carbon or hydrogen. For example, heteroatoms include nitrogen, oxygen, phosphorus, and sulfur.

The term “heterocyclyl” or “heterocyclic group” refers to substituted or unsubstituted non-aromatic 3- to 10-membered ring structures, for example, 3- to 7-membered rings, whose ring structures include one to four heteroatoms. The term “heterocyclyl” or “heterocyclic group” also includes polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls.

Heterocyclyl groups include moieties containing two to thirteen carbons. Heterocyclyl groups include, for example, piperidine, piperazine, pyrrolidine, morpholine, lactones, lactams, and the like.

The term “C₁₋₆haloalkyl” refers to a C₁₋₆alkyl group substituted with one or more halogen group(s). Representative C₁₋₆haloalkyl groups include, for example, CF₃, CF₂CF₃, CH₂CF₃, and CF₂CH₃.

The term “C₁₋₆haloalkoxy” refers to a C₁₋₆alkoxy group substituted with one or more halogen group(s). Representative C₁₋₆haloalkoxy groups include, for example, OCF₃, OCH₂CH₂CF₃, OCH₂CF₃, and OCF₂CF₃.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include, for example, an alkyl, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, a carbamoyl, a guanidine, an imine, a cyano, a nitro, an azido, a sulfhydryl, an alkylthio, a sulfate, a sulfonate, a sulfamoyl, a sulfonamido, a sulfonyl, a carbocyclyl, a heterocyclyl, an aralkyl, a heteroaralkyl, or an aromatic or heteroaromatic moiety. It will be understood by those skilled in the art that the moieties substituted on the hydrocarbon chain can themselves be substituted, if appropriate.

In some embodiments, the compounds provided herein, or salts thereof, are substantially isolated or purified. By “substantially purified” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds, or salt thereof. Methods for purifying compounds and their salts are routine in the art.

The term “prophylactic or therapeutic” treatment is art-recognized and includes administration to a patient of one or more of the compounds provided herein or a pharmaceutical composition including the same. If the compound(s) is administered prior to clinical manifestation of the unwanted condition (e.g., disease or other unwanted state of the host patient) then the treatment is prophylactic, (i.e. it protects the host against developing the unwanted condition), whereas if it is administered after manifestation of the unwanted condition, the treatment is therapeutic, (i.e. it is intended to diminish, ameliorate, or stabilize the existing unwanted condition or side effects thereof).

As used herein, the term “activator” is meant to describe a compound that increases an activity of an enzyme or system of enzymes, receptors, or other pharmacological target (for example, eIF2α kinase 3). An activator can modify one or more sites on or near the active site of the enzyme, or it can cause a conformational change elsewhere on the enzyme. The term activator is used more broadly herein than scientific literature so as to also encompass other classes of pharmacologically or therapeutically useful agents, such as agonists, antagonists, stimulants, co-factors, and the like.

The term “Emax” refers to the maximal response that is produced by the compound.

As used herein, the term “IC₅₀” is meant to describe the dose at which 50% of the maximal effect is observed.

As used herein, the term “treating” or “treatment” includes reversing, reducing, or arresting one or more symptoms, clinical signs, and/or underlying pathologies of a condition in a manner to improve or stabilize a patient's condition.

### Compounds

Provided herein is a compound having a structure of Formula (I),

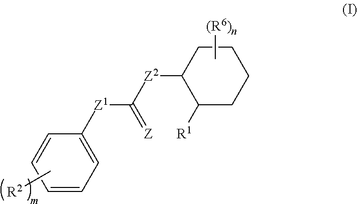

or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₁₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR4R5, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

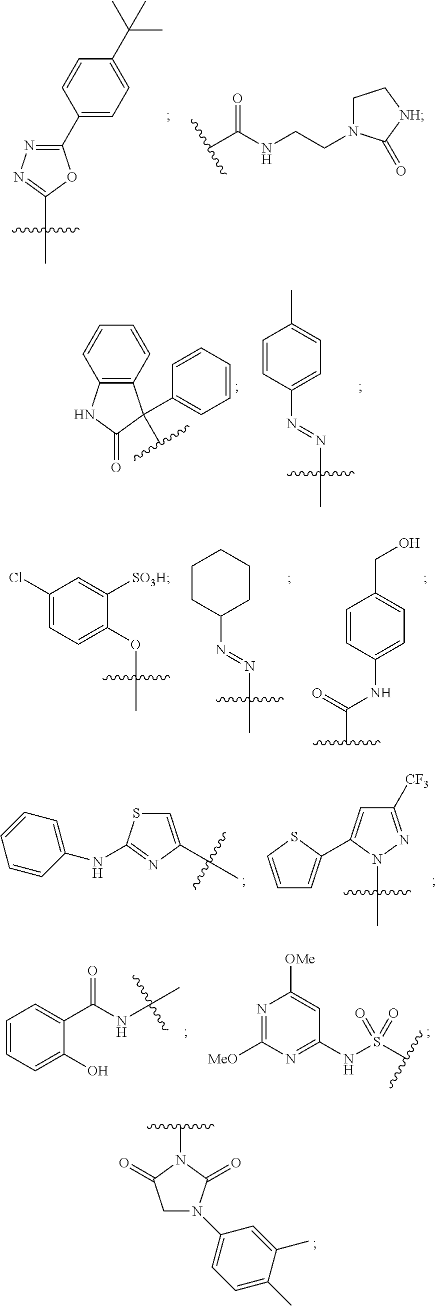

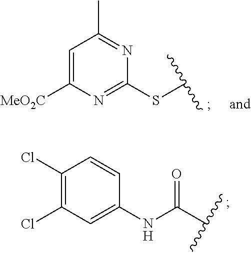 [SMILES: *Sc1nc(C)cc(C(=O)OC)n1 ; *C(=O)Nc1ccc(Cl)c(Cl)c1]

X is selected from the group consisting of: NR⁴, O, and S(O)ₚ;
m is an integer from 1 to 5;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl and unsubstituted or substituted heteroaryl;
each R⁴ and R⁵ is independently selected from the group consisting of: H and unsubstituted or substituted C₁₋₆alkyl;
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR4R5, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

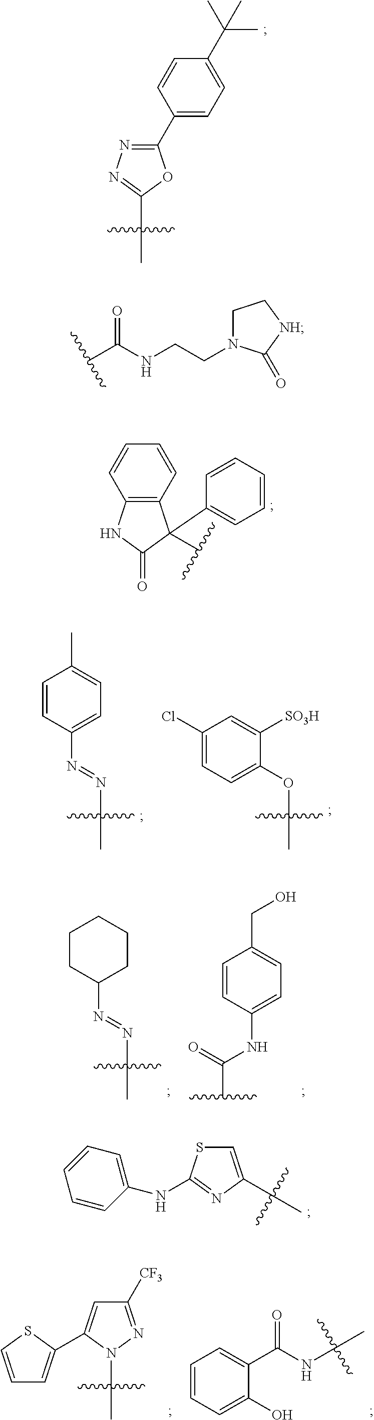

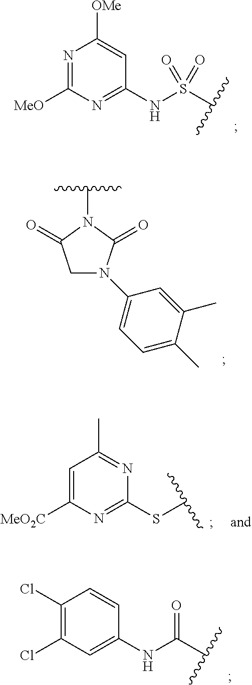 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

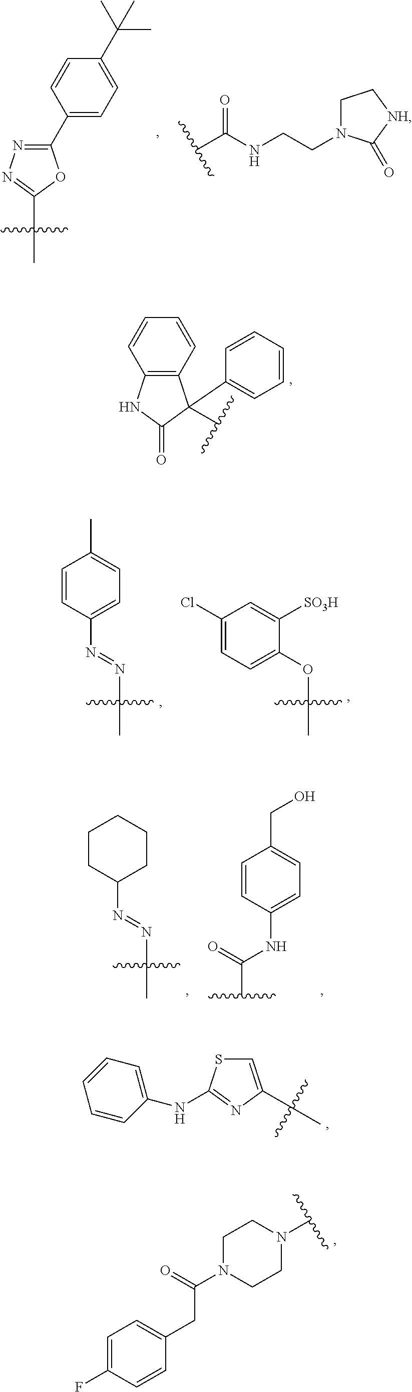

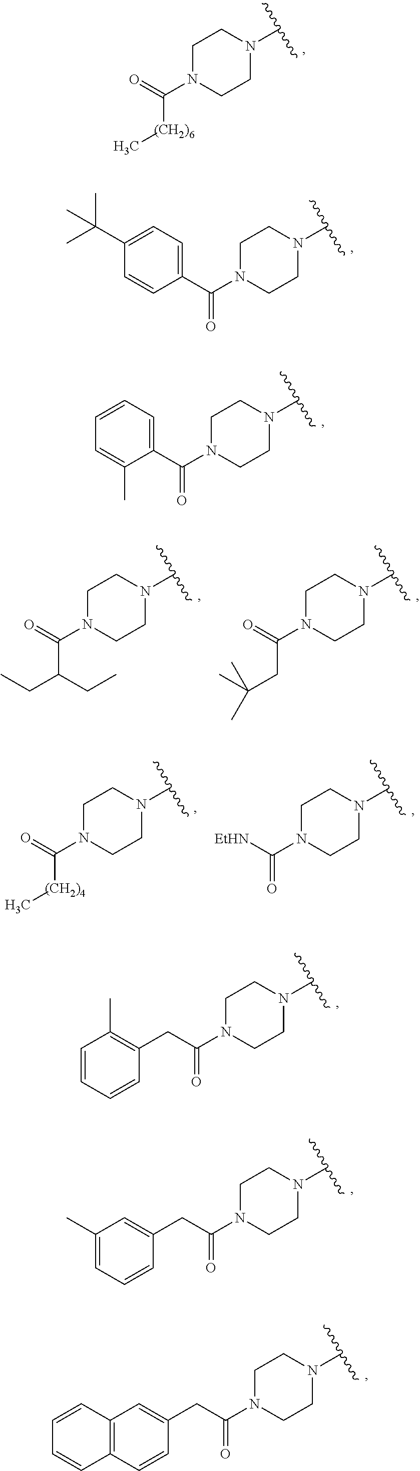

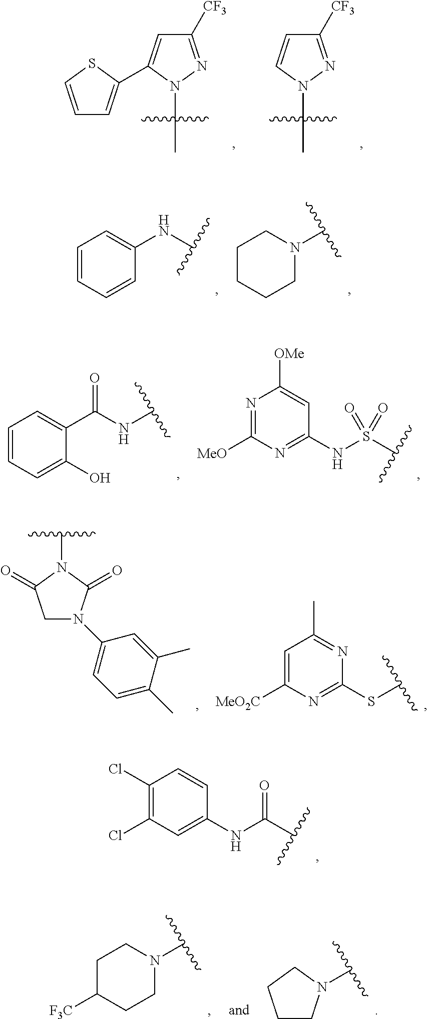

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some embodiments, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be O.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, W is absent or [C(R⁵)₂]_{q}. For example, W can be CH₂. In some embodiments, W is absent.

In some embodiments, q is an integer from 1 to 5. In some embodiments, q is 1.

In some embodiments, R³ is

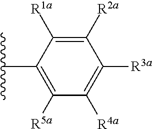

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, a compound of Formula (I) is:

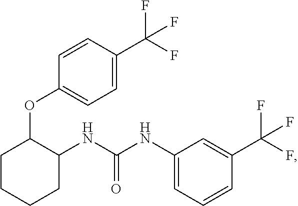 [SMILES: O=C(Nc1cccc(C(F)(F)F)c1)NC1CCCCC1Oc1ccc(C(F)(F)F)cc1]

or a pharmaceutically acceptable salt thereof.

Also provided herein is a compound having the structure of Formula (II):

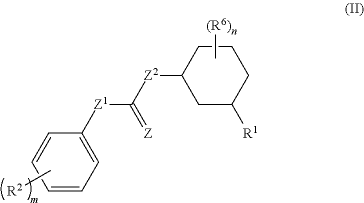

or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle, O—(CH₂)₂₋₄—NR⁴R⁵; O—(CH₂)₂₋₄-heteroaryl;

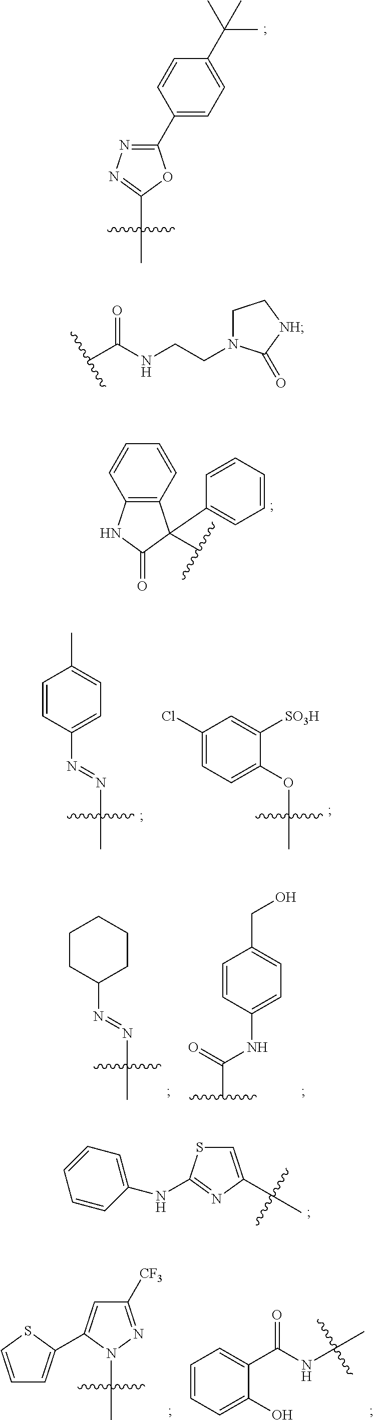

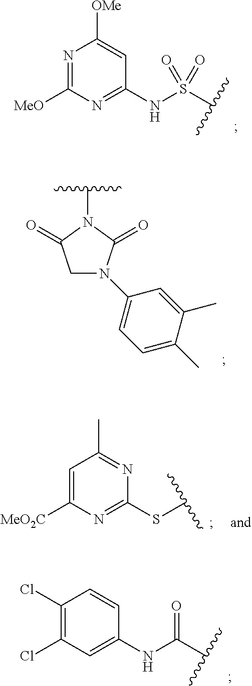 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1]

m is an integer from 1 to 5;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl;
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

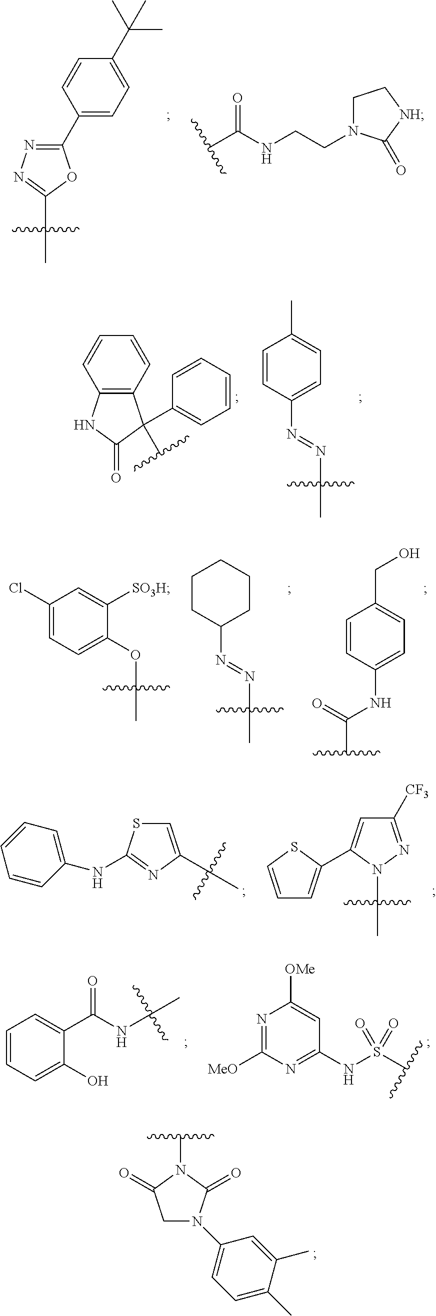

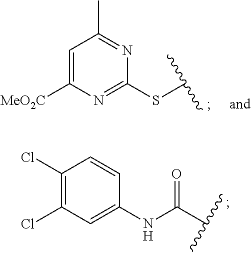 [SMILES: *Sc1nc(C)cc(C(=O)OC)n1 ; *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]-triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

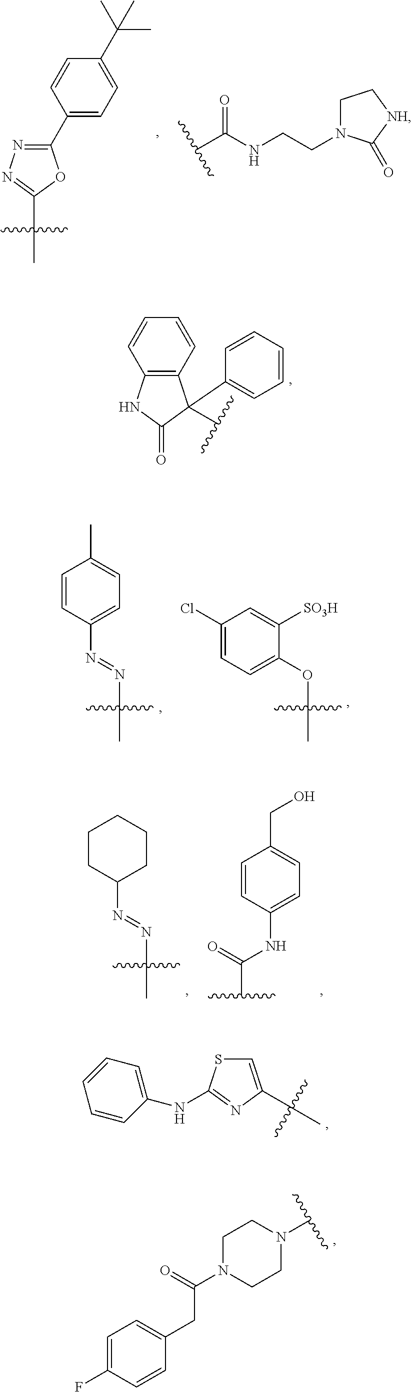

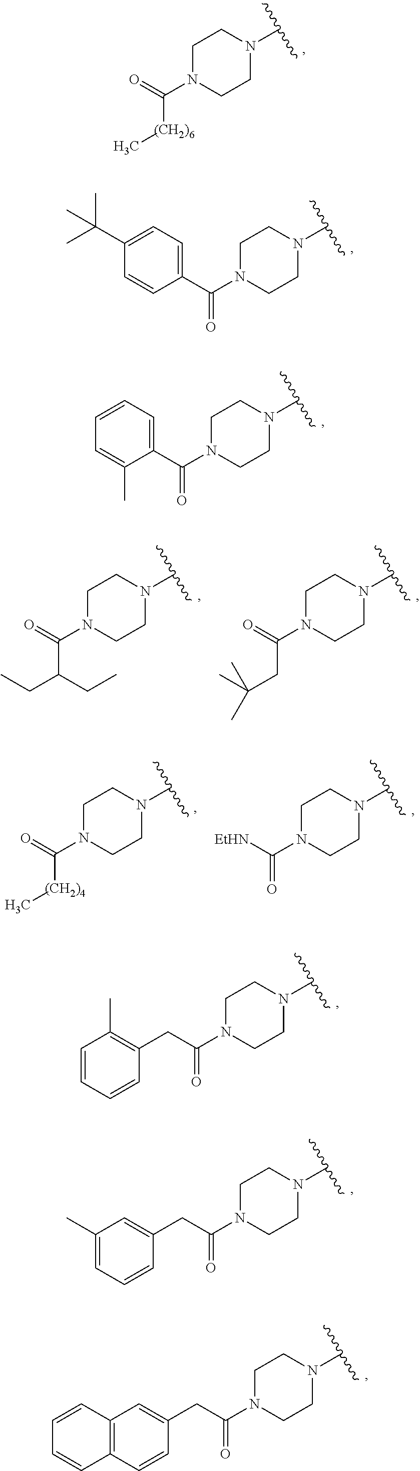

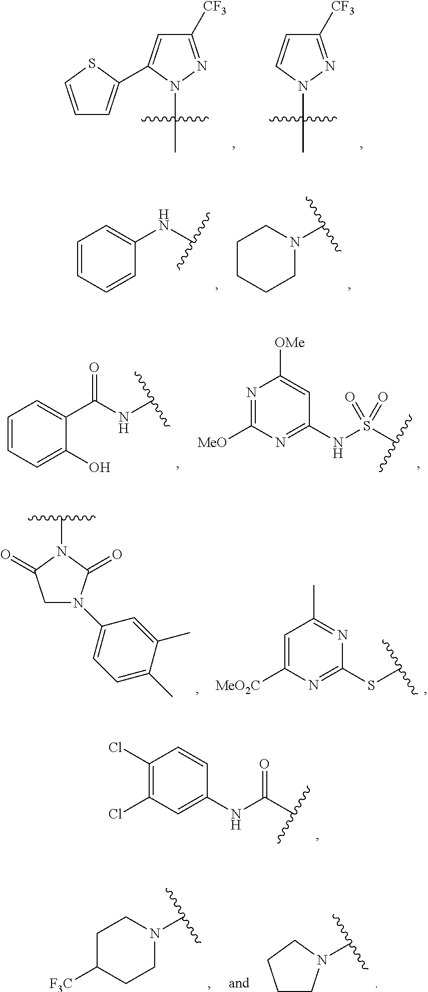

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be O.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, W is absent or [C(R⁵)₂]_{q}. For example, W can be CH₂. In some embodiments, W is absent.

In some embodiments, q is an integer from 1 to 5. In some embodiments, q is 1.

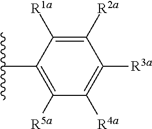

In some embodiments, R³ is

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, a compound of Formula (II) is:

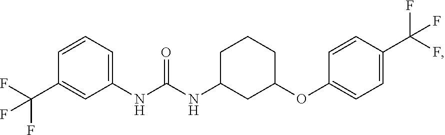 [SMILES: O=C(Nc1cccc(C(F)(F)F)c1)NC1CCCC(Oc2ccc(C(F)(F)F)cc2)C1]

or a pharmaceutically acceptable salt thereof.

Another aspect provided herein is a compound having the structure of Formula (III):

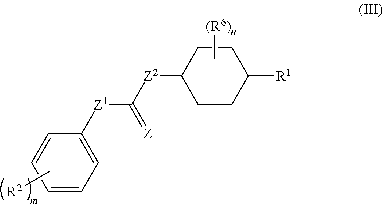

or a pharmaceutically acceptable salt thereof,

wherein:
Z, Z¹, and Z² are each independently selected from the group consisting of: NH, O, and S;
R¹ is XR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

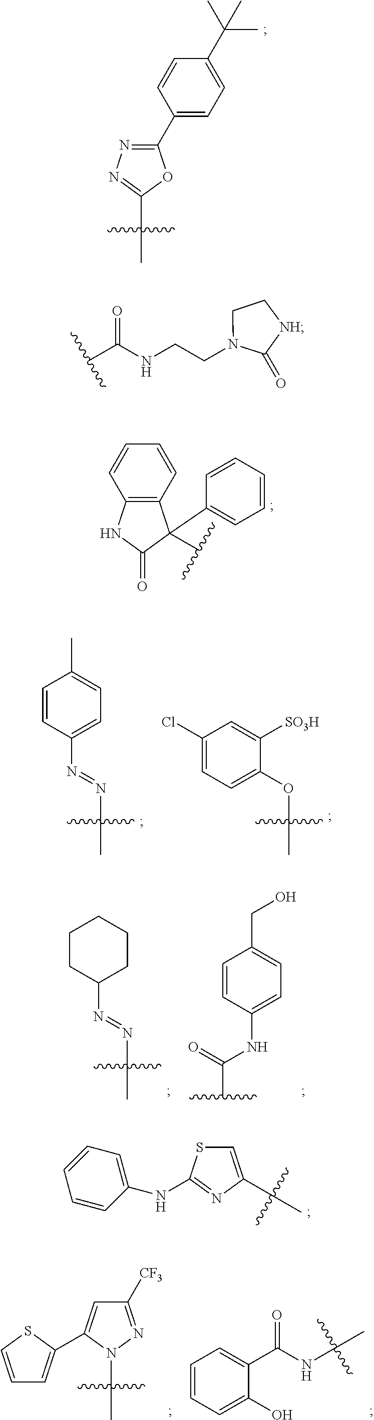

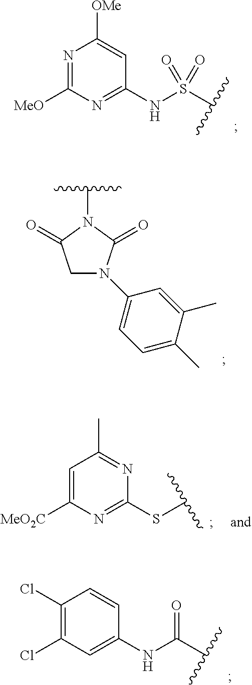 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1]

m is an integer from 1 to 5;
n is an integer from 0 to 2;
p is an integer from 0 to 2;
R³ is selected from the group consisting of: unsubstituted or substituted heteroaryl; and

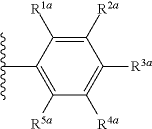

wherein
R^{2a} and R^{4a} are independently selected from the group consisting of: H; halo; unsubstituted or substituted C₁₋₆alkyl; unsubstituted or substituted C₁₋₆alkenyl; unsubstituted or substituted C₁₋₆alkynyl; C₁₋₆haloalkyl; CONR⁴R⁵; CONH(C₁₋₆alkyl)heterocyclyl; CONH(C₁₋₆alkyl)carbocyclyl; CONH(C₁₋₆alkyl)aryl; CONH(C₁₋₆alkyl)heteroaryl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkyl)carbocyclyl; C₁₋₆aralkyl; C₁₋₆heteroaralkyl; (C₁₋₆alkoxy)heterocyclyl; (C₁₋₆alkoxyl)carbocyclyl; (C₁₋₆alkoxyl)aryl; (C₁₋₆alkoxyl)heteroaryl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

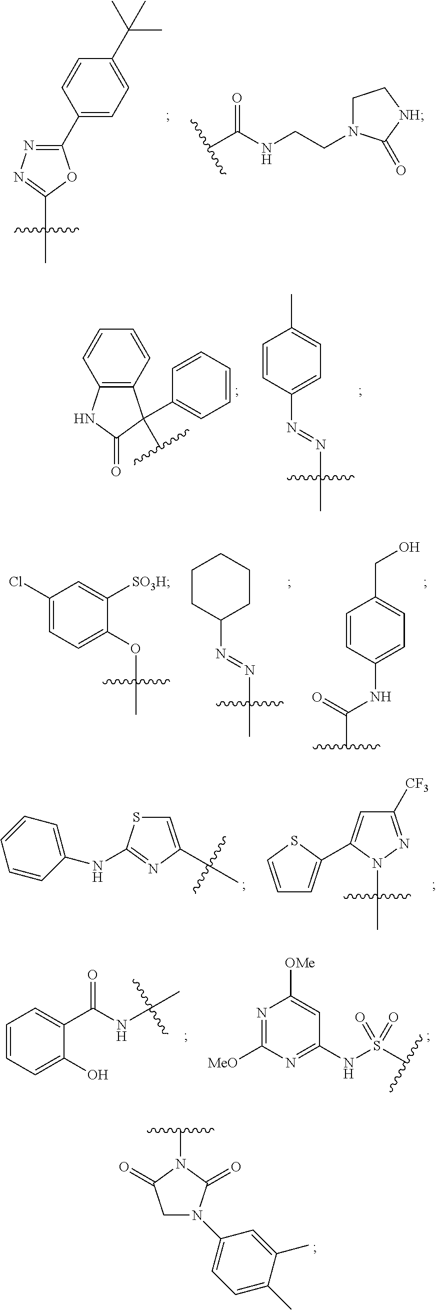

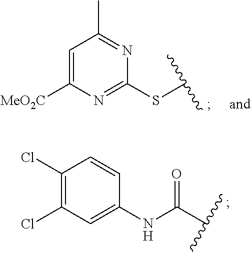 [SMILES: *Sc1nc(C)cc(C(=O)OC)n1 ; *C(=O)Nc1ccc(Cl)c(Cl)c1]

R^{1a}, R^{3a}, and R^{5a} are independently selected from the group consisting of: H; Cl; Br; I; —NO₂; —CN; unsubstituted or substituted C₁₋₆alkyl; C₁₋₆haloalkyl; CONR⁴R⁵; CONH(C₁₋₆alkyl)heterocyclyl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkoxy)heterocyclyl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

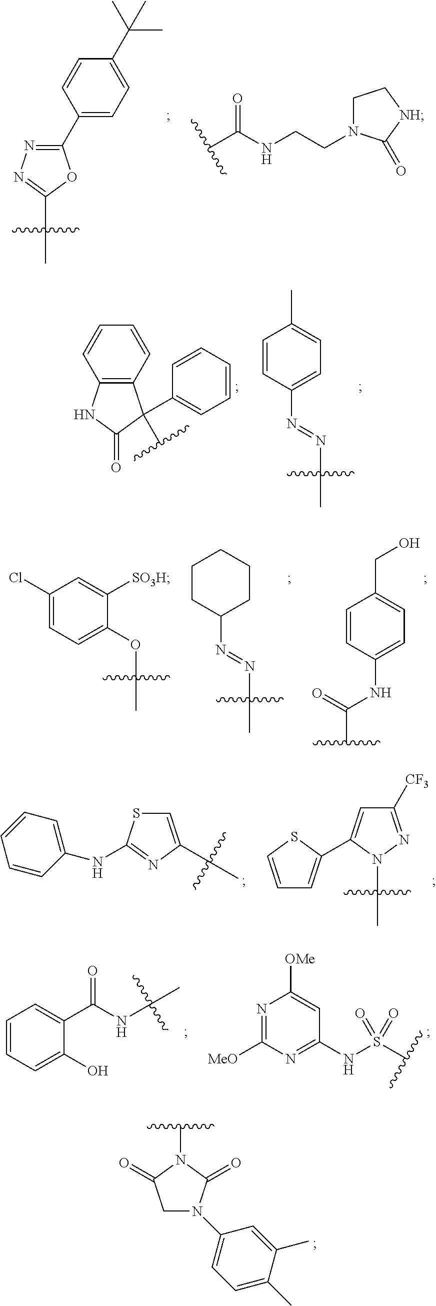

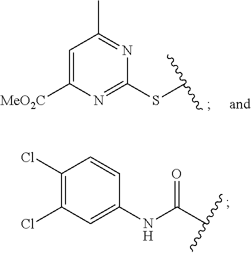 [SMILES: *Sc1nc(C)cc(C(=O)OC)n1 ; *C(=O)Nc1ccc(Cl)c(Cl)c1]

each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

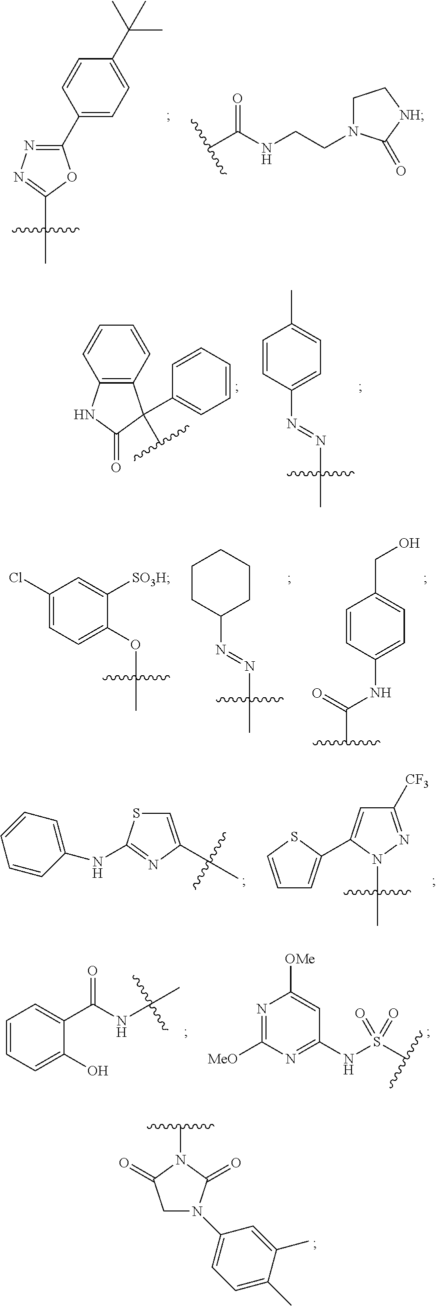

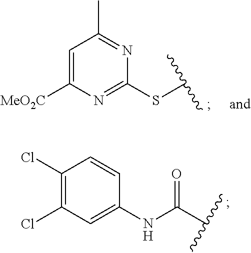 [SMILES: *Sc1nc(C)cc(C(=O)OC)n1 ; *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, the compound of Formula (III) has the structure:

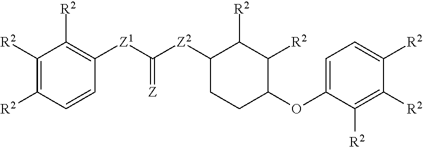

wherein R², Z, Z¹, and Z² are defined herein.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

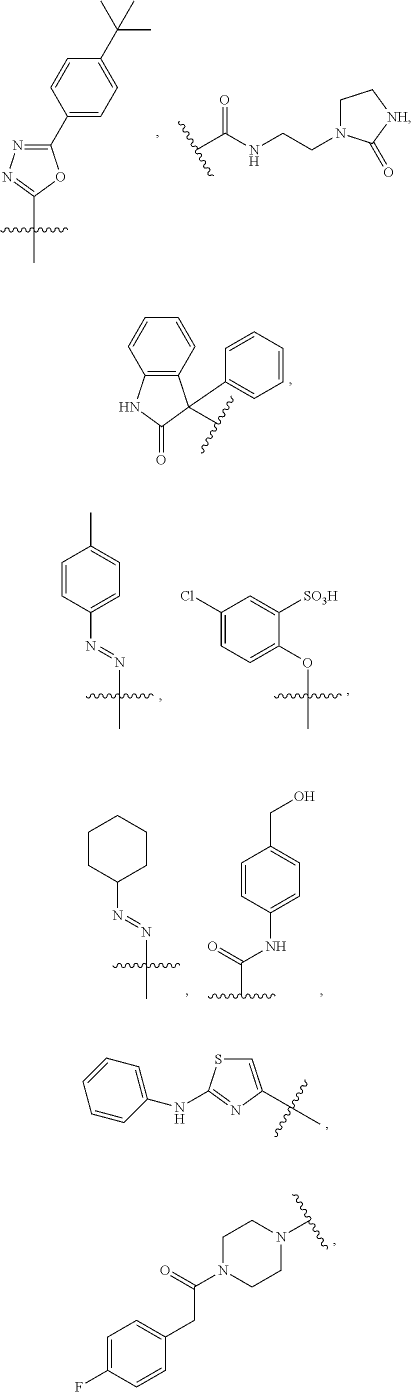

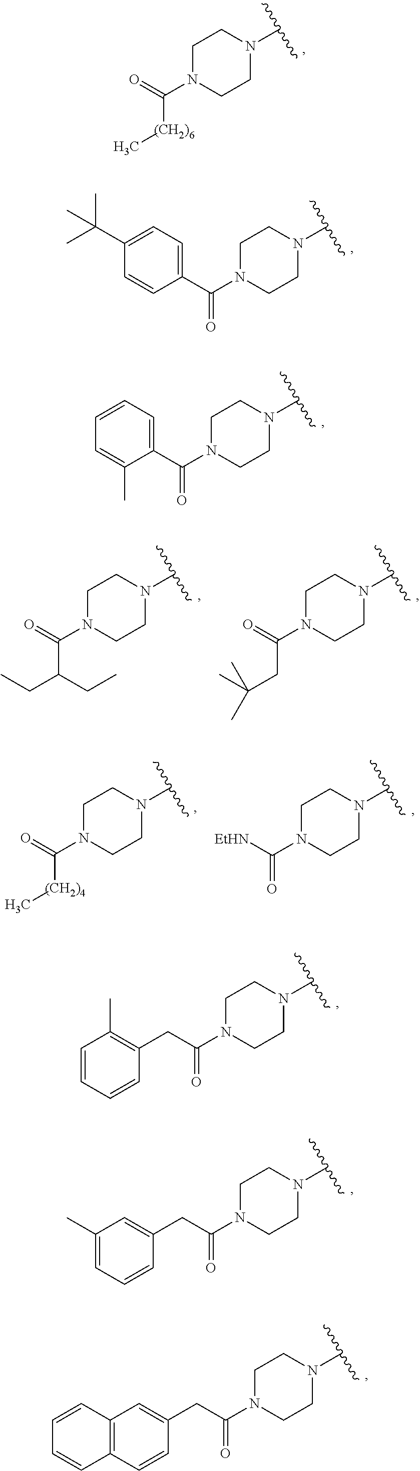

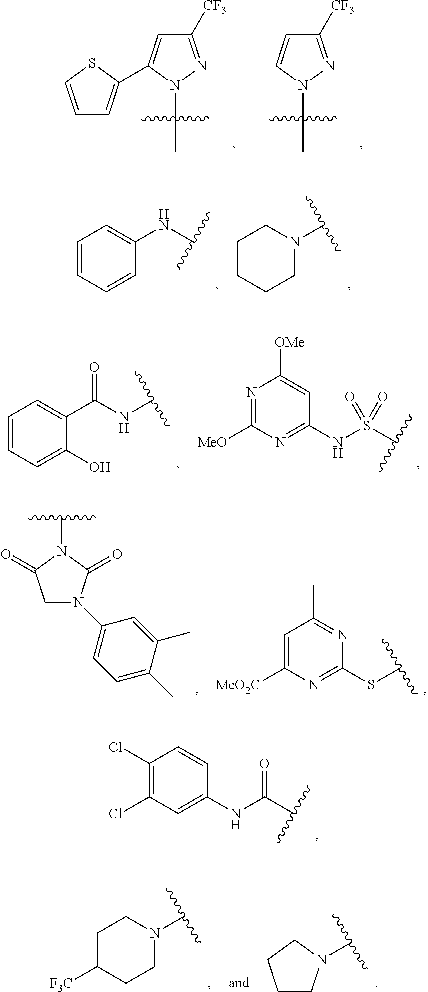

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be Q.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, W is absent or [C(R⁵)₂]_{q}. For example, W can be CH₂. In some embodiments, W is absent.

In some embodiments, q is an integer from 1 to 5. In some embodiments, q is 1.

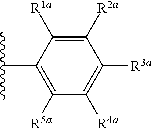

In some embodiments, R³ is

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, —NO₂; —CN; unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

A further aspect provided herein is a compound having the structure of Formula (IV):

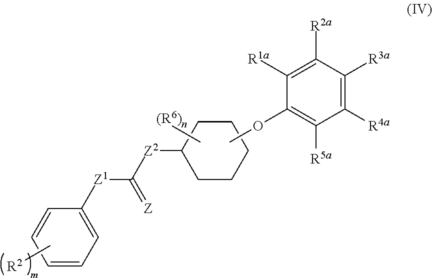

or a pharmaceutically acceptable salt thereof,
wherein:
Z is selected from the group consisting of: O and S;
Z¹ and Z² are each NH;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

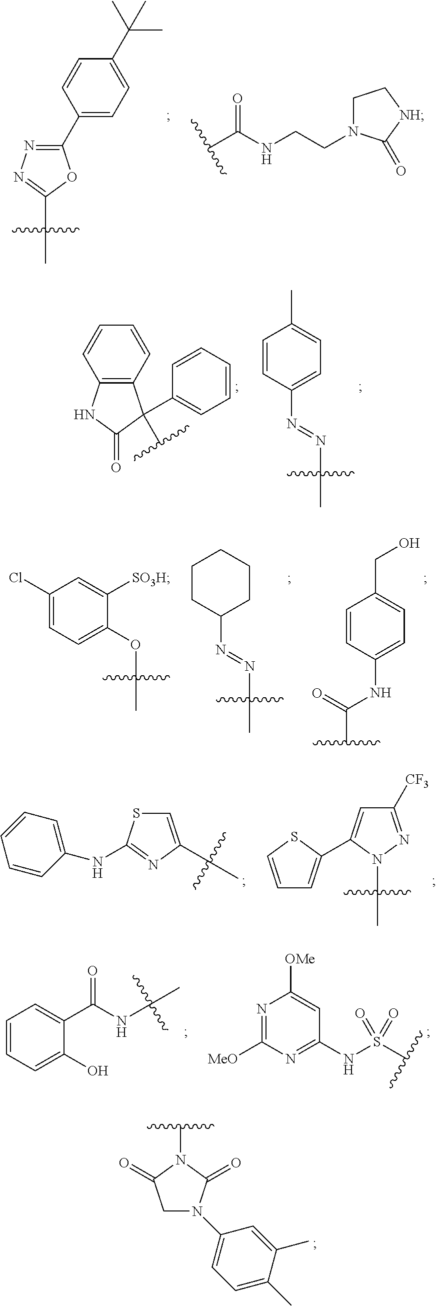

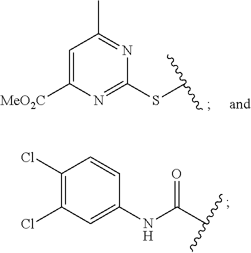 [SMILES: *Sc1nc(C)cc(C(=O)OC)n1 ; *C(=O)Nc1ccc(Cl)c(Cl)c1]

m is an integer from 1 to 5;
n is an integer from 0 to 2;
R^{2a} and R^{4a} are independently selected from the group consisting of: H; halo; unsubstituted or substituted C₁₋₆alkyl; unsubstituted or substituted C₁₋₆alkenyl; unsubstituted or substituted C₁₋₆alkynyl; C₁₋₆haloalkyl; CONR⁴R⁵; CONH(C₁₋₆alkyl)heterocyclyl; CONH(C₁₋₆alkyl)carbocyclyl; CONH(C₁₋₆alkyl)aryl; CONH(C₁₋₆alkyl)heteroaryl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkyl)carbocyclyl; C₁₋₆aralkyl; C₁₋₆heteroaralkyl; (C₁₋₆alkoxy)heterocyclyl; (C₁₋₆alkoxyl)carbocyclyl; (C₁₋₆alkoxyl)aryl; (C₁₋₆alkoxyl)heteroaryl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

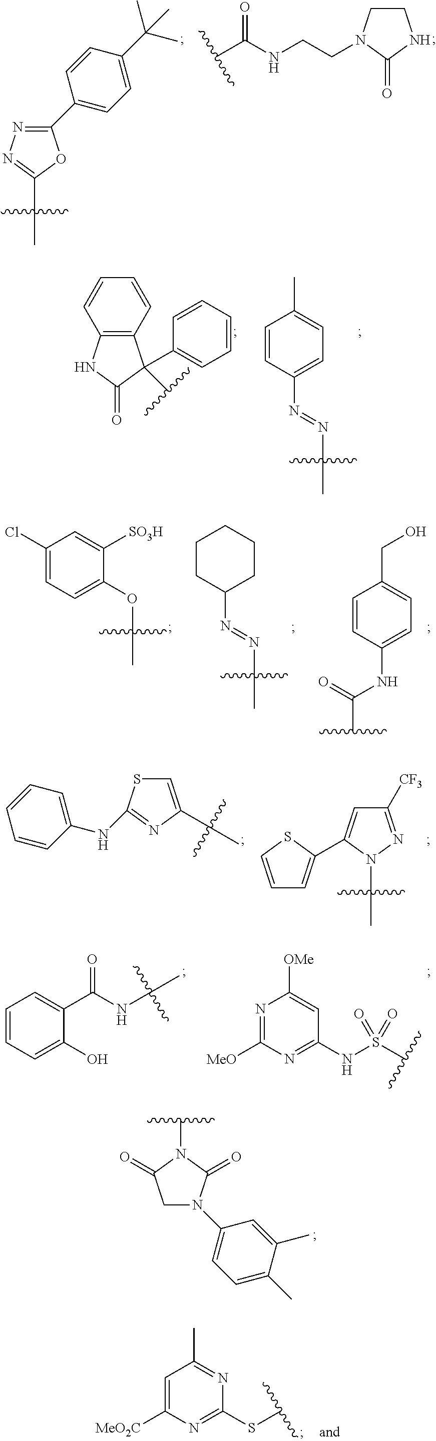

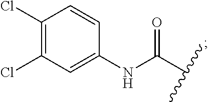 [SMILES: *C(=O)Nc1ccc(Cl)c(Cl)c1]

R^{1a}, R^{3a}, and R^{5a} are independently selected from the group consisting of: H; Cl; Br; I; —NO₂; —CN; unsubstituted or substituted C₁₋₆alkyl; C₁₋₆haloalkyl; CONR⁴R⁵; CONH(C₁₋₆alkyl)heterocyclyl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkoxy)heterocyclyl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

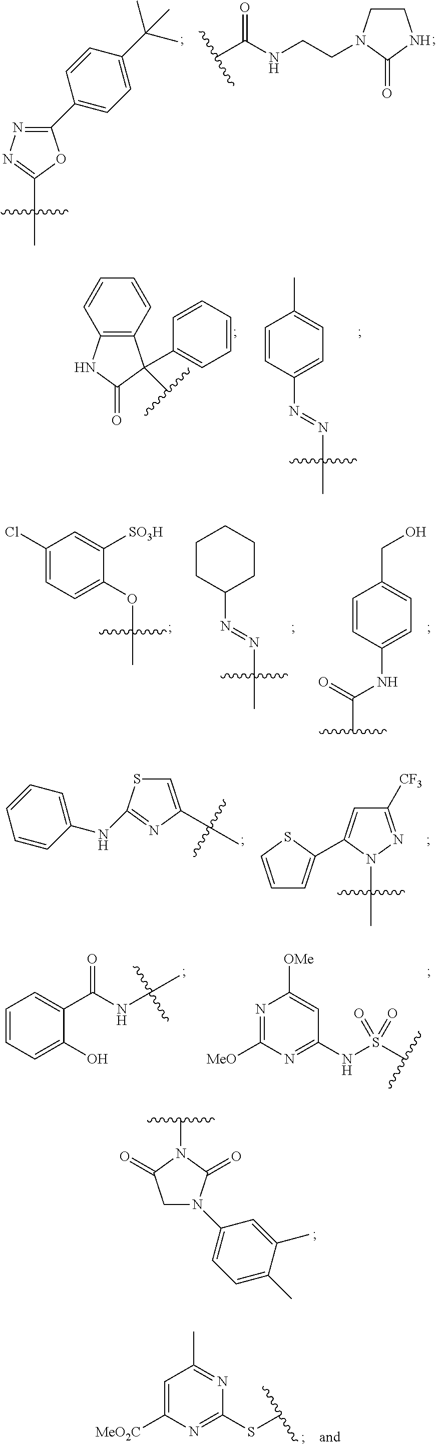

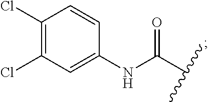 [SMILES: *C(=O)Nc1ccc(Cl)c(Cl)c1]

each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

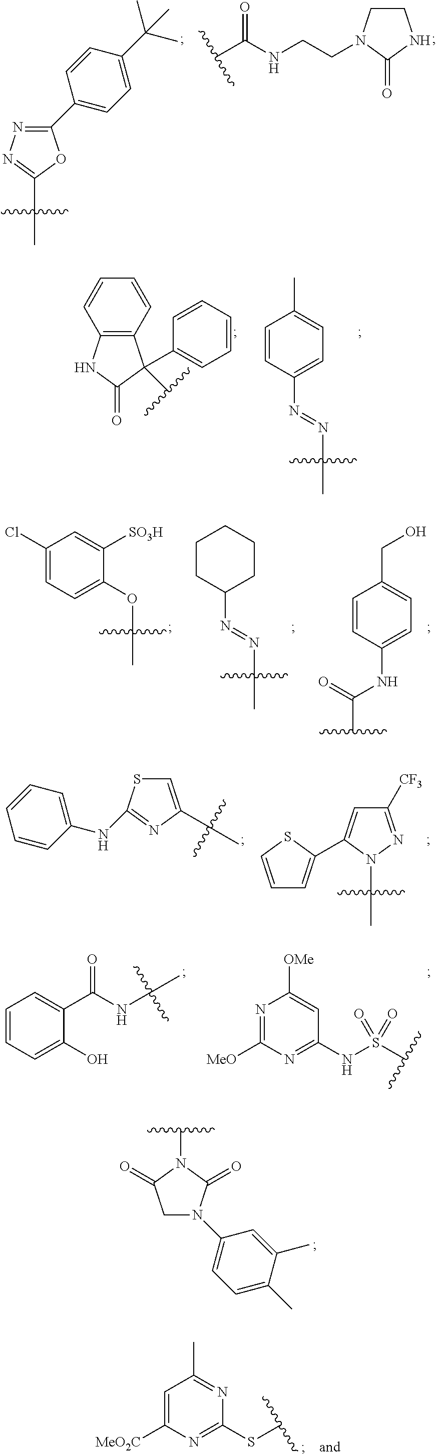

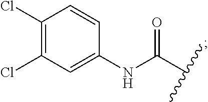 [SMILES: *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

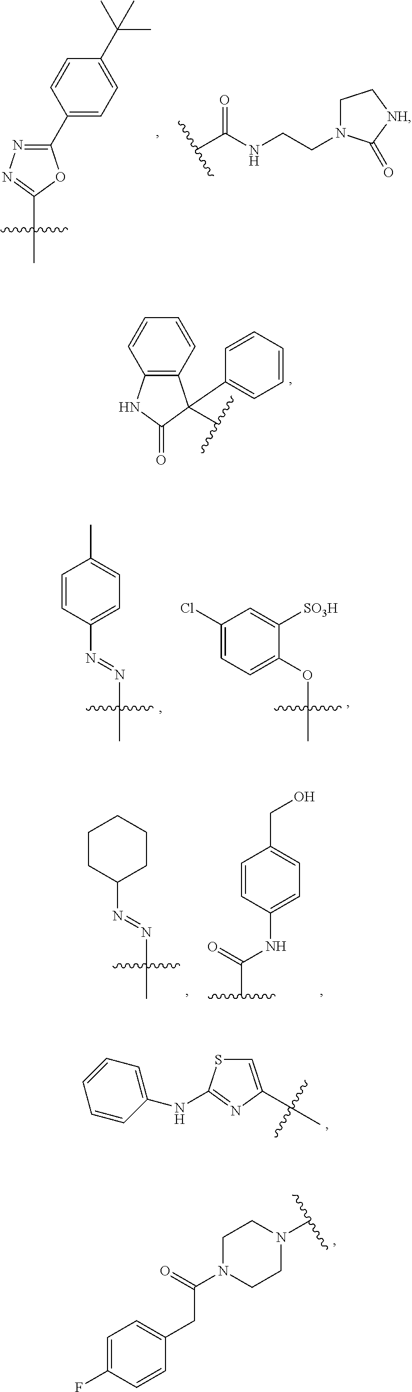

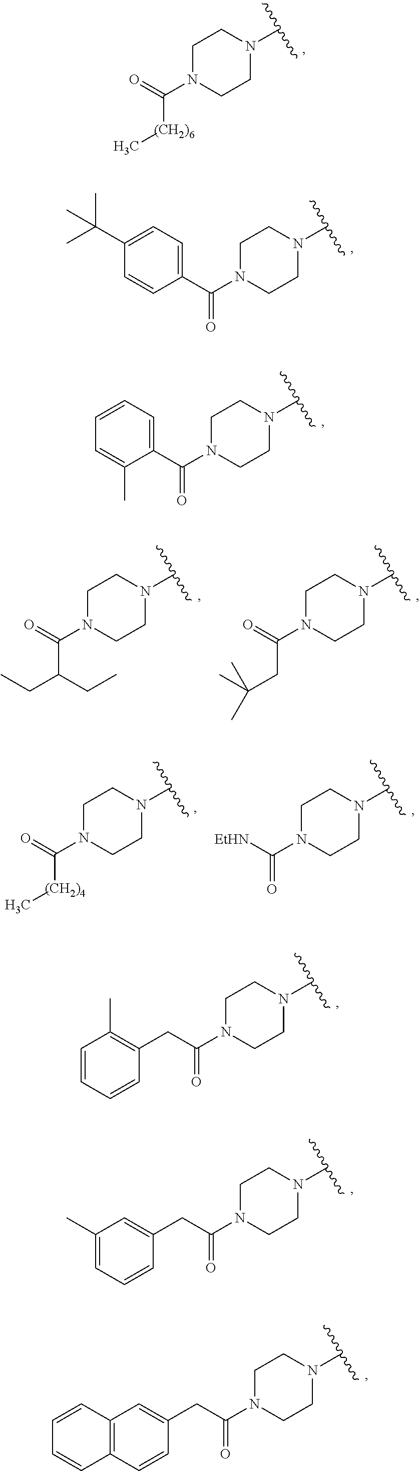

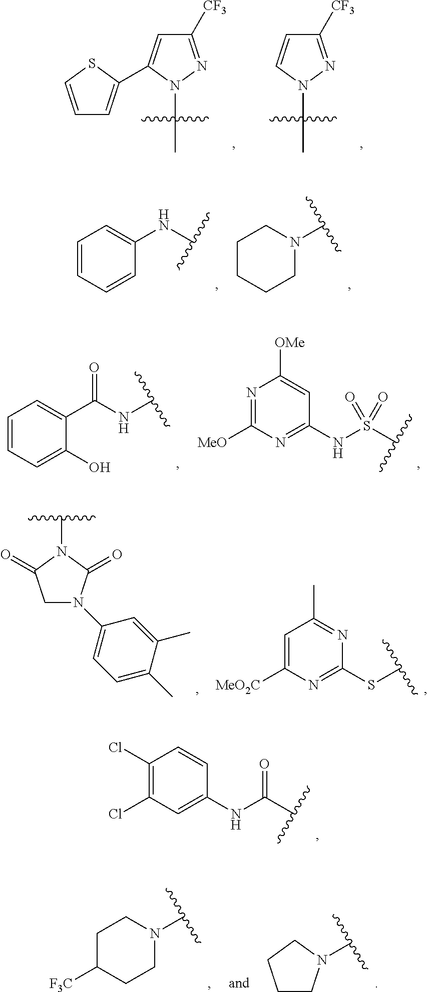

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be 0.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, W is absent or [C(R⁵)₂]_{q}. For example, W can be CH₂. In some embodiments, W is absent.

In some embodiments, q is an integer from 1 to 5. In some embodiments, q is 1.

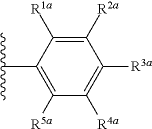

In some embodiments, R³ is

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, a compound of Formulas (III), and/or (IV) is selected from the group consisting of:

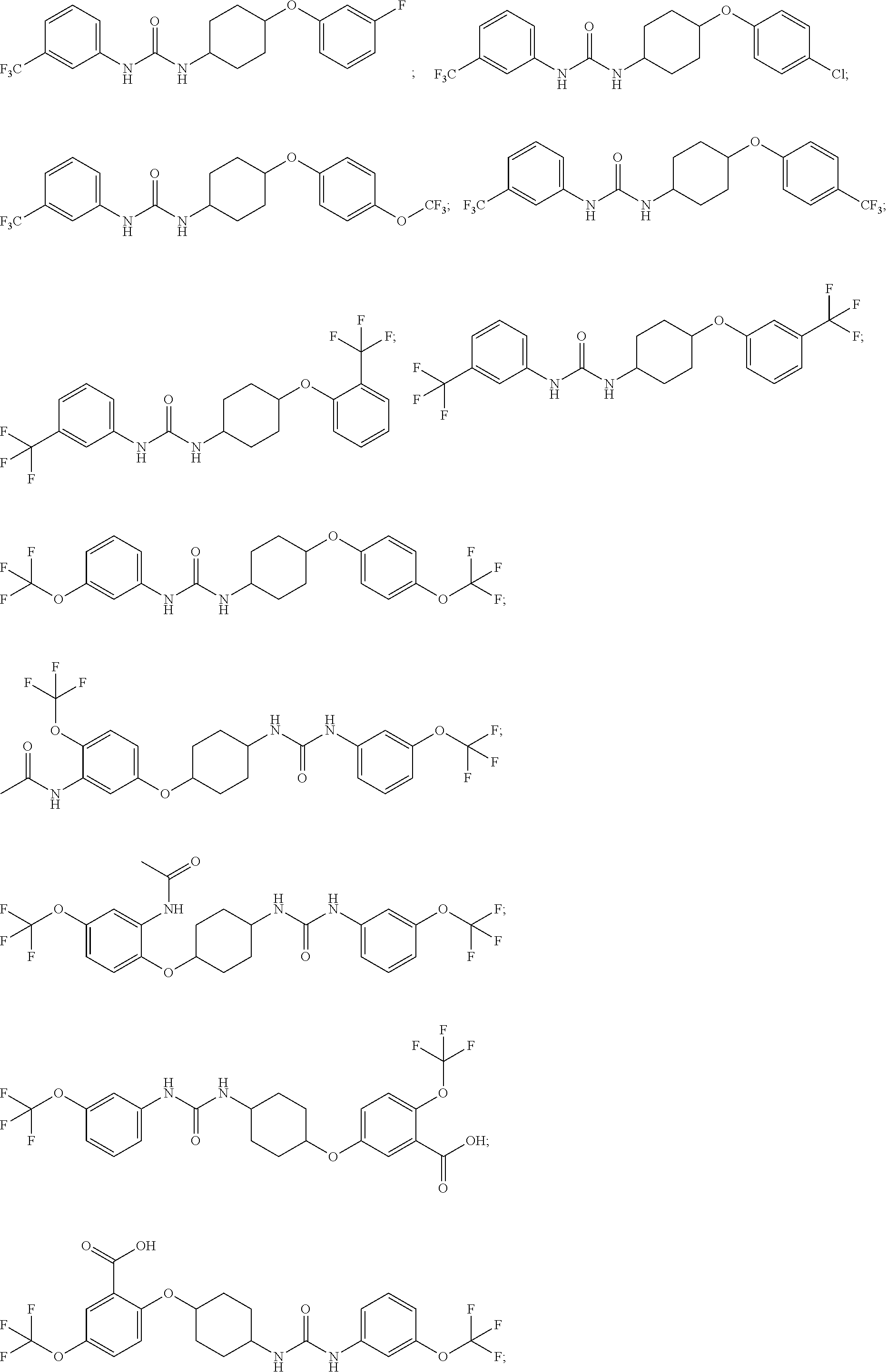

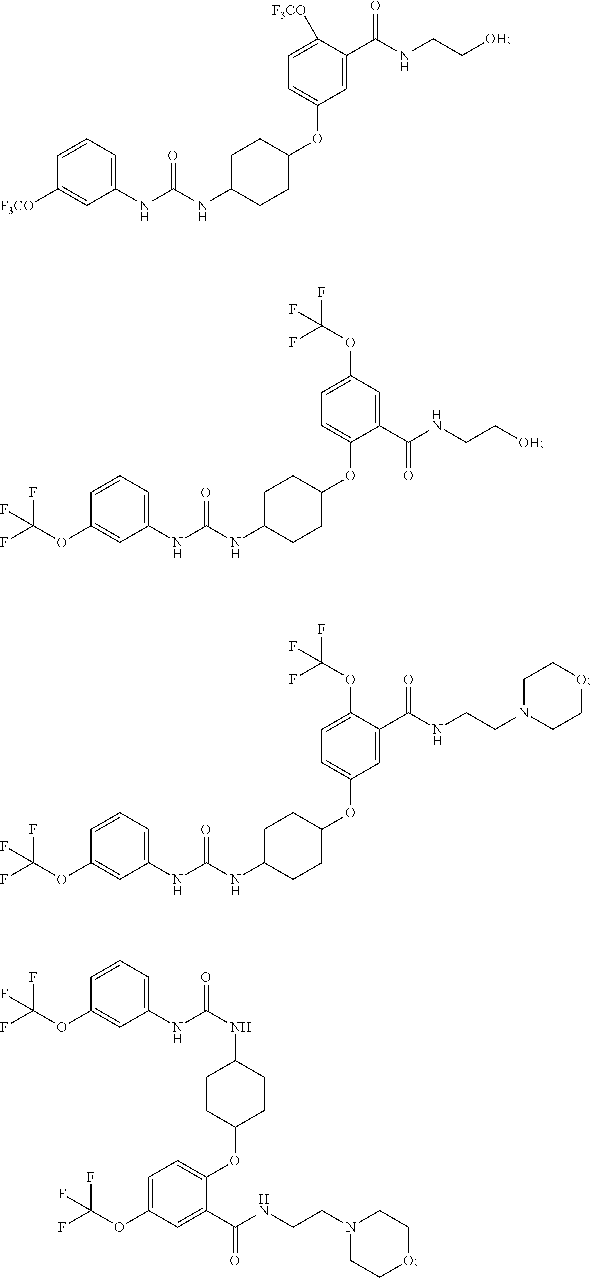 [SMILES: O=C(Nc1cccc(OC(F)(F)F)c1)NC1CCC(Oc2ccc(OC(F)(F)F)c(C(=O)NCCO)c2)CC1 ; O=C(Nc1cccc(OC(F)(F)F)c1)NC1CCC(Oc2ccc(OC(F)(F)F)cc2C(=O)NCCO)CC1 ; O=C(Nc1cccc(OC(F)(F)F)c1)NC1CCC(Oc2ccc(OC(F)(F)F)c(C(=O)NCCN3CCOCC3)c2)CC1 ; O=C(Nc1cccc(OC(F)(F)F)c1)NC1CCC(Oc2ccc(OC(F)(F)F)cc2C(=O)NCCN2CCOCC2)CC1]

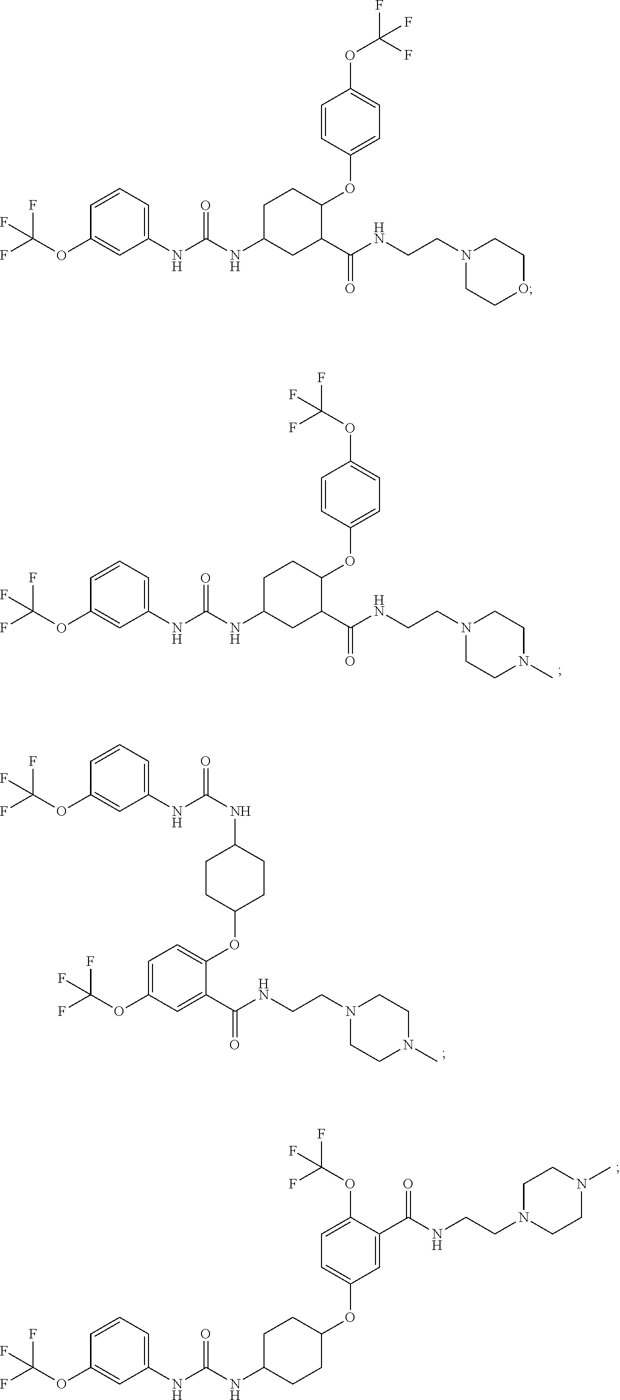 [SMILES: O=C(Nc1cccc(OC(F)(F)F)c1)NC1CCC(Oc2ccc(OC(F)(F)F)cc2)C(C(=O)NCCN2CCOCC2)C1 ; CN1CCN(CCNC(=O)C2CC(NC(=O)Nc3cccc(OC(F)(F)F)c3)CCC2Oc2ccc(OC(F)(F)F)cc2)CC1 ; CN1CCN(CCNC(=O)c2cc(OC(F)(F)F)ccc2OC2CCC(NC(=O)Nc3cccc(OC(F)(F)F)c3)CC2)CC1 ; CN1CCN(CCNC(=O)c2cc(OC3CCC(NC(=O)Nc4cccc(OC(F)(F)F)c4)CC3)ccc2OC(F)(F)F)CC1]

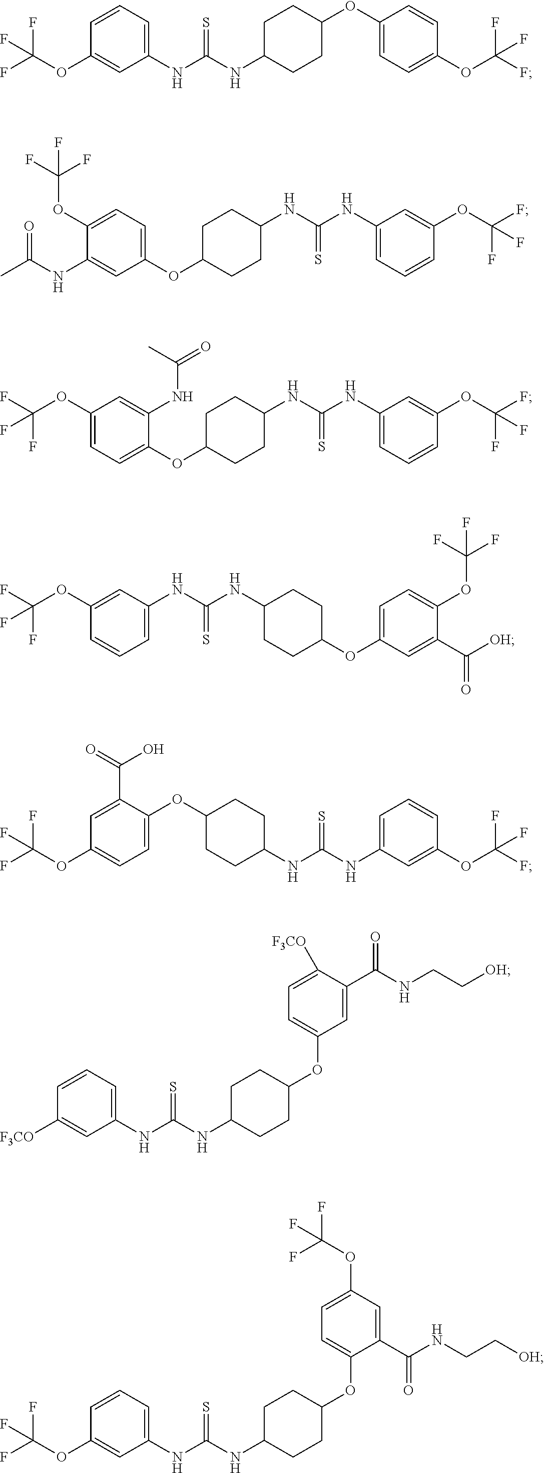

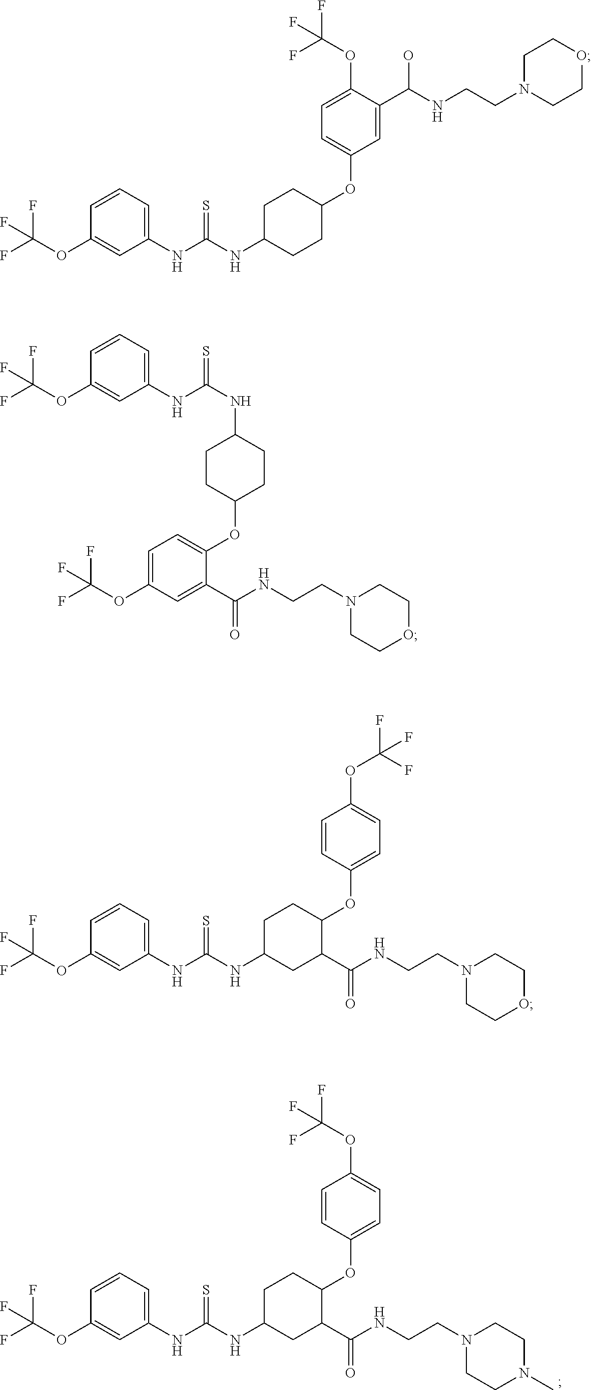 [SMILES: OC(NCCN1CCOCC1)c1cc(OC2CCC(NC(=S)Nc3cccc(OC(F)(F)F)c3)CC2)ccc1OC(F)(F)F ; O=C(NCCN1CCOCC1)c1cc(OC(F)(F)F)ccc1OC1CCC(NC(=S)Nc2cccc(OC(F)(F)F)c2)CC1 ; O=C(NCCN1CCOCC1)C1CC(NC(=S)Nc2cccc(OC(F)(F)F)c2)CCC1Oc1ccc(OC(F)(F)F)cc1 ; CN1CCN(CCNC(=O)C2CC(NC(=S)Nc3cccc(OC(F)(F)F)c3)CCC2Oc2ccc(OC(F)(F)F)cc2)CC1]

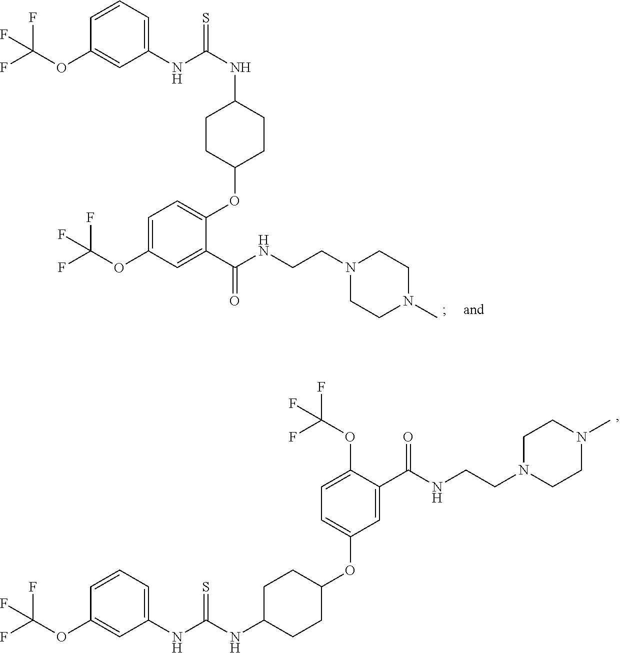 [SMILES: CN1CCN(CCNC(=O)c2cc(OC(F)(F)F)ccc2OC2CCC(NC(=S)Nc3cccc(OC(F)(F)F)c3)CC2)CC1 ; CN1CCN(CCNC(=O)c2cc(OC3CCC(NC(=S)Nc4cccc(OC(F)(F)F)c4)CC3)ccc2OC(F)(F)F)CC1]

or a pharmaceutically acceptable salt thereof.

Still another aspect provided herein is a compound having the structure of Formula (V):

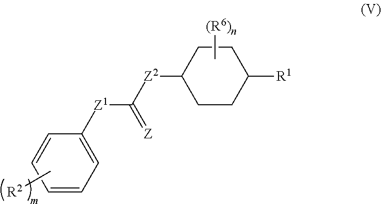

or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

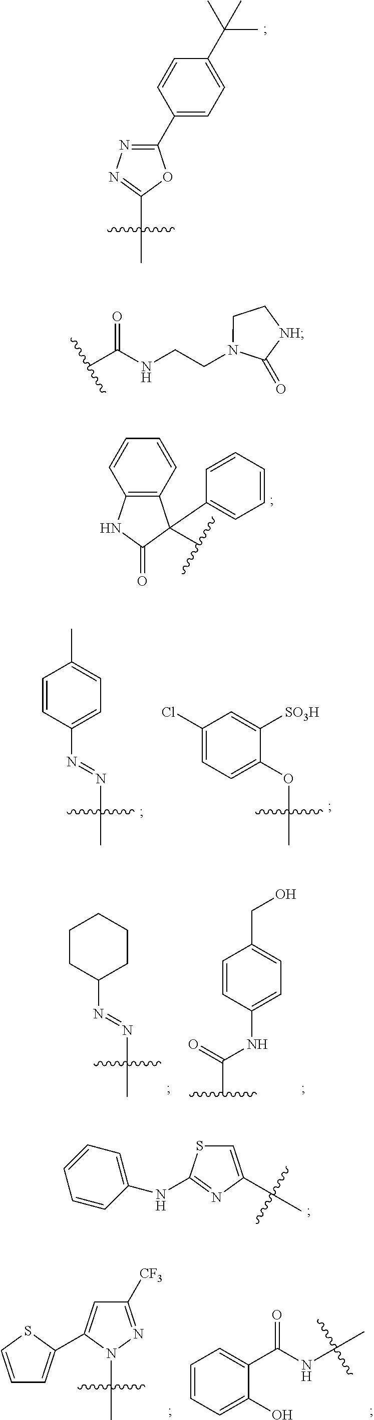

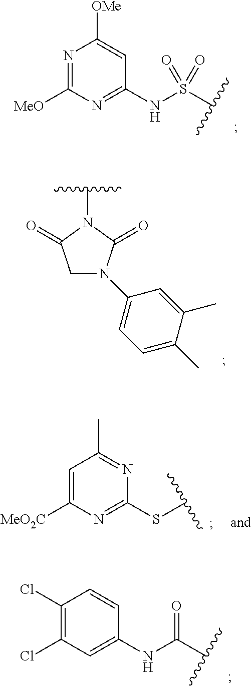 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1]

m is an integer from 1 to 5;
n is an integer from 0 to 2;
p is an integer from 0 to 2;
W is [C(R⁵)₂]_{q};
q is an integer from 1 to 5;
R³ is selected from the group consisting of: unsubstituted or substituted heteroaryl; and

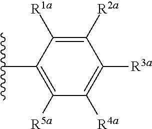

wherein
R^{2a}, R^{3a}, and R^{4a} are independently selected from the group consisting of: H; halo; unsubstituted or substituted C₁₋₆alkyl; unsubstituted or substituted C₁₋₆alkenyl; unsubstituted or substituted C₁₋₆alkynyl; C₁₋₆haloalkyl; CONR⁴R⁵; CONH(C₁₋₆alkyl)heterocyclyl; CONH(C₁₋₆alkyl)carbocyclyl; CONH(C₁₋₆alkyl)aryl; CONH(C₁₋₆alkyl)heteroaryl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkyl)carbocyclyl; C₁₋₆aralkyl; C₁₋₆heteroaralkyl; (C₁₋₆alkoxy)heterocyclyl; (C₁₋₆alkoxyl)carbocyclyl; (C₁₋₆alkoxyl)aryl; (C₁₋₆alkoxyl)heteroaryl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

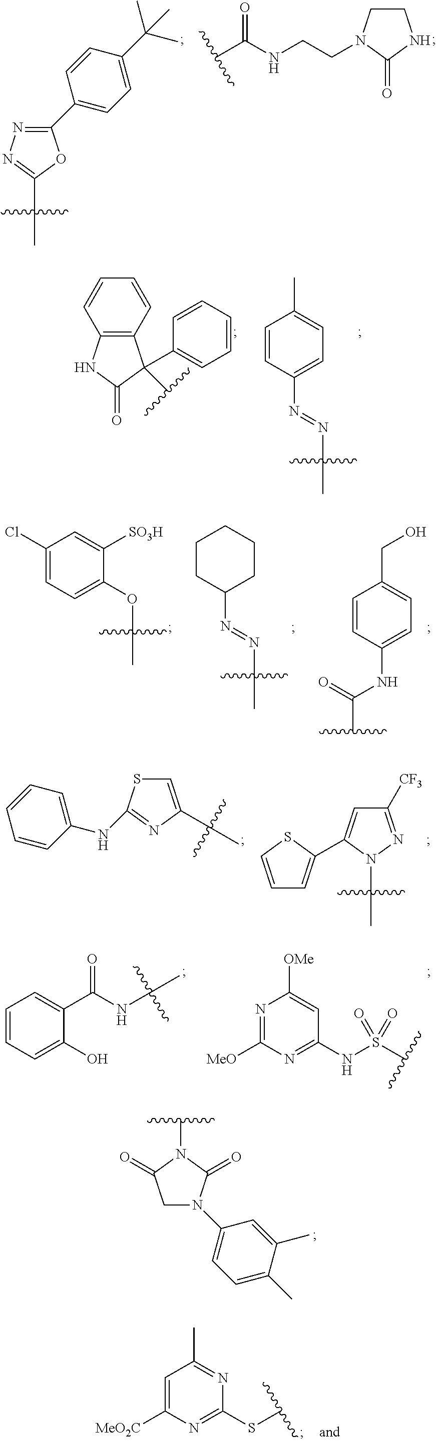

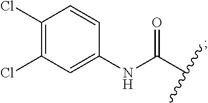 [SMILES: *C(=O)Nc1ccc(Cl)c(Cl)c1]

R^{1a} and R^{5a} are independently selected from the group consisting of: H; Cl; Br; I; unsubstituted or substituted C₁₋₆alkyl; C₁₋₆haloalkyl; CONR⁴R⁵; CONH(C₁₋₆alkyl)heterocyclyl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkoxy)heterocyclyl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

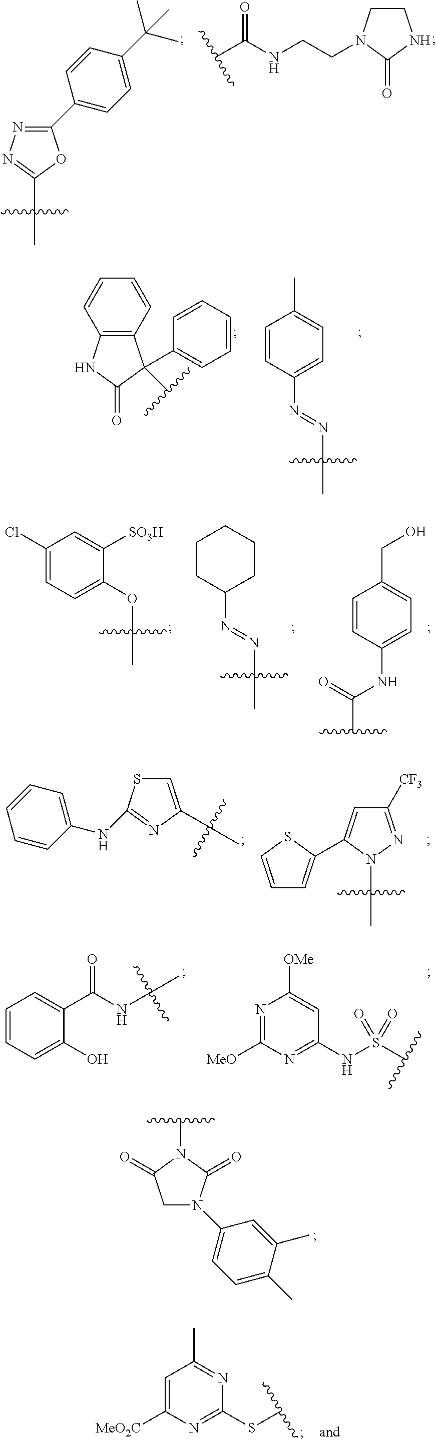

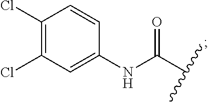 [SMILES: *C(=O)Nc1ccc(Cl)c(Cl)c1]

each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl) SO₂NR⁴—; O—(CH₂)₂₋₄-heterocycle; O—(CH₂)₂₋₄—NR⁴R⁵; O—(CH₂)₂₋₄-heteroaryl;

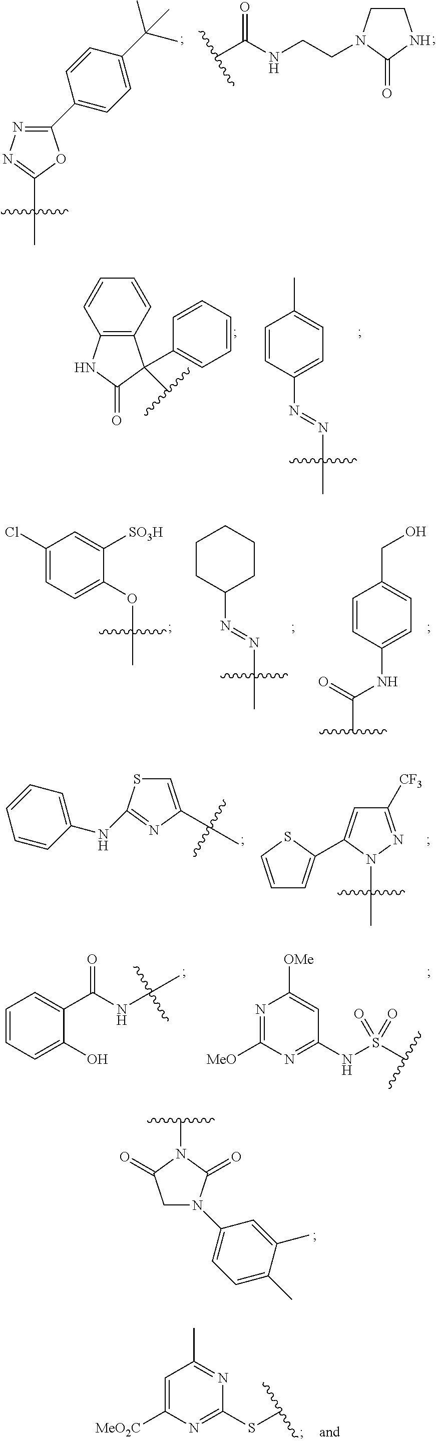

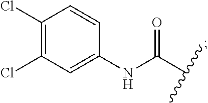 [SMILES: *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]triazol-1-yl), O—(CH₂)₂₋₄-4(1H-[1,2,3]-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

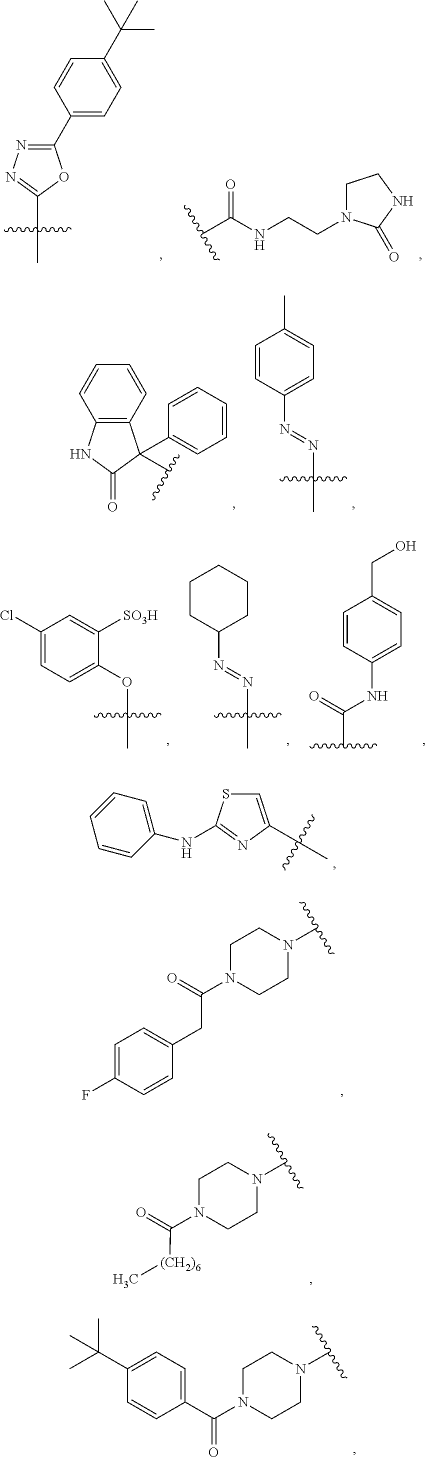

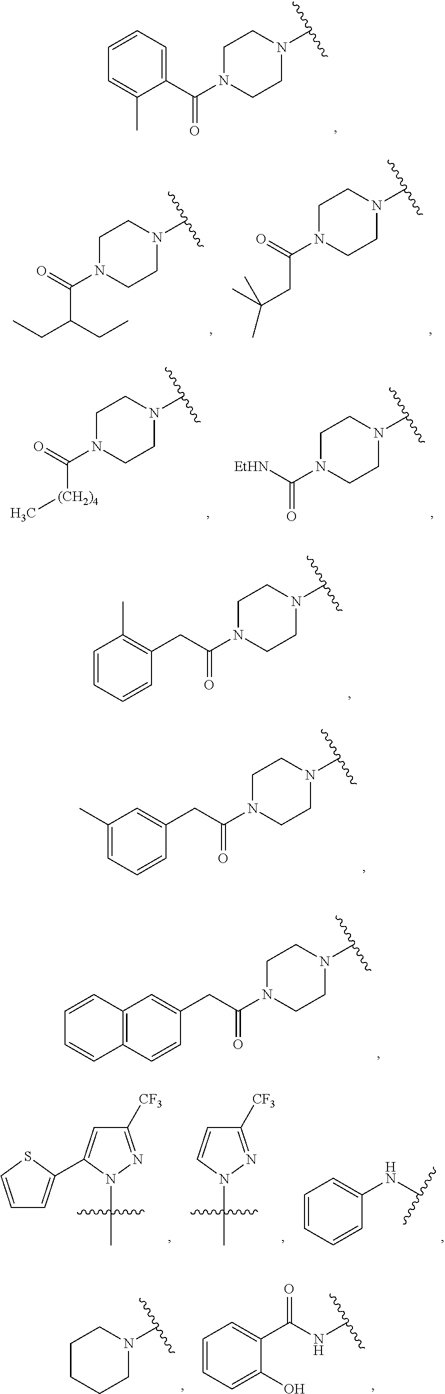

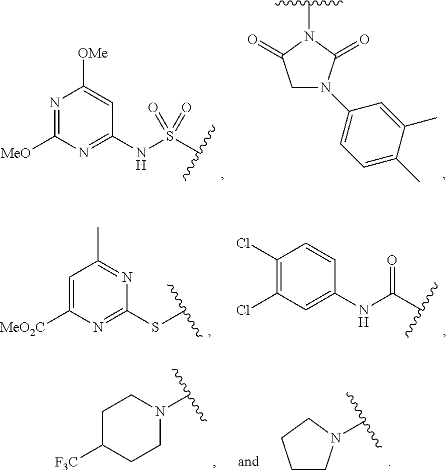 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1 ; *N1CCC(C(F)(F)F)CC1 ; *N1CCCC1]

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be O.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, W is absent or [C(R⁵)₂]_{q} For example, W can be CH₂. In some embodiments, W is absent.

In some embodiments, q is an integer from 1 to 5. In some embodiments, q is 1.

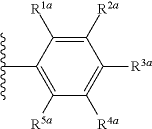

In some embodiments, R³ is

wherein:
R^{2a} and R^{4a} are independently selected from the group consisting of: H, halo, unsubstituted or substituted C₁₋₆alkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, a compound of Formula (V) is:

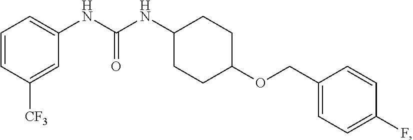 [SMILES: O=C(Nc1cccc(C(F)(F)F)c1)NC1CCC(OCc2ccc(F)cc2)CC1]

or a pharmaceutically acceptable salt thereof.

Yet another aspect provided herein is a compound having the structure of Formula (VI):

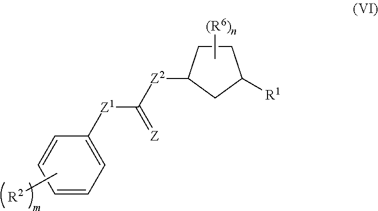

or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

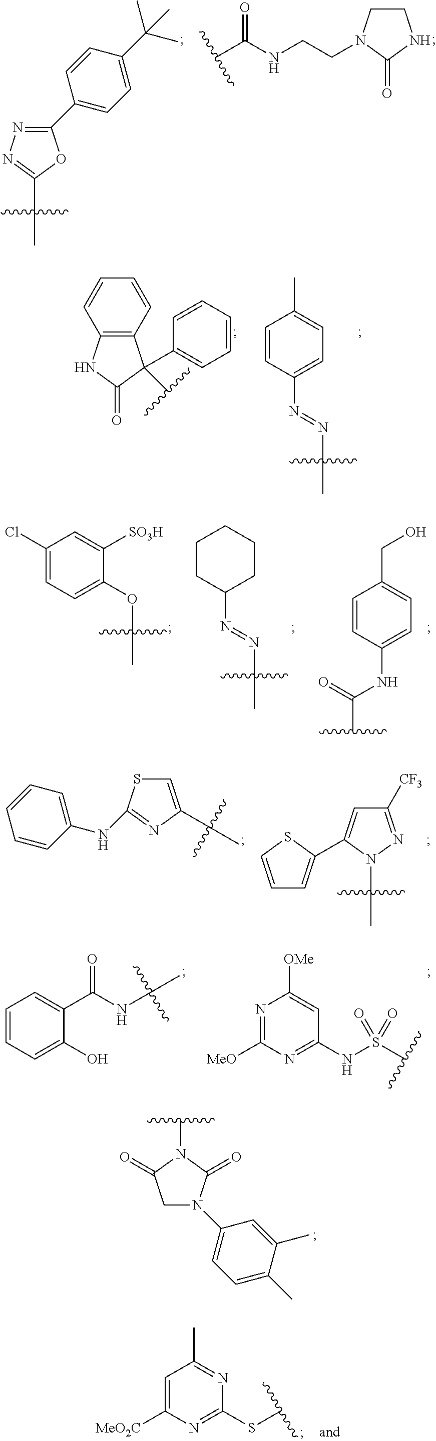

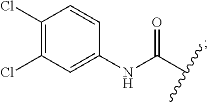 [SMILES: *C(=O)Nc1ccc(Cl)c(Cl)c1]

X is selected from the group consisting of: NR⁴, O, and S(O)ₚ;
m is an integer from 1 to 5;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

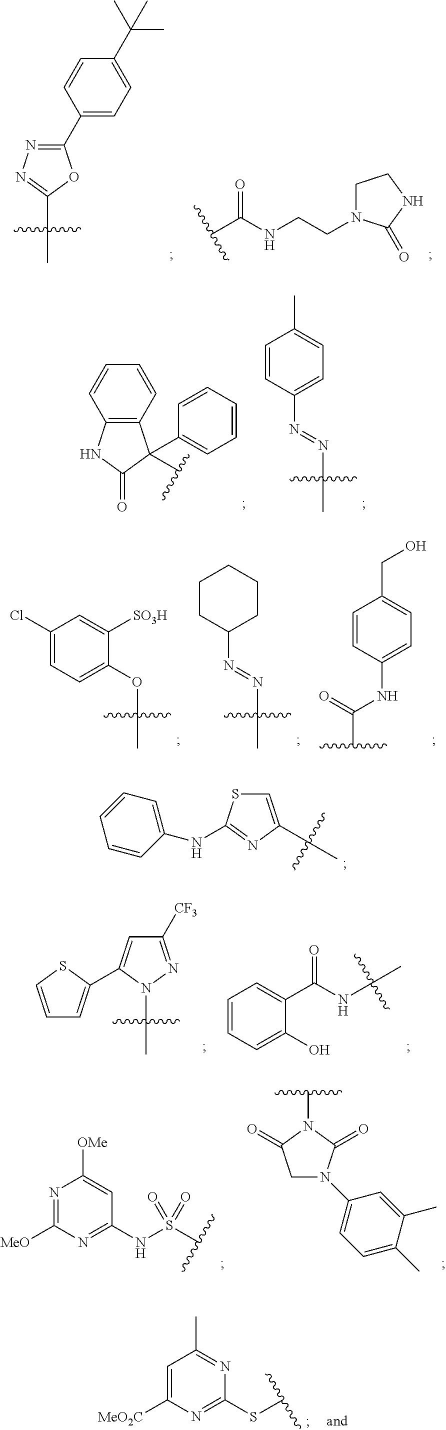

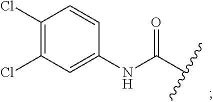 [SMILES: *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, the compound of Formula (VI) has the structure:

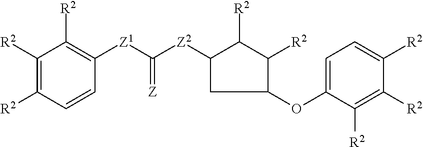

wherein R², Z, Z¹, and Z² are defined herein.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

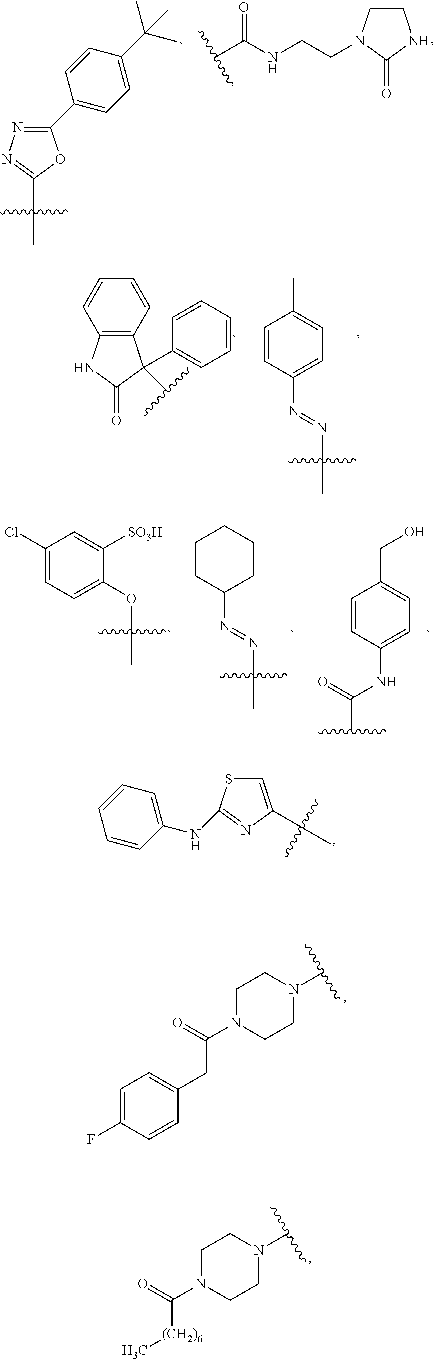

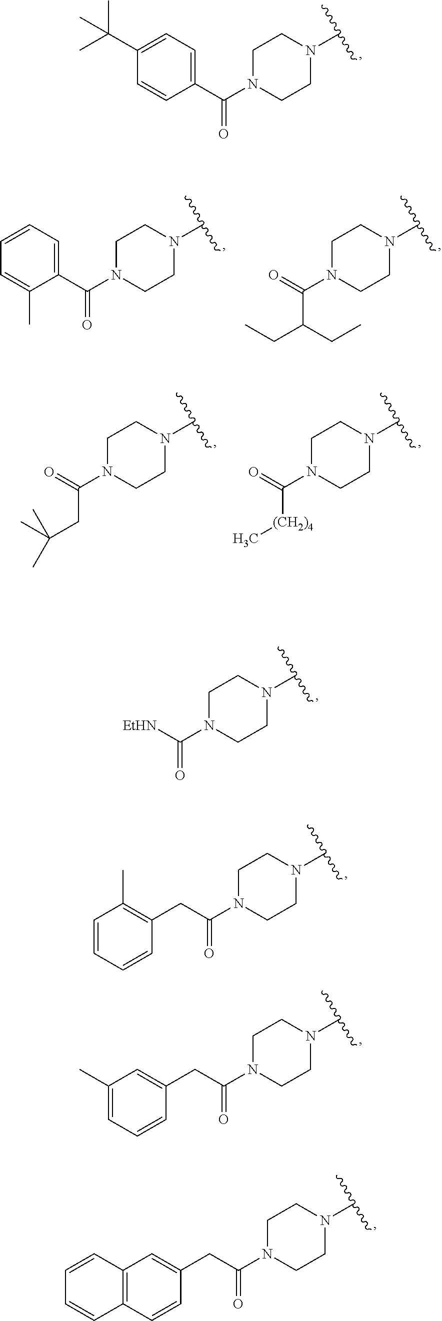

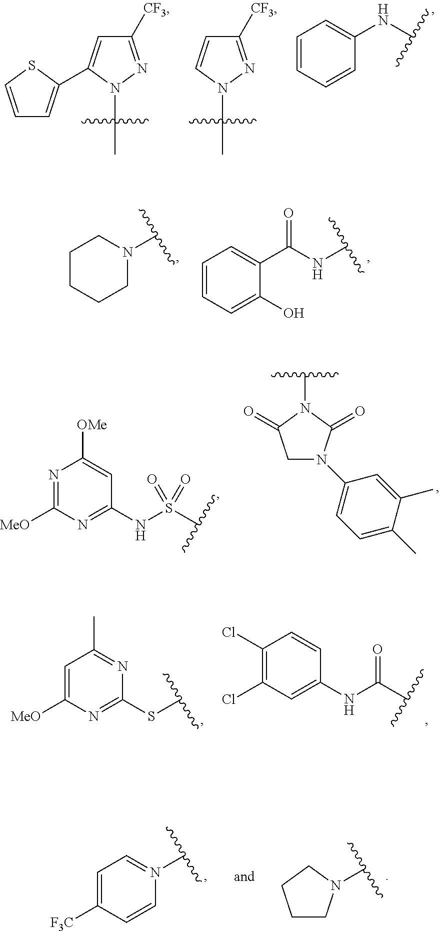

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be O.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, W is absent or [C(R⁵)₂]_{q} For example, W can be CH₂. In some embodiments, W is absent.

In some embodiments, q is an integer from 1 to 5. In some embodiments, q is 1.

In some embodiments, R³ is

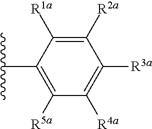

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, a compound of Formula (VI) is selected from the group consisting of:

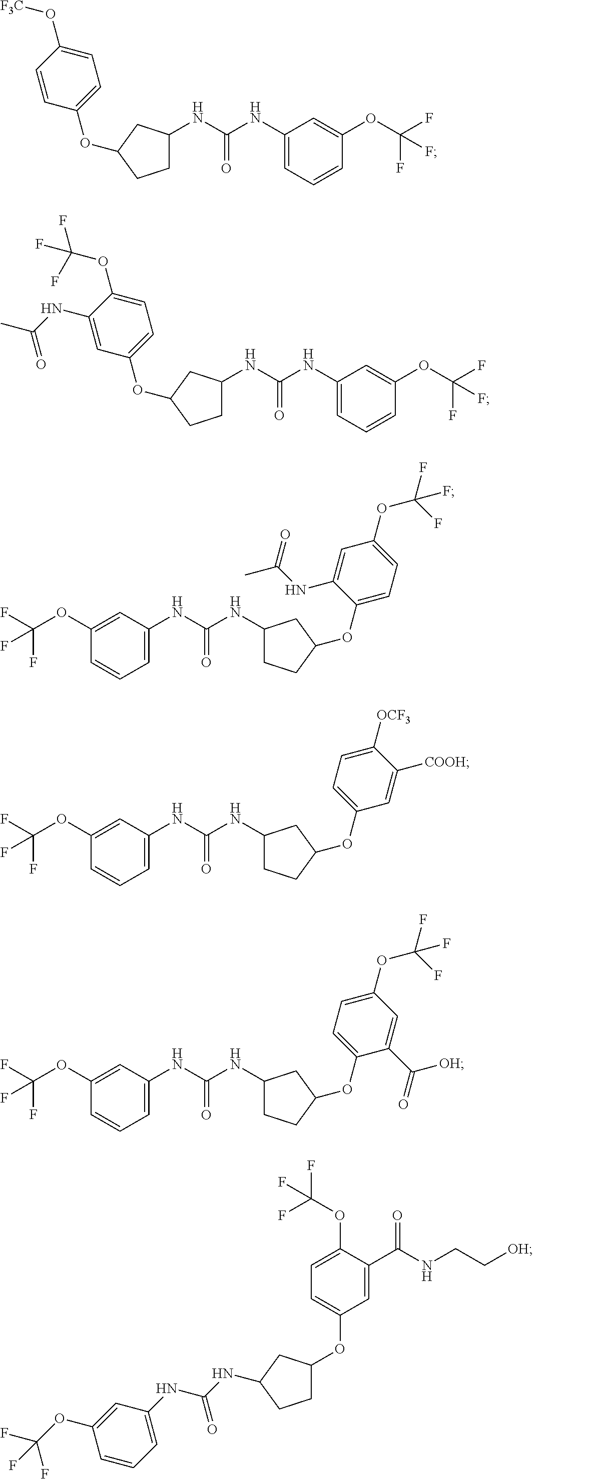

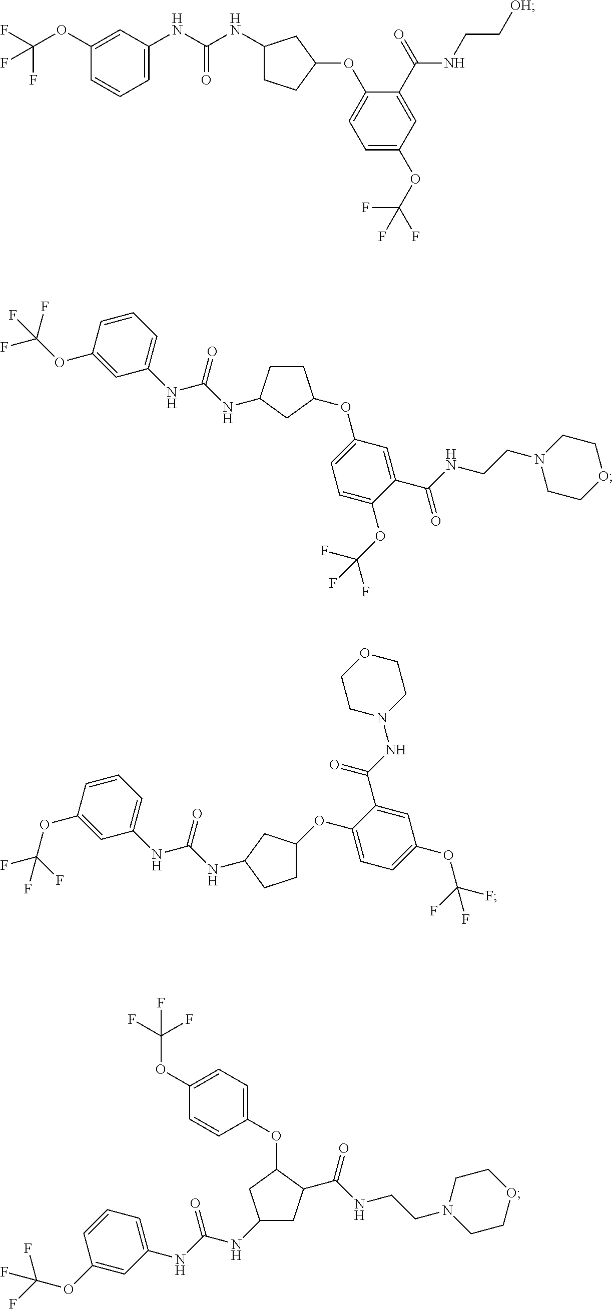 [SMILES: O=C(Nc1cccc(OC(F)(F)F)c1)NC1CCC(Oc2ccc(OC(F)(F)F)cc2C(=O)NCCO)C1 ; O=C(Nc1cccc(OC(F)(F)F)c1)NC1CCC(Oc2ccc(OC(F)(F)F)c(C(=O)NCCN3CCOCC3)c2)C1 ; O=C(Nc1cccc(OC(F)(F)F)c1)NC1CCC(Oc2ccc(OC(F)(F)F)cc2C(=O)NN2CCOCC2)C1 ; O=C(Nc1cccc(OC(F)(F)F)c1)NC1CC(Oc2ccc(OC(F)(F)F)cc2)C(C(=O)NCCN2CCOCC2)C1]

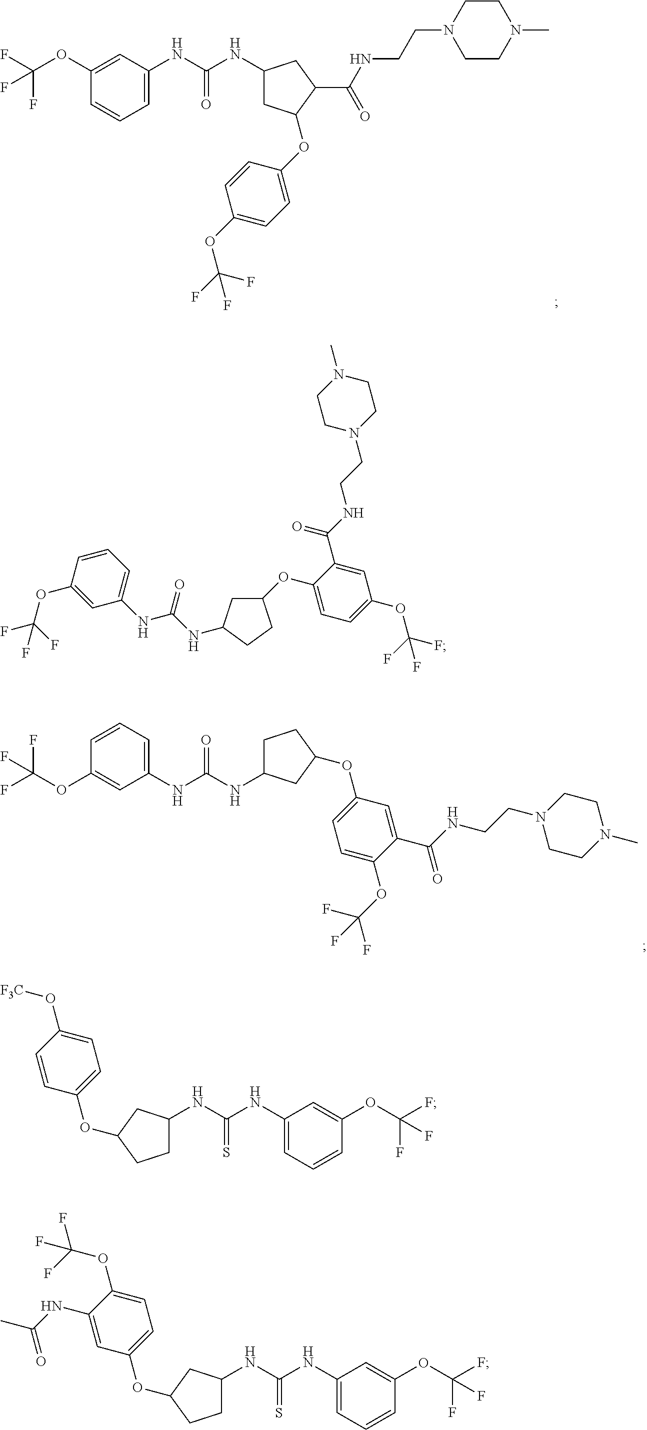

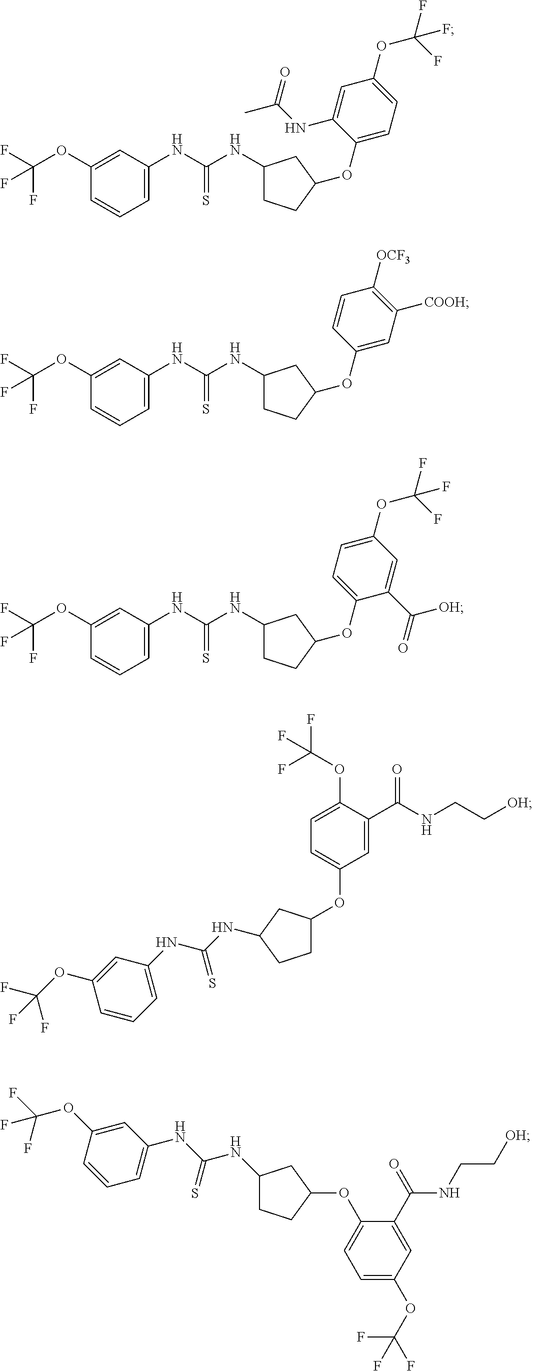 [SMILES: CC(=O)Nc1cc(OC(F)(F)F)ccc1OC1CCC(NC(=S)Nc2cccc(OC(F)(F)F)c2)C1 ; O=C(O)c1cc(OC2CCC(NC(=S)Nc3cccc(OC(F)(F)F)c3)C2)ccc1OC(F)(F)F ; O=C(O)c1cc(OC(F)(F)F)ccc1OC1CCC(NC(=S)Nc2cccc(OC(F)(F)F)c2)C1 ; O=C(NCCO)c1cc(OC2CCC(NC(=S)Nc3cccc(OC(F)(F)F)c3)C2)ccc1OC(F)(F)F ; O=C(NCCO)c1cc(OC(F)(F)F)ccc1OC1CCC(NC(=S)Nc2cccc(OC(F)(F)F)c2)C1]

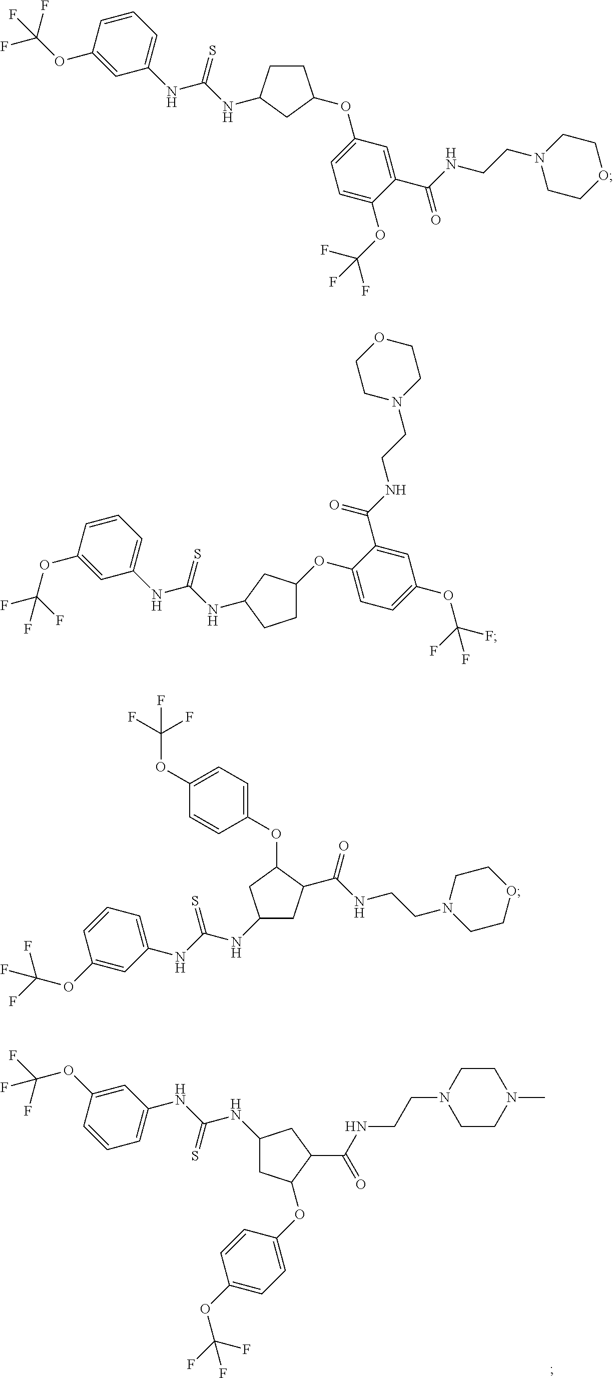 [SMILES: O=C(NCCN1CCOCC1)c1cc(OC2CCC(NC(=S)Nc3cccc(OC(F)(F)F)c3)C2)ccc1OC(F)(F)F ; O=C(NCCN1CCOCC1)c1cc(OC(F)(F)F)ccc1OC1CCC(NC(=S)Nc2cccc(OC(F)(F)F)c2)C1 ; O=C(NCCN1CCOCC1)C1CC(NC(=S)Nc2cccc(OC(F)(F)F)c2)CC1Oc1ccc(OC(F)(F)F)cc1 ; CN1CCN(CCNC(=O)C2CC(NC(=S)Nc3cccc(OC(F)(F)F)c3)CC2Oc2ccc(OC(F)(F)F)cc2)CC1]

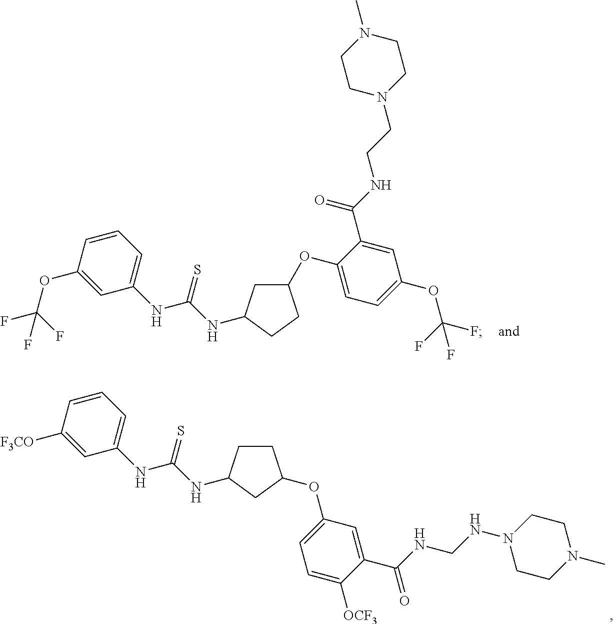 [SMILES: CN1CCN(CCNC(=O)c2cc(OC(F)(F)F)ccc2OC2CCC(NC(=S)Nc3cccc(OC(F)(F)F)c3)C2)CC1 ; CN1CCN(NCNC(=O)c2cc(OC3CCC(NC(=S)Nc4cccc(OC(F)(F)F)c4)C3)ccc2OC(F)(F)F)CC1]

or a pharmaceutically acceptable salt thereof.

Also provided herein is a compound having the structure of Formula (VII):

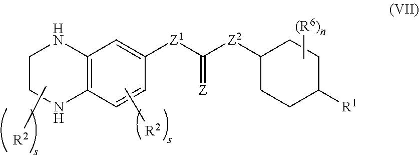

or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted Cₘalkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

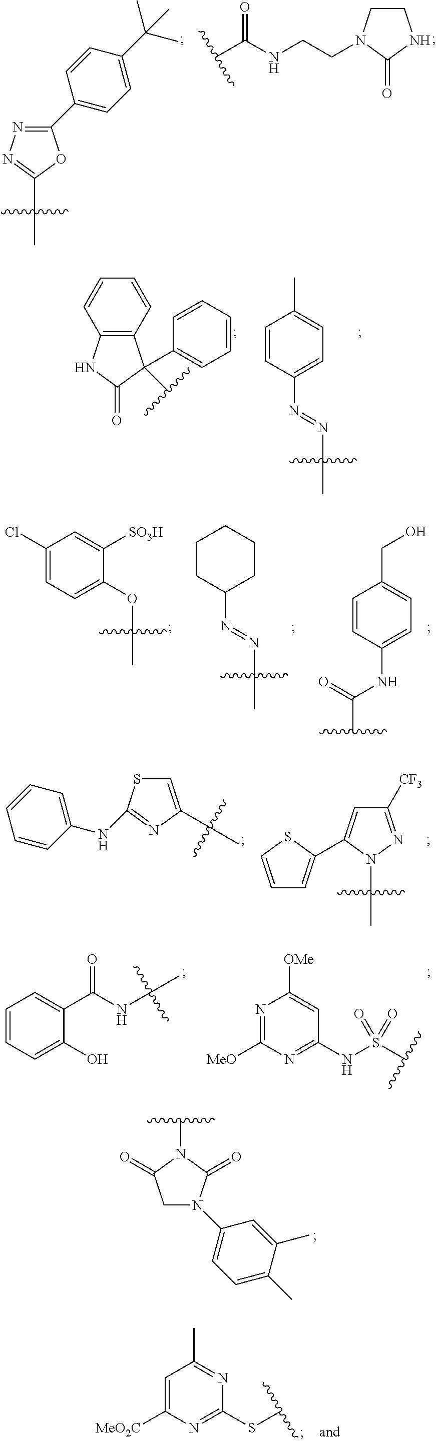

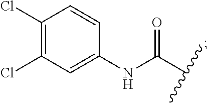 [SMILES: *C(=O)Nc1ccc(Cl)c(Cl)c1]

each s is an integer from 0 to 2;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

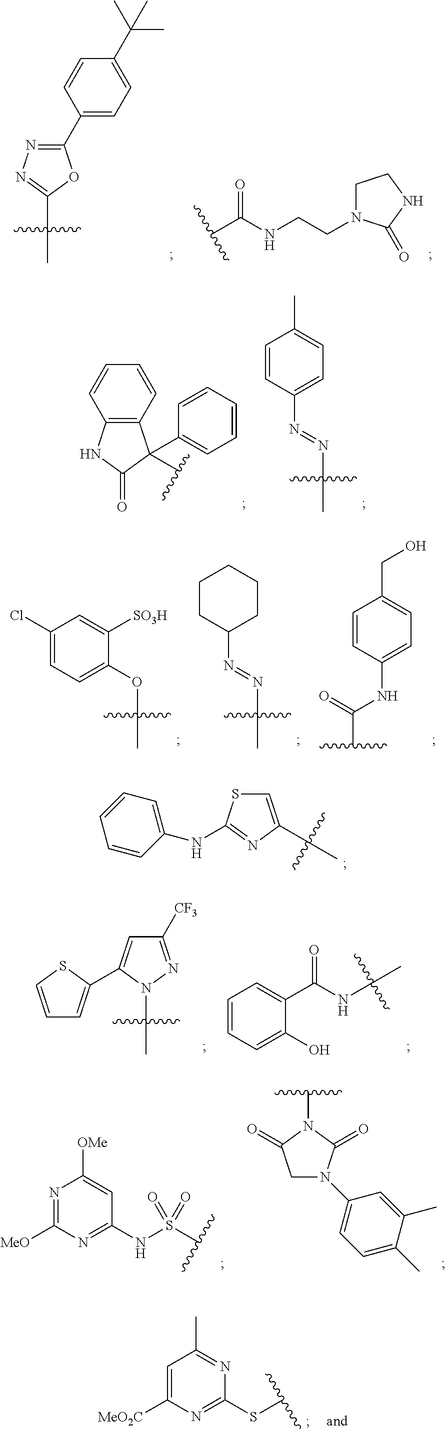

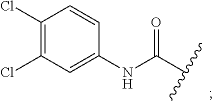 [SMILES: *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, the compound of Formula (VII) has the structure:

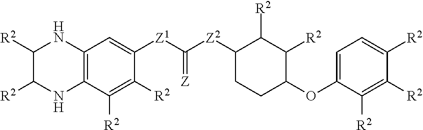

wherein R², Z, Z¹, and Z² are as defined herein.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkylamino, O—(CH₂)₂₋₄-1H-[1,2,3]triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

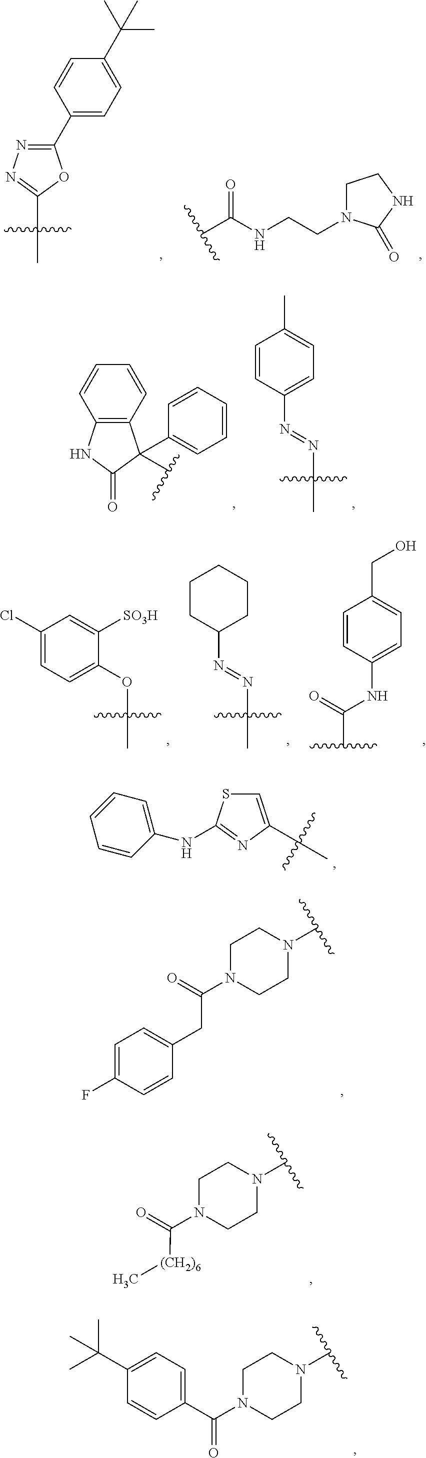

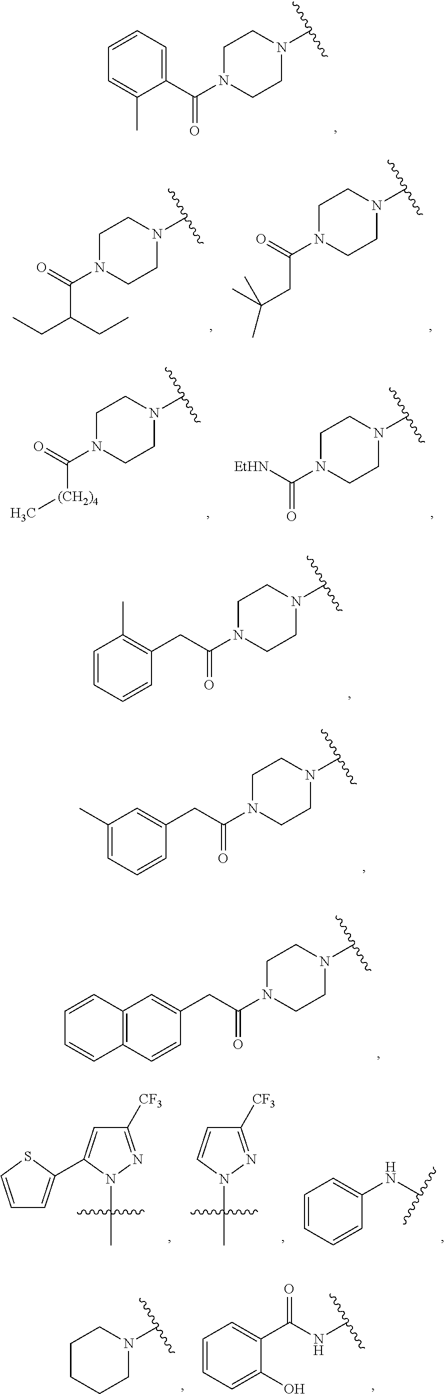

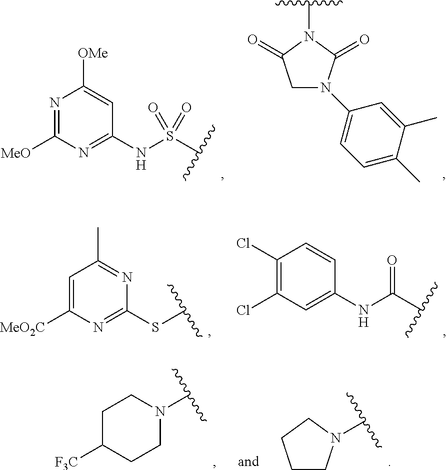 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1 ; *N1CCC(C(F)(F)F)CC1 ; *N1CCCC1]

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be 0.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, s is an integer from 0 to 2.

In some embodiments, W is absent or [C(R⁵)₂]_{q}. For example, W can be CH₂. In some embodiments, W is absent.

In some embodiments, q is an integer from 1 to 5. In some embodiments, q is 1.

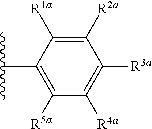

In some embodiments, R³ is

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, C₁₋₆haloalkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

An additional aspect provided herein is a compound having the structure of Formula (VIII):

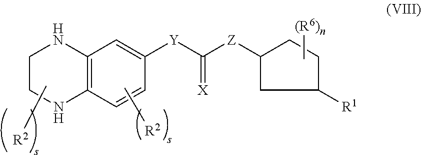

or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

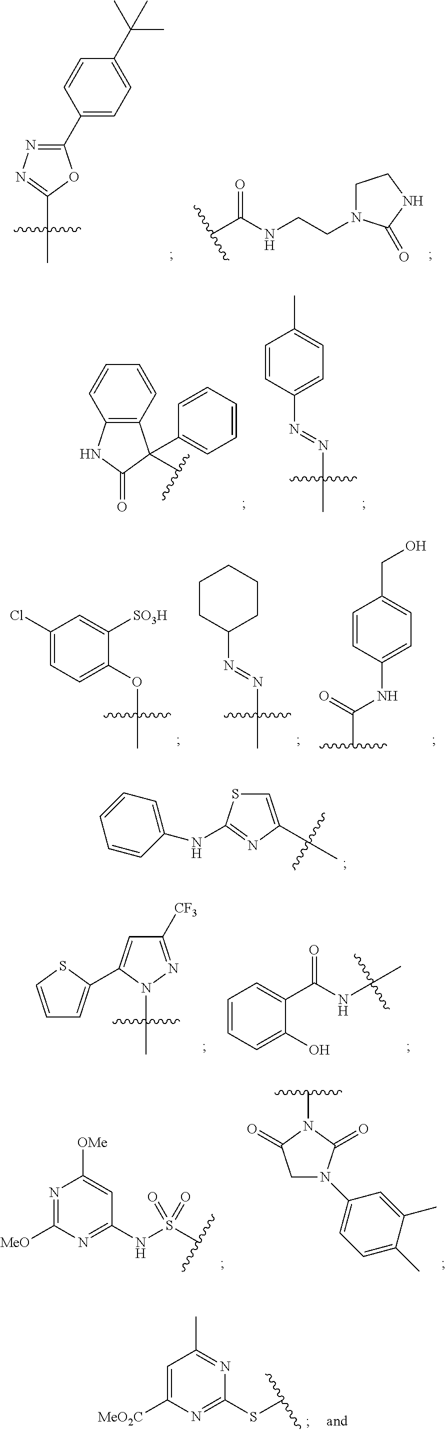

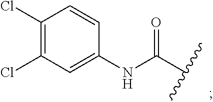 [SMILES: *C(=O)Nc1ccc(Cl)c(Cl)c1]

each s is an integer from 0 to 2;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COW, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

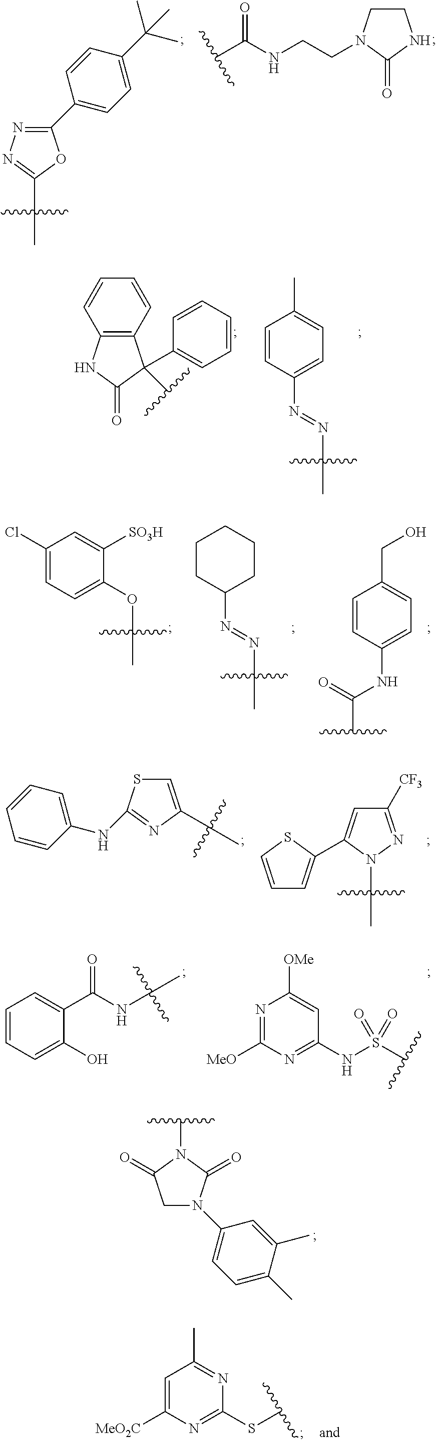

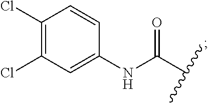 [SMILES: *C(=O)Nc1ccc(Cl)c(Cl)c1]

each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

Another aspect provided herein is a compound having the structure of Formula (IX):

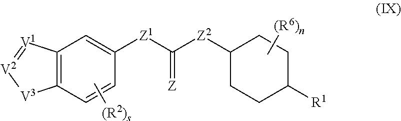

or a pharmaceutically acceptable salt thereof,

wherein:

Z, Z¹, and Z² are each independently selected from the group consisting of: NH, O, and S;

V¹, V², and V³ are each independently selected from the group consisting of: N, O, and S, such that the 5-membered ring is a heteroaryl ring;

R¹ is XWR³;

each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

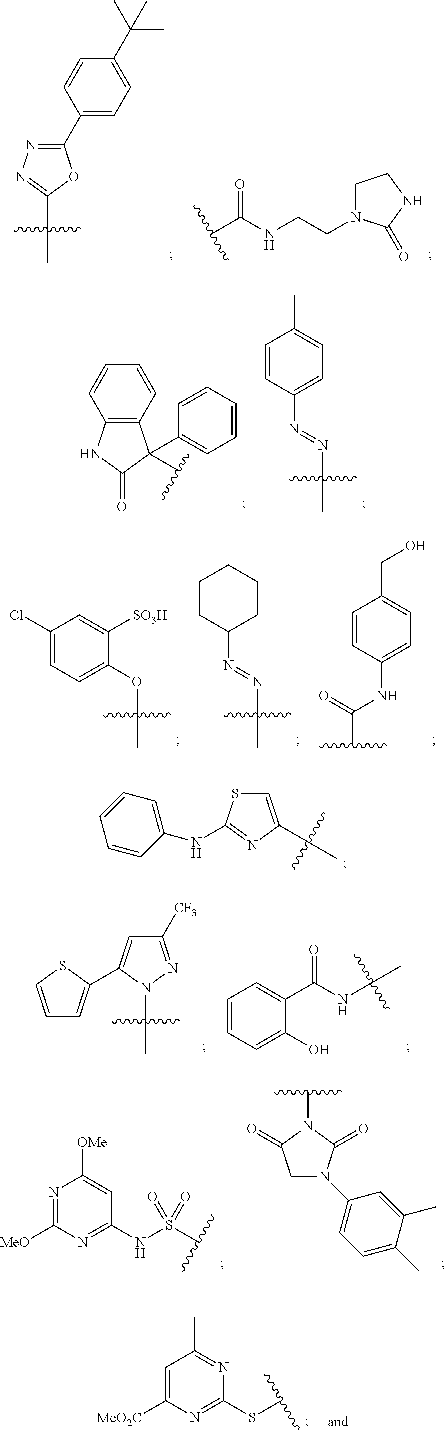

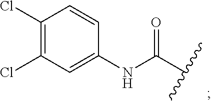 [SMILES: *C(=O)Nc1ccc(Cl)c(Cl)c1]

X is selected from the group consisting of: NR⁴, O, and S(O)ₚ;

each s is an integer from 0 to 2;

n is an integer from 0 to 2;

p is an integer from 0 to 2;

W is absent or [C(R⁵)₂]_{q};

q is an integer from 1 to 5;

R³ is selected from the group consisting of: unsubstituted or substituted aryl, and unsubstituted or substituted heteroaryl;

each R⁴ and R⁵ is independently selected from the group consisting of: H and unsubstituted or substituted C₁₋₆alkyl; and

each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

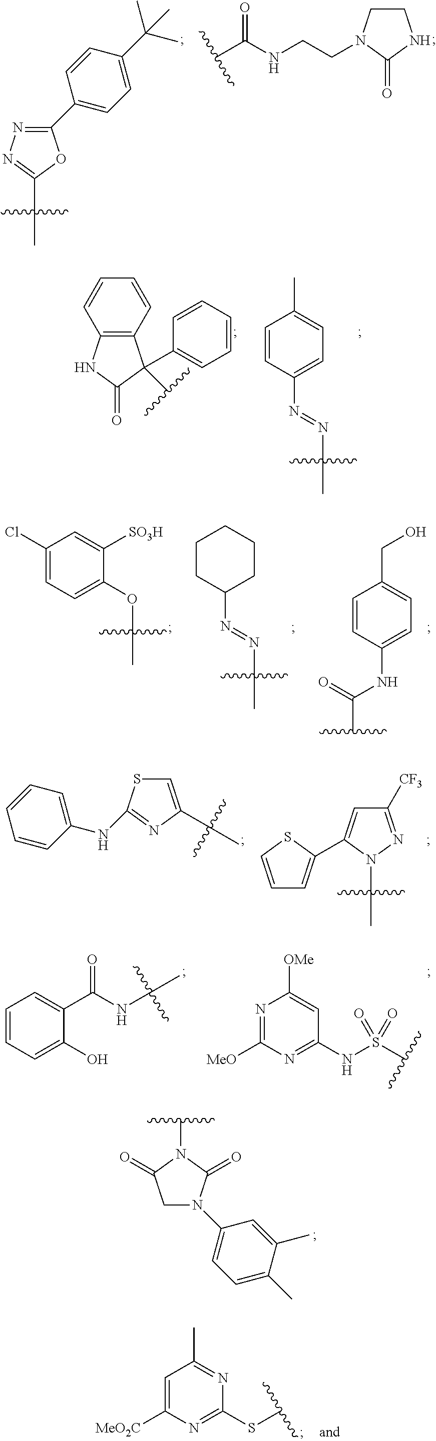

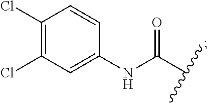 [SMILES: *C(=O)Nc1ccc(Cl)c(Cl)c1]

each R⁷ is independently selected from the group consisting of: H and unsubstituted or substituted C₁₋₆alkyl; and

each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

An additional aspect provided herein is a compound having the structure of Formula (X):

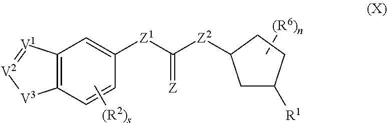

or a pharmaceutically acceptable salt thereof,

wherein:

Z, Z¹, and Z² are each independently selected from the group consisting of: NH, O, and S;

V¹, V², and V³ are each independently selected from the group consisting of: N, O, and S, such that the 5-membered ring is a heteroaryl ring;

R¹ is XWR³;

each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl) SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

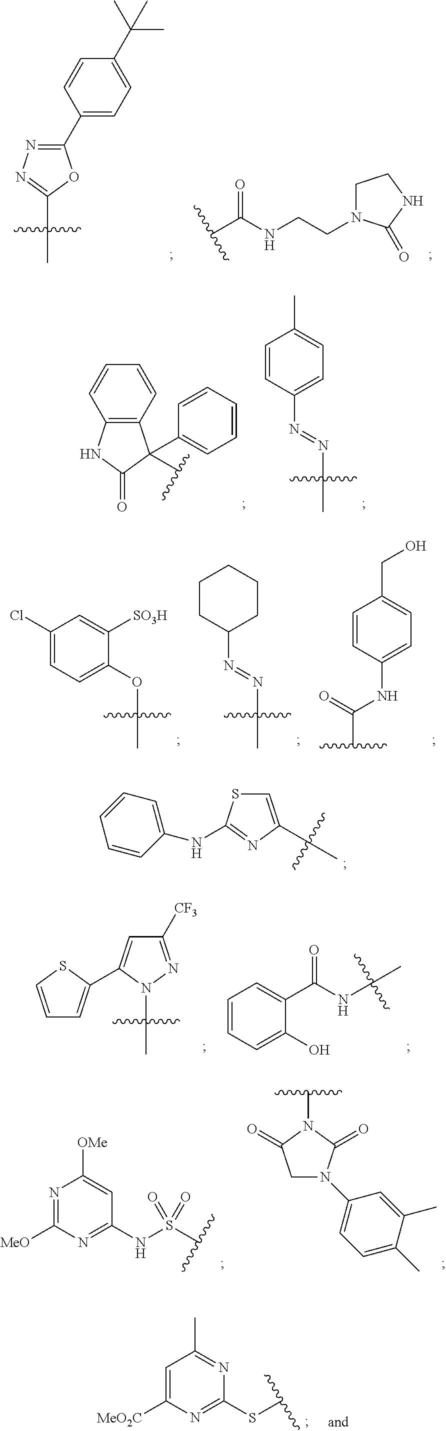

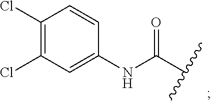 [SMILES: *C(=O)Nc1ccc(Cl)c(Cl)c1]

X is selected from the group consisting of: NR⁴, O, and S(O)ₚ;

each s is an integer from 0 to 2;

n is an integer from 0 to 2;

p is an integer from 0 to 2;

W is absent or [C(R⁵)₂]_{q};

q is an integer from 1 to 5;

R³ is selected from the group consisting of: unsubstituted or substituted aryl, and unsubstituted or substituted heteroaryl;

each R⁴ and R⁵ is independently selected from the group consisting of: H and unsubstituted or substituted C₁₋₆alkyl; and

each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl;

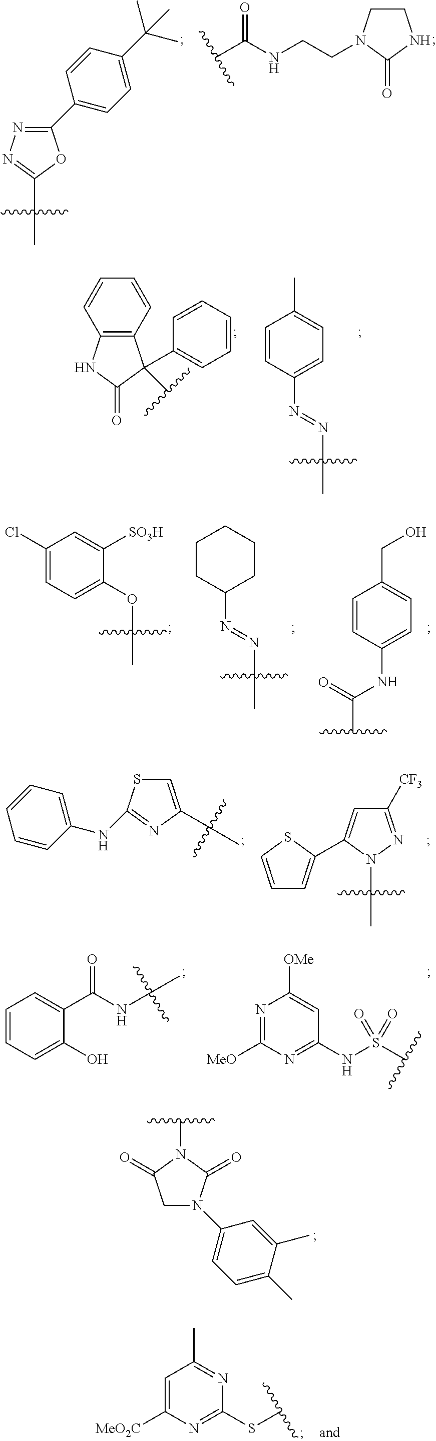

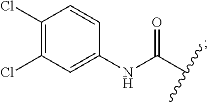 [SMILES: *C(=O)Nc1ccc(Cl)c(Cl)c1]

each R⁷ is independently selected from the group consisting of: H and unsubstituted or substituted C₁₋₆alkyl; and

each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

In some embodiments, the compound of Formula (VIII) has the structure:

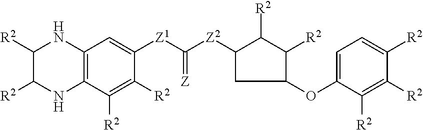

wherein R², Z, Z¹, and Z² are defined herein.

In some embodiments, at least one R² is a substituent meta to the Z¹ attachment on the aryl ring. In some embodiments, each R² is selected from the group consisting of: H, Cl, CH₃, OCH₃, NO₂, OH, F, CF₃, OCF₃, Br, CH₃S, AcHN, (CH₃)₂N, CO—NH—NH₂, SO₂NH₂, C(CH₃)₃, COOCH₂CH₃, COCH₃, O(CH₂)₂CH₃, CHO, CO₂H, OCONH₂, CN, C≡CH, 2-furanol, N-methylacetamido, 1-[1,2,3]triazolyl, 4-[1,2,3]triazolyl, 5-[1,2,3,4]tetrazolyl, guanidine, O—(CH₂)₂₋₄-morpholino, O—(CH₂)₂₋₄-(piperazin-1-yl), O—(CH₂)₂₋₄-(4-methylpiperazin-1-yl), O—(CH₂)₂₋₄-mono- and di-(C₁₋₆-alkyl)amino, O—(CH₂)₂₋₄-1H-[1,2,3]triazol-1-yl), O—(CH₂)₂₋₄-4(1H-[1,2,3]triazol-1-yl), O—(CH₂)₂₋₄-(4-(C₁₋₆-alkyl)-1H-[1,2,3]triazol-1-yl), O—(CH₂)₂₋₄-4-(1-(C₁₋₆-alkyl)-1H-[1,2,3]triazol-1-yl),

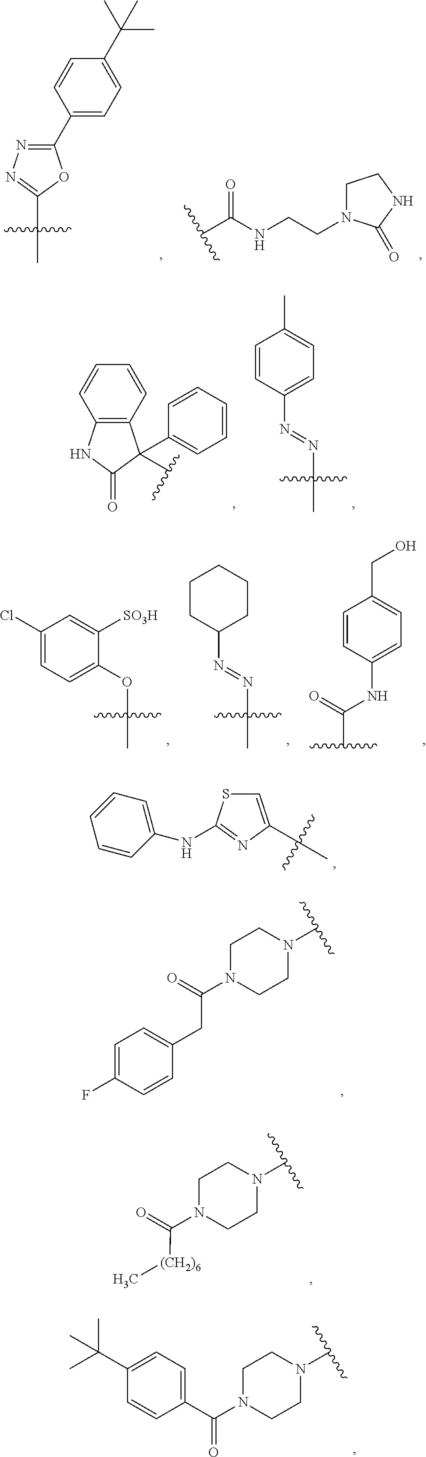

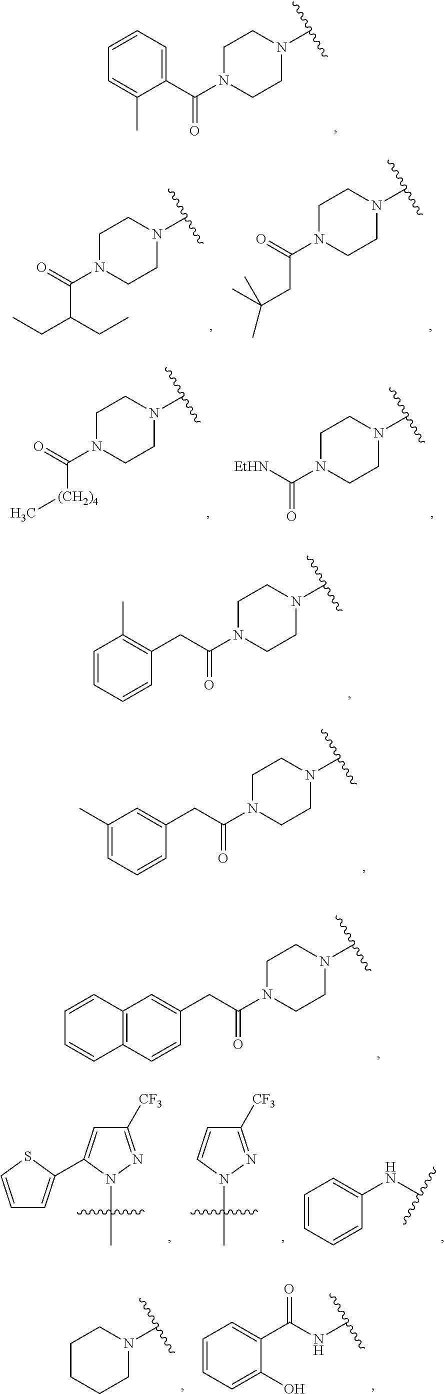

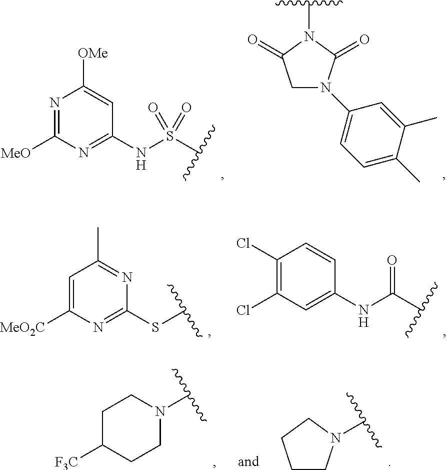 [SMILES: *S(=O)(=O)Nc1cc(OC)nc(OC)n1 ; *N1C(=O)CN(c2ccc(C)c(C)c2)C1=O ; *Sc1nc(C)cc(C(=O)OC)n1 ; *C(=O)Nc1ccc(Cl)c(Cl)c1 ; *N1CCC(C(F)(F)F)CC1 ; *N1CCCC1]

In some embodiments, R² is selected from the group consisting of: unsubstituted or substituted C₁₋₆alkyl, unsubstituted or substituted C₂₋₆alkenyl, unsubstituted or substituted C₂₋₆alkynyl, C₁₋₆haloalkyl, C₁₋₆alkoxy, C₁₋₆haloalkoxy, halo, —SR⁴, —CO₂R⁴, —NO₂, —NR⁴R⁵, and —OH. In some instances, R² is selected from the group consisting of: unsubstituted or substituted C₁₋₆alkyl, C₁₋₆haloalkyl, and C₁₋₆haloalkoxy. In some embodiments, R² is selected from the group consisting of: C₁₋₆haloalkyl and C₁₋₆haloalkoxy. For example, R² can be CF₃ or OCF₃.

In some embodiments, X is selected from the group consisting of: NR⁴, O, and S(O)ₚ. For instance, X can be O.

In some embodiments, m is an integer from 1 to 5. In some embodiments, m is an integer from 1 to 3. For instance, m can be 1.

In some embodiments, n is an integer from 0 to 2.

In some embodiments, p is an integer from 0 to 2. For example, p can be 2.

In some embodiments, s is an integer from 0 to 2.

In some embodiments, W is absent or [C(R⁵)₂]_{q}. For example, W can be CH₂. In some embodiments, W is absent.

In some embodiments, q is an integer from 1 to 5. In some embodiments, q is 1.

In some embodiments, R³ is

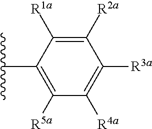

wherein:
R^{2a} and R^{4a} are independently selected from the group consisting of: H, halo, unsubstituted or substituted C₁₋₆alkyl, C₁₋₆haloalkyl, CONR⁴R⁵, CONH(C₁₋₆alkyl)heterocyclyl, CONH(C₁₋₆alkyl)carbocyclyl, CONH(C₁₋₆alkyl)aryl, CONH(C₁₋₆alkyl)heteroaryl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkyl)aryl, (C₁₋₆alkyl)heteroaryl, (C₁₋₆alkoxy)heterocyclyl, (C₁₋₆alkoxyl)aryl, (C₁₋₆alkoxyl)heteroaryl, NR⁴COR⁵, COOR⁴, and C₁₋₆haloalkoxy;
R^{1a}, R^{3a}, and R^{5a} are independently selected from the group consisting of: H, Cl, Br, I, unsubstituted or substituted C₁₋₆alkyl, CONR⁴R⁵, CONH(C₁₋₆alkyl)heterocyclyl, NR⁴R⁵, (C₁₋₆alkyl)NR⁴R⁵, (C₁₋₆alkoxy)NR⁴R⁵, (C₁₋₆alkyl)heterocyclyl, (C₁₋₆alkoxy)heterocyclyl, NR⁴COR⁵, COOR⁴, and C₁₋₆haloalkoxy; and
each R⁴ and R⁵ is independently selected from the group consisting of: H and unsubstituted or substituted C₁₋₆alkyl.

In some embodiments, each R⁴ and R⁵ is independently selected from the group consisting of: H and unsubstituted or substituted C₁₋₆alkyl.

In some embodiments, R⁶ is selected from the group consisting of: H, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —NR⁴R⁵, (C₁₋₆alkyl)NR⁴R⁵, (C₁₋₆alkoxy)NR⁴R⁵, unsubstituted or substituted carbocyclyl, unsubstituted or substituted heterocyclyl, unsubstituted or substituted aryl, and unsubstituted or substituted heteroaryl. In some embodiments, R⁶ is —CONR⁴R⁵ or —CONR⁷R⁸; and n is 1. In some embodiments, R⁶ is —CONH(C₁₋₆alkyl)heterocyclyl. In some instances, R⁶ is —CONHR⁴, wherein R⁴ is an unsubstituted or substituted C₁₋₆alkyl, or —CONH(C₁₋₃alkyl)heterocyclyl.

In some embodiments, each R⁷ is independently selected from the group consisting of: H and unsubstituted or substituted C₁₋₆alkyl.

In some embodiments, Z is O. In some embodiments, Z is S.

In some embodiments, Z¹ and Z² are each NH.

In some embodiments, each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

Non-limiting examples of a compound of Formulas (I), (II), (III), (IV), (V), and/or (VI) include:

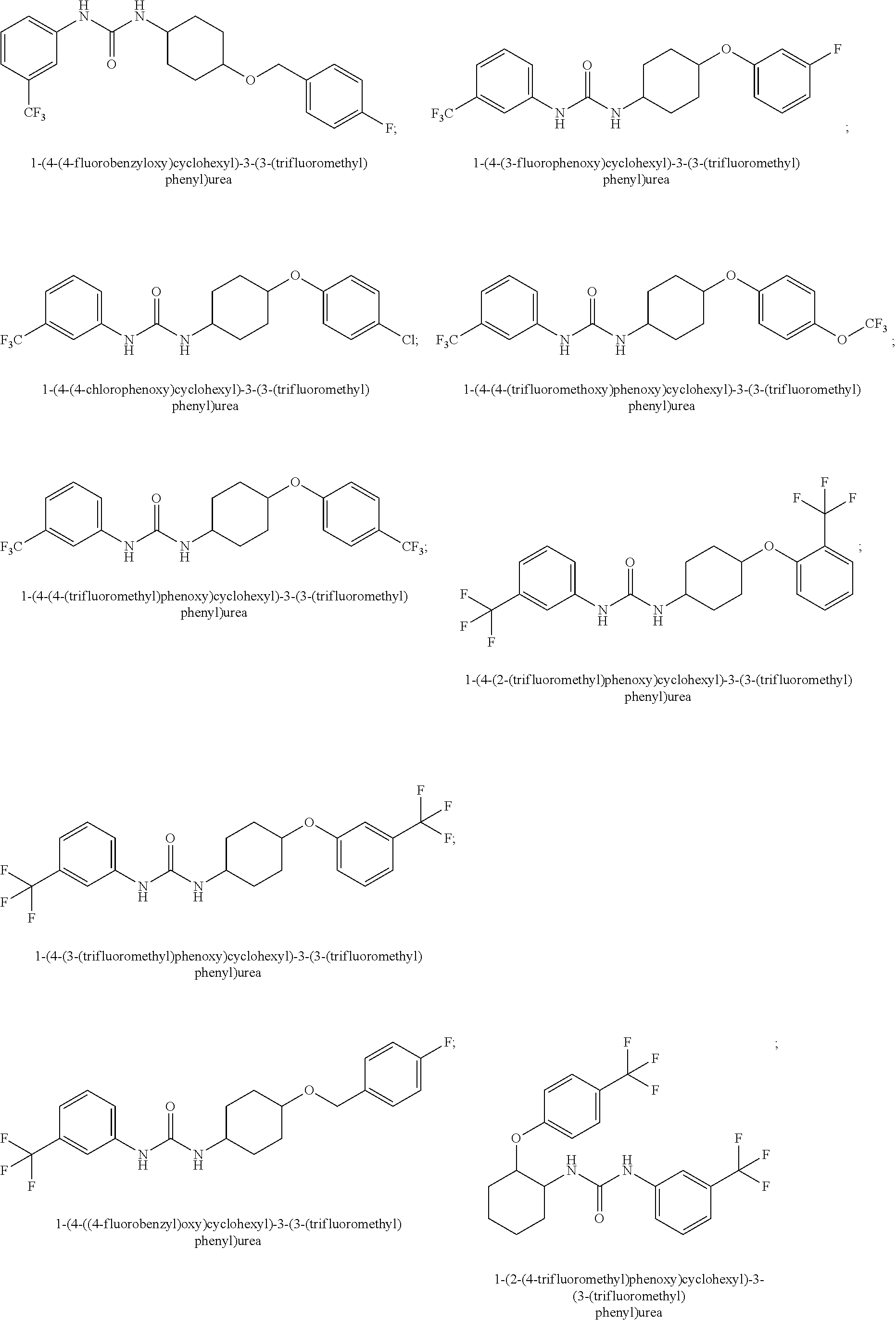

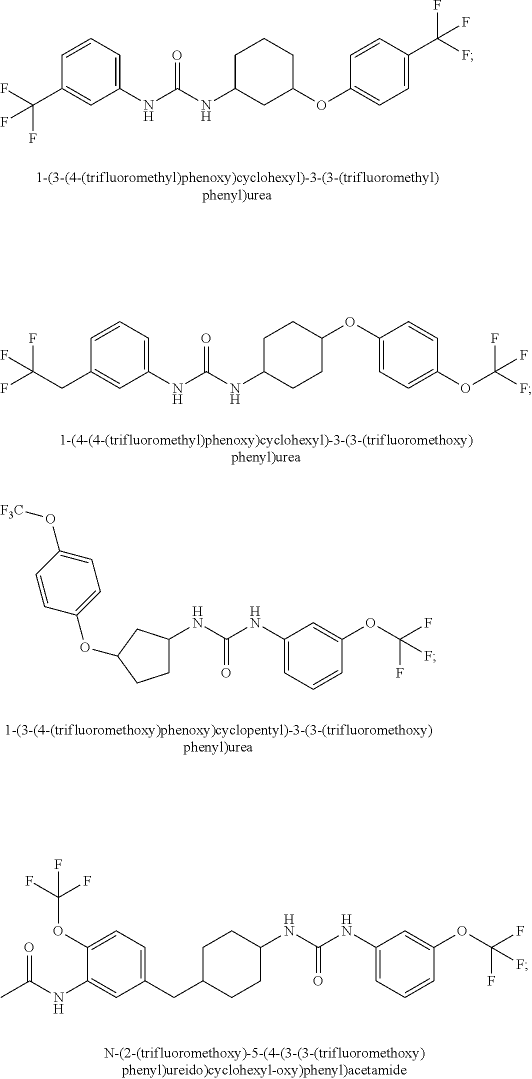 [SMILES: O=C(Nc1cccc(C(F)(F)F)c1)NC1CCCC(Oc2ccc(C(F)(F)F)cc2)C1 ; O=C(Nc1cccc(CC(F)(F)F)c1)NC1CCC(Oc2ccc(OC(F)(F)F)cc2)CC1 ; O=C(Nc1cccc(OC(F)(F)F)c1)NC1CCC(Oc2ccc(OC(F)(F)F)cc2)C1 ; CC(=O)Nc1cc(CC2CCC(NC(=O)Nc3cccc(OC(F)(F)F)c3)CC2)ccc1OC(F)(F)F]

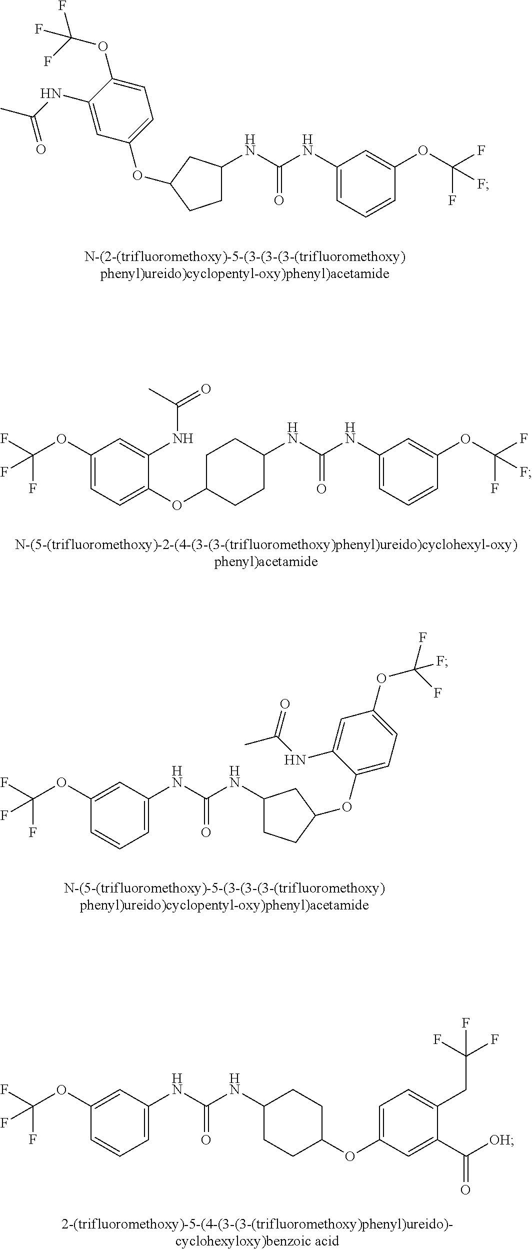 [SMILES: CC(=O)Nc1cc(OC2CCC(NC(=O)Nc3cccc(OC(F)(F)F)c3)C2)ccc1OC(F)(F)F ; CC(=O)Nc1cc(OC(F)(F)F)ccc1OC1CCC(NC(=O)Nc2cccc(OC(F)(F)F)c2)CC1 ; CC(=O)Nc1cc(OC(F)(F)F)ccc1OC1CCC(NC(=O)Nc2cccc(OC(F)(F)F)c2)C1 ; O=C(Nc1cccc(OC(F)(F)F)c1)NC1CCC(Oc2ccc(CC(F)(F)F)c(C(=O)O)c2)CC1]

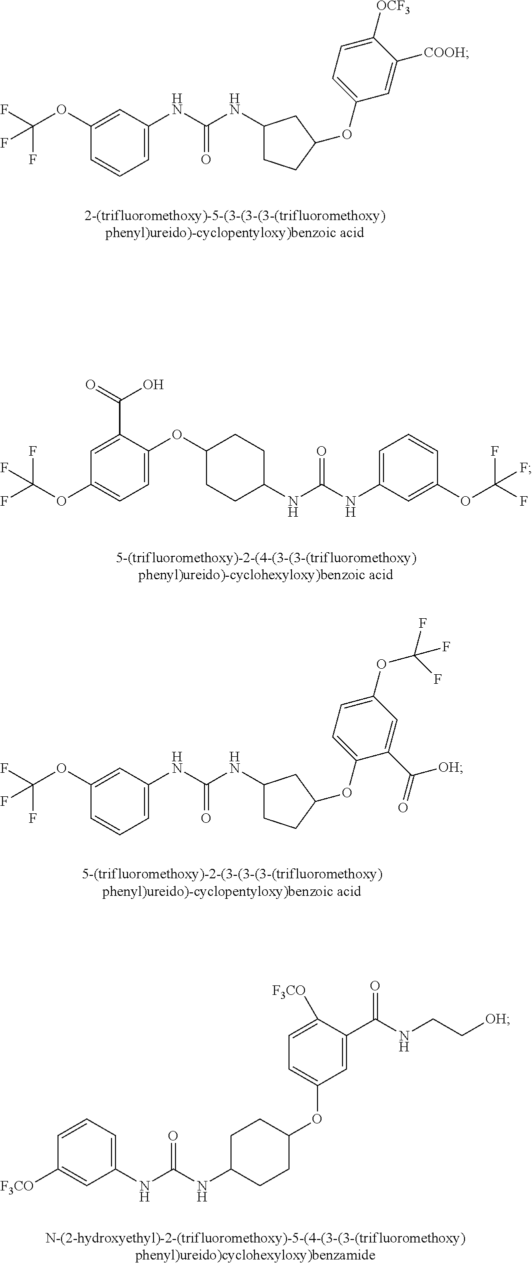 [SMILES: O=C(Nc1cccc(OC(F)(F)F)c1)NC1CCC(Oc2ccc(OC(F)(F)F)c(C(=O)O)c2)C1 ; O=C(Nc1cccc(OC(F)(F)F)c1)NC1CCC(Oc2ccc(OC(F)(F)F)cc2C(=O)O)CC1 ; O=C(Nc1cccc(OC(F)(F)F)c1)NC1CCC(Oc2ccc(OC(F)(F)F)cc2C(=O)O)C1 ; O=C(Nc1cccc(OC(F)(F)F)c1)NC1CCC(Oc2ccc(OC(F)(F)F)c(C(=O)NCCO)c2)CC1]

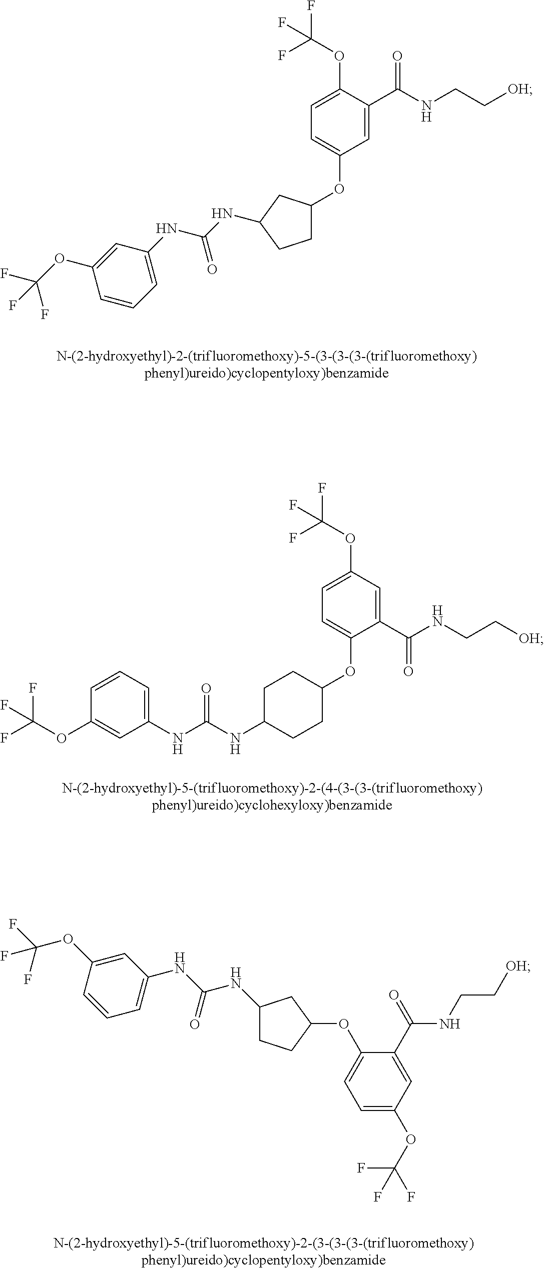 [SMILES: O=C(Nc1cccc(OC(F)(F)F)c1)NC1CCC(Oc2ccc(OC(F)(F)F)c(C(=O)NCCO)c2)C1 ; O=C(Nc1cccc(OC(F)(F)F)c1)NC1CCC(Oc2ccc(OC(F)(F)F)cc2C(=O)NCCO)CC1 ; O=C(Nc1cccc(OC(F)(F)F)c1)NC1CCC(Oc2ccc(OC(F)(F)F)cc2C(=O)NCCO)C1]

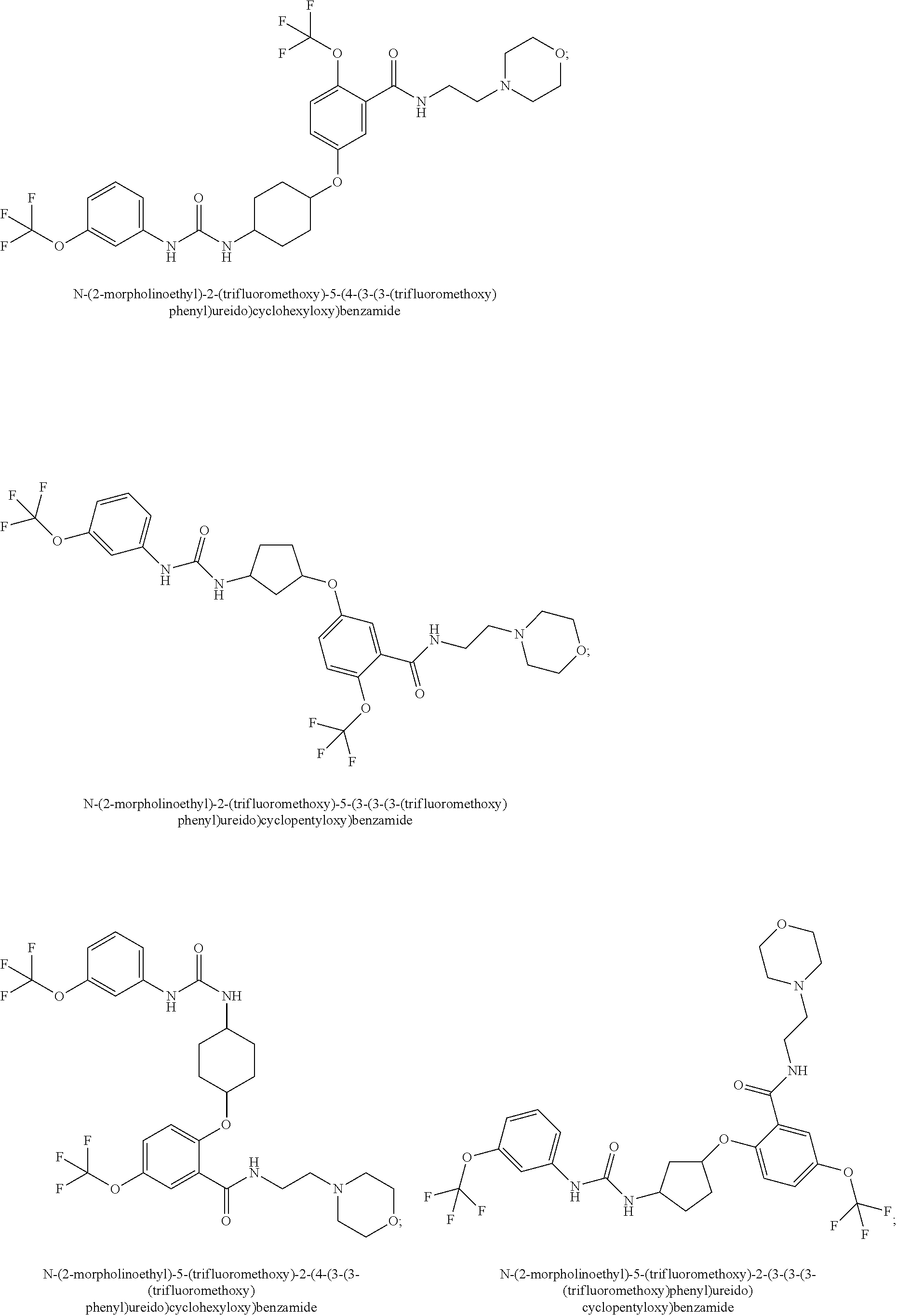

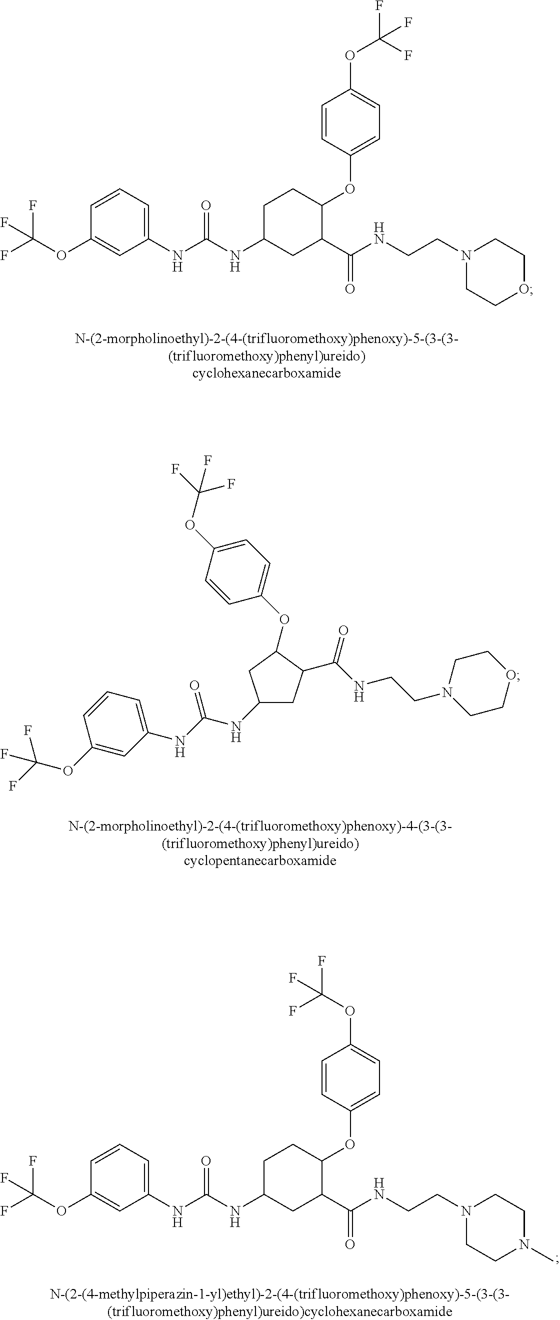 [SMILES: O=C(Nc1cccc(OC(F)(F)F)c1)NC1CCC(Oc2ccc(OC(F)(F)F)cc2)C(C(=O)NCCN2CCOCC2)C1 ; O=C(Nc1cccc(OC(F)(F)F)c1)NC1CC(Oc2ccc(OC(F)(F)F)cc2)C(C(=O)NCCN2CCOCC2)C1 ; CN1CCN(CCNC(=O)C2CC(NC(=O)Nc3cccc(OC(F)(F)F)c3)CCC2Oc2ccc(OC(F)(F)F)cc2)CC1]

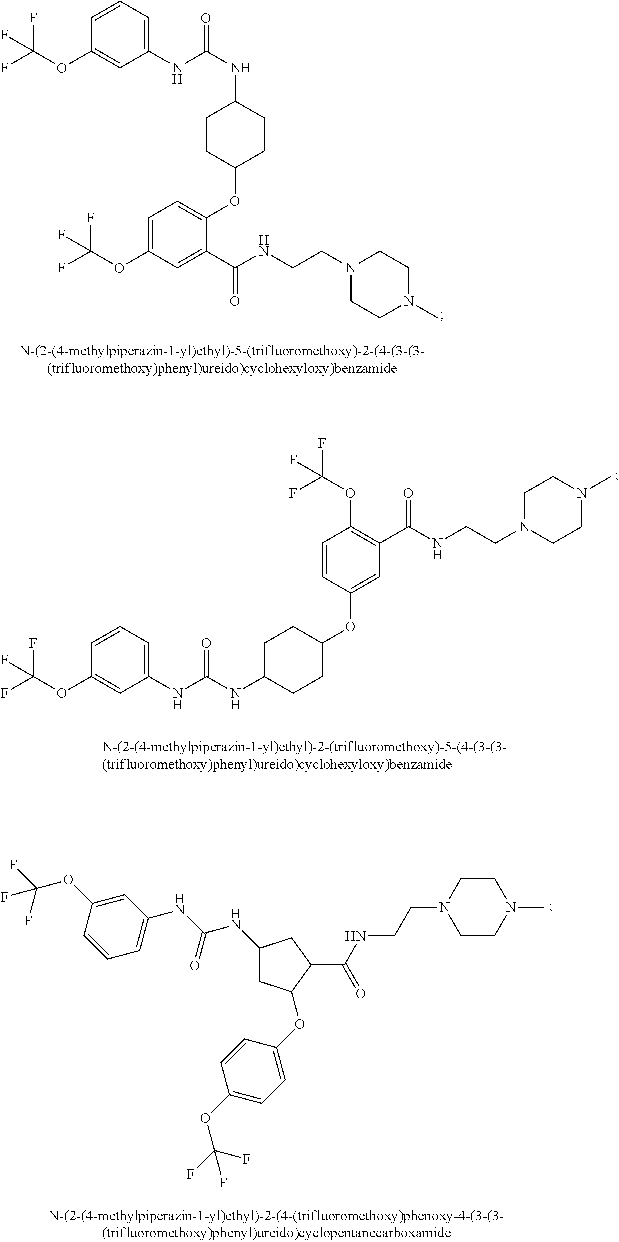 [SMILES: CN1CCN(CCNC(=O)c2cc(OC(F)(F)F)ccc2OC2CCC(NC(=O)Nc3cccc(OC(F)(F)F)c3)CC2)CC1 ; CN1CCN(CCNC(=O)c2cc(OC3CCC(NC(=O)Nc4cccc(OC(F)(F)F)c4)CC3)ccc2OC(F)(F)F)CC1 ; CN1CCN(CCNC(=O)C2CC(NC(=O)Nc3cccc(OC(F)(F)F)c3)CC2Oc2ccc(OC(F)(F)F)cc2)CC1]

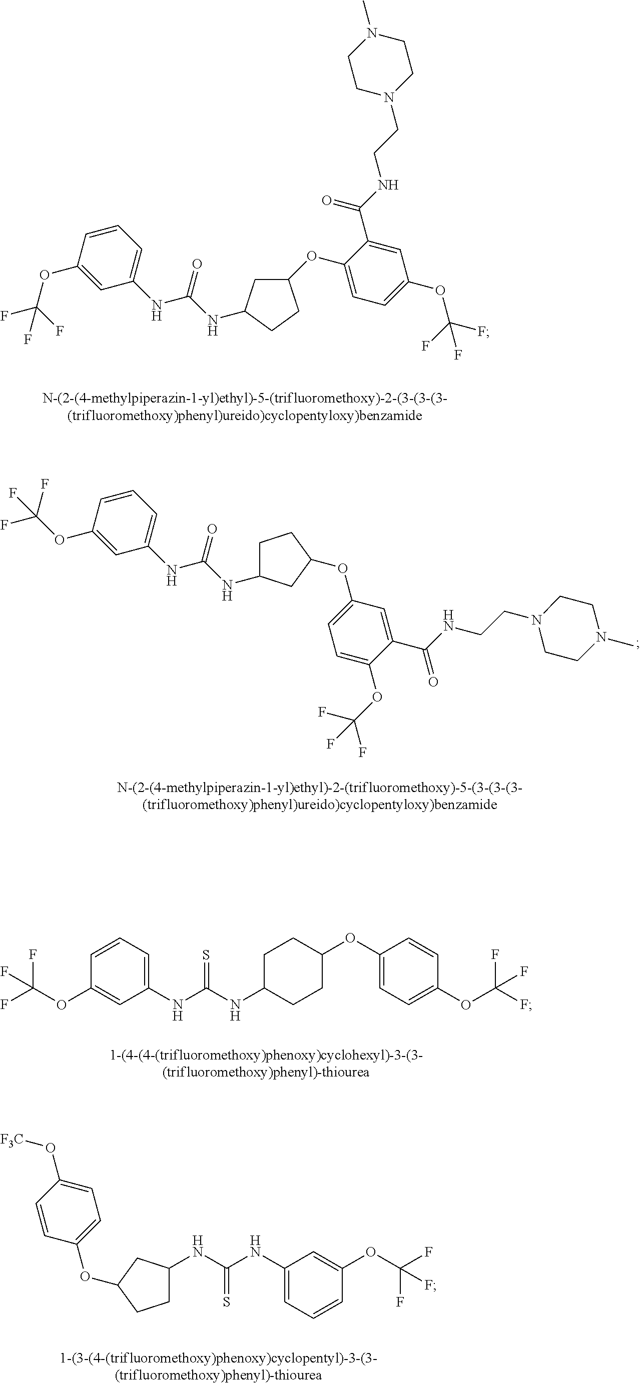 [SMILES: CN1CCN(CCNC(=O)c2cc(OC(F)(F)F)ccc2OC2CCC(NC(=O)Nc3cccc(OC(F)(F)F)c3)C2)CC1 ; CN1CCN(CCNC(=O)c2cc(OC3CCC(NC(=O)Nc4cccc(OC(F)(F)F)c4)C3)ccc2OC(F)(F)F)CC1 ; FC(F)(F)Oc1ccc(OC2CCC(NC(=S)Nc3cccc(OC(F)(F)F)c3)CC2)cc1 ; FC(F)(F)Oc1ccc(OC2CCC(NC(=S)Nc3cccc(OC(F)(F)F)c3)C2)cc1]

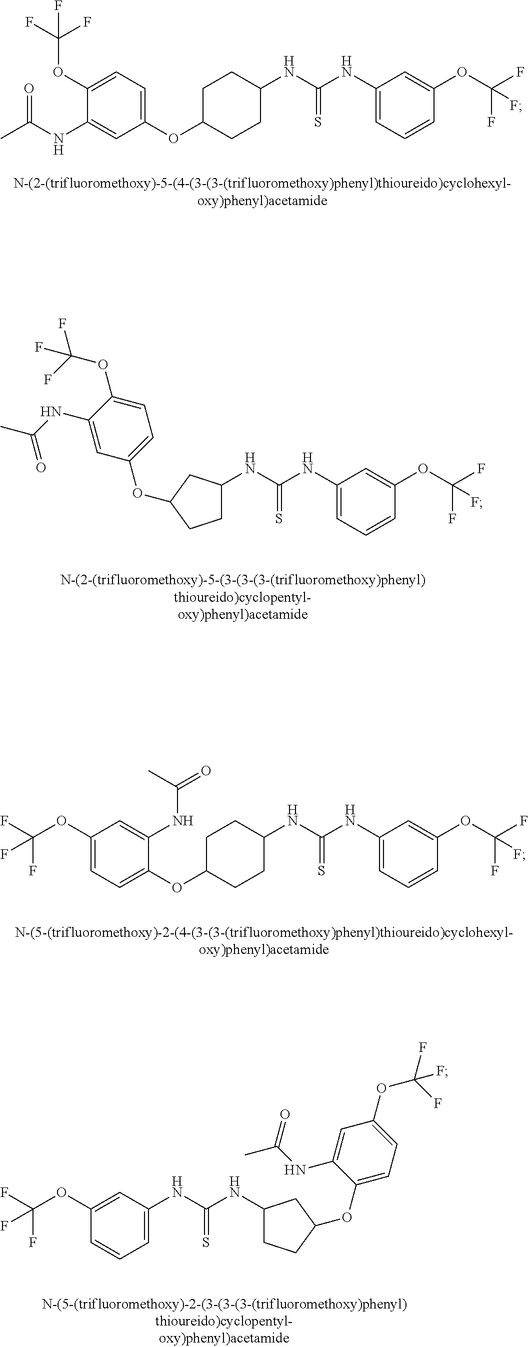 [SMILES: CC(=O)Nc1cc(OC2CCC(NC(=S)Nc3cccc(OC(F)(F)F)c3)CC2)ccc1OC(F)(F)F ; CC(=O)Nc1cc(OC2CCC(NC(=S)Nc3cccc(OC(F)(F)F)c3)C2)ccc1OC(F)(F)F ; CC(=O)Nc1cc(OC(F)(F)F)ccc1OC1CCC(NC(=S)Nc2cccc(OC(F)(F)F)c2)CC1 ; CC(=O)Nc1cc(OC(F)(F)F)ccc1OC1CCC(NC(=S)Nc2cccc(OC(F)(F)F)c2)C1]

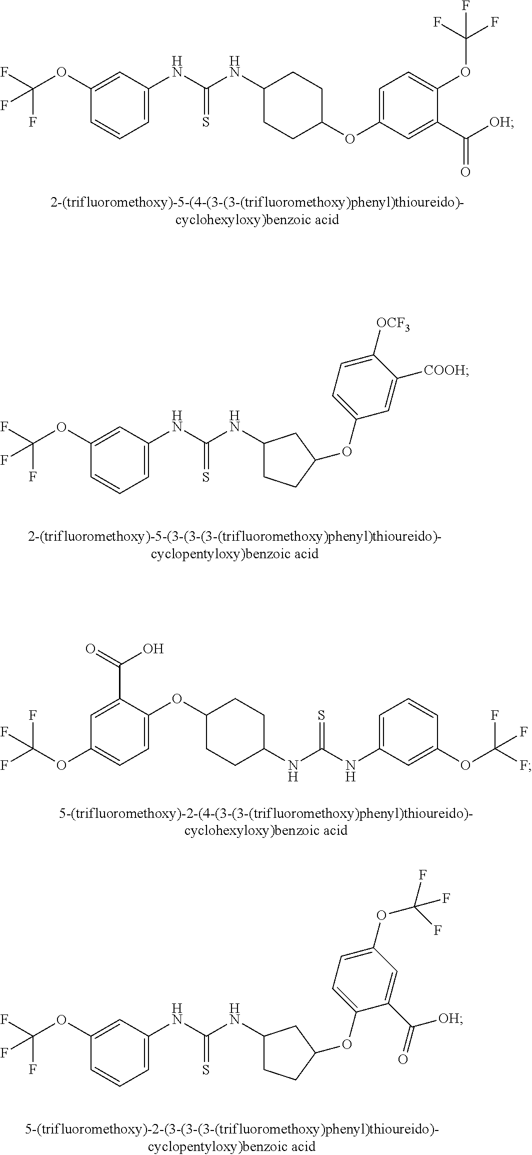 [SMILES: O=C(O)c1cc(OC2CCC(NC(=S)Nc3cccc(OC(F)(F)F)c3)CC2)ccc1OC(F)(F)F ; O=C(O)c1cc(OC2CCC(NC(=S)Nc3cccc(OC(F)(F)F)c3)C2)ccc1OC(F)(F)F ; O=C(O)c1cc(OC(F)(F)F)ccc1OC1CCC(NC(=S)Nc2cccc(OC(F)(F)F)c2)CC1 ; O=C(O)c1cc(OC(F)(F)F)ccc1OC1CCC(NC(=S)Nc2cccc(OC(F)(F)F)c2)C1]

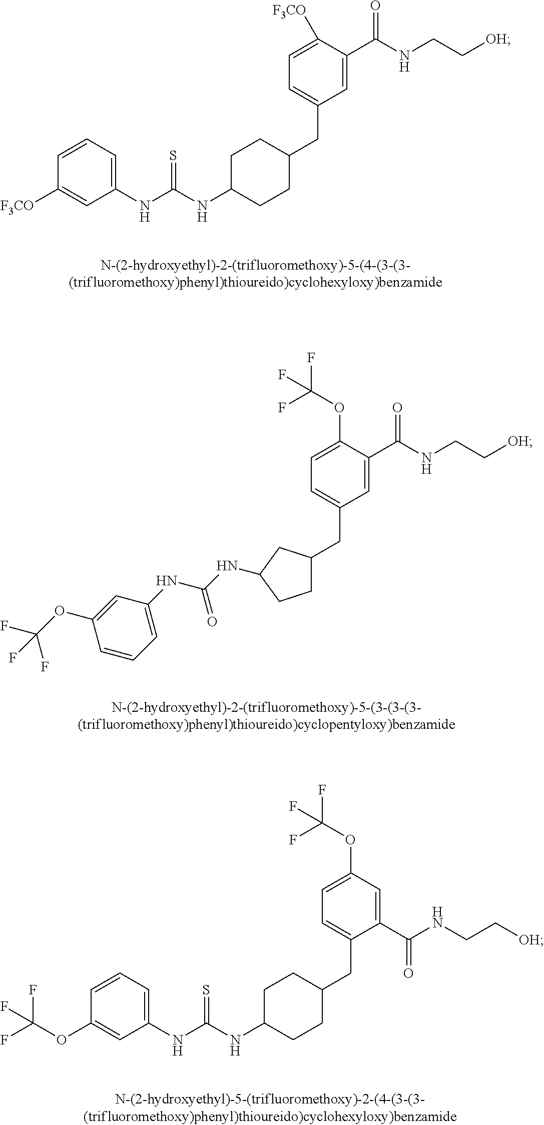 [SMILES: O=C(NCCO)c1cc(CC2CCC(NC(=S)Nc3cccc(OC(F)(F)F)c3)CC2)ccc1OC(F)(F)F ; O=C(Nc1cccc(OC(F)(F)F)c1)NC1CCC(Cc2ccc(OC(F)(F)F)c(C(=O)NCCO)c2)C1 ; O=C(NCCO)c1cc(OC(F)(F)F)ccc1CC1CCC(NC(=S)Nc2cccc(OC(F)(F)F)c2)CC1]

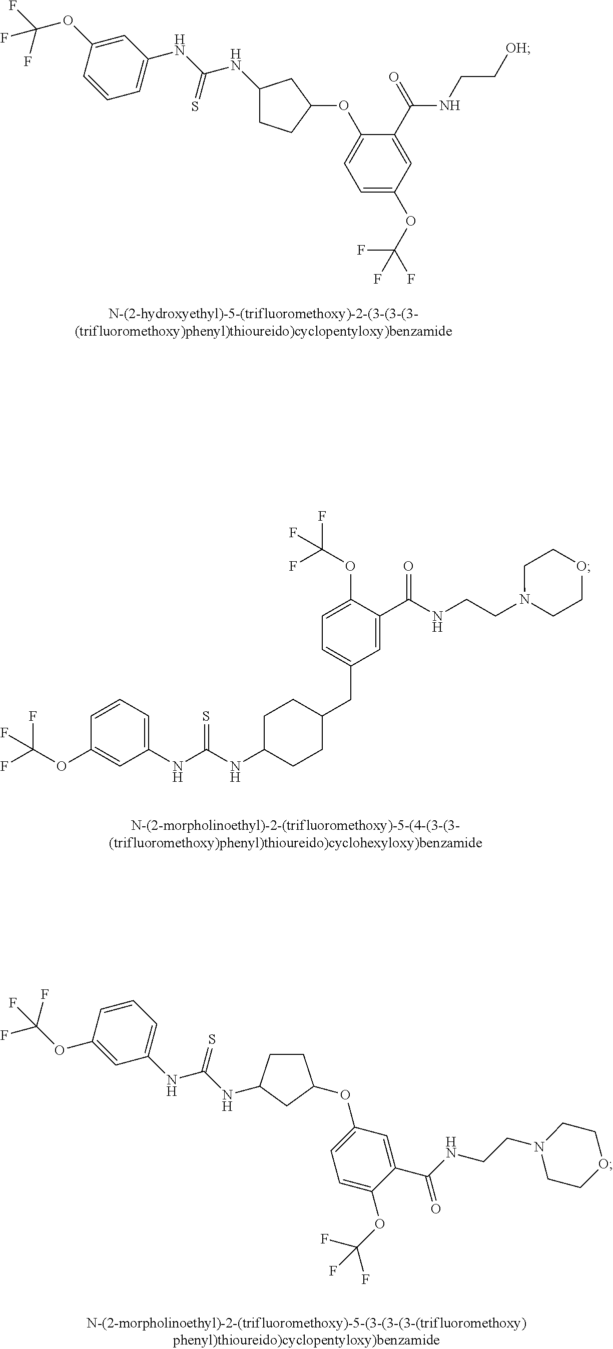 [SMILES: O=C(NCCO)c1cc(OC(F)(F)F)ccc1OC1CCC(NC(=S)Nc2cccc(OC(F)(F)F)c2)C1 ; O=C(NCCN1CCOCC1)c1cc(CC2CCC(NC(=S)Nc3cccc(OC(F)(F)F)c3)CC2)ccc1OC(F)(F)F ; O=C(NCCN1CCOCC1)c1cc(OC2CCC(NC(=S)Nc3cccc(OC(F)(F)F)c3)C2)ccc1OC(F)(F)F]

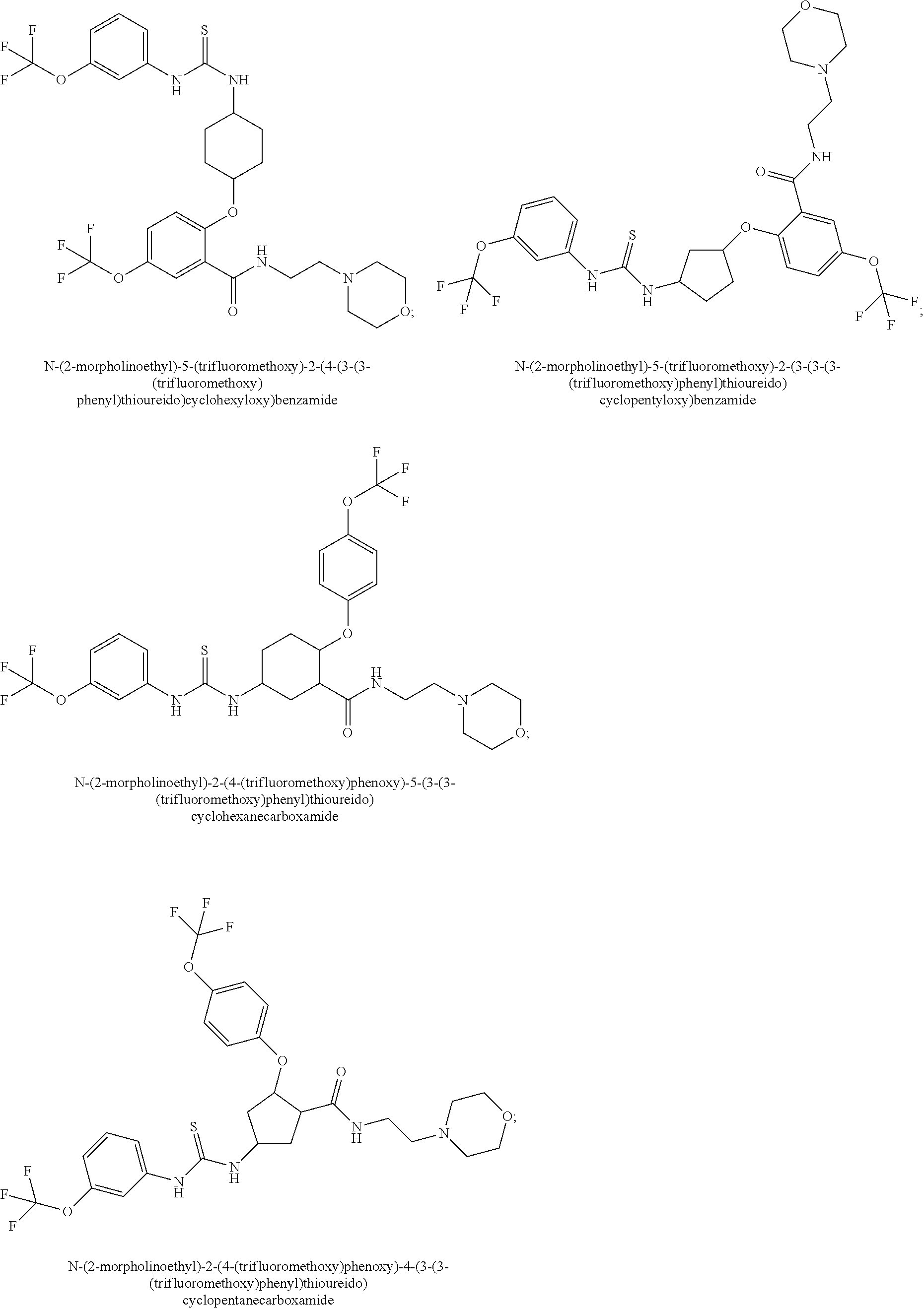

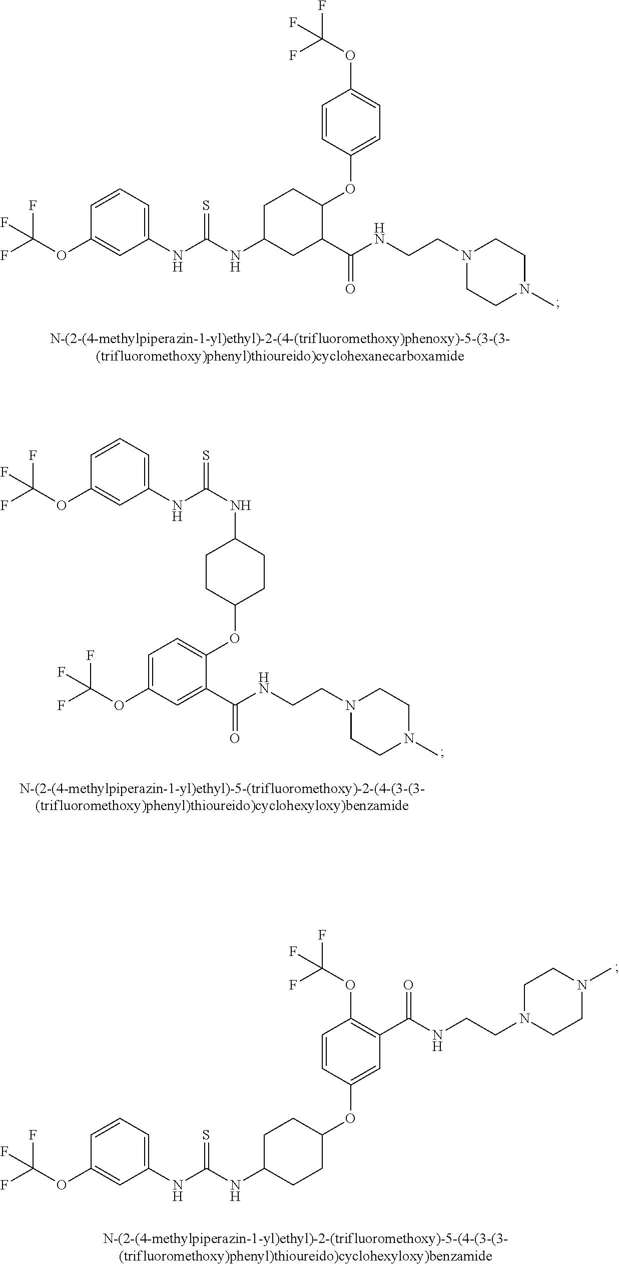 [SMILES: CN1CCN(CCNC(=O)C2CC(NC(=S)Nc3cccc(OC(F)(F)F)c3)CCC2Oc2ccc(OC(F)(F)F)cc2)CC1 ; CN1CCN(CCNC(=O)c2cc(OC(F)(F)F)ccc2OC2CCC(NC(=S)Nc3cccc(OC(F)(F)F)c3)CC2)CC1 ; CN1CCN(CCNC(=O)c2cc(OC3CCC(NC(=S)Nc4cccc(OC(F)(F)F)c4)CC3)ccc2OC(F)(F)F)CC1]

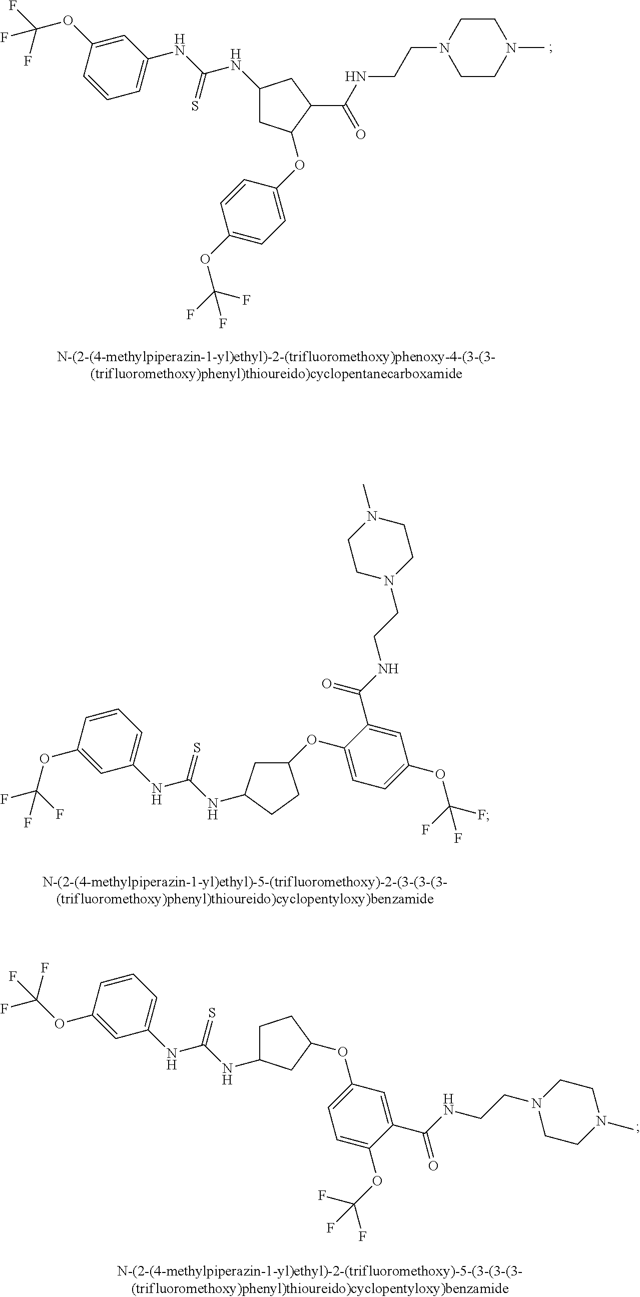 [SMILES: CN1CCN(CCNC(=O)C2CC(NC(=S)Nc3cccc(OC(F)(F)F)c3)CC2Oc2ccc(OC(F)(F)F)cc2)CC1 ; CN1CCN(CCNC(=O)c2cc(OC(F)(F)F)ccc2OC2CCC(NC(=S)Nc3cccc(OC(F)(F)F)c3)C2)CC1 ; CN1CCN(CCNC(=O)c2cc(OC3CCC(NC(=S)Nc4cccc(OC(F)(F)F)c4)C3)ccc2OC(F)(F)F)CC1]

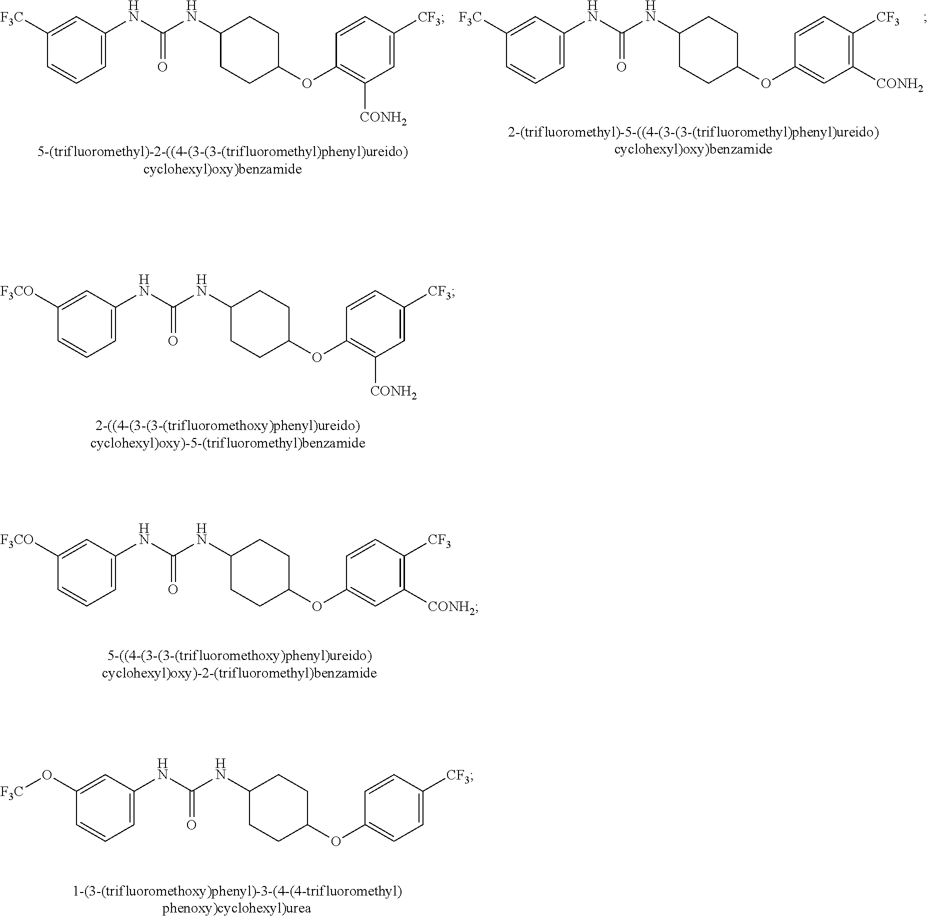 [SMILES: NC(=O)c1cc(C(F)(F)F)ccc1OC1CCC(NC(=O)Nc2cccc(C(F)(F)F)c2)CC1 ; NC(=O)c1cc(OC2CCC(NC(=O)Nc3cccc(C(F)(F)F)c3)CC2)ccc1C(F)(F)F ; NC(=O)c1cc(C(F)(F)F)ccc1OC1CCC(NC(=O)Nc2cccc(OC(F)(F)F)c2)CC1 ; NC(=O)c1cc(OC2CCC(NC(=O)Nc3cccc(OC(F)(F)F)c3)CC2)ccc1C(F)(F)F ; O=C(Nc1cccc(OC(F)(F)F)c1)NC1CCC(Oc2ccc(C(F)(F)F)cc2)CC1]

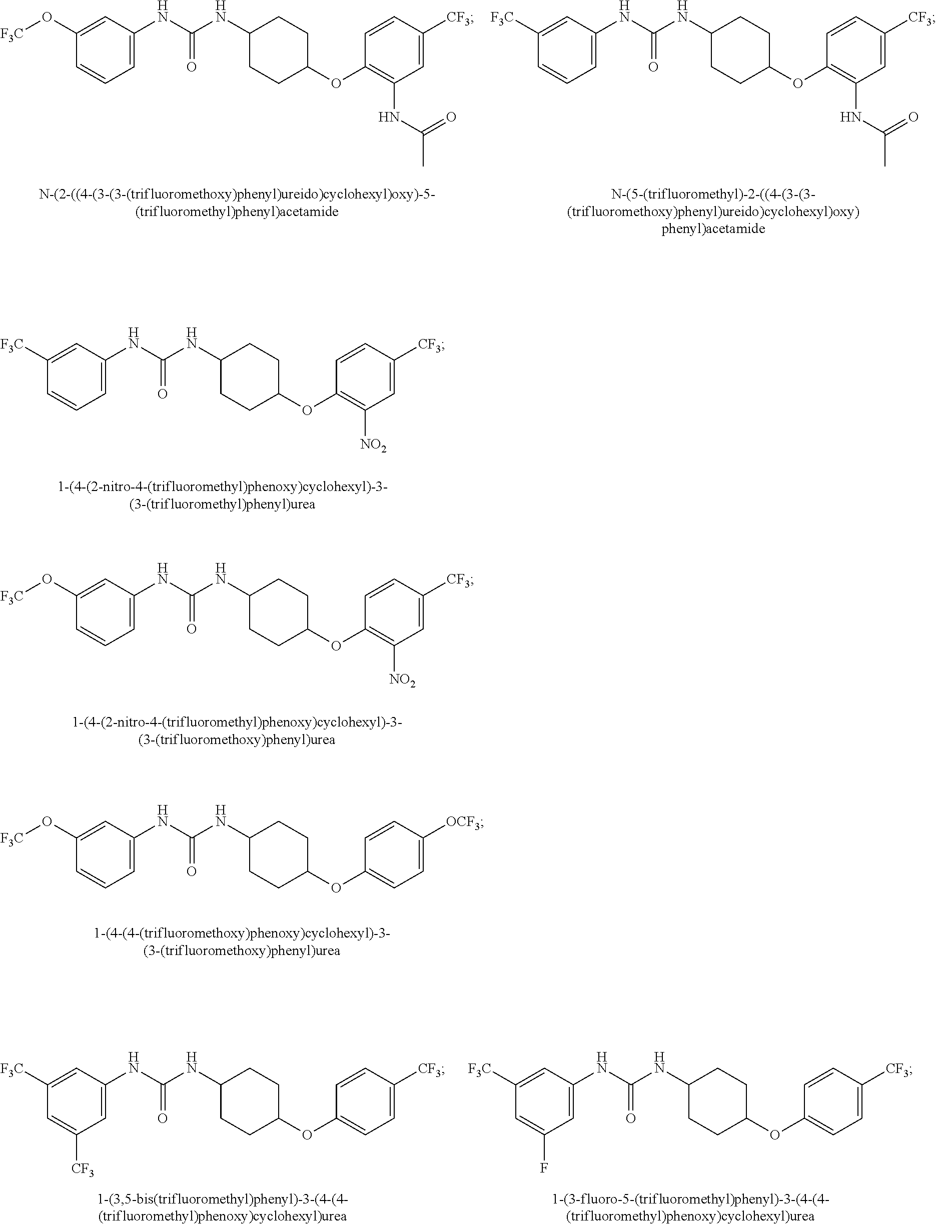

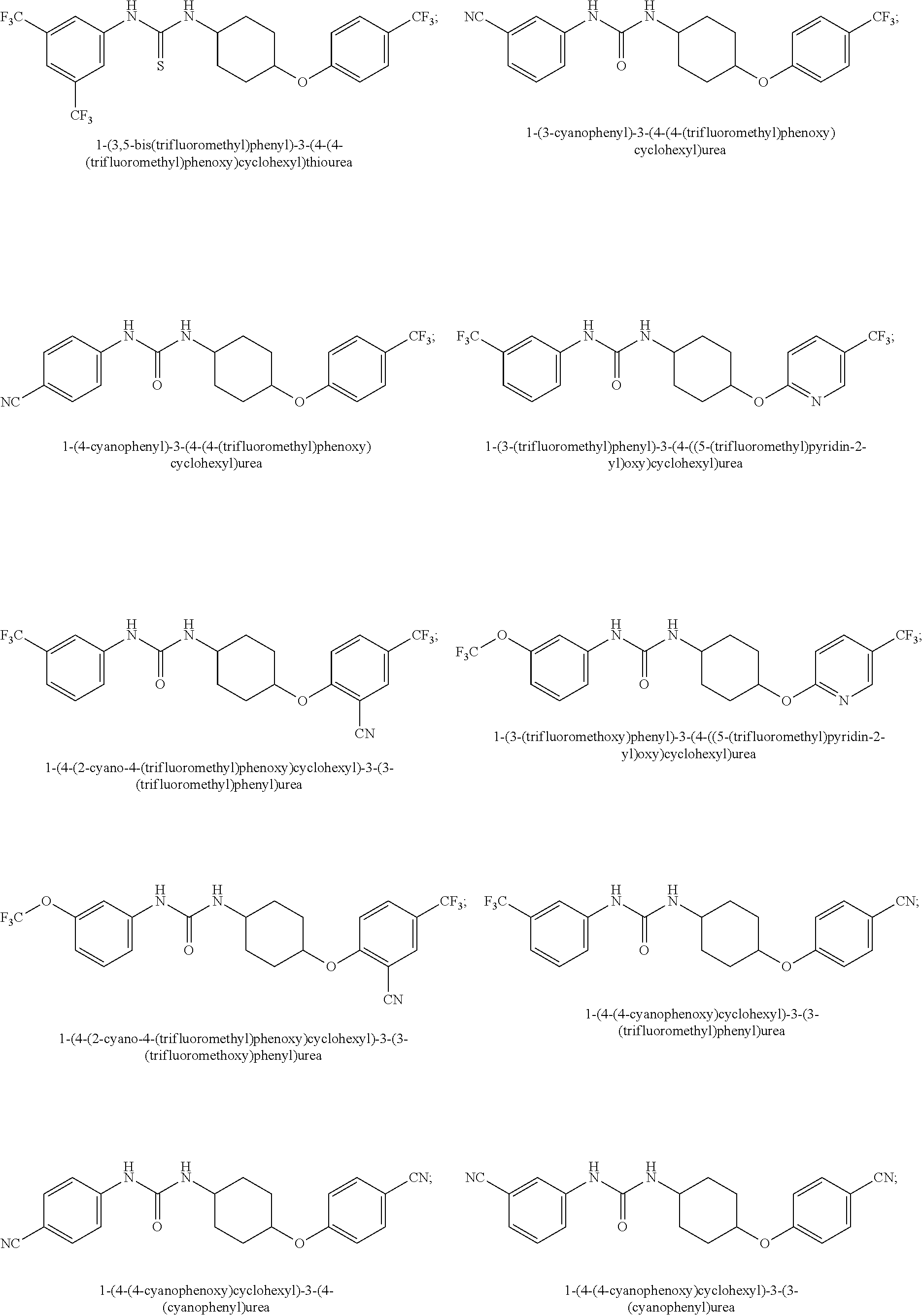

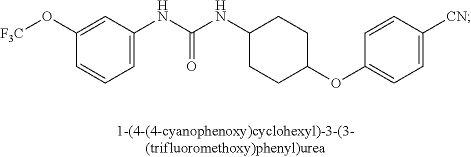 [SMILES: N#Cc1ccc(OC2CCC(NC(=O)Nc3cccc(OC(F)(F)F)c3)CC2)cc1]

or a pharmaceutically acceptable salt thereof.

In some embodiments, a compound of Formulas (I), (II), (III), and/or (IV) is selected from the group consisting of:

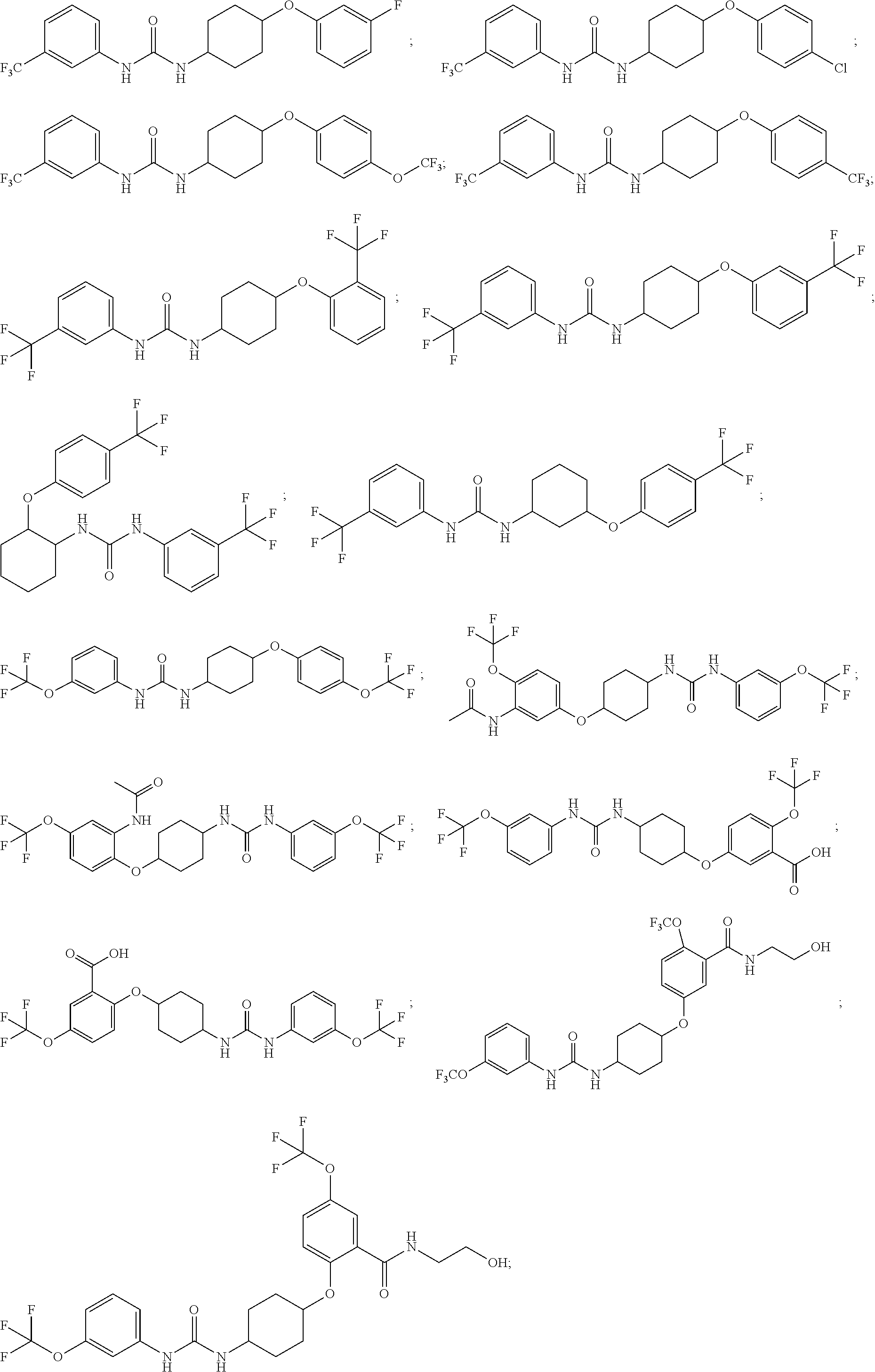

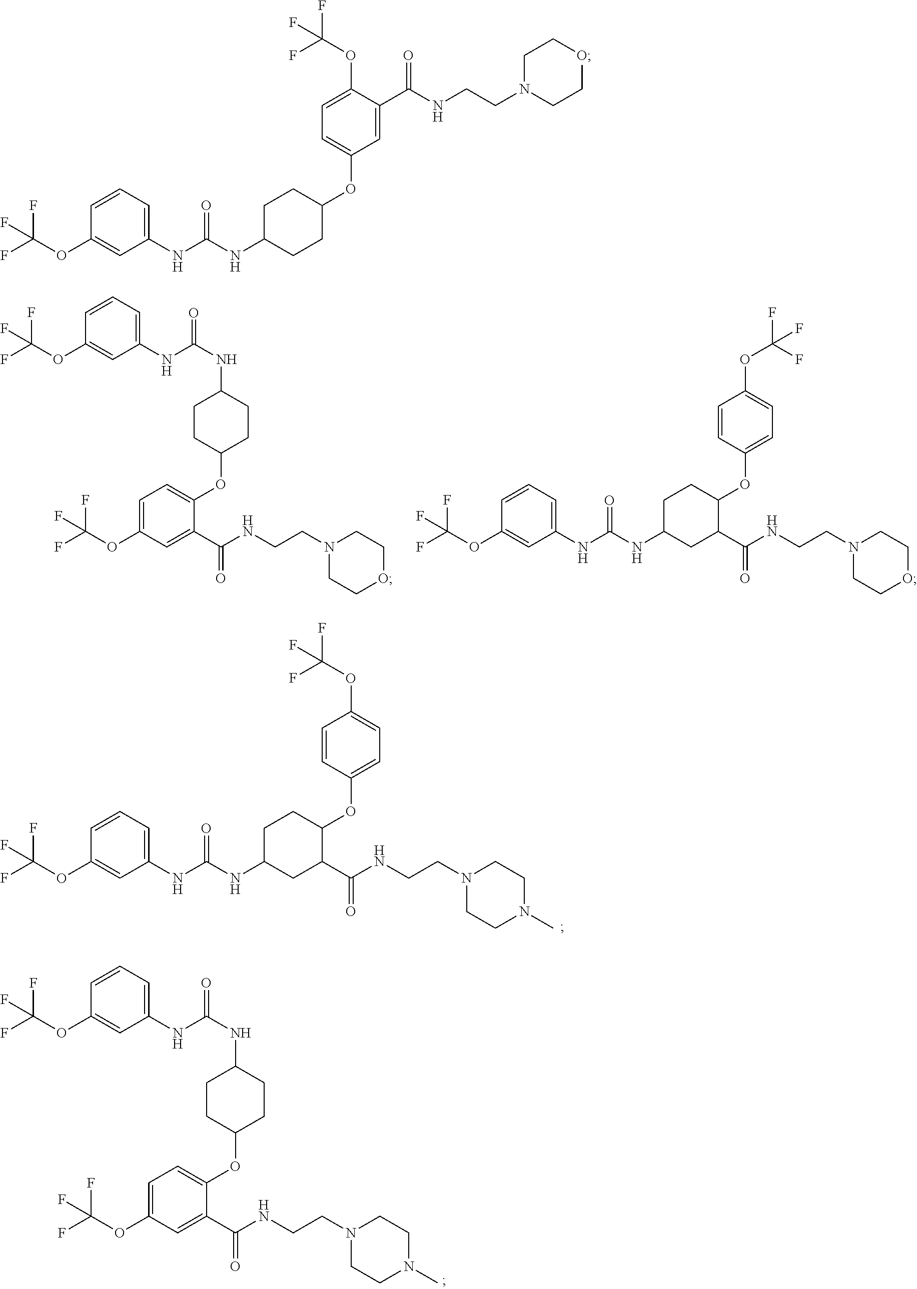

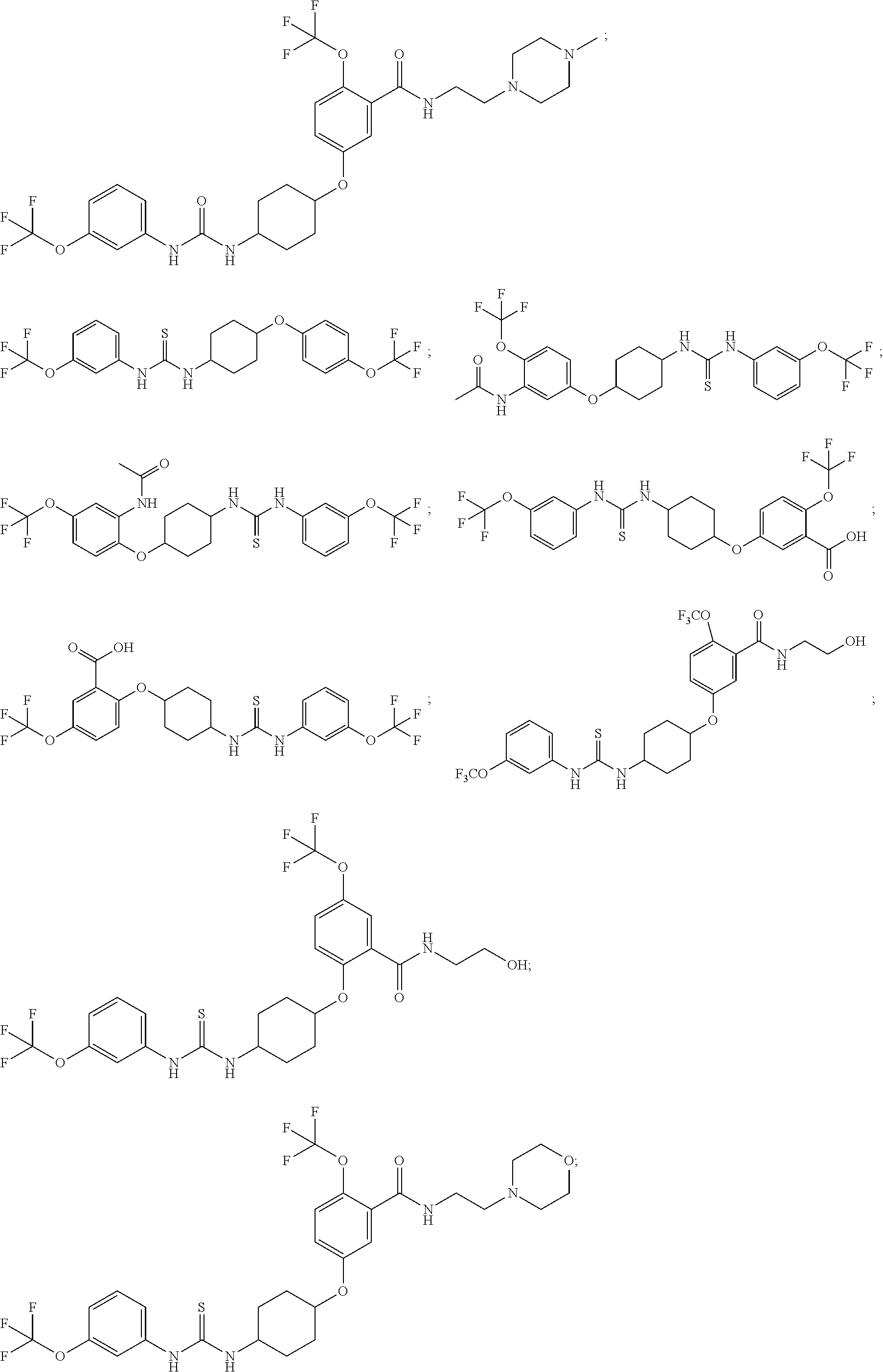

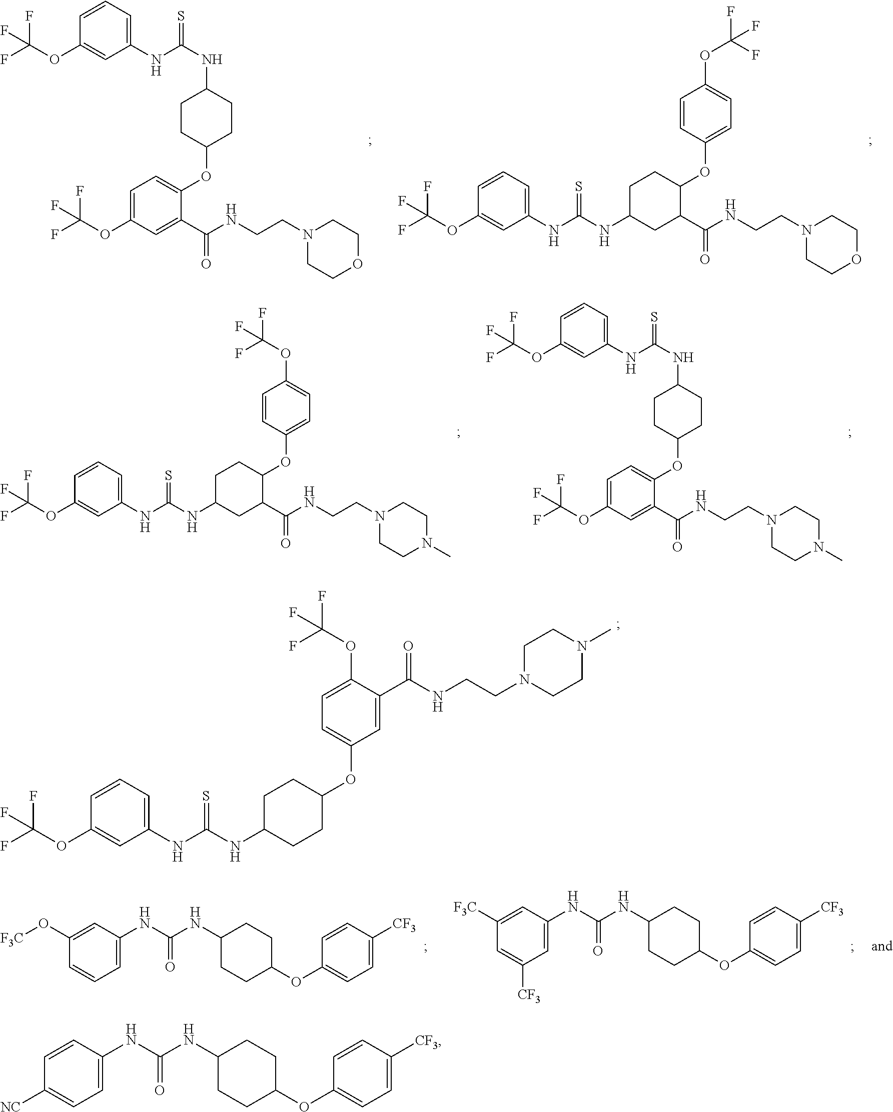

or a pharmaceutically acceptable salt thereof.

In some embodiments, a compound of Formulas (I), (II), (III), and/or (IV) is selected from the group consisting of:

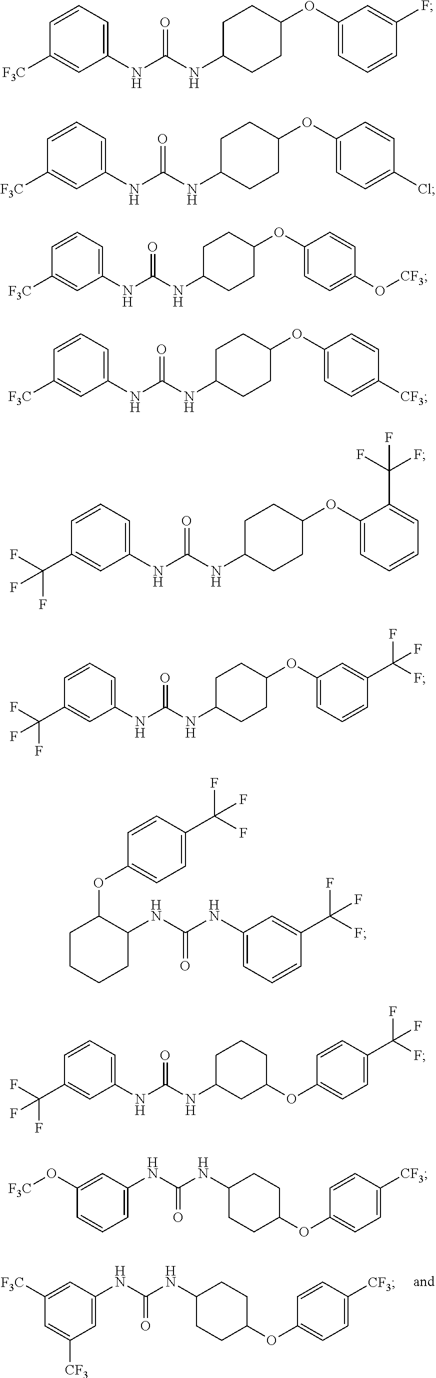

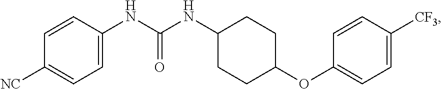 [SMILES: N#Cc1ccc(NC(=O)NC2CCC(Oc3ccc(C(F)(F)F)cc3)CC2)cc1]

or a pharmaceutically acceptable salt thereof.

The compound provided herein can be synthesized as described herein (see, e.g., Schemes 1 and 2 in the Examples) or may be prepared using conventional techniques and readily available starting materials. For example, the compounds provided herein may be 1.0 prepared using procedures modified from those described in WO 2010/138820.

### Methods of Treatment

The methods described herein include methods for the treatment of disorders associated with an eIF2α kinase, eIF2α phosphorylation, uncontrolled translation initiation, or disorders that may be treated by inducing eIF2α phosphorylation. Generally, the methods include administering a therapeutically effective amount of a compound as described herein, to a patient who is in need of, or who has been determined to be in need of such treatment. As used in this context, to “treat” means to ameliorate at least one symptom of the disorder associated with an eIF2α kinase, eIF2α phosphorylation, uncontrolled translation initiation, or disorders that may be treated by inducing eIF2α phosphorylation. In some embodiments, the disorder is selected from the group consisting of: a cancer, a hemolytic anemia, Wolcott-Rallison syndrome, a neurodegenerative disease, a motor neuron disease, tuberous sclerosis complex, an autism spectrum disorder, and a ribosomal defect disease.

In some embodiments, the disorder is a cancer. In some embodiments, the cancer is selected from the group consisting of: cervical cancer, liver cancer, bile duct cancer, eye cancer, esophageal cancer, head and neck cancer, brain cancer, prostate cancer, pancreatic cancer, skin cancer, testicular cancer, breast cancer, uterine cancer, penile cancer, small intestine cancer, colon cancer, stomach cancer, bladder cancer, anal cancer, lung cancer, lymphoma, leukemia, thyroid cancer, bone cancer, kidney cancer, and ovarian cancer. In some embodiments, the cancer is selected from the group consisting of: cervical cancer, liver cancer, glioblastoma, prostate cancer, pancreatic cancer, skin cancer, breast cancer, colon cancer, lung cancer, lymphoma, leukemia, kidney cancer, and ovarian cancer. In some embodiments, the cancer is selected from the group consisting of: breast cancer and skin cancer.

A method for selection of cancer patients for treatment is also provided. In accordance with certain examples, methods are provided of identifying cancer patients for treatment with compounds of Formulas (I)-(X). In some embodiments, cancer cells from a patient are assayed to determine the expression level of HRI. Based on the expression level of HRI, the patient is identified as a candidate for treatment with compounds Formula (I) and/or Formula (II) and/or Formula (III) and/or Formula (IV) and/or Formula (V) and/or Formula (VI) and/or Formula (VII) and/or Formula (VIII) and/or Formula (IX) and/or Formula (X).

Once the HRI expression level of cancer cells from an individual is determined, such as by methods described herein, the individual may be identified as a suitable candidate for treatment with compounds Formula (I) and/or Formula (II) and/or Formula (III) and/or Formula (IV) and/or Formula (V) and/or Formula (VI) and/or Formula (VII) and/or Formula (VIII) and/or Formula (IX) and/or Formula (X). According to one aspect, the compounds are administered to an individual in a manner to activate HRI thereby causing phosphorylation of eIF2α and inhibition of translation initiation.

In some embodiments, one or more compounds provided herein are used for the treatment of noncancereous cellular proliferative disorders. Examples of noncancerous cellular proliferative disorders includes fibroadenoma, adenoma, intraductal papilloma, nipple adenoma, adenosis, fibrocystic disease or changes of breast, plasma cell proliferative disorder (PCPD), restenosis, atherosclerosis, rheumatoid arthritis, myofibromatosis, fibrous hamartoma, granular lymphocyte proliferative disorders, benign hyperplasia of prostate, heavy chain diseases (HCDs), lymphoproliferative disorders, psoriasis, lung fibrosis (e.g., idiopathic pulmonary fibrosis), scleroderma, cirrhosis of the liver, IgA nephropathy, mesangial proliferative glomerulonephritis, membranoproliferative glomerulonephritis, hemangiomas, vascular and non-vascular intraocular proliferative disorders, polycteme vera, pulmonary hypertension, and in-stent restenosis. See. e.g., Grimminger F. et al., Nat Rev Drug Discov. (2010) 9(12):956-70.

The language “treatment of cellular proliferative disorders” is intended to include, but is not limited to, the prevention of the growth of neoplasms in a subject or a reduction in the growth of pre-existing neoplasms in a subject, as well as the prevention or reduction of increased or uncontrollable cell growth. The inhibition also can be the inhibition of the metastasis of a neoplasm from one site to another.

In some embodiments, the disorder is a hemolytic anemia, for example, a hemolytic anemia not caused by an infectious agent. In some embodiments, the hemolytic anemia is selected from erythropoietic protoporphyria, α-thalassemia, β-thalassemia, δ-thalassemia, sideroblastic anemia, and unstable hemoglobin hemolytic anemia. In some embodiments, the hemolytic anemia is β-thalassemia.

An assay for determining the effectiveness of a compound provided herein in treating a hemolytic anemia may be performed by contacting a cell with a compound provided herein, or a pharmaceutically acceptable salt form thereof, in vitro, and determining the effectiveness of the compound in inducing enhanced oxygen-carrying capacity in a cell in vitro. For example, human red blood progenitor cells may be obtained from human placenta cords discarded after birth or from β-thalassemia patients. CD34(+) cells may be separated by FACS (Fluorescent activated cell sorting), and induced to differentiate using erythropoietin. The cells may be treated with the compound or vehicle, and then evaluated at various stages of differentiation to red blood cells. The cell morphology, the ratio of mutant vs. wild-type hemoglobin, and the oxygen-carrying capacity of the differentiated red blood cells would be determined. A therapeutically effective amount would increase expression of wild-type hemoglobin and/or oxygen-carrying capacity of the cells treated with the compound compared to vehicle.

In some embodiments, the compounds may not change the ratio of mutant to wild type hemoglobin but may induce cells to fold the mutant protein similar to wild type configuration.

An assay for determining the effectiveness of a compound provided herein in treating a hemolytic anemia may be performed with an appropriate animal model and a compound provided herein, or a pharmaceutically acceptable salt form thereof, in vivo, and determining the effectiveness in inducing enhanced oxygen-carrying capacity in an animal in vivo. For example, several models of hemolytic anemia may be used, such as mutant β-thalassemia expressing cells, for in vivo studies. In such a mouse colony, mutant and wild-type pups would be obtained by breeding heterozygous mice. Mouse pups would be fed milk containing the compound or vehicle. The cell morphology, the ratio of mutant vs. wild-type hemoglobin, and the oxygen-carrying capacity of the animals' red blood cells would be determined. A therapeutically effective amount would increase expression of wild-type hemoglobin and/or oxygen-carrying capacity with the compound compared to vehicle.

In some embodiments, the disorder is Wolcott-Rallison syndrome.

An assay for determining the effectiveness of a compound provided herein in treating Wolcott-Rallison syndrome may be determined with an appropriate animal model and a compound provided herein, or a pharmaceutically acceptable salt form thereof, in vivo. Mice deficient in PERK, the human gene inactivated in patients suffering from Walcott-Rallison syndrome, or Akita mice, exhibiting a mutation in the insulin gene, may be used in the in vivo assay. PERK mice colonies would be provided with wild-type, heterozygous, and homozygous PERK knockout genotypes. Each genotype group would be split into two groups, and each group treated with milk or food containing either the compound or the vehicle. The weight and growth parameters of the mouse pups would be recorded weekly. Blood glucose and insulin levels would be determined at various times after feeding. Glucose processing capacity would be determined via a glucose tolerance test. Populations would be sacrificed on days 20, 40, 60 and 80 after birth. The pancreas, liver, and bones would be examined for morphology and presence of pancreatic β-cells. Homozygous PERK gene knockout mice will be smaller, fail to thrive, and die off quicker if fed vehicle containing milk or food compared to those fed milk or food containing the compound. The vehicle-treated pups will have greater impaired glucose tolerance, reduced insulin secretion, diminished numbers of pancreatic β-cells, and display greater skeletal abnormalities compared with the compound-treated pups.

In some embodiments, the disorder is a neurodegenerative or motor neuron disease. In some embodiments, the neurodegenerative or motor neuron disease is selected from the group consisting of: amyotrophic lateral sclerosis, Alzheimer's disease, Amytrophic Lateral Sclerosis, Parkinson's disease, and Huntington's disease. In some embodiments, the neurodegenerative disease is Alzheimer's disease.

In some embodiments, the disorder is tuberous sclerosis complex.

Synaptic transmission, long term memory formation and consolidation are highly dependent on regulated protein synthesis, including protein synthesis regulated by eIF2α kinases. Deregulation of protein synthesis may lead to abnormalities in long term memory formation, consolidation, and reconsolidation leading to autism spectrum disorders in a context dependent manner.

In some embodiments, the disorder is autism spectrum disorder. In some embodiments, the autism spectrum disorder is selected from the group consisting of: Asperger's syndrome, autistic disorder, Rett syndrome, childhood disintegrative disorder, and pervasive developmental disorder, not otherwise specified (PDD-NOS).

Unregulated protein synthesis has also been implicated in defective long term memory formation, consolidation, and reconsolidation. Inability to break protein synthesis underlies mental retardation disorders such as fragile-X syndrome.

In some embodiments, the disorder is a mental retardation disorder. In some embodiments, the mental retardation disorder is fragile-X syndrome.

In some embodiments, the disorder is a ribosomal defect disease. In some embodiments, the ribosomal defect disease is selected from the group consisting of: Shwachman-Bodian-Diamond syndrome, Diamond Blackfan anemia, and cartilage hair hypoplasia.

A method for activating an eIF2α kinase in a cell is also provided herein, the method comprising contacting the cell with an effective amount of a compound provided herein. In some embodiments, the binding and activation of an eIF2α kinase results in higher phosphorylation of an eIF2α to balance hemoglobin synthesis to the hemoglobin folding capacity of the cells which, in turn, leads to increased oxygen-carrying capacity in the cell. The method of activating an eIF2α kinase in a cell may be performed by contacting the cell with an effective amount of a compound provided herein, or a pharmaceutically acceptable salt form thereof, in vitro, thereby inducing activation of an eIF2α kinase in a cell in vitro. Uses of such an in vitro methods of activating an eIF2α kinase include, but are not limited to use in a screening assay (for example, wherein a compound provided herein is used as a positive control or standard compared to compounds of unknown activity or potency in activating an eIF2α kinase). In some embodiments thereof, activating of an eIF2α kinase is performed in a red blood cell progenitor.

The method of activating an eIF2α kinase in a cell may be performed, for example, by contacting a cell (e.g., a CD34+ progenitor cell) with a compound provided herein, in vivo, thereby activating an eIF2α kinase in a patient in vivo. The contacting is achieved by causing a compound as provided herein, or a pharmaceutically acceptable salt form thereof, to be present in the patient in an amount effective to achieve activation of an eIF2α kinase. This may be achieved, for example, by administering an effective amount of a compound provided herein, or a pharmaceutically acceptable salt form thereof, to a patient. Uses of such an in vivo methods of activating an eIF2α kinase include, but are not limited to, use in methods of treating a disease or condition, wherein activating an eIF2α kinase is beneficial. In some embodiments thereof, activation of an eIF2α kinase results in increased phosphorylation of an eIF2α kinase, and thereby greater oxygen-carrying capacity in a red blood cell, for example in a patient suffering from β-thalassemia or a related disorder. The method is preferably performed by administering a therapeutically effective amount of a compound provided herein, or a pharmaceutically acceptable salt form thereof, to a patient who is suffering from β-thalassemia or a related disorder.

### Pharmaceutical Compositions and Methods of Administration

The methods described herein include the manufacture and use of pharmaceutical compositions, which include one or more compounds provided herein. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary pharmaceutically active compounds can also be incorporated into the compositions.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, compositions comprising a LOXL1 enhancer for transdermal application can further comprise cosmetically-acceptable carriers or vehicles and any optional components. A number of such cosmetically acceptable carriers, vehicles and optional components are known in the art and include carriers and vehicles suitable for application to skin (e.g., sunscreens, creams, milks, lotions, masks, serums, etc.), see, e.g., U.S. Pat. Nos. 6,645,512 and 6,641,824. In particular, optional components that may be desirable include, but are not limited to absorbents, anti-acne actives, anti-caking agents, anti-cellulite agents, anti-foaming agents, anti-fungal actives, anti-inflammatory actives, anti-microbial actives, anti-oxidants, antiperspirant/deodorant actives, anti-skin atrophy actives, anti-viral agents, anti-wrinkle actives, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, nail enamels, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin thickeners, sunscreen actives, topical anesthetics, vitamin compounds, and combinations thereof.

The LOXL1 enhancer compositions can also be prepared in the form of suppositories (e.g., with conventional suppository bases such as cocoa butter and other glycerides) or retention enemas for rectal or vaginal delivery. Such suppositories can be used particularly for the treatment of conditions associated with the loss of in elastic fibers that affect the pelvic organs, e.g., pelvic organ prolapse and/or urinary incontinence, inter alia.

The pharmaceutical compositions can also be prepared in the form of suppositories (e.g., with conventional suppository bases such as cocoa butter and other glycerides) or retention enemas for rectal delivery.

In one embodiment, the compounds provided herein are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

In some embodiments, a compound provided herein can be conjugated to an antibody or a similar targeting moiety known in the art so that will aid in delivery of the compound to diseased cells, tissues and/or organs.

In some embodiments, a compound provided herein can be made into a prodrug such that the compound can be preferentially activated by the intended target cells. For example, a compound provided herein may be conjugated such that the active compound will be released only in cells that produce prostate specific antigens, thus facilitating and targeting the treatment of prostate cancer.

The pharmaceutical compositions can be included in a container, pack, or dispenser together with instructions for administration.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a compound provided herein (i.e., an effective dosage) depends on the compounds selected. The compositions can be administered one from one or more times per day to one or more times per week; including once every other day. The skilled artisan will appreciate that certain factors may influence the dosage and timing required to effectively treat a patient, including but not limited to the severity of the disease or disorder, previous treatments, the general health and/or age of the patient, and other diseases present. Moreover, treatment of a patient with a therapeutically effective amount of a compound described herein can include a single treatment or a series of treatments.

Dosage, toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods provided herein, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Materials and Methods

### General Methods

All reagents and solvents were purchased from commercial sources and used without further purification. All the reaction solvents were anhydrous and the reactions were maintained under inert atmosphere. Thin layer chromatography (TLC) were run on pre-coated EMD silica gel 60 F254 plates and observed under UV light at 254 nm and with basic potassium permanganate dip. All the reactions were monitored by LC-MS analysis on reverse phase (column Waters Symmetry C18, 2.1×100 mm, 3.5 μm particle size) using a Waters Alliance 2695/Micromass ZQ or Agilent 1200 series system with UV detector (214 nm and 254 nm) and an Agilent 6130 quadrupole mass detector, with the binary system water/acetonitrile containing 0.1% of formic acid as eluent. The purity of all final compounds was determined by analytical HPLC on reverse phase (column XBridge BEH130 C18, 4.6×100 mm, 5 μm particle size) using a Waters Alliance 2695 with the binary system water/acetonitrile containing 0.1% trifluoroacetic acid (TFA) as eluent. Column chromatography was performed with silica gel (230-400 mesh, grade 60, Fisher scientific, USA). Purifications by flash chromatography were performed on Biotage SP1 using silica gel pre-packed columns, (200-400 mesh), and were monitored by UV at 254 and 280 nm. Melting points were determined using a Mel-Temp Electrothermal apparatus and were uncorrected. Proton, carbon, and fluorine NMR analyses were performed on Topspin 300 MHz, Varian-400 and Varian-500 MHz spectrometers using DMSO-d₆ or CDCl₃ as solvent. Chemical shifts (δ) are reported in ppm relative to TMS as internal standard.

### Synthesis

Referring to Schemes 1 and 2, ureas of interest that were not commercially available (I-11o and I-12o), and designed for the initial hit-to-lead optimization (I-14-20 and III-1-6), were synthesized. Reacting equimolar amounts of the appropriate cyclohexylamine and phenyl isocyantate yielded the anticipated N-phenyl,N′-cyclohexylaryl ureas. The substituted trans-(4-phenoxy)cyclohexylamines (1-7) were generated from trans-4-aminocyclohexanol and the appropriately substituted fluorobenzenes (see, for example, U.S. Patent application No. 2005/0215784). The urea incorporating the cis-1,4-disubstituted cyclohexyl moiety, I-11o was obtained by reacting methyl 2-isocyanatobenzoate with a small excess of cis-4-(4-fluorophenoxy)cyclohexanamine, 1b in DCM/DMSO at room temperature (Scheme 1 B). The amine 1b was synthesized from trans-4-aminocyclohexanol and 4-fluorophenol via Mitsunobu coupling reaction (see, for example, Zhang, Y. et al. ACS Med Chem Lett 2010, 1, 460-465).

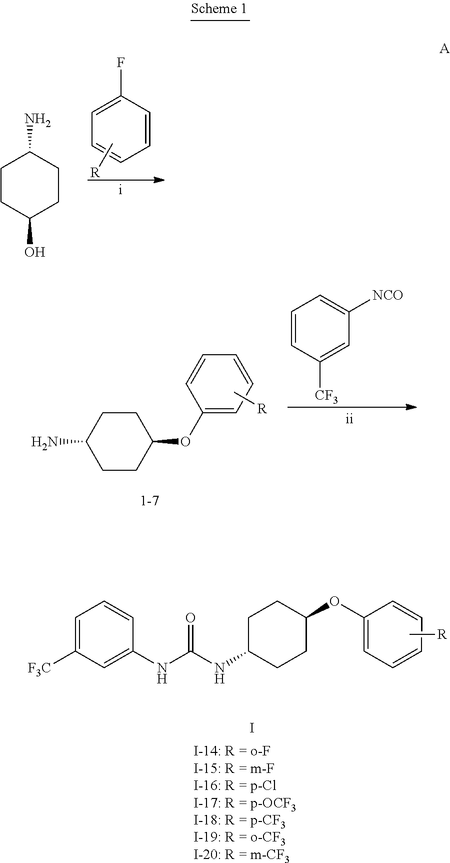

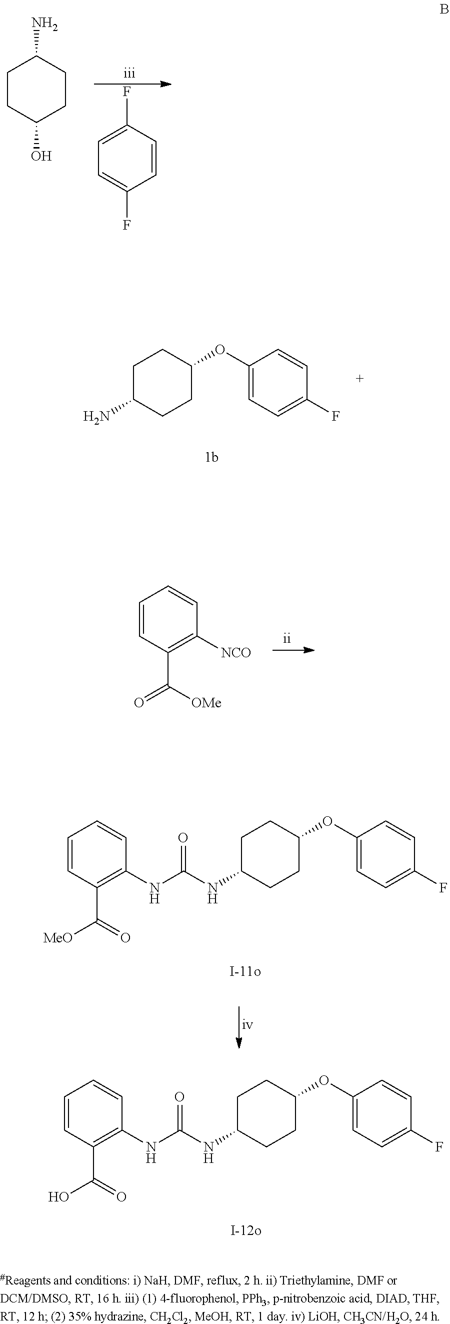

Referring to Scheme 2, modifications of position of the phenoxy substituent on the cyclohexyl ring of the ureas (III-1-3, 5, 6) were obtained by reacting the 3-(trifluromethyl)phenyl isocyanate with the properly substituted cyclohexylamines (8-11), which were either commercially available or synthesized. The substituted cyclohexylamines were obtained by the O-alkylation of meta-aminocyclohexanol using 4-substituted fluorobenzenes, as in 8-9, or by O-alkylation of 4-trans-Boc-aminocyclohexanol using alkyl halides as in 10-11 followed by de-protection of the amine. The obtained amines were then reacted with 3-(trifluromethyl)phenyl isocyanate to produce compounds III-1-3, 5, and 6 as described herein.

Based on analytical reversed-phase high performance liquid chromatography (RP-HPLC) analysis the purity of all final N-aryl, N′-cyclohexylarylureas submitted to biological characterization and reported inhere equaled or exceeded 95%. Some of the pure compounds comprise of racemic mixtures originating from the non-symmetrically disubstituted cyclohexyl moieties. Their structural identity and integrity was confirmed by HR- or LC-MS, ¹H-, ¹³C-, and ¹⁹F-NMR.

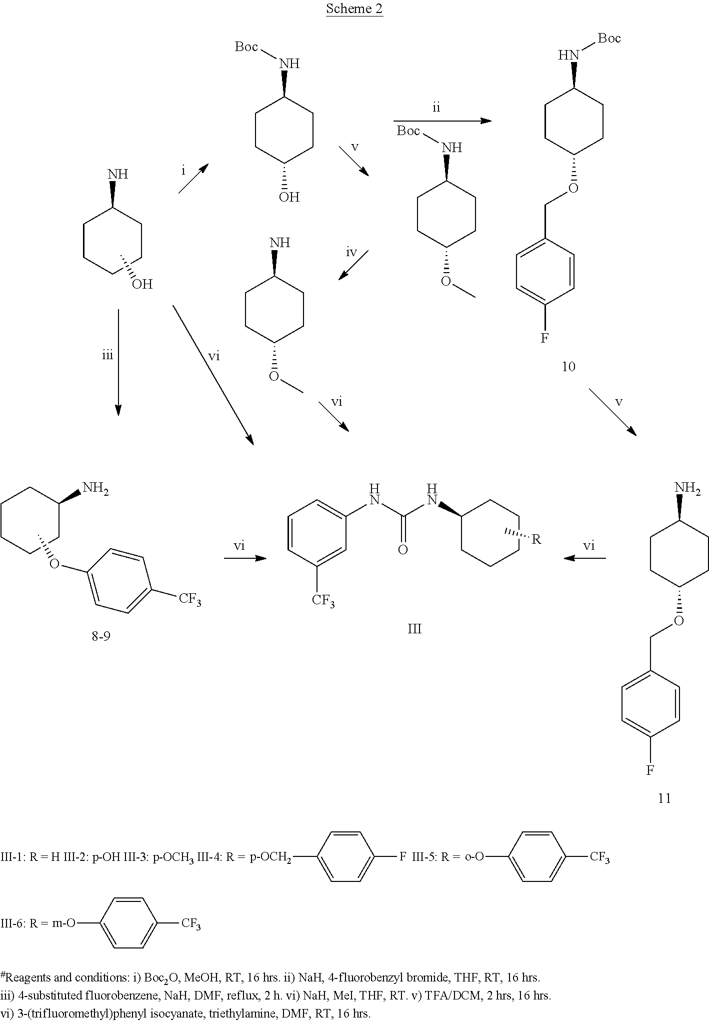

### Plasmids and Ternary Complex Assay

The dual luciferase expression vector and other plasmids used for these studies were previously described (see, for example, Ziegeler, G. et al. Journal of Biological Chemistry 2010, 285(20), 15408-19). The ternary complex assay, surrogate of eIF2α phosphorylation, has been described elsewhere (see, for example, Chen, T. et al. Nature Chemical Biology 2011, 7(9), 610-6). Briefly, the pBISA vector, which contains seven copies of the tetracycline-regulated transactivator response element (TRE), is flanked on both sides by minimal human cytomegalovirus (CMV) minimal promoters allowing bi-directional transcription and two MCSs (multiple cloning sites). Firefly and renilla luciferases were subcloned into MCS-I and MCS-II, respectively. This plasmid, designated pBISA-DL, transcribes two mRNAs that contain the 90 nucleotide plasmid derived 5′UTR (same sequence in both mRNAs), and the ORF encoding either firefly or renilla luciferase followed by a polyadenylation sequence. This plasmid was further modified by inserting the 5′UTR of ATF-4 mRNA into MCS-I in front of the firefly luciferase mRNA. Transcription from this unit generates an mRNA that contains the firefly luciferase ORF preceded by a 5′UTR composed of 90 nucleotides derived from the plasmid and 267 nucleotides derived from the 5′UTR of ATF-4 mRNA. Transcription from the other unit generates an mRNA that contains the renilla luciferase ORF proceeded only by the 90-nucleotide plasmid-derived sequence in the 5′UTR (pBISA-DL (ATF-4)) (see, for example, Chen, T. et al. Nature Chemical Biology 2011, 7(9), 610-6).

### Dual Luciferase (DLR) Assay

Cells expressing firefly and renilla luciferases were assayed with a dual glow luciferase assay kit, per manufacturer's instruction (Promega Inc., Madison, Wis.). The data calculations were carried out as the ratio of firefly to renilla luciferase signal.

### Stable and Transient Transfection

Stable cell lines utilized in this study were generated as described elsewhere (see, for example, Chen, T. et al. Nature Chemical Biology 2011, 7(9), 610-6). Briefly, cells were seeded at the density of 105 in 60-mm dish (stable transfection) or 104 cells per well of 96-well plate (transient transfection) and transfected one day later using the Lipofectamine 2000 (Invitrogen). For selection of stable cell lines, transfected cells were transferred to 100-mm plates and selected with appropriate antibiotics.

### Western Blots

Cells cultured under recommended media conditions, were plated and maintained in serum-containing media without antibiotics in 14-cm plates (Nunc) until reaching 70% confluence. Cells were then treated with compounds for 6 hours, washed with cold PBS once, and lysed with M-PER Mammalian Protein Extraction Reagent (Pierce) for 30 minutes on ice. The cell lysates were centrifuged at 12,000 RPM for 15 min and the supernatants were transferred to fresh tubes and the concentrations were determined by BCA (Pierce). Equal amount of proteins were mixed with Laemmli Sample Buffer, heated at 100° C. for 5 min and separated by SDS-PAGE and probed with antiphosphoserine-51-eIF2α (Phos-eIF2α), anti-total eIF2α-specific antibodies (Total-eIF2α) (Biosource International, Hopkinton, Mass.), anti-CHOP, anti-Cyclin D1 or anti Actin (Santa Cruz Biotechnology, CA) as described previously (see, for example, Aktas, H. et al. Molecular and Cellular Biology 1997, 17(7), 3850-7).

### Cell Growth Assay

Cells were seeded in 96-well plates and maintained for 5 days in the presence of 0.5 to 20 μM of individual compound, and cell proliferation was measured by the sulforhodamine B (SRB) assay as described previously (see, for example, Palakurthi, S. S. et al. Cancer Research 2000, 60(11), 2919-2925). Briefly, at the end of a 5-day treatment, cells were fixed in 10% cold trichloroacetic acid. Cell number was estimated by measuring the remaining bound dye of sulforhodamine B after washing. The percentage of growth was calculated by using the equation: 100×[(T−T0)/(C−T0)], where T and C represent the absorbance in treated and control cultures at Day 5, and T0 at time zero, respectively. If T is less than T0, cell death has occurred and can be calculated from 100×[(T−T0)/T0].

### Abbreviations Used

HRI, Heme-regulated inhibitor kinase; eIF2α, eukaryotic translation initiation factor 2 alpha; Met-tRNAi, methionine transfer RNA; Pi, inorganic phosphate; PKR, protein kinase R; PERK, PKR-like endoplasmic reticulum kinase; GCN2, general control non-derepressible-2; AUDA, 12-(3-adamantane-1-ylureido)dodecanoic acid; sEH, soluble epoxide hydrolase; CHOP, CCAAT/enhancer-binding protein homologous protein; SRB, sulforhodamine B; RP-HPLC, high performance liquid chromatography; DLR, dual luciferase; uORF, upstream open reading frame; UTR, untranslated region; F/R, firely to renilla luciferase ratio; ATF-4, activating transcription factor 4; ππₓ, high lipophilicity parameters; P-eIF2α, phosphorylated eIF2α; T-eIF2α, total eIF2α; TLC, thin layer chromatography; TFA, trifluoracetic acid; δ, delta ppm (chemical shifts); TRE, transactivator response element; CMV, minimal human cytomegalovirus; MCS, multiple cloning sites

### Example 1

### Compound Screening

A library of 1900 urea compounds was screened in the surrogate dual luciferase eIF2α phosphorylation (ternary complex) assay. This library was originally assembled and screened for inhibition of sEH by Hammock's group at UC-Davis (see, for example, Chen, T. et al. Nature Chem Biol 2011, 7, 610-616; Ziegeler, G et al. J Biol Chem 2010, 285, 15408-15419). We developed this assay in order to identify compounds that activate HRI, taking advantage of the fact that activated HRI phosphorylates eIF2α thereby reducing the amount of the eIF2•GTP•Met-tRNAi ternary complex that results in inhibition of translation of many mRNAs but paradoxically increases translation of some mRNAs that contain multiple upstream ORF (uORF) in their 5′ untranslated regions (UTRs). In our assay, firefly (F) luciferase mRNA is fused to 5′UTR of activating transcription factor 4 (ATF-4) mRNA that has multiple uORFs while renilla (R) luciferase mRNA is fused to a 5′UTR lacking any uORFs. Compounds that reduce the amount of the ternary complex, such as those activating HRI, would increase F luciferase expression while decreasing the R luciferase expression, resulting in an increased F/R luciferase ratio. To calculate the activity scores, the F/R ratios for every compound-treated wells were normalized to vehicle-treated (DMSO) wells that was arbitrarily set at 1 (F/R=1).

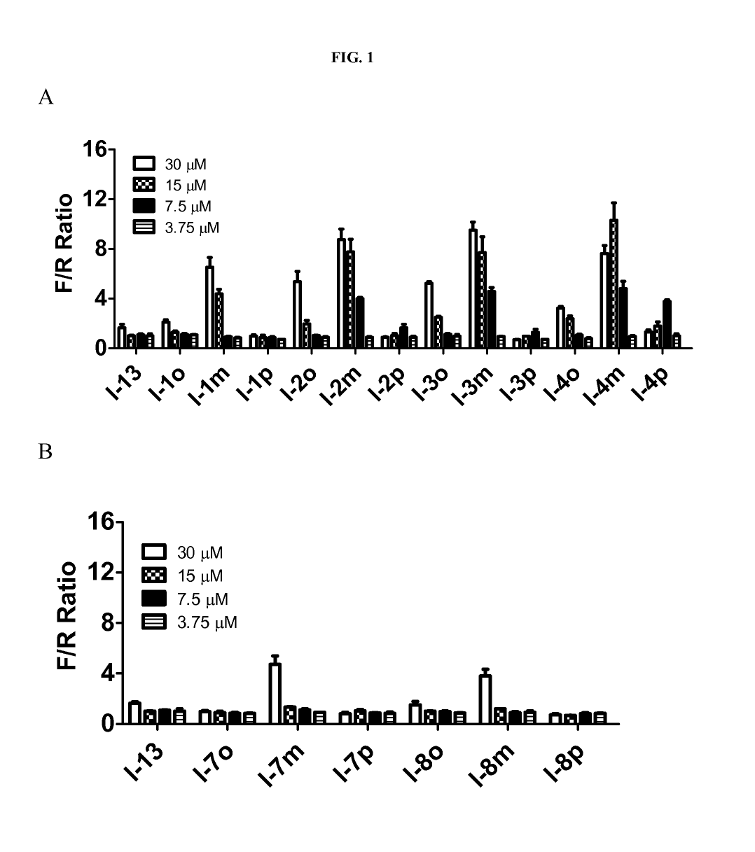

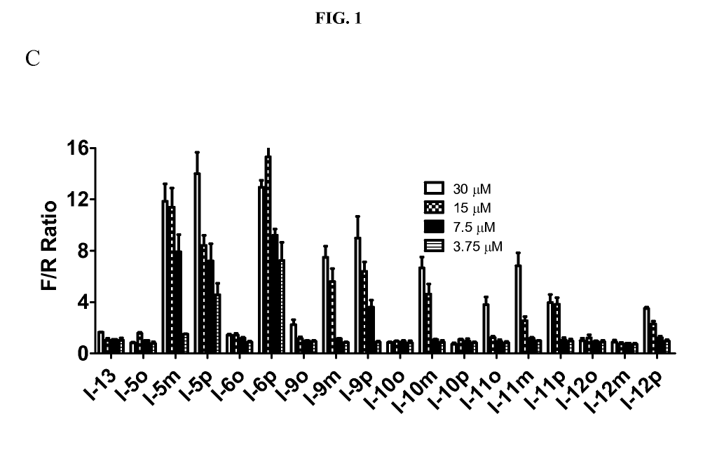

We screened compound library at 33 μM concentration in 384-well plate format. Of interest were the N-phenyl-N′-(4-(4-fluorophenoxy)cyclohexyl)ureas (I) and N-phenyl-N′-(4-((2,6-difluorobenzyl)oxy)cyclohexyl)ureas (II) scaffolds, which were re-evaluated in the dual luciferase surrogate eIF2α phosphorylation assay (see, for example, Chen, T. et al. Nature Chem Biol 2011, 7, 610-616) and for inhibition of cell proliferation in the SRB assay (see, for example, Palakurthi, S. S. et al. Cancer Res 2000, 60, 2919-2925) (Table 1, FIG. 1, and Table 2). This re-testing served the dual purpose: to confirm the results of the primary screen and to establish the dose-response relationship. There appears to be no statistically significant correlation (r²<0.15) between induction of eIF2 phosphorylation and the previously reported inhibition of sEH by these series of compounds (see, for example, Hwang, S. H. et al. Bioorg Med Chem Lett 2006, 16, 5773-5777) suggesting distinct structure-activity relationships for both enzymes.

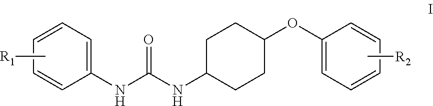

TABLE 1
Activity of N-phenyl-N′-(4-(4-fluorophenoxy)cyclohexyl)ureas, I (R₂ = p-F), in
the ternary complex and cell proliferation assay.

| | | | IC₅₀ | Emax | | | | IC₅₀ | Emax |
|---|---|---|---|---|---|---|---|---|---|
| | R₁ | CLogP | (μM) | (F/R) | | R₁ | CLogP | (μM) | (F/R) |
| I-1o | o-F | 4.15 | 17.5 ± 1.1 | 2.10 | I-7o | o-Me | 4.09 | >20 | 0.98 |
| I-1m | m-F | 4.60 | 8.3 ± 0.9 | 6.54 | I-7m | m-Me | 4.65 | 14.4 ± 0.5 | 4.73 |
| I-1p | p-F | 4.60 | 20 ± 2.1 | 0.98 | I-7p | p-Me | 4.65 | >20 | 1.05 |
| I-2o | o-Cl | 4.61 | 9.1 ± 1.0 | 5.37 | I-8o | o-MeO | 4.26 | >20 | 1.51 |
| I-2m | m-Cl | 5.17 | 5.9 ± 0.6 | 8.76 | I-8m | m-MeO | 4.26 | 14 ± 1.3 | 3.81 |
| I-2p | p-Cl | 5.17 | >20 | 1.65 | I-8p | p-MeO | 4.26 | >20 | 0.83 |
| I-3o | o-Br | 4.76 | 7.7 ± 0.1 | 5.23 | I-9o | o-NO₂ | 4.54 | 3.5 ± 0.3 | 2.23 |
| I-3m | m-Br | 5.32 | 6.2 ± 0.3 | 9.52 | I-9m | m-NO₂ | 4.54 | 6.7 ± 0.7 | 7.47 |
| I-3p | p-Br | 5.32 | >20 | 1.29 | I-9p | p-NO₂ | 4.54 | 2.3 ± 0.1 | 9.00 |
| I-4o | o-I | 4.92 | >20 | 3.25 | I-10o | o-MeS | 4.71 | >20 | 0.92 |
| I-4m | m-I | 5.58 | 5.2 ± 0.2 | 10.32 | I-10m | m-MeS | 4.71 | 11.1 ± 1.1 | 6.67 |
| I-4p | p-I | 5.58 | 10.2 ± 1.5 | 3.77 | I-10p | p-MeS | 4.71 | >20 | 1.07 |
| I-5o | o-CF₃ | 4.69 | >20 | 1.56 | I-11o* | o-CO₂Me | 4.67 | 14.5 ± 1.6 | 3.79 |
| I-5m | m-CF₃ | 5.57 | 2.6 ± 0.3 | 11.84 | I-11m | m-CO₂Me | 4.67 | 9.2 ± 0.8 | 6.83 |
| I-5p | p-CF₃ | 5.57 | 2.8 ± 0.1 | 13.99 | I-11p | p-CO₂Me | 4.67 | 11.6± | 3.97 |
| I-6o | o-CF₃O | 5.36 | >20 | 1.41 | I-12o* | o-CO₂H | 4.87 | >20 | 1.20 |
| I-6p | p-CF₃O | 3.81 | 2.7 ± 0.1 | 15.32 | I-12m | m-CO₂H | 4.24 | >20 | 0.88 |
| I-13 | H | 4.15 | 20 ± 2.0 | 1.65 | I-12p | p-CO₂H | 4.24 | 11.6 ± 1.8 | 3.49 |

*Compounds synthesized as described in Scheme 1, all other compounds were part of University of California at Davis library.

Referring to FIG. 1, the graph shows the activity of N-phenyl-N′-(4-phenoxy)cyclohexylureas in the surrogate eIF2α phosphorylation assay. Activity of the compounds was measured by dual luciferase (DLR) assay, the F/R ratio normalized to vehicle treated cells, and expressed as a function of the compound concentration. N-phenyl,N′-cyclohexylarylureas were sorted into three groups based on the nature of the R₁ substituent: FIG. 1A—halogen substituent; FIG. 1B—electron donating groups; and FIG. 1C—electron withdrawing groups. The experiment was conducted in triplicate and each experiment was independently performed three times; data are shown as Mean±S.E.M. (S.E.M.=standard error of the mean)

TABLE 2
Activity of N-phenyl-N′-(4-((2,6-difluorobenzyl)oxy)cyclohexyl)
ureas I in the ternary complex and cell proliferation assay.
II

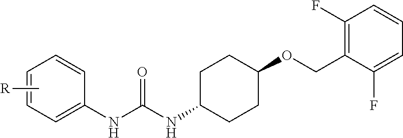

| | | Emax (F/R) (μM) | | | IC₅₀ |
|---|---|---|---|---|---|
| Compound | R | 30 | 15 | 7.5 | (μM) |
| II-1o | o-F | 0.89 | 0.98 | 0.96 | >20 |
| II-1m | m-F | 0.67 | 0.77 | 0.72 | >20 |
| II-2o | o-Cl | 0.81 | 0.69 | 0.81 | >20 |
| II-2m | m-Cl | 0.75 | 0.78 | 0.79 | >20 |
| II-2p | p-Cl | 0.74 | 0.96 | 0.95 | >20 |
| II-3o | o-Br | 0.96 | 0.99 | 0.96 | >20 |
| II-3m | m-Br | 0.98 | 1.04 | 1.07 | >20 |
| II-3p | p-Br | 0.73 | 0.85 | 0.76 | >20 |
| II-4o | o-I | 0.81 | 0.74 | 0.81 | >20 |
| II-4m | m-I | 0.72 | 0.75 | 0.69 | >20 |
| II-4p | p-I | 0.67 | 0.82 | 0.76 | >20 |
| II-5o | o-CF₃ | 0.64 | 0.80 | 0.95 | >20 |
| II-5m | m-CF₃ | 1.11 | 1.15 | 1.13 | >20 |
| II-5p | p-CF₃ | 0.85 | 0.79 | 0.91 | >20 |
| II-6o | o-CF₃O | 0.76 | 0.93 | 0.93 | >20 |
| II-6p | p-CF₃O | 0.98 | 0.80 | 0.81 | >20 |
| II-7o | o-Me | 0.83 | 0.79 | 0.78 | >20 |
| II-7m | m-Me | 0.81 | 0.89 | 0.82 | >20 |
| II-7p | p-Me | 0.77 | 0.81 | 0.81 | >20 |
| II-8o | o-MeO | 0.84 | 0.88 | 0.94 | >20 |
| II-8m | m-MeO | 0.74 | 0.97 | 0.84 | >20 |
| II-8p | p-MeO | 0.68 | 0.71 | 0.74 | >20 |
| II-9o | o-NO₂ | 0.74 | 0.90 | 0.96 | >20 |
| II-9m | m-NO₂ | 0.94 | 0.76 | 0.96 | >20 |
| II-9p | p-NO₂ | 0.83 | 0.83 | 0.87 | >20 |
| II-10o | o-MeS | 0.83 | 0.90 | 0.82 | >20 |
| II-10m | m-MeS | 1.06 | 0.73 | 1.01 | >20 |
| II-10p | p-MeS | 1.07 | 0.98 | 0.94 | >20 |
| II-11 | H | 0.81 | 0.88 | 0.89 | >20 |

### Example 2

### General Procedure for the Synthesis of N-Substituted Cyclohexyl Amines, 1-9 (General Procedure A)

To a stirred solution of aminocyclohexanol, n=2, 3, or 4, (230 mg, 2 mmol) in DMF (10 mL) at 0° C., was added 60% NaH in mineral oil (240 mg, 6 mmol). The reaction mixture was stirred at room temperature for 1 hour and then 1-fluoro-n-substituted benzene (2.4 mmol) was added. The vessel was heated to 60° C. for 2 hours then stirred for 16 hours at room temperature, whereupon the reaction mixture was diluted with ethyl acetate and washed with 10 mL of water (3 times) and brine. The organic layer was dried using sodium sulfate, filtered, and concentrated under vacuum. The product was purified using reversed phase column chromatography in gradient increase of methanol in 0.1% formic acid solution (see, for example, U.S. Patent Application No. 2005/0215784.) The compounds prepared herein and some exemplary characterization data are described in detail below.

### 4-(3-Fluorophenoxy)cyclohexanamine

1: Using General Procedure A employing trans-4-aminocyclohexanol and 1,3-difluorobenzene to afford 1 as a white solid, 94% (394 mg) yield, mp. 176° C. RP-HPLC (C18): 0 to 100% (ACN/Water/0.1% TFA) in 25 min, t_{R}=9.966 min, purity of 99.2%; ¹H NMR (399 MHz, DMSO-d₆) δ 8.44 (s, 1H), 7.25 (q, J=7.9 Hz, 1H), 6.75 (dddd, J=31.0, 17.0, 8.8, 2.5 Hz, 2H), 4.63 (bs, 2H), 4.28 (tt, J=9.3, 4.3 Hz, 1H), 3.07-2.78 (m, 1H), 1.99 (dd, J=42.9, 11.4 Hz, 4H), 1.42 (dtd, J=22.6, 12.9, 6.1 Hz, 4H). ¹³C NMR (100 MHz, DMSO) δ 166.63, 159.40, 131.38, 131.28, 112.60, 112.58, 107.88, 107.67, 103.79, 103.55, 75.00, 48.85, 29.82, 29.44. ¹⁹F NMR (376 MHz, DMSO) δ −112.16. LCMS(ESI) for C₁₂H₁₆FNO [M+H]⁺: m/z calcd: 210.12. found: 209.90.

### 4-(2-Fluorophenoxy)cyclohexanamine

2: Using General Procedure A employing trans-4-aminocyclohexanol and 1,2-difluorobenzene to afford 2 as a white solid, 90% (376 mg) yield, mp. 165° C. RP-HPLC (C18): 0 to 100% (ACN/Water/0.1% TFA) in 25 min, t_{R}=9.543 min, purity of 99.7%; ¹H NMR (399 MHz, DMSO-d₆) δ 8.44 (s, 1H), 7.25-7.11 (m, 1H), 7.07 (t, J=7.8 Hz, 1H), 7.04-6.83 (m, 1H), 4.99 (s, 2H), 4.35-4.03 (m, 1H), 3.14-2.71 (m, 1H), 2.22-1.71 (m, 4H), 1.43 (h, J=11.5, 10.3 Hz, 4H). ¹³C NMR (100 MHz, DMSO) δ 166.73, 154.61, 125.43, 125.39, 122.24, 118.31, 116.99, 116.81, 76.59, 48.76, 29.93, 29.34. ¹⁹F NMR (376 MHz, DMSO) δ −134.32. LCMS(ESI) for C₁₂H₁₆FNO [M+H]⁺: m/z calcd: 210.12. found: 209.84.

### 4-(4-Chlorophenoxy)cyclohexanamine

3: Using General Procedure A employing trans-4-aminocyclohexanol and 1-chloro-4-fluorobenzene to afford 3 as a white solid, 85% (383 mg) yield, mp. 198° C. RP-HPLC (C18): 0 to 100% (ACN/Water/0.1% TFA) in 25 min, t_{R}=19.302 min, purity of 100%; ¹H NMR (399 MHz, DMSO-d₆) δ 8.40 (s, 111), 7.39-7.15 (m, 2H), 7.12-6.80 (m, 2H), 4.22 (dd, J=9.8, 4.9 Hz, 1H), 3.63 (bs, 2H), 3.09-2.80 (m, 1H), 2.13-1.92 (m, 4H), 1.51-1.36 (m, 4H). ¹³C NMR (100 MHz, DMSO) δ 156.78, 129.94, 124.67, 118.17, 75.08, 48.95, 29.84, 29.68. LCMS(ESI) for C₁₂H₁₆ClNO [M+H]⁺: m/z calcd: 226.09. found: 225.99.

### 4-(4-(Trifluoromethyl)phenoxy)cyclohexanamine

4: Using General Procedure A employing trans-4-aminocyclohexanol and 1-fluoro-4-(trifluoromethyl)benzene to afford 4 as a white solid, 95% (493 mg) yield, mp. 158° C. RP-HPLC (C18): 0 to 100% (ACN/Water/0.1% TFA) in 25 min, t_{R}=11.864 min, purity of 99.8%; ¹H NMR (399 MHz, DMSO-d₆) δ 8.44 (s, 1H), 7.58 (d, J=8.4 Hz, 2H), 7.11 (d, J=8.5 Hz, 2H), 4.80 (s, 2H), 4.43-4.30 (m, 1H), 2.95 (dq, J=11.1, 5.6, 5.1 Hz, 1H), 2.00 (dt, J=47.0, 13.6 Hz, 4H), 1.43 (p, J=16.1, 14.4 Hz, 4H). ¹³C NMR (100 MHz, DMSO) δ 166.70, 160.88, 127.58, 127.57, 116.56, 74.95, 48.77, 29.70, 29.35. ¹⁹F NMR (376 MHz, DMSO) δ −60.28. LCMS(ESI) for C₁₃H₁₆F₃NO [M+H]⁺: m/z calcd: 260.12. found: 260.01.

### 4-(4-(Trifluoromethoxy)phenoxy)cyclohexanamine

5: Using General Procedure A employing trans-4-aminocyclohexanol and 1-fluoro-4-(trifluoromethoxy)benzene to afford 5 as a white solid, 93% (512 mg) yield, mp. 147° C. RP-HPLC (C18): 0 to 100% (ACN/Water/0.1% TFA) in 25 min, t_{R}=12.283 min, purity of 100%; ¹H NMR (399 MHz, DMSO-d₆) δ 8.44 (s, 1H), 7.21 (d, J=8.6 Hz, 2H), 7.01 (d, J=9.1 Hz, 2H), 5.58 (s, 2H), 4.25 (tt, J=8.7, 4.2 Hz, 1H), 2.97 (dq, J=12.5, 5.8 Hz, 1H), 2.34-1.77 (m, 4H), 1.43 (dt, J=22.4, 12.3 Hz, 4H). ¹³C NMR (100 MHz, DMSO) δ 166.73, 156.76, 142.35, 123.09, 117.55, 75.14, 48.75, 29.76, 29.00. ¹⁹F NMR (376 MHz, DMSO) δ −57.76. LCMS(ESI) for C₁₃H₁₆F₃NO₂ [M+H]⁺: m/z calcd: 276.11. found: 276.16.

### 4-(2-(Trifluoromethyl)phenoxy)cyclohexanamine

6: Using General Procedure A employing trans-4-aminocyclohexanol and 1-fluoro-2-(trifluoromethyl)benzene to afford 6 as a white solid, 90% (467 mg) yield, mp. 140° C. RP-HPLC (C18): 0 to 100% (ACN/Water/0.1% TFA) in 25 min, t_{R}=11.595 min, purity of 99%; ¹H NMR (399 MHz, DMSO-d₆) δ 8.45 (s, 1H), 7.53 (d, J=7.7 Hz, 2H), 7.31 (d, J=8.6 Hz, 1H), 7.01 (t, J=7.6 Hz, 1H), 6.56 (bs, 2H), 4.45 (ddt, J=10.1, 7.9, 4.0 Hz, 1H), 3.04 (ddt, J=10.5, 7.6, 3.8 Hz, 1H), 2.14-1.82 (m, 4H), 1.47 (dddd, J=25.2, 15.9, 12.8, 6.5 Hz, 4H). ¹³C NMR (100 MHz, DMSO) δ 166.90, 155.92, 134.62, 128.50, 127.42, 127.36, 125.79, 123.08, 120.82, 118.79, 118.49, 115.67, 75.43, 48.46, 29.38, 28.43. ¹⁹F NMR (376 MHz, DMSO) δ −61.23. LCMS(ESI) for C₁₃H₁₆F₃NO [M+H]⁺: m/z calcd: 260.12. found: 260.08.

### 4-(3-(Trifluoromethyl)phenoxy)cyclohexanamine

7: Using General Procedure A employing trans-4-aminocyclohexanol and 1-fluoro-3-(trifluoromethyl)benzene to afford 7 as a white solid, 92% (477 mg) yield, mp. 133° C. RP-HPLC (C18): 0 to 100% (ACN/Water/0.1% TFA) in 25 min, t_{R}=11.761 min, purity of 99%; ¹H NMR (399 MHz, DMSO-d₆) δ 8.44 (s, 1H), 7.47 (t, J=8.0 Hz, 1H), 7.33-7.05 (m, 2H), 6.00 (s, 2H), 4.39 (td, J=9.9, 5.0 Hz, 1H), 3.15-2.85 (m, 1H), 2.29-1.77 (m, 4H), 1.67-1.24 (m, 4H).

¹³C NMR (100 MHz, DMSO) δ 166.72, 158.22, 131.38, 120.29, 117.66, 113.04, 74.94, 48.71, 29.67, 28.81. ¹⁹F NMR (376 MHz, DMSO) δ −61.62. LCMS(ESI) for C₁₃H₁₆F₃NO [M+H]⁺: m/z calcd: 260.12. found: 260.08.

### 2-(4-(trifluoromethyl)phenoxy)cyclohexanamine

8: Using General Procedure A employing 2-aminocyclohexanol and 1-fluoro-4-(trifluoromethyl)benzene to afford 8 as a white solid, 47% (246 mg) yield, mp. 131° C. RP-HPLC (C18): 0 to 100% (ACN/Water/0.1% TFA) in 25 min, t_{R}=11.895 min, purity of 100%; ¹H NMR (399 MHz, DMSO-d₆) δ 8.41 (s, 1H), 7.62 (d, J=8.0 Hz, 2H), 7.18 (d, J=8.1 Hz, 2H), 6.09 (bs, 2H), 4.92-4.75 (m, 1H), 3.29 (d, J=8.0 Hz, 1H), 2.03-1.89 (m, 1H), 1.88-1.59 (m, 3H), 1.52 (q, J=7.0, 6.6 Hz, 1H), 1.44-1.24 (m, 3H). ¹³C NMR (100 MHz, DMSO) δ 166.57, 160.66, 127.54, 127.49, 117.33, 117.01, 74.25, 51.13, 27.00, 26.84, 23.26, 19.82. ¹⁹F NMR (376 MHz, DMSO) δ −60.45. LCMS(ESI) for C₁₃H₁₆F₃NO [M+H]⁺: m/z calcd: 260.12. found: 260.01.

### 3-(4-(Trifluoromethyl)phenoxy)cyclohexanamine

9: Using General Procedure A employing 3-aminocyclohexanol and 1-fluoro-4-(trifluoromethyl)benzene to afford 9 as a white solid, 38% (197 mg) yield, mp. 117° C. RP-HPLC (C18): 0 to 100% (ACN/Water/0.1% TFA) in 25 min, t_{R}=11.877 min, purity of 100%; ¹H NMR (399 MHz, DMSO-d₆) δ 8.42 (s, 1H), 7.59 (d, J=8.5 Hz, 2H), 7.11 (d, J=8.5 Hz, 2H), 6.53 (s, 2H), 5.08-4.73 (m, 1H), 3.26 (td, J=9.5, 8.1, 4.5 Hz, 1H), 2.35-1.42 (m, 8H). ¹³C NMR (100 MHz, DMSO) δ 166.74, 160.45, 127.59, 127.45, 122.02, 121.71, 116.74, 72.04, 45.90, 34.21, 30.15, 28.59, 19.07. ¹⁹F NMR (376 MHz, DMSO) δ −60.48. LCMS(ESI) for C₁₃H₁₆F₃NO [M+H]⁺: m/z calcd: 260.12. found: 260.08.

### Example 3

### Synthesis of 4-Methoxycyclohexanamine, 10

trans-4-Aminocyclohexanol (2 mmol) was dissolved in 5 mL of dry methanol, then 1.1 equiv of di-tert-butyl dicarbonate were dissolved in 5 mL methanol and added dropwise at room temperature. The reaction was stirred until deemed complete by TLC, whereupon the solvent was removed by evaporation. The crude residue was dissolved in 5 mL of dry THF, and cooled to 0° C. Sodium hydride (60% in mineral oil, 2.2 mmol) was added. The resulting mixture was stirred at room temperature for 30 min, whereupon 1.5 equiv of iodomethane was added. The reaction was stirred for 16 hours at room temperature, then diluted with ethyl acetate and washed with 10 mL of water (3 times) and brine. The organic layer was dried over sodium sulfate, the solids were removed by filtration, and the organics were concentrated under vacuum. The crude product was dissolved in 2 mL of DCM/TFA (dichloromethane/trifluoroacetic acid), 1:1, solvent mixture and stirred for 1 hr. Solvent was removed by evaporation, and the obtained oil was purified using reversed phase column chromatography in gradient increase of methanol in 0.1% Formic acid solution. Yellow oil, 75% (163 mg) yield; ¹H NMR (400 MHz, DMSO-d₆) δ 8.94 (s, 1H), 3.28 (t, J=2.2 Hz, 3H), 3.19-3.07 (m, 1H), 2.97 (t, J=11.8 Hz, 1H), 2.09 (d, J=11.1 Hz, 4H), 1.40 (q, J=11.8 Hz, 2H), 1.26-1.12 (m, 2H). ¹³C NMR (100 MHz, DMSO) δ 77.46, 56.44, 55.52, 29.39, 26.42. LCMS(ESI) for C₇H₁₅NO [M+H]⁺: m/z calcd: 130.12. found: 129.91.

### Example 4

### Synthesis of 4-((4-Fluorobenzyl)oxy)cyclohexanamine, 11

trans-4-Aminocyclohexanol (2 mmol) was dissolved in 5 mL of dry methanol, then 1.1 equiv of di-tert-butyl dicarbonate were dissolved in 5 mL methanol and added dropwise at room temperature. The reaction was stirred until completion by TLC, then solvent was evaporated to dryness. The produced crude was redissolved in 5 mL of dry THF, and cooled to 0° C. Then 2.2 mmol of 60% sodium hydride in mineral oil were added. The reaction mixture was stirred at room temperature for 30 min and then 1.5 equiv of 4-fluorobenzylbromide was added. The reaction was stirred for 16 hours at room temperature. The reaction mixture was diluted with ethyl acetate and washed with 10 mL of water (3 times) and brine. The organic layer was dried using sodium sulfate, filtered, and concentrated under vacuum. Then the product was dissolved in 2 mL of DCM/TFA, 1:1, solvent mixture and stirred for 1 hr. Then solvent was evaporated to dryness and the obtained solid was purified using reversed phase column chromatography in gradient increase of methanol in 0.1% formic acid solution. White solid, 96% (429 mg) yield, mp. 166° C. RP-HPLC (C18): 0 to 100% (ACN/Water/0.1% TFA) in 25 min, t_{R}=9.528 min, purity of 97.8%; ¹H NMR (399 MHz, DMSO-d₆) δ 7.33 (dd, J=8.1, 5.5 Hz, 2H), 7.13 (t, J=8.6 Hz, 2H), 6.90 (bs, 2H), 4.46 (s, 2H), 4.28-4.21 (m, 1H), 3.31-3.28 (m, 1H), 2.98 (dt, J=10.9, 5.9 Hz, 1H), 1.97 (dd, J=43.0, 12.0 Hz, 4H), 1.37-1.15 (m, 4H). ¹³C NMR (100 MHz, DMSO) δ 130.06, 129.98, 115.71, 115.50, 76.05, 75.87, 69.07, 49.23, 49.03, 30.07, 29.85, 28.84, 28.51. ¹⁹F NMR (376 MHz, DMSO) δ −115.88. LCMS(ESI) for C₁₃H₁₈FNO [M+H]⁺: m/z calcd: 224.14. found: 223.81.

### Example 5

### Synthesis of 1-(4-(4-fluorophenoxy)cyclohexyl)-3-(3-(trifluoromethoxy)phenyl)urea, I-6m

To a stirring solution of 4-(4-fluorophenoxy)cyclohexanamine (see Hwang, S. H. et al. Journal of Medicinal Chemistry 2007, 50(16), 3825-40) (70 mg, 0.33 mmol) in dichloromethane (3 mL) under nitrogen was added freshly prepared 1-isocyanato-3-(trifluoromethoxy)benzene (see Zhang, Y. et al. ACS Medicinal Chemistry Letters 2010, 1(9), 460-465) (61 mg, 0.30 mmol) in DMSO, followed by triethylamine (46 μL, 0.33 mmol). The mixture was maintained at room temperature for 16 h and the reaction monitored for completion by TLC. Crude mixture was washed with brine and extracted using dichloromethane, dried under magnesium sulfate and purified using column chromatography using hexane and ethyl acetate gradient to obtain desired product as white amorphous solid (26 mg, 19% yield). ¹H NMR (500 MHz, CDCl₃) δ 7.34 (s, 1H), 7.31-7.28 (m, 1H), 7.19-7.17 (m, 1H), 6.98-6.94 (m, 2H), 6.91-6.89 (m, 1H), 6.84-6.82 (m, 2H), 6.36 (s, 1H), 4.66 (d, J=7.5 Hz, 1H), 4.37 (s, 1H), 3.87-3.76 (m, 1H), 1.99-1.97 (m, 2H), 1.84-1.82 (m, 2H), 1.69-1.56 (m, 4H); ¹³C NMR δ 158.46, 156.56, 155.52, 153.59, 149.93, 140.44, 130.33, 121.62, 17.60, 116.10, 115.99, 115.42, 112.66, 72.42, 60.84, 48.14, 28.44. ¹⁹F δ 59.20 (s, 3H), −6.53 (s, 1H); LCMS for C₂₀H₂₀F₄N₂O₃ [M+H]⁺: m/z calcd: 413.37. found: 413.15.

### Example 6

### Synthesis of methyl 2-(3-(4-(4-fluorophenoxy)cyclohexyl)ureido)benzoate, I-11o

To a stirring solution of 1 (see Hwang, S. H. et al. Journal of Medicinal Chemistry 2007, 50(16), 3825-40) (20 mg, 0.09 mmol) in dichloromethane (2 mL) under nitrogen was added methyl 2-isocyanatobenzoate (15 mg, 0.08 mmol). The mixture was maintained at room temperature for 16 h and the reaction monitored for completion by TLC. Crude mixture was washed with brine and extracted using dichloromethane, dried under magnesium sulfate and column chromatographed using hexane and ethyl acetate gradient to obtain desired product as white amorphous solid (22 mg, 59% yield). ¹H NMR (500 MHz, CDCl₃) δ 10.33 (s, 1H), 8.53 (d, J=8.5 Hz, 1H), 7.98 (d, J=8.0 Hz, 1H), 7.49 (dd, J=8.5 Hz, 7.5 Hz, 1H), 6.99-6.94 (m, 3H), 6.86-6.84 (m, 2H), 4.72 (d, J=8.0 Hz, 1H), 4.40 (s, 1H), 3.90 (s, 3H), 3.79-3.75 (m, 1H), 2.04-2.00 (m, 2H), 1.86-1.84 (m, 2H), 1.74-1.66 (m, 4H); ¹³C NMR δ 169.46, 158.41, 156.51, 154.35, 153.62, 143.59, 134.84, 130.90, 120.75, 119.54, 117.60, 117.54, 116.16, 115.98, 113.73, 72.16, 52.35, 48.35, 28.56, 28.36, 28.04; ¹⁹F NMR (376 MHz, DMSO) δ −40.61. LCMS for C₂₁H₂₃FN₂O₄ [M+H]⁺: m/z calcd: 387.41. found: 387.20.

### Example 7

### Synthesis of 2-(3-(4-(4-fluorophenoxy)cyclohexyl)ureido)benzoic acid, I-12o

To a solution of urea I-11o (14 mg, 0.04 mmol) in 1 mL of acetonitrile was added lithium hydroxide (3 mg, 0.11 mmol) in water (0.3 mL) and reaction mixture was stirred at room temperature for 24 hours. Solvent was evaporated and the crude mixture was dissolved in methanol and filter through a silica pipette to obtain pure product as white solid (10 mg, 74%). ¹H NMR (500 MHz, CDCl₃) δ 10.69 (s, 1H), 8.13 (d, J=8.0 Hz, 1H), 7.54 (t, J=8.0 Hz, 1H), 7.21 (t, J=7.5 Hz, 1H), 7.14 (d, J=8.5 Hz, 1H), 6.98 (d, J=7.0 Hz, 4H), 6.04 (s, 1H), 5.56 (s, 1H), 5.03 (t, J=12.5 Hz, 1H), 4.51 (s, 1H), 3.05-2.98 (m, 2H), 2.22-2.19 (m, 2H), 1.71-1.66 (m, 2H), 1.56-1.54 (m, 2H); ¹³C NMR δ 163.53, 158.44, 153.76, 152.56, 139.08, 135.03, 128.68, 123.38, 118.04, 117.98, 116.13, 115.95, 115.17, 114.99, 71.41, 53.18, 29.76, 23.09; LCMS for C₂₀H₂₁FN₂O₄ [M+H]⁺: m/z calcd: 373.39. found: 373.15.

### Example 8

### General procedure for synthesis of the N-aryl,N′-cyclohexylureas, I-14-20 and III-1-3, 5, 6 (General Procedure B)

To a stirred solution of 3-(trifluoromethyl)phenylisocyanate (1 mmol) in dry DMF (2 mL) and triethylamine (1 mmol), the desired amine (1 mmol) was added and the reaction was left at RT for 16 hrs. The reaction mixture was treated with DDW (5 mL) and the formed precipitate was collected, washed with water, and re-crystallized twice from methanol/water. In some cases further purification by reversed phase column chromatography employing a linear gradient of (0.1% formic acid/water) in methanol, (0%-100%) was needed to achieve purity>95%.

### 1-(4-(2-Fluorophenoxy)cyclohexyl)-3-(3-(trifluoromethyl)phenyl)urea, I-14

Using General Procedure B employing trans-4-(2-fluorophenoxy)cyclohexylamine 2, to afford I-14 as a white solid, 56% (220 mg) yield, mp. 171° C. RP-HPLC (C18): 0 to 100% (ACN/Water/0.1% TFA) in 25 min, t_{R}=18.365 min, purity of 99%; ¹H NMR (300 MHz, DMSO-d₆) δ 8.63 (s, 1H), 7.94 (s, 1H), 7.51-7.33 (m, 2H), 7.24 (dq, J=20.4, 7.1, 6.4 Hz, 2H), 6.73 ((dt, J=16.5, 11.0 Hz, 2H m, 3H), 6.24 (d, J=5.5 Hz, 1H), 4.34 (q, J=9.0, 8.4 Hz, 1H), 3.56-3.45 (m, 1H), 2.03-1.87 (m, 4H), 1.49-1.31 (m, 4H). ¹³C NMR (100 MHz, DMSO) δ 155.08, 141.97, 130.38, 125.38, 122.11, 121.74, 118.26, 117.00, 116.82, 114.18, 76.74, 47.91, 30.43. ¹⁹F NMR (376 MHz, DMSO) δ −61.80, −134.26. HRMS(ESI) for C₂₀H₂₀F₄N₂O₂ [M+H]⁺: m/z calcd: 397.15337. found: 397.15317.

### 1-(4-(3-Fluorophenoxy)cyclohexyl)-3-(3-(trifluoromethyl)phenyl)urea, I-15

Using General Procedure B employing trans-4-(3-fluorophenoxy)cyclohexylamine 1, to afford I-15 as a white solid, 66% (260 mg) yield, mp. 193° C. RP-HPLC (C18): 0 to 100% (ACN/Water/0.1% TFA) in 25 min, t_{R}=18.857 min, purity of 100%; ¹H NMR (300 MHz, DMSO-d₆) δ 8.64 (s, 1H), 7.94 (s, 1H), 7.58 (d, J=8.4 Hz, 2H), 7.41 (d, J=5.5 Hz, 2H), 7.18 (d, J=5.9 Hz, 1H), 7.10 (d, J=8.4 Hz, 2H), 6.24 (d, J=7.5 Hz, 1H), 4.57-4.33 (m, 1H), 3.55-3.46 (m, 1H), 2.13-1.75 (m, 4H), 1.41 (dp, J=23.6, 12.0 Hz, 4H). ¹³C NMR (100 MHz, DMSO) δ 155.06, 141.96, 131.38, 131.26, 130.39, 121.74, 117.83, 114.14, 112.59, 107.58, 103.49, 75.16, 47.96, 30.54, 30.29. ¹⁹F NMR (376 MHz, DMSO) δ −61.78, −112.16. HRMS(ESI) for C₂₀H₂₀F₄N₂O₂ [M+H]⁺: m/z calcd: 397.15337. found: 397.15429.

### 1-(4-(4-Chlorophenoxy)cyclohexyl)-3-(3-(trifluoromethyl)phenyl)urea, I-16

Using General Procedure B employing trans-4-(4-chlorophenoxy)cyclohexylamine 3, to afford I-16 as a white solid, 50% (206 mg) yield, mp. 244° C. RP-HPLC (C18): 0 to 100% (ACN/Water/0.1% TFA) in 25 min, t_{R}=19.617 min, purity of 100%; ¹H NMR (300 MHz, DMSO-d₆): δ 8.65 (s, 1H), 7.95 (s, 1H), 7.42 (d, J=7.9 Hz, 2H), 7.27 (dd, J=9.0, 3.3 Hz, 2H), 7.19 (d, J=6.8 Hz, 1H), 6.95 (dd, J=8.7, 3.5 Hz, 2H), 6.25 (d, J=4.0 Hz, 1H), 4.32-4.27 (m, 1H), 3.55-3.48 (m, 1H), 2.50-1.90 (m, 4H), 1.48-1.29 (m, 4H). ¹³C NMR (100 MHz, DMSO) δ 156.84, 155.06, 141.96, 130.40, 129.92, 124.70, 121.73, 118.08, 117.85, 114.14, 75.13, 47.96, 30.57, 30.30. ¹⁹F NMR (376 MHz, DMSO) δ −61.79. HRMS(ESI) for C₂₀H₂₀ClF₃N₂O₂ [M+H]⁺: m/z calcd: 413.12382. found: 413.12507.

### 1-(4-(4-(trifluoromethoxy)phenoxy)cyclohexyl)-3-(3-(trifluoromethyl)phenyl)urea, I-17

Using General Procedure B employing trans-4-(4-(trifluoromethoxy)phenoxy)cyclohexylamine 5, to afford I-17 as a white solid, 54% (251 mg) yield, mp. 190° C. RP-HPLC (C18): 0 to 100% (ACN/Water/0.1% TFA) in 25 min, t_{R}=20.231 min, purity of 100%; ¹H NMR (399 MHz, DMSO-d₆) δ 8.66 (s, 1H), 7.96 (s, 1H), 7.46-7.41 (m, 2H), 7.39-7.04 (m, 3H), 7.01 (d, J=8.5 Hz, 2H), 6.25 (d, J=7.6 Hz, 1H), 4.35-4.30 (m, 1H), 3.54-3.49 (dd, J=13.8, 7.3 Hz, 1H), 2.04-1.90 (m, 4H), 1.47-1.33 (m, 4H). ¹³C NMR (100 MHz, DMSO) δ 156.88, 155.07, 141.96, 130.36, 123.14, 121.71, 117.47, 114.14, 75.32, 47.96, 30.56, 30.30. ¹⁹F NMR (376 MHz, DMSO) δ −57.78, −61.87. LCMS(ESI) for C₂₁H₂₀F₆N₂O₃ [M+H]⁺: m/z calcd: 463.13. found: 463.11.

### 1-(4-(4-(Trifluoromethyl)phenoxy)cyclohexyl)-3-(3-(trifluoromethyl)phenyl)urea, I-18

Using General Procedure B employing trans-4-(4-(trifluoromethyl)phenoxy)cyclohexylamine 4, to afford I-18 as a white solid, 43% (194 mg) yield, mp. 176° C. RP-HPLC (C18): 0 to 100% (ACN/Water/0.1% TFA) in 25 min, t_{R}=19.617 min, purity of 100%; ¹H NMR (399 MHz, DMSO-d₆) δ 8.64 (s, 1H), 7.94 (s, 1H), 7.58 (d, J=8.1 Hz, 2H), 7.41 (d, 2H), 7.25-7.15 (m, 1H), 7.09 (t, J=9.3 Hz, 2H), 6.26 (d, J=7.5 Hz, 1H), 4.58-4.31 (m, 1H), 3.72-3.39 (m, 1H), 2.25-1.73 (m, 4H), 1.53-1.29 (m, 4H). ¹³C NMR (100 MHz, DMSO) δ 160.97, 155.06, 141.94, 138.79, 130.44, 127.64, 121.76, 116.00, 114.13, 75.08, 47.91, 30.53, 30.22. ¹⁹F NMR (376 MHz, DMSO) δ −60.21, −61.78. HRMS(ESI) for C₂₁H₂₀F₆N₂O₂ [M+H]⁺: m/z calcd: 447.15017. found: 447.15126.

### 1-(4-(2-(Trifluoromethyl)phenoxy)cyclohexyl)-3-(3-(trifluoromethyl)phenyl)urea, I-19

Using General Procedure B employing trans-4-(2-(trifluoromethyl)phenoxy)cyclohexylamine 6, to afford I-19 as a white solid, 69% (308 mg) yield, mp. 121° C. RP-HPLC (C18): 0 to 100% (ACN/Water/0.1% TFA) in 25 min, t_{R}=19.868 min, purity of 99%; ¹H NMR (400 MHz, DMSO-d₆) δ 8.69 (s, 1H), 7.96 (s, 1H), 7.59 (d, J=7.7 Hz, 2H), 7.45-7.41 (m, 2H), 7.31 (d, J=8.6 Hz, 1H), 7.20 (d, J=7.7 Hz, 1H), 7.06 (t, J=7.6 Hz, 1H), 6.36 (d, J=7.8 Hz, 1H), 4.56 (td, J=9.4, 4.8 Hz, 1H), 3.56 (dtt, J=15.2, 9.9, 4.9 Hz, 1H), 2.05-1.87 (m, 4H), 1.54-1.32 (m, 4H). ¹³C NMR (100 MHz, DMSO) δ 154.19, 153.28, 147.42, 146.89, 140.15, 132.83, 128.55, 128.14, 125.66, 119.89, 118.88, 116.01, 113.71, 112.30, 73.64, 45.71, 28.09, 27.88. ¹⁹F NMR (376 MHz, DMSO) δ −62.10, −62.71. HRMS(ESI) for C₂₁H₂₀F₆N₂O₂ [M+H]⁺: m/z calcd: 447.15017. found: 447.15147.

### 1-(4-(3-(Trifluoromethyl)phenoxy)cyclohexyl)-3-(3-(trifluoromethyl)phenyl)urea, I-20

Using General Procedure B employing trans-4-(3-(trifluoromethyl)phenoxy)cyclohexylamine 7, to afford I-20 as a white solid, 59% (264 mg) yield, mp. 160° C. RP-HPLC (C18): 0 to 100% (ACN/Water/0.1% TFA) in 25 min, t_{R}=20.083 min, purity of 99%; ¹H NMR (300 MHz, DMSO-d₆) δ 8.67 (s, 1H), 7.94 (s, 1H), 7.64-7.31 (m, 3H), 7.35-7.07 (m, 4H), 6.26 (d, J=7.5 Hz, 1H), 4.45 (tt, J=9.5, 4.0 Hz, 1H), 3.72-3.38 (m, 1H), 2.01-1.76 (m, 4H), 1.63-1.21 (m, 4H). ¹³C NMR (100 MHz, DMSO) δ 155.10, 142.00, 131.37, 130.40, 121.75, 120.37, 117.73, 114.23, 112.97, 75.24, 47.93, 30.48, 30.24. ¹⁹F NMR (376 MHz, DMSO) δ −61.50, −61.77. HRMS(ESI) for C₂₁H₂₀F₆N₂O₂ [M+H]⁺: m/z calcd: 447.15017. found: 447.15145.

### Example 9

### 1-Cyclohexyl-3-(3-(trifluoromethyl)phenyl)urea, III-1

Using General Procedure B employing, cyclohexylamine to afford III-1 as a white solid, 63% (180 mg) yield, mp. 180° C. RP-HPLC (C18): 0 to 100% (ACN/Water/0.1% TFA) in 25 min, t_{R}=16.943 min, purity of 98%; ¹H NMR (399 MHz, DMSO-d₆) δ 8.62 (s, 1H), 7.95 (s, 1H), 7.42 (t, J=7.4 Hz, 2H), 7.16 (d, J=7.4 Hz, 1H), 6.14 (d, J=8.3 Hz, 1H), 3.47 (m, 1H), 1.78 (d, J=12.0 Hz, 2H), 1.62 (d, J=13.2 Hz, 3H), 1.50 (d, J=13.4 Hz, 1H), 1.21 (dp, J=34.6, 12.4, 11.8 Hz, 5H). ¹³C NMR (100 MHz, DMSO) δ 154.92, 142.04, 130.30, 121.63, 117.73, 114.11, 48.43, 33.49, 25.85, 25.01. ¹⁹F NMR (376 MHz, DMSO) δ −61.91. HRMS(ESI) for C₁₄H₁₇F₃N₂O [M+H]⁺: m/z calcd: 287.13657. found: 287.13707.

### Example 10

### 1-(4-Hydroxycyclohexyl)-3-(3-(trifluoromethyl)phenyl)urea, III-2

Using General Procedure B employing trans-4-aminocyclohexanol, to afford III-2 as a white solid, 59% (178 mg) yield, mp. 249° C. RP-HPLC (C18): 0 to 100% (ACN/Water/0.1% TFA) in 25 min, t_{R}=12.467 min, purity of 100%; ¹H NMR (399 MHz, DMSO-d₆) δ 8.62 (s, 1H), 7.94 (s, 1H), 7.41 (td, J=10.8, 7.2 Hz, 2H), 7.17 (d, J=7.1 Hz, 1H), 6.09 (d, J=8.3 Hz, 1H), 4.52-4.47 (m, 1H), 3.39-3.20 (m, 2H), 1.85-1.78 (m, 4H), 1.24-1.13 (m, 4H). ¹³C NMR (100 MHz, DMSO) δ 155.07, 142.01, 130.31, 130.00, 121.71, 117.79, 114.19, 68.68, 48.43, 34.45, 31.32. ¹⁹F NMR (376 MHz, DMSO) δ −61.88. HRMS(ESI) for C₁₄H₁₇F₃N₂O₂ [M+H]⁺: m/z calcd: 303.13149. found: 303.13204.

### Example 11

### 1-(4-Methoxycyclohexyl)-3-(3-(trifluoromethyl)phenyl)urea, III-3

Using General Procedure B employing trans-4-methoxycyclohexanamine 10, to afford III-3 as a white solid, 32% (100 mg) yield, mp. 209° C. RP-HPLC (C18): 0 to 100% (ACN/Water/0.1% TFA) in 25 min, t_{R}=15.151 min, purity of 97%; ¹H NMR (399 MHz, DMSO-d₆) δ 8.53 (s, 1H), 7.91 (s, 1H), 7.73 (d, J=8.6 Hz, 1H), 7.43 (t, J=8.1 Hz, 1H), 7.23 (d, J=7.9 Hz, 1H), 4.00 (dq, J=12.9, 7.0, 5.6 Hz, 1H), 3.25-3.16 (m, 3H), 3.11-3.03 (m, 1H), 2.85-2.70 (m, 3H), 2.03 (d, J=12.6 Hz, 2H), 1.66-1.44 (m, 4H), 1.18 (td, J=12.9, 12.2, 5.2 Hz, 2H). ¹³C NMR (100 MHz, DMSO) δ 155.66, 142.30, 129.98, 123.76, 118.39, 78.38, 55.81, 53.50, 31.31, 29.18, 27.91. ¹⁹F NMR (376 MHz, DMSO) δ −61.65. HRMS(ESI) for C₁₅H₁₉F₃N₂O₂ [M−H]⁺: m/z calcd: 315.13259. found: 315.13314.

### Example 12

### 1-(4-((4-Fluorobenzyl)oxy)cyclohexyl)-3-(3-(trifluoromethyl)phenyl)urea, III-4

Using General Procedure B employing trans-4-((4-fluorobenzyl)oxy)cyclohexylamine 10, to afford III-4 as a white solid, 33% (136 mg) yield, mp. 162° C. RP-HPLC (C18): 0 to 100% (ACN/Water/0.1% TFA) in 25 min, t_{R}=18.230 min, purity of 95%; ¹H NMR (300 MHz, DMSO-d₆) δ 8.63 (s, 1H), 7.93 (s, 1H), 7.41-7.39 (m, 2H), 7.34-7.30 (m, 1H), 7.18-7.09 (m, 3H), 6.14 (d, J=7.7 Hz, 1H), 4.44 (s, 2H), 3.42 (dd, J=12.3, 5.4 Hz, 1H), 3.49-3.36 (m, 1H), 3.35-3.33 (m, 1H), 2.09-1.71 (m, 4H), 1.24 (dq, J=21.0, 11.0, 4H). ¹³C NMR (100 MHz, DMSO) δ 155.12, 142.09, 136.18, 130.36, 130.03, 129.95, 121.68, 117.71, 115.70, 115.49, 76.42, 68.94, 48.21, 30.90, 30.85. ¹⁹F NMR (376 MHz, DMSO) δ −61.78, −116.01. LCMS(ESI) for C₂₁H₂₂F₄N₂O₂ [M+H]⁺: m/z calcd: 411.16. found: 411.12.

### Example 13

### 1-(2-(4-(Trifluoromethyl)phenoxy)cyclohexyl)-3-(3-(trifluoromethyl)phenyl)urea, III-5

Using General Procedure B employing trans-2-(4-(trifluoromethyl)phenoxy)cyclohexylamine 8, to afford III-5 as a white solid, 79% (354 mg) yield, mp. 58° C. RP-HPLC (C18): 0 to 100% (ACN/Water/0.1% TFA) in 25 min, t_{R}=20.047 min, purity of 97%; ¹H NMR (500 MHz, DMSO-d₆) δ 8.84 (s, 1H), 7.92 (s, 1H), 7.61 (d, J=8.4 Hz, 2H), 7.45-7.31 (m, 2H), 7.18 (d, J=8.4 Hz, 3H), 6.38 (d, J=8.0 Hz, 1H), 4.70 (dt, J=5.1, 2.58 Hz, 1H), 3.96-3.77 (m, 1H), 1.99-1.84 (m, 1H), 1.67 (q, J=4.5, 4.0 Hz, 3H), 1.65 (dd, J=14.5, 7.8 Hz, 1H), 1.46-1.31 (m, 3H). ¹³C NMR (100 MHz, DMSO) δ 16.77, 154.70, 147.32, 141.57, 130.21, 127.36, 121.33, 116.72, 75.26, 49.83, 28.15, 27.56, 23.83, 19.91. ¹⁹F NMR (376 MHz, DMSO) δ −59.88, −61.42. HRMS(ESI) for C₂₁H₂₀F₆N₂O₂ [M+H]⁺: m/z calcd: 447.15017. found: 447.15122.

### Example 14

### 1-(3-(4-(Trifluoromethyl)phenoxy)cyclohexyl)-3-(3-(trifluoromethyl)phenyl)urea, III-6

Using General Procedure B employing trans-3-(4-(trifluoromethyl)phenoxy)cyclohexylamine 9, to afford III-6 as a white solid, 69% (306 mg) yield, mp. 62° C. RP-HPLC (C18): 0 to 100% (ACN/Water/0.1% TFA) in 25 min, t_{R}=20.183 min, purity of 100%; ¹H NMR (399 MHz, DMSO-d₆) δ 8.74 (s, 1H), 7.94 (s, 1H), 7.60 (d, J=8.6 Hz, 2H), 7.42 (dt, J=15.5, 8.2 Hz, 2H), 7.17 (d, J=7.7 Hz, 1H), 7.10 (d, J=8.5 Hz, 2H), 6.35 (d, J=8.1 Hz, 1H), 4.80 (s, 1H), 3.90 (dp, J=14.0, 5.4, 4.6 Hz, 1H), 2.01-1.91 (m, 1H), 1.87-1.69 (m, 2H), 1.68-1.50 (m, 4H), 1.44-1.21 (m, 1H). ¹³C NMR (100 MHz, DMSO) δ 160.76, 155.01, 142.00, 130.33, 127.66, 127.75, 117.82, 116.61, 114.19, 73.09, 44.70, 36.55, 32.45, 29.33, 19.90. ¹⁹F NMR (376 MHz, DMSO) δ −60.34, −61.86. HRMS(ESI) for C₂₁H₂₀F₆N₂O₂ [M+H]⁺: m/z calcd: 447.15017. found: 447.15159.

### Example 15

### Structure-Activity Relationship

A parallel SAR study was carried out on N-phenyl-N′-(4-((2,6-difluorobenzyl)oxy)cyclohexyl)ureas II (27 analogs, Table 2), which differ from ureas in series I by replacing the 4-fluorophenoxy with the (2,6-difluorobenzyl)oxy, demonstrated to be lacking measureable activity in both the surrogate eIF2α phosphorylation and cell proliferation assays.

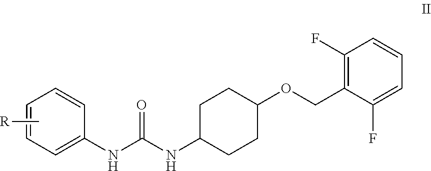

Based on the screening described above, a preliminary hit-to-lead optimization on the N-phenyl,N′-(4-phenoxy)cyclohexylurea scaffold I was performed, in which the N′-(4-phenoxy)cyclohexyl part of the molecule was modified while the N-(m-CF₃-phenyl) part was maintained. This process included two steps: 1) modification of substituents on the N-phenoxy ring (I-14-20, Table 3) and 2) exploration of substitution permutation on the phenoxy moiety substituting the cyclohexyl ring (III-1-6, Table 4).

TABLE 3
Activity of the phenoxy substituted 1-(4-phenoxycyclohexyl)-3-(3-(trifluoromethyl)phenyl)ureas,
I-14-20, in the surrogate eIF2α phosphorylation and cell proliferation assays.

| | | | | Emax |
|---|---|---|---|---|
| Compound | Structure | CLogP | IC₅₀ (μM) | (F/R) |
| I-5m | 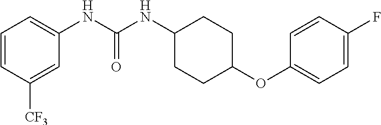 [SMILES: O=C(Nc1cccc(C(F)(F)F)c1)NC1CCC(Oc2ccc(F)cc2)CC1] | 5.57 | 2.6 ± 0.6 | 11.84 |
| I-14 | 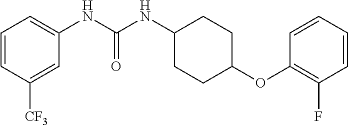 [SMILES: O=C(Nc1cccc(C(F)(F)F)c1)NC1CCC(Oc2ccccc2F)CC1] | 5.37 | 2.45 ± 0.9 | 7.98 |
| I-15 | 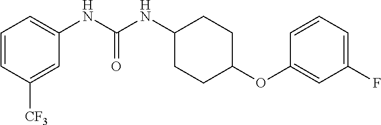 [SMILES: O=C(Nc1cccc(C(F)(F)F)c1)NC1CCC(Oc2cccc(F)c2)CC1] | 5.57 | 2.4 ± 0.6 | 7.23 |
| I-16 | 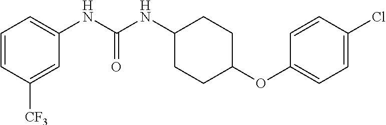 [SMILES: O=C(Nc1cccc(C(F)(F)F)c1)NC1CCC(Oc2ccc(Cl)cc2)CC1] | 6.14 | 2.0 ± 0.1 | 6.73 |
| I-17 | 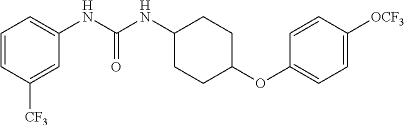 [SMILES: O=C(Nc1cccc(C(F)(F)F)c1)NC1CCC(Oc2ccc(OC(F)(F)F)cc2)CC1] | 6.48 | 0.69 ± 0.1 | 15.42 |
| I-18 | 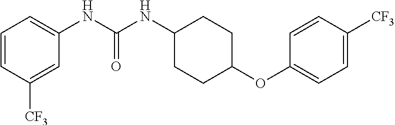 [SMILES: O=C(Nc1cccc(C(F)(F)F)c1)NC1CCC(Oc2ccc(C(F)(F)F)cc2)CC1] | 6.41 | 0.46 ± 0.1 | 25.12 |
| I-19 | 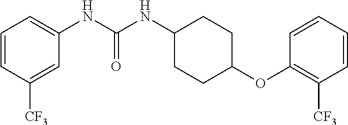 [SMILES: O=C(Nc1cccc(C(F)(F)F)c1)NC1CCC(Oc2ccccc2C(F)(F)F)CC1] | 6.41 | 1.4 ± 0.3 | 15.74 |
| I-20 | 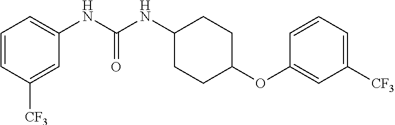 [SMILES: O=C(Nc1cccc(C(F)(F)F)c1)NC1CCC(Oc2cccc(C(F)(F)F)c2)CC1] | 6.41 | 1.3 ± 0.1 | 26.00 |

TABLE 4
Structure-activity study. Data for compounds III-1 through III-6 in
the surrogate eIF2α phosphorylation and cell proliferation assays.
III

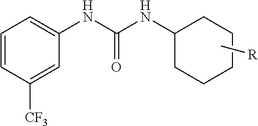

| | | | | Emax |
|---|---|---|---|---|
| Compound | Structure | CLogP | IC₅₀ (μM) | (F/R) |
| I-18 | 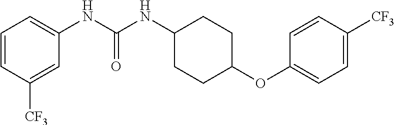 [SMILES: O=C(Nc1cccc(C(F)(F)F)c1)NC1CCC(Oc2ccc(C(F)(F)F)cc2)CC1] | 6.41 | 0.46 ± 0.1 | 25.12 |
| III-1 | 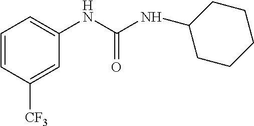 [SMILES: O=C(Nc1cccc(C(F)(F)F)c1)NC1CCCCC1] | 4.54 | 13.2 ± 2.3 | 1.87 |
| III-2 | 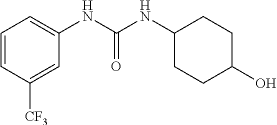 [SMILES: O=C(Nc1cccc(C(F)(F)F)c1)NC1CCC(O)CC1] | 2.46 | >20 | 0.89 |
| III-3 | 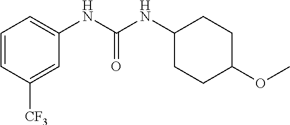 [SMILES: COC1CCC(NC(=O)Nc2cccc(C(F)(F)F)c2)CC1] | 3.17 | >20 | 1.16 |
| III-4 | 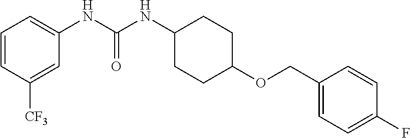 [SMILES: O=C(Nc1cccc(C(F)(F)F)c1)NC1CCC(OCc2ccc(F)cc2)CC1] | 5.16 | 2.65 ± 0.8 | 9.58 |
| III-5 | 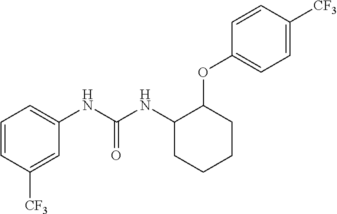 [SMILES: O=C(Nc1cccc(C(F)(F)F)c1)NC1CCCCC1Oc1ccc(C(F)(F)F)cc1] | 6.98 | 3.1 ± 1.1 | 5.89 |
| III-6 | 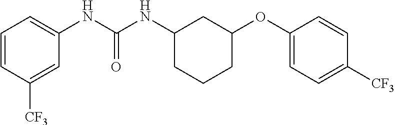 [SMILES: O=C(Nc1cccc(C(F)(F)F)c1)NC1CCCC(Oc2ccc(C(F)(F)F)cc2)C1] | 6.85 | 3.8 ± 1.2 | 4.52 |

### Example 16

### Activities in Secondary Mechanistic Assays

To confirm that the compounds activate HRI and thereby induce phosphorylation of eIF2α, representative compounds from Table 1, (N-phenyl,N′-(4-phenoxy)cyclohexylureas (I), were selected and tested in secondary mechanistic assays, namely endogenous eIF2α phosphorylation and expression of the transcription factor C/EBP homologous protein (CHOP) and mRNA. The surrogate eIF2α phosphorylation assay utilized for screening is a reporter gene assay that relates to a downstream event, namely up-regulating translation of a reporter fused to the 5′UTR of ATF-4 mRNA in response to the reduced abundance of the ternary complex. Phosphorylation of endogenous eIF2α, which inhibits the GDP-GTP exchange on eIF2 is the direct target of HRI and upstream regulator of the ternary complex abundance while the expression of CHOP is a downstream effector of ternary complex abundance. The selected N-phenyl,N′-(4-phenoxy)cyclohexylureas (I) cover a range of potencies in the surrogate eIF2α phosphorylation and cell proliferation assays.

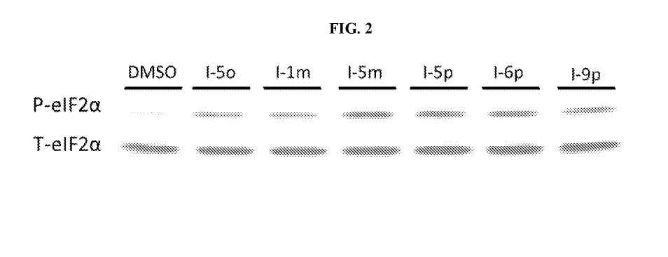

Referring to FIG. 2, their effect on the phosphorylation of eIF2α were determined in CRL-2813 cells by the Western blot analysis in which expression of the total (T-eIF2α) and the phosphorylated eIF2α (P-eIF2α) was determined using specific antibodies. N-phenyl,N′-(4-phenoxy)cyclohexylureas and their increased effects on the phosphorylation of eIF2α are shown. CRL-2813 human melanoma cells were incubated with selected N-phenyl,N′-(4-phenoxy)cyclohexylureas and the amount of phosphorylated (P-eIF2α) and total eIF2α (T-eIF2α) was determined by Western blot analysis with pS51-eIF2α-specific rabbit monoclonal antibodies or total eIF2α-specific mouse monoclonal antibodies, respectively. The experiment was independently performed three times.

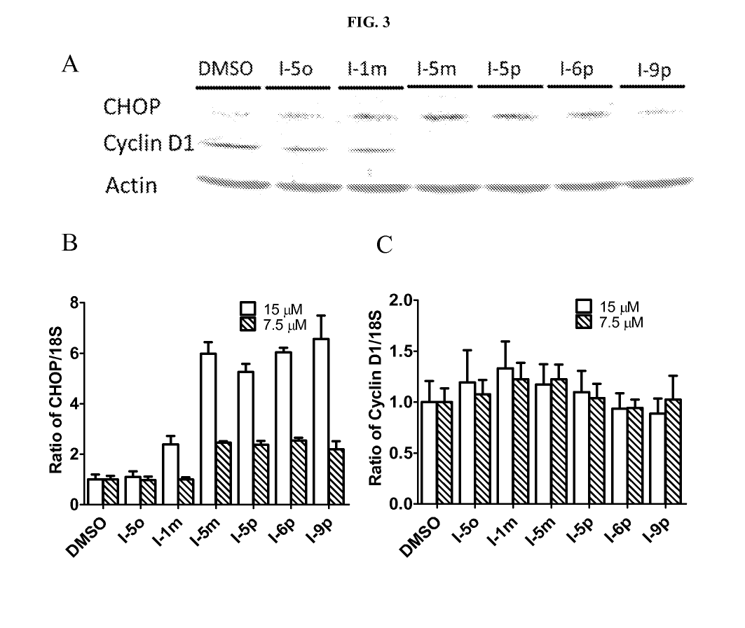

Referring to FIG. 3, the protein and mRNA expression of CHOP, a downstream effector of eIF2α phosphorylation, and Cyclin D1, an oncogenic protein whose expression is reduced in response to eIF2α phosphorylation, were also studied by Western blot and real-time PCR. The effect of N-phenyl,N′-(4-phenoxy)cyclohexylureas on protein and mRNA expressions of CHOP and Cyclin D1 are shown. Human CRL-2813 melanoma cells (FIG. 3A) were incubated with 10 μM of each compound for 6 hours. Expression of CHOP and Cyclin D1 were determined by Western blot analysis. A representative gel from three independent experiments is shown CRL-2813 cells were incubated at 7.5 or 15 μM of each compound for 6 hours. Expression of (FIG. 3B) CHOP and (FIG. 3C) Cyclin D1 mRNAs were determined by real time PCR analysis. Each experiment was carried out in triplicate, data shown are mean of three independent experiments, and error bars are ±S.E.M.

All compounds resulted in increased phosphorylation of endogenous eIF2α and enhanced expression of CHOP protein and mRNA roughly proportional to their activity in the surrogate eIF2α phosphorylation assay (FIG. 3A and FIG. 3B). Consistently, N-phenyl, N′-(4-phenoxy) cyclohexylureas led to reduced expression of cyclin D1 protein with minimal effect on its mRNA expression (FIGS. 3A and 3C). Activity on Cyclin D1 protein expression correlated well with activity in the surrogate eIF2α phosphorylation assay. These data show a correlation between the activity of the compounds in the surrogate eIF2α phosphorylation assay, their ability to induce the phosphorylation of endogenous eIF2α, expression of CHOP protein and mRNA, and inhibit expression of oncogenic proteins.

### Example 17

### Dependence of Activity of N-phenyl,N′-(4-phenoxy)cyclohexylureas on HRI

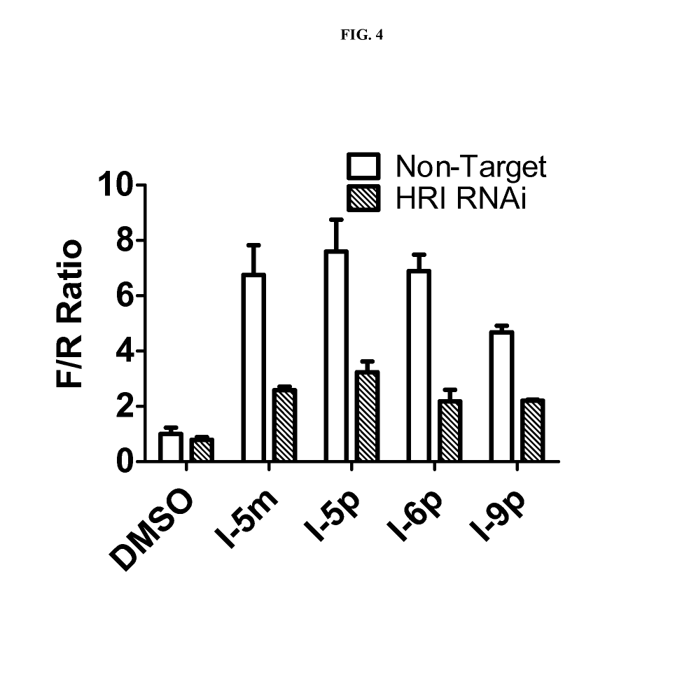

To determine if the molecular and cellular effects of N-phenyl,N′-(4-phenoxy)cyclohexylureas are mediated by HRI, its expression was knocked down by transfecting reporter CRL-2813-pBISA-DL(ATF-4) cells used in the surrogate eIF2α phosphorylation assay with siRNA targeting HRI or non-targeting siRNA. Cells were treated with selected compounds, and the F/R ratios were measured. Referring to FIG. 4, siRNA targeting HRI significantly blunted activity of N-phenyl,N′-(4-phenoxy)cyclohexylureas in the surrogate eIF2α phosphorylation assay compared to non-targeting siRNA. The effect of N-phenyl,N′-phenoxycyclohexylureas (I) in reducing the abundance of the ternary complex by activating HRI is shown. Stably transfected CRL-2813-pBISA-DL(ATF-4) cells were transiently transfected with either non-targeting siRNA, or siRNA targeting HRI for 48 hours. Cells were treated with N-phenyl,N′-phenoxycyclohexylurea compounds or DMSO for 10 hours, and the normalized F/R ratio was determined by DLR. The experiment was conducted in triplicate and each experiment was independently performed three times; Data are shown as Mean±S.E.M.

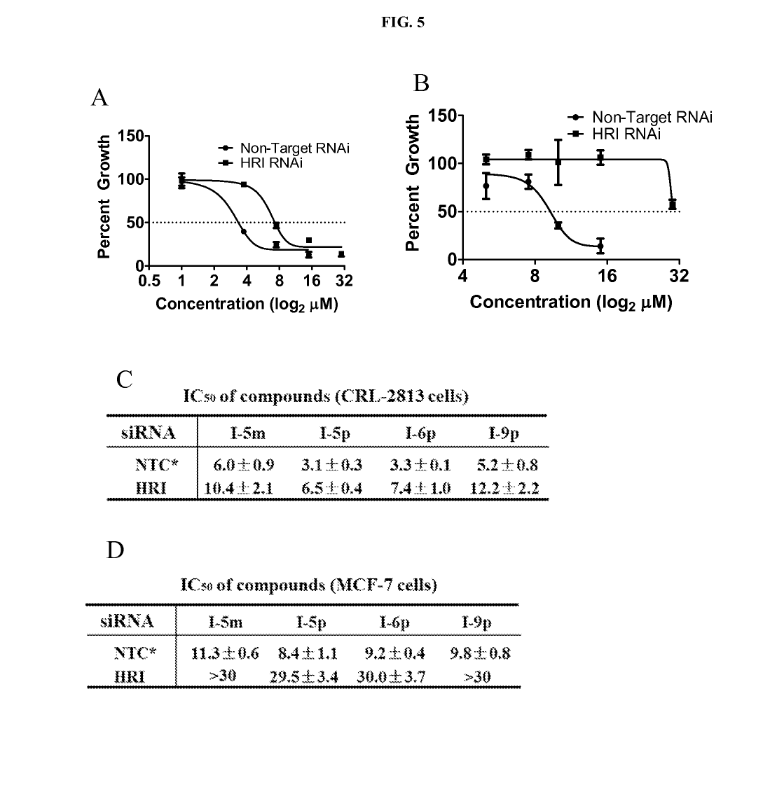

Cell proliferation is a meaningful read-out for determining the specificity of a compound as it captures both on-target and off-target effects of the compound under study. Cell proliferation was used as a biological response paradigm to demonstrate target specificity of N-phenyl,N′-(4-phenoxy)cyclohexylureas. If these compounds specifically activate HRI and this activity is required for the inhibition of cell proliferation, then reducing the expression of HRI should blunt their effect on cell proliferation. For this purpose, expression of HRI was knocked down in CRL-2813 human melanoma (about 70% knockdown efficiency) and MCF-7 human breast cancer cells (about 90% knockdown efficiency). These two cell lines were chosen because the HRI knockdown efficiency is lower in CRL-2813 than in MCF-7 cells providing a sort of dose-response data that correlates knockdown efficiency with the resistance of cells to inhibition of cell proliferation. Referring to FIG. 5, these data indicate that inhibition of cell proliferation by the N-phenyl,N′-(4-phenoxy)cyclohexylureas is dependent on HRI and that it correlates with the HRI knockdown efficiency. The data show that HRI mediates inhibition of cancer cell proliferation by N-phenyl,N-phenoxycyclohexylureas. Referring to FIG. 5A, CRL-2813 human melanoma cancer cells, and (FIG. 5B) MCF-7 human breast cancer cells were transfected with HRI targeted or non-targeted siRNA, treated with the indicated concentrations of I-6p and cell proliferation was measured by SRB assay. Calculated IC₅₀ for all four compounds tested in CRL-2813 human melanoma cancer cells and MCF-7 human breast cancer cells transfected with non-target siRNA control (NTC) or HRI is shown in FIG. 5C and FIG. 5D, respectively. The experiment was conducted in triplicate and each experiment was independently performed three times. Data are shown as Mean±S.E.M. *NTC=non-target siRNA control.

Taken together, these data demonstrate that the N-phenyl,N′-(4-phenoxy)cyclohexylureas activate HRI, induce eIF2α phosphorylation and reduce the amount of the ternary complex thereby inhibiting translation initiation and cell proliferation.

### Example 18

### Dose Response Studies of Ureas I-14 Through I-20

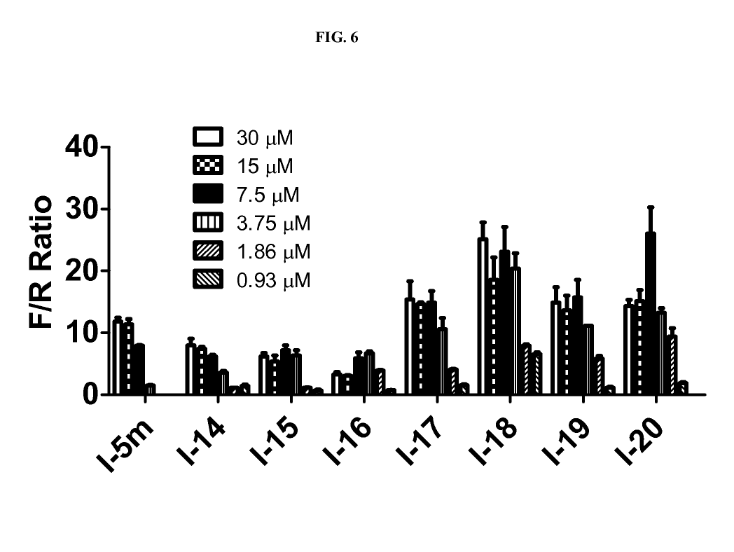

Referring to FIG. 6, the dose response studies for the phenoxy substituted 1-(4-phenoxycyclohexyl)-3-(3-(trifluoromethyl)phenyl)ureas, I-14-20, in the surrogate eIF2α phosphorylation assays are shown. The activity of compounds in Table 2 was measured by determining the F/R normalized to vehicle treated cells, and expressed as a function of the concentration. The experiment was conducted in triplicate and each experiment was independently performed three times. Data are shown as Mean±S.E.M.

### Example 19

### Activity of Ureas III-1 Through III-6

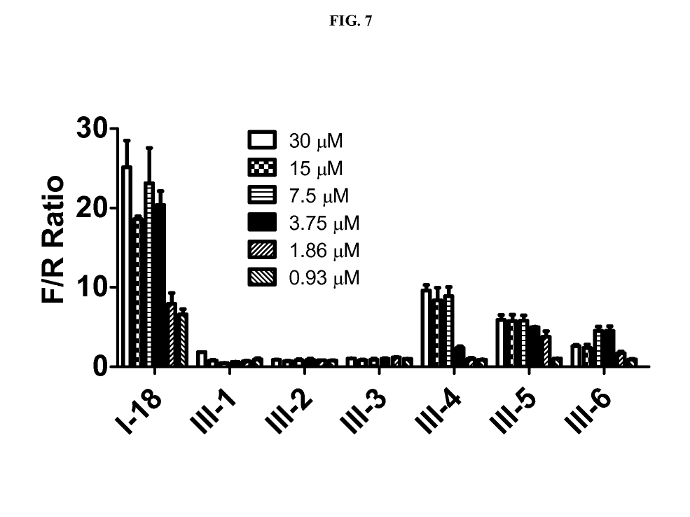

Referring to FIG. 7, activity of ureas in which the phenoxy moiety is either missing or relocated to another position on the cyclohexyl in surrogate eIF2α phosphorylation assays are shown. Activity of compounds was measured by the F/R luciferase ratio (F/R) compared to vehicle treated cells, and expressed as a function of the concentration. The experiment was conducted in triplicate and each experiment was independently performed three times; Data are shown as Mean±S.E.M.

### Example 20

### Activity of Ureas on Cells Containing HRI

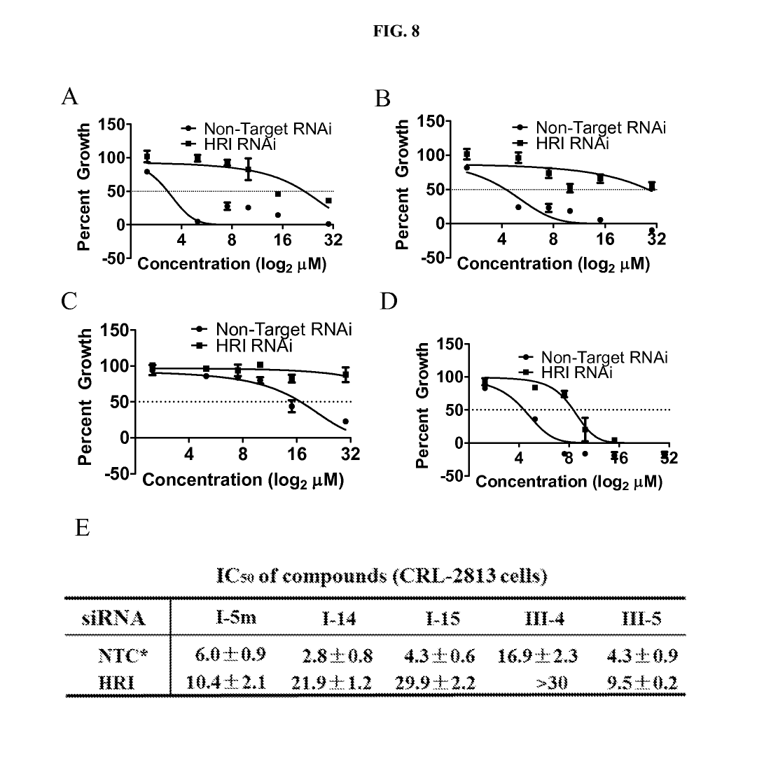

Referring to FIG. 8, CRL-2813 human melanoma cancer cells were transfected with HRI targeted or non-targeted siRNA, treated with the indicated concentrations of (A) I-14, (B) I-15, (C) III-4 and (D) III-5 and cell proliferation was measured by SRB assay. Calculated IC₅₀ for all four compounds tested in CRL-2813 human melanoma cancer cells transfected with non-target siRNA control (NTC) or HRI is shown in (E). The experiment was conducted in triplicate and each experiment was independently performed three times. Data are shown as Mean±S.E.M. *NTC=non-target siRNA control, IC₅₀=effective dose of drug that inhibit cell proliferation by 50%.

As shown in FIG. 8, inhibition of cell proliferation by the 1-(4-phenoxycyclohexyl)-3-(3-(trifluoromethyl)phenyl)ureas (1-14 and 1-15) and pharmacophore (III-4 and III-5) is dependent on HRI. In particular, I-14 and I-15 exhibit better differentiation in the resistance of cells to inhibition of cell proliferation upon HRI knockdown (average of ˜8 fold difference v.s. average of ˜2.5 fold difference in N-phenyl,N′-phenoxycyclohexylureas). These data demonstrate that 1-(4-phenoxycyclohexyl)-3-(3-(trifluoromethyl)phenyl)urea is a scaffold with potent cell growth inhibition activity and good specificity (FIGS. 8A, 8B, and 8E).

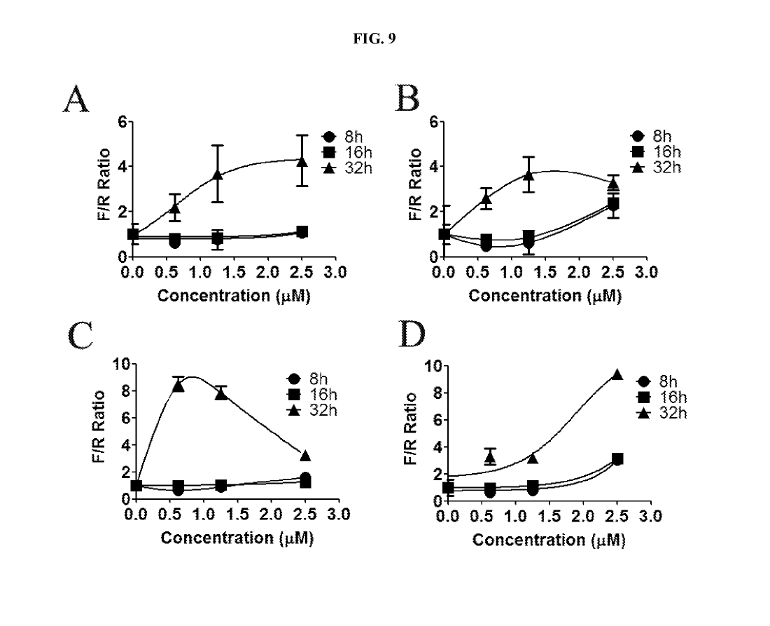

To determine if the discrepancy in the active concentrations of some 1-(4-phenoxycyclohexyl)-3-(3-(trifluoromethyl)phenyl)urea in the surrogate eIF2α phosphorylation and cell proliferation assays is due to differences in the length of these two assays we determined the activity of four compounds which such discrepancy in the surrogate eIF2α phosphorylation assay after 8, 16, or 32 hour incubation. As shown in FIG. 9, all four compounds displayed much higher activity after 32 hours of incubation compared to 8 or 16 hours of incubation. These data indicate that much of the discrepancy in the activity of compounds in the two primary assays used in these studies are due to the differences in the duration of the assays and support our contention that there is a very good correlation between the activity of compounds in the surrogate eIF2α phosphorylation and cell proliferation assays.

One test of suitability of a compound for anti-cancer therapy is the selectivity for cancer cells compared to normal cells. We choose one moderately active, I-6p, and one highly active, I-17, compounds to determine if 1-(4-phenoxycyclohexyl)-3-(3-(trifluoromethyl)phenyl)urea compounds selectively inhibit proliferation of cancer cells. Dose response studies in non-transformed NIH-3T3 fibroblast and CRL-2813 human melanoma cancer cell lines show that both compounds inhibit proliferation of cancer cells with five-fold higher potency compared to that of non-transformed cells. Taken together, data in FIG. 8 and Table 4 demonstrate that 1-(4-phenoxycyclohexyl)-3-(3-(trifluoromethyl)phenyl)urea compounds reported here are highly potent and specific inducer of HRI activity.

### Example 21

### Effect of N-aryl,N′-cyclohexylarylureas on proliferation of non-transformed (NIH 3T3) and cancer cell (CRL-2813)

As shown in Table 5, IC₅₀ were determined for two N-aryl,N′-cyclohexylarylureas tested in NIH/3T3 fibroblast cells and CRL-2813 melanoma cells. The experiments were conducted in triplicate, and each experiment was independently performed three times. IC₅₀=effective dose of drug that inhibit cell proliferation by 50%. Data are shown as Mean±S.E.M.

TABLE 5
IC₅₀ on certain cell lines

| | I-6p | I-17 |
|---|---|---|
| NIH/3T3 | 13.6 ± 0.8 | 3.4 ± 0.2 |
| CRL-2813 | 2.7 ± 0.1 | 0.69 ± 0.1 |

### Example 22

### Activity of Compound I-17 on Various Cancer Cell Lines

Table 6 shows the anti-proliferation effects of the urea 1-17 on a number of cancer cell lines.

TABLE 6
Effect on proliferation in vitro against certain cancer cell lines

| Cell line | Tissue Origin | IC₅₀ (μM) |
|---|---|---|
| T47D | Breast | 1.2 |
| RAMOS | Lymphoma | 0.8 |
| ACHN | Kidney | 1.4 |
| Huh-7 | Liver | 2.1 |
| HEP B3 | Liver | 1.8 |
| A-549 | Lung | 0.9 |
| PC-3 | Prostate | 2.9 |
| ASPC-1 | Pancreas | 2.4 |
| HT-29 | Colon | 2.7 |
| BAF-3 | Leukemia | 0.8 |
| CaSki | Cervix | 2.8 |
| MCF-7 | Breast | 1.3 |

### Example 23

### Activities in Secondary Mechanistic Assays

The compounds shown in Table 7 were evaluated using for activation of HRI using the dual luciferase surrogate eIF2α phosphorylation assay (see, for example, Chen, T. et al. Nature Chem Biol 2011, 7, 610-616) and for inhibition of cell proliferation using the SRB assay (see, for example, Palakurthi, S. S. et al. Cancer Res 2000, 60, 2919-2925) as described above. As shown in Table 7, the tested compounds cover a range of potencies in the surrogate eIF2α phosphorylation and cell proliferation assays.

TABLE 7

| | | | | IC50 | IC50 | IC50 |
|---|---|---|---|---|---|---|
| | | | | (μM) | (μM) | (μM) |
| | | | C_{3X}** | ASPC- | Panc- | CRL- |
| Structure | cLogP | Emax* | (μM) | 1 | 1 | 2813 |
| 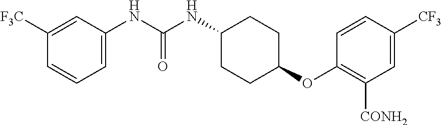 [SMILES: NC(=O)c1cc(C(F)(F)F)ccc1O[C@H]1CC[C@H](NC(=O)Nc2cccc(C(F)(F)F)c2)CC1] | 5.0674 | 8 | 7 | 4 | 2.7 | 1.9 |
| 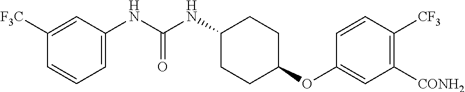 [SMILES: NC(=O)c1cc(O[C@H]2CC[C@H](NC(=O)Nc3cccc(C(F)(F)F)c3)CC2)ccc1C(F)(F)F] | 3.9274 | 2.8 | >20 | 3 | 2.5 | 2.1 |
| 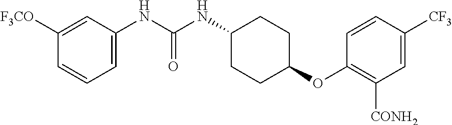 [SMILES: NC(=O)c1cc(C(F)(F)F)ccc1O[C@H]1CC[C@H](NC(=O)Nc2cccc(OC(F)(F)F)c2)CC1] | 4.8668 | 6 | 8 | 3.2 | 1.9 | 1.4 |
| 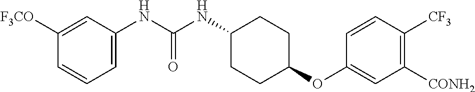 [SMILES: NC(=O)c1cc(O[C@H]2CC[C@H](NC(=O)Nc3cccc(OC(F)(F)F)c3)CC2)ccc1C(F)(F)F] | 3.7268 | 5 | 14 | 2.9 | 3 | 3 |
| 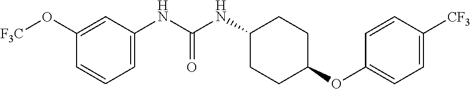 [SMILES: O=C(Nc1cccc(OC(F)(F)F)c1)N[C@H]1CC[C@H](Oc2ccc(C(F)(F)F)cc2)CC1] | 6.2096 | 28 | 0.2 | 1 | 1.5 | 0.9 |
| 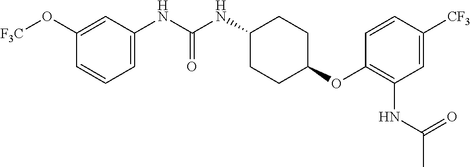 [SMILES: CC(=O)Nc1cc(C(F)(F)F)ccc1O[C@H]1CC[C@H](NC(=O)Nc2cccc(OC(F)(F)F)c2)CC1] | 4.9726 | 5 | 4 | 2.8 | 2.5 | 1.7 |
| 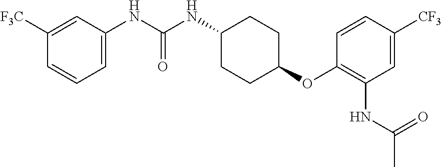 [SMILES: CC(=O)Nc1cc(C(F)(F)F)ccc1O[C@H]1CC[C@H](NC(=O)Nc2cccc(C(F)(F)F)c2)CC1] | 5.1732 | 4.2 | 3.5 | 2.6 | 1.7 | 1.9 |
| 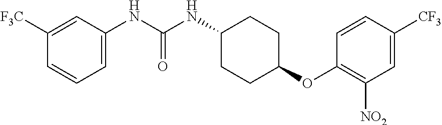 [SMILES: O=C(Nc1cccc(C(F)(F)F)c1)N[C@H]1CC[C@H](Oc2ccc(C(F)(F)F)cc2[N+](=O)[O-])CC1] | 6.0139 | 13 | 5 | >10 | 4 | 4 |
| 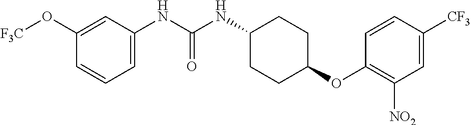 [SMILES: O=C(Nc1cccc(OC(F)(F)F)c1)N[C@H]1CC[C@H](Oc2ccc(C(F)(F)F)cc2[N+](=O)[O-])CC1] | 5.8133 | 12 | 0.5 | 0.8 | 1.1 | 0.5 |
| 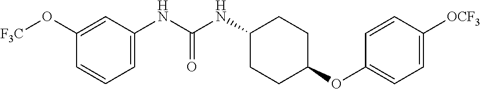 [SMILES: O=C(Nc1cccc(OC(F)(F)F)c1)N[C@H]1CC[C@H](Oc2ccc(OC(F)(F)F)cc2)CC1] | 6.2796 | 14 | 1.3 | ND | 3 | 1.2 |
| 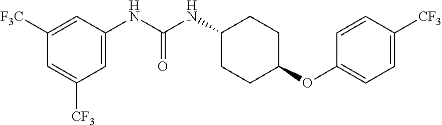 [SMILES: O=C(Nc1cc(C(F)(F)F)cc(C(F)(F)F)c1)N[C@H]1CC[C@H](Oc2ccc(C(F)(F)F)cc2)CC1] | 7.4784 | 38 | 0.15 | ND | 0.7 | 0.15 |
| 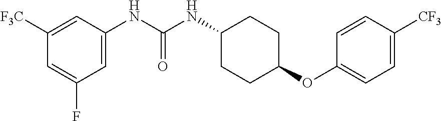 [SMILES: O=C(Nc1cc(F)cc(C(F)(F)F)c1)N[C@H]1CC[C@H](Oc2ccc(C(F)(F)F)cc2)CC1] | 6.6590 | | | | | |
| 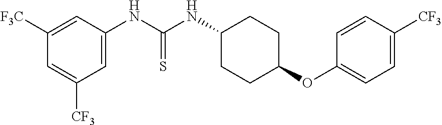 [SMILES: FC(F)(F)c1ccc(O[C@H]2CC[C@H](NC(=S)Nc3cc(C(F)(F)F)cc(C(F)(F)F)c3)CC2)cc1] | 7.2584 | | | | | |
| 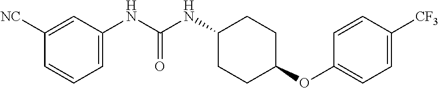 [SMILES: N#Cc1cccc(NC(=O)N[C@H]2CC[C@H](Oc3ccc(C(F)(F)F)cc3)CC2)c1] | 5.133 | 10 | 2 | ND | 2.5 | 0.8 |
| 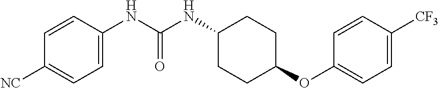 [SMILES: N#Cc1ccc(NC(=O)N[C@H]2CC[C@H](Oc3ccc(C(F)(F)F)cc3)CC2)cc1] | 5.133 | 9 | 0.6 | ND | 2 | 0.9 |
| 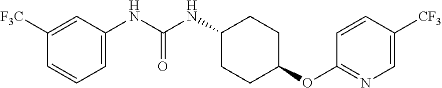 [SMILES: O=C(Nc1cccc(C(F)(F)F)c1)N[C@H]1CC[C@H](Oc2ccc(C(F)(F)F)cn2)CC1] | 5.6659 | 6 | 3.5 | ND | 2.8 | 1.5 |
| 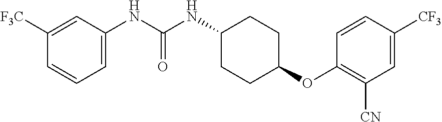 [SMILES: N#Cc1cc(C(F)(F)F)ccc1O[C@H]1CC[C@H](NC(=O)Nc2cccc(C(F)(F)F)c2)CC1] | 6.0089 | 8 | 2.5 | ND | 1.8 | 1.3 |
| 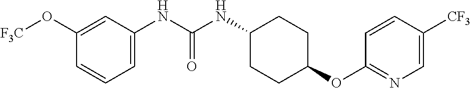 [SMILES: O=C(Nc1cccc(OC(F)(F)F)c1)N[C@H]1CC[C@H](Oc2ccc(C(F)(F)F)cn2)CC1] | 5.4653 | | | | | |
| 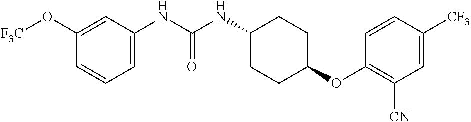 [SMILES: N#Cc1cc(C(F)(F)F)ccc1O[C@H]1CC[C@H](NC(=O)Nc2cccc(OC(F)(F)F)c2)CC1] | 5.8083 | | | | | |
| 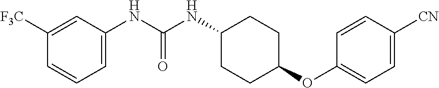 [SMILES: N#Cc1ccc(O[C@H]2CC[C@H](NC(=O)Nc3cccc(C(F)(F)F)c3)CC2)cc1] | 4.8369 | 4 | 5 | | 3 | 4 |
| 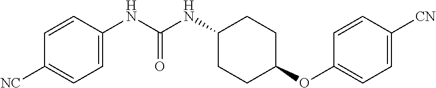 [SMILES: N#Cc1ccc(NC(=O)N[C@H]2CC[C@H](Oc3ccc(C#N)cc3)CC2)cc1] | | | | ND | 10 | 10 |
| 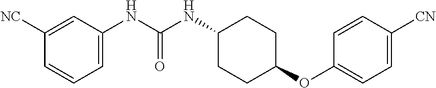 [SMILES: N#Cc1ccc(O[C@H]2CC[C@H](NC(=O)Nc3cccc(C#N)c3)CC2)cc1] | | | | ND | >10 | 7 |
| 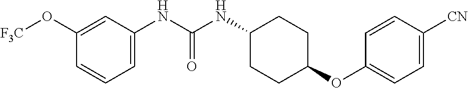 [SMILES: N#Cc1ccc(O[C@H]2CC[C@H](NC(=O)Nc3cccc(OC(F)(F)F)c3)CC2)cc1] | 5.0402 | | | | | |
| 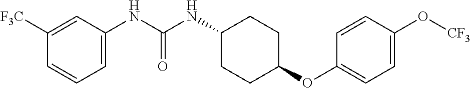 [SMILES: O=C(Nc1cccc(C(F)(F)F)c1)N[C@H]1CC[C@H](Oc2ccc(OC(F)(F)F)cc2)CC1] | 6.4802 | | | | | |
| 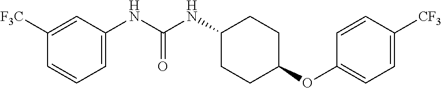 [SMILES: O=C(Nc1cccc(C(F)(F)F)c1)N[C@H]1CC[C@H](Oc2ccc(C(F)(F)F)cc2)CC1] | 6.4102 | 25 | 0.4 | 0.9 | 0.9 | 0.5 |
| 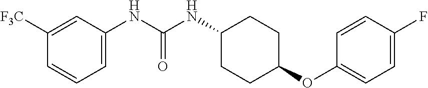 [SMILES: O=C(Nc1cccc(C(F)(F)F)c1)N[C@H]1CC[C@H](Oc2ccc(F)cc2)CC1] | 5.5652 | 11 | 2.5 | ND | 3 | 2.1 |

*Emax: Maximum activity of compound expressed as fold over vehicle (DMSO = 1) in the surrogate eIF2α phosphorylation assay. Maximum tested concentration is 20 μM
C_{3X}: Calculated concentration of compound that will cause three-fold increase in the surrogate eIF2α phosphorylation assay.
ND = Not determined

### Example 24

### Specificity of novel N-phenyl,N′-(4-phenoxy)cyclohexylurea compounds

The improved activity of newly synthesized compounds in the surrogate eIF2α phosphorylation and cell proliferations assays could either be due to better cell-penetration or higher affinity for their molecular target. It is also possible that the increased activity in the cell proliferation assay may be due, at least in part, to the increased off-target effects.

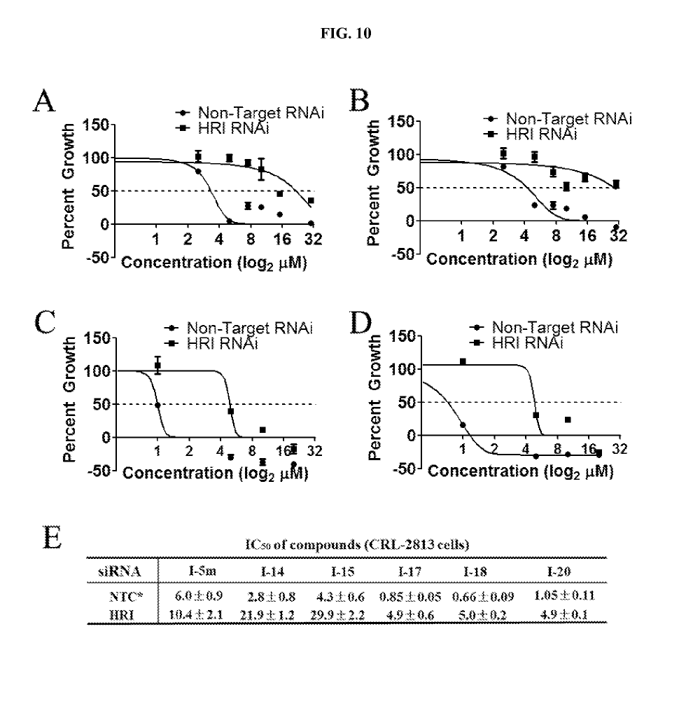

In general, if the affinity of compounds for their molecular target is increased this should result in higher specificity of the compounds in the cell proliferation assay. To demonstrate that this was indeed the case, the dependence of the anti-proliferative effects of five selected compounds in these series on HRI was determined. These five compounds, two with average and three with high potency, were tested in cell proliferation assay using CRL-2813 cells transfected with non-targeted siRNA or HRI-targeted siRNA as described above. As shown in FIG. 10, two average potency compounds, I-14 and I-15, displayed a significant dependence on HRI for inhibition of cell proliferation (˜7 fold higher IC₅₀ values in cells transfected with HRI-targeting siRNA vs. in those transfected with non-targeting siRNA). This is a significant improvement in specificity toward the molecular target over initial hits that displayed 1.7-2.2 fold higher IC₅₀ values in CRL-2813 cells transfected with HRI-targeting siRNA vs. those transfected with non-targeting siRNA. Compounds with higher potency in the surrogate eIF2α phosphorylation and SRB assays also showed a very good target dependencies/specificities (˜6, 7.5, and 5 fold higher IC₅₀ values in cells transfected with HRI-targeting siRNA vs. in those transfected with non-targeting siRNA for I-17, I-18, and I-20, respectively). These data indicate that our SAR studies resulted in the generation of more specific compounds. These data suggest that compounds' Eₘₐₓ in the surrogate eIF2α phosphorylation assay alone may not be sufficient to predict their specificity and that additional parameters such as C_{3X} may also need to be considered.

To determine if the discrepancy in the active concentrations of some 1-(4-phenoxycyclohexyl)-3-(3-(trifluoromethyl)phenyl)urea in the surrogate eIF2α phosphorylation and cell proliferation assays is due to differences in the length of these two assays we determined the activity of four compounds which display such discrepancy in the surrogate eIF2α phosphorylation assay after 8, 16, or 32 hour incubation. As shown in FIG. 9, all four compounds displayed much higher activity after 32 hours of incubation compared to 8 or 16 hours. These data indicate that much of the discrepancy in the activity of some compounds in the two primary assays used in these studies are due to the differences in the duration of the assays and support our contention that there is a very good correlation between the activity of compounds in the surrogate eIF2 α phosphorylation and cell proliferation assays.

One test of suitability of a compound for anti-cancer therapy is the selectivity for cancer cells compared to normal cells. We choose one moderately active, I-6p, and three highly active compounds, I-17, I-18, I-20, to determine if these series of N-phenyl,N′-cyclohexylphenoxy ureas can selectively inhibit proliferation of cancer cells. Dose-response studies in non-transformed NIH-3T3 fibroblast and CRL-2813 human melanoma cancer cell lines show that these compounds inhibit proliferation of cancer cells with 2.5- to 7-fold higher potency compared to that of non-transformed cells (Table 8). Among these compound I-18 displayed the highest discrimination between cancer and non-cancerous cells.

Taken together, data in FIG. 10, Table 8 and FIG. 9 demonstrate that some of the more potent N-phenyl,N-cyclohexylphenoxy ureas reported herein are highly potent and specific inducers of HRI activity.

TABLE 8
Selective inhibition of cancer cell proliferation by N-aryl,N′-
cyclohexylarylureas.

IC₅₀ of compounds (μM)

| | I-6p | I-17 | I-18 | I-20 |
|---|---|---|---|---|
| NIH/3T3 | 13.6 ± 0.8 | 3.4 ± 0.2 | 3.4 ± 0.1 | 3.3 ± 0.2 |
| CRL-2813 | 2.7 ± 0.1 | 0.69 ± 0.1 | 0.46 ± 0.1 | 1.30 ± 0.1 |

Compounds I-6p, I-17, I-18 and I-20 were tested in NIH/3T3 fibroblast and CRL-2813 melanoma cancer cells in cell proliferation assay. The experiment was conducted in triplicate and each experiment was independently performed three times. Data are shown as Mean IC₅₀±S.E.M.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula (III): 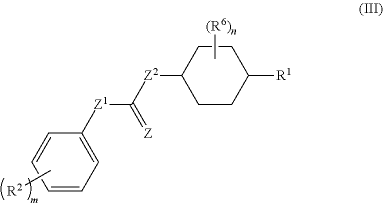
or a pharmaceutically acceptable salt thereof,
wherein:
Z, Z¹, and Z² are each independently selected from the group consisting of: NH, O, and S;
R¹ is XR³;
each R² is independently selected from the group consisting of: unsubstituted or substituted C₁₋₆alkyl; unsubstituted or substituted C₂₋₆alkenyl; unsubstituted or substituted C₂₋₆alkynyl;
unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 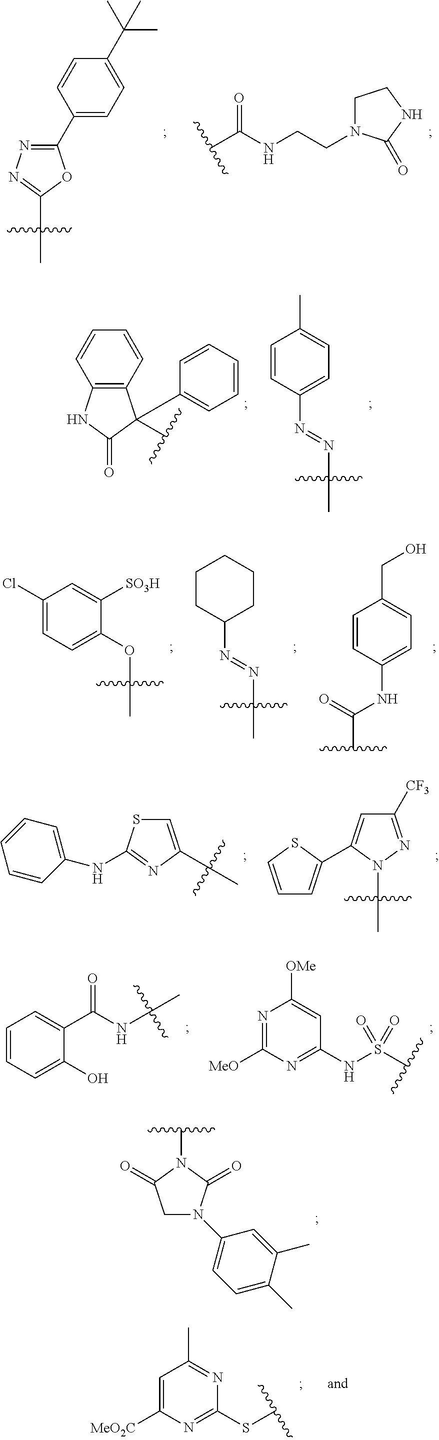 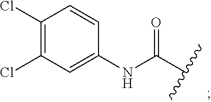 [SMILES: *C(=O)Nc1ccc(Cl)c(Cl)c1]
X is selected from the group consisting of: NR⁴, O, and S(O)ₚ;
m is an integer from 1 to 5;
n is an integer from 0 to 2;
p is an integer from 0 to 2;
R³ is selected from the group consisting of: unsubstituted or substituted heteroaryl; and 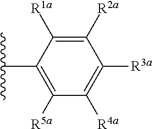
wherein
R^{2a} and R^{4a} are independently selected from the group consisting of: H; halo; unsubstituted or substituted C₁₋₆alkyl; unsubstituted or substituted C₁₋₆alkenyl; unsubstituted or substituted C₁₋₆alkynyl; C₁₋₆haloalkyl; CONR⁴R⁵; CONH(C₁₋₆alkyl)heterocyclyl; CONH(C₁₋₆alkyl)carbocyclyl; CONH(C₁₋₆alkyl)aryl; CONH(C₁₋₆alkyl)heteroaryl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkyl)carbocyclyl; C₁₋₆aralkyl; C₁₋₆heteroaralkyl; (C₁₋₆alkoxy)heterocyclyl; (C₁₋₆alkoxyl)carbocyclyl; (C₁₋₆alkoxyl)aryl; (C₁₋₆alkoxyl)heteroaryl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 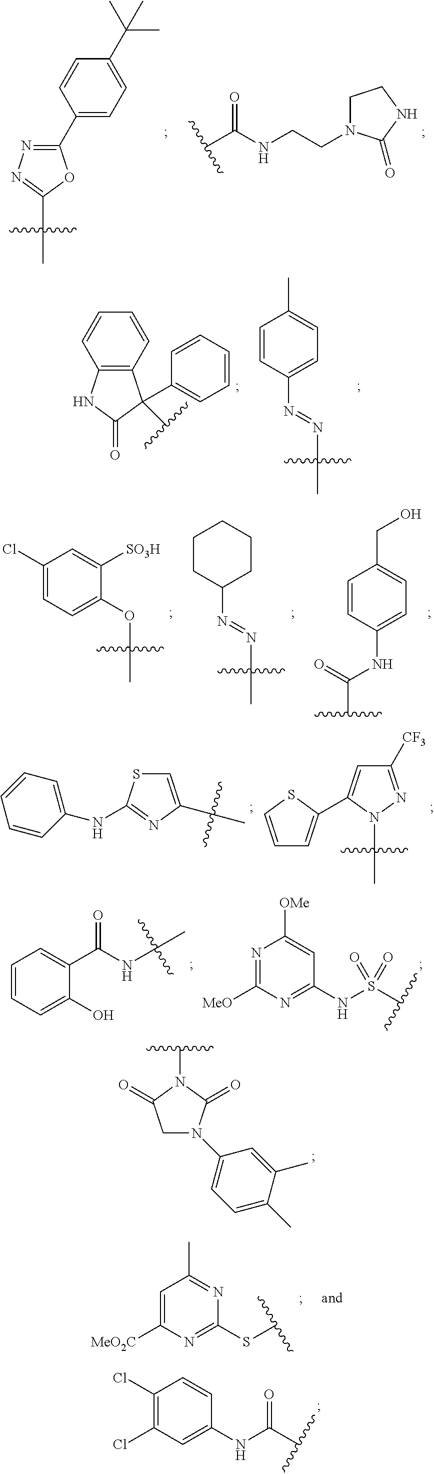
R^{1a}, R^{3a}, and R^{5a} are independently selected from the group consisting of: H; Cl; Br; I; unsubstituted or substituted C₁₋₆alkyl; C₁₋₆haloalkyl; CONR⁴R⁵; CONH(C₁₋₆alkyl)heterocyclyl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkoxy)heterocyclyl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 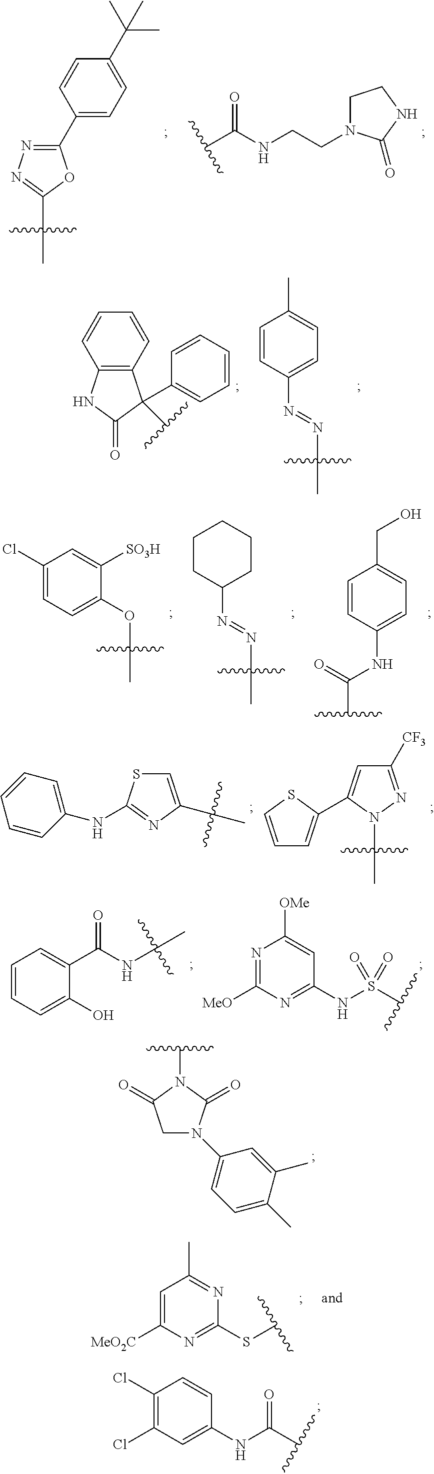
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 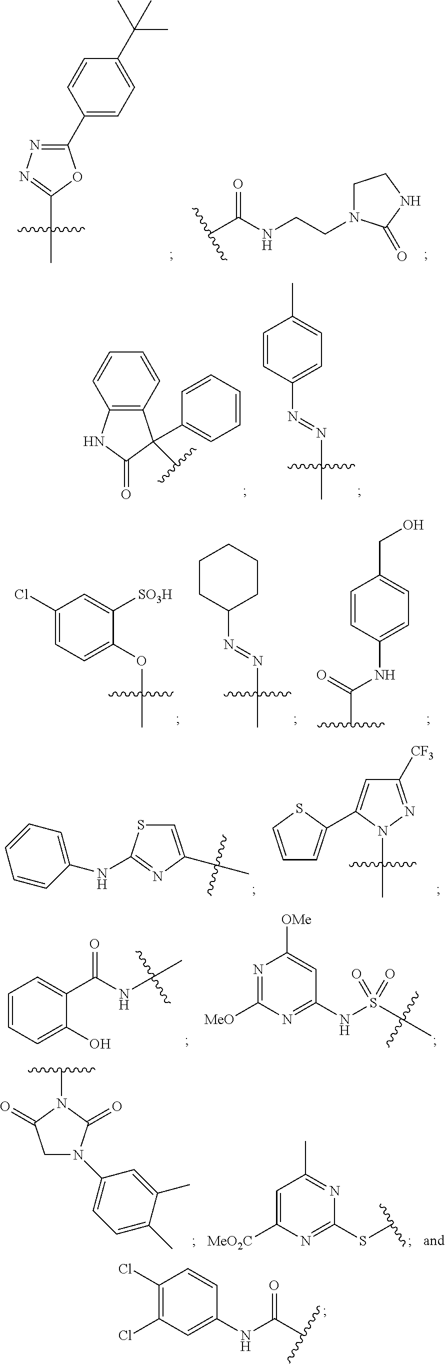
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

2. A compound of Formula (I): 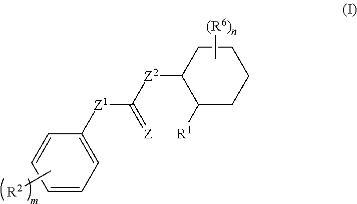
or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 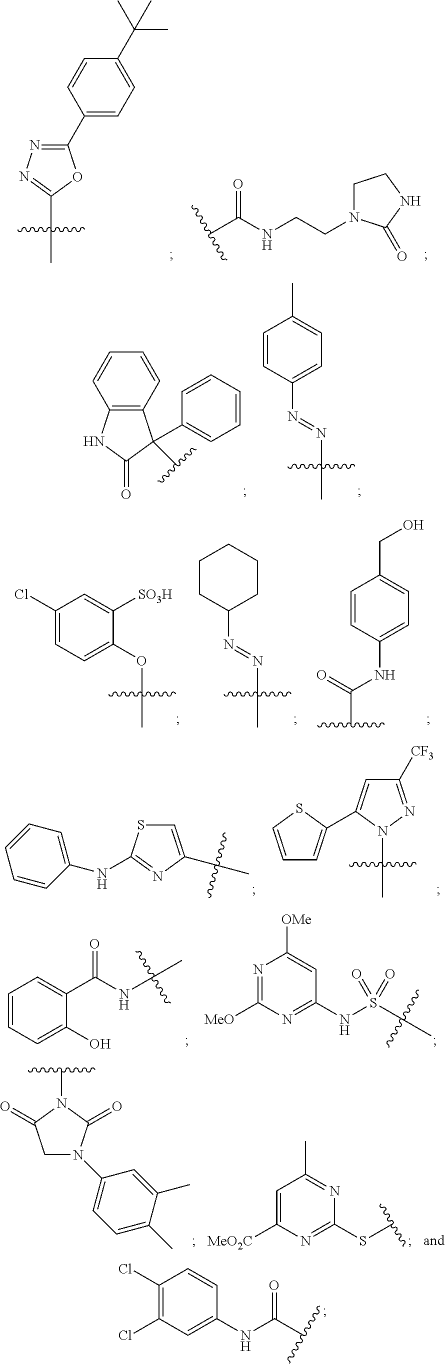
m is an integer from 1 to 5;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl and unsubstituted or substituted heteroaryl;
each R⁴ and R⁵ is independently selected from the group consisting of: H and unsubstituted or substituted C₁₋₆alkyl;
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—;
(aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 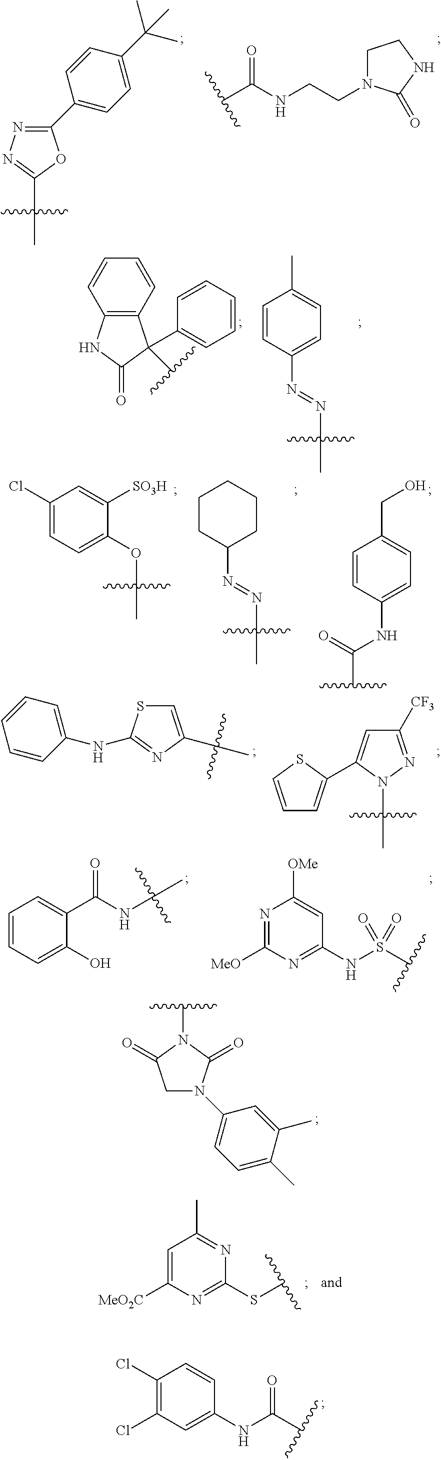
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

3. A compound of Formula (II): 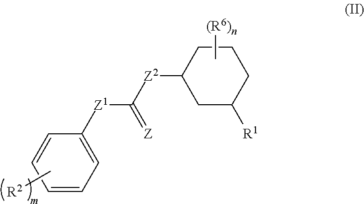
or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 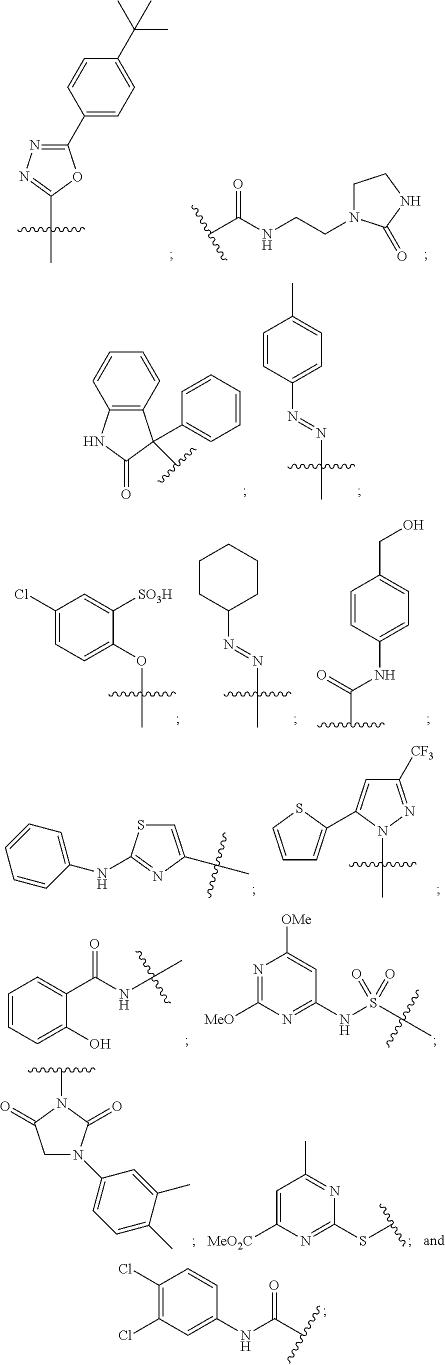
X is selected from the group consisting of: NR⁴, O, and S(O)ₚ;
m is an integer from 1 to 5;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl;
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—;
(aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 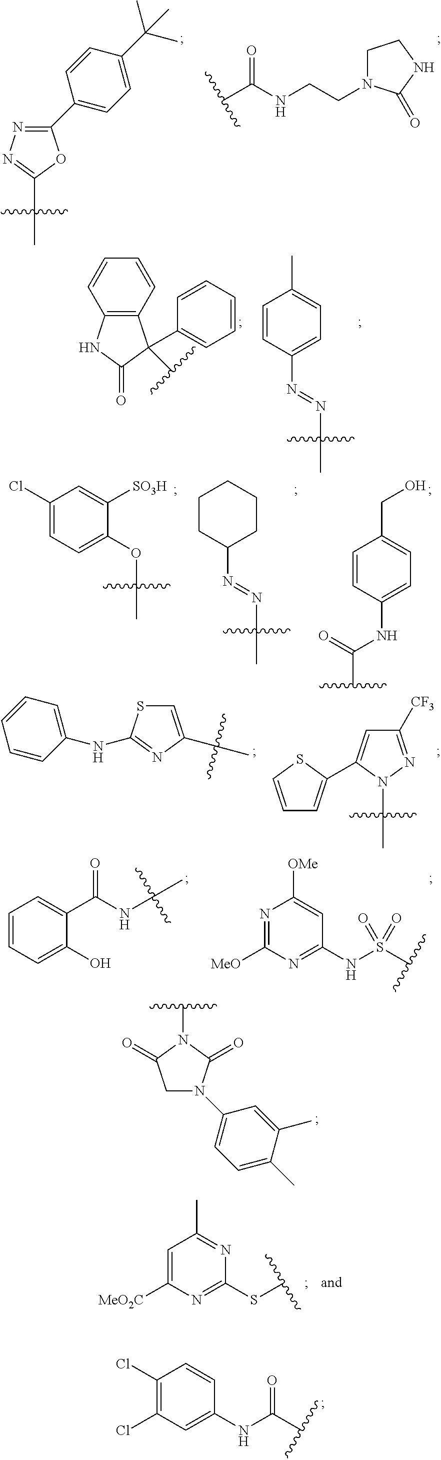
each R⁷ is independently selected from the group consisting of: H and unsubstituted or substituted C₁₋₆ alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

4. A compound of Formula (IV): 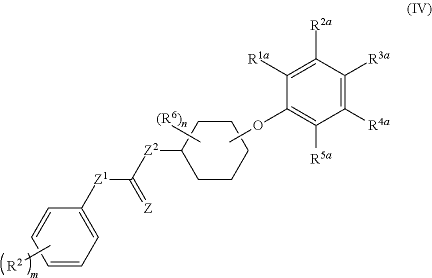
or a pharmaceutically acceptable salt thereof,
wherein:
Z is selected from the group consisting of: O and S;
Z¹ and Z² are each NH;
each R² is independently selected from the group consisting of: unsubstituted or substituted C₁₋₆alkyl; unsubstituted or substituted C₂₋₆alkenyl; unsubstituted or substituted C₂₋₆alkynyl;
unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 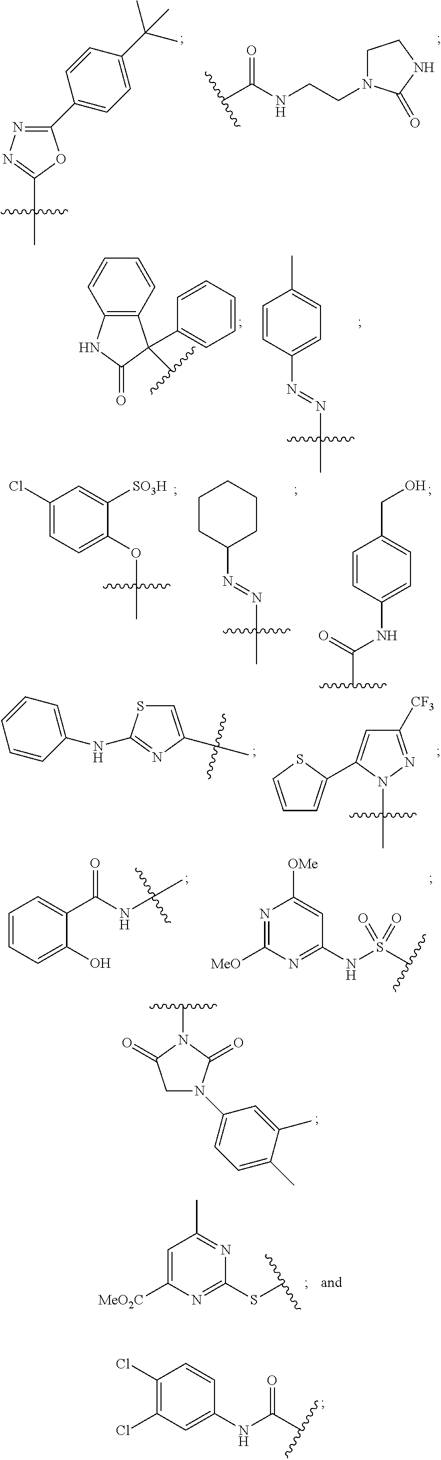
m is an integer from 1 to 5;
n is an integer from 0 to 2;
R^{2a} and R^{4a} are independently selected from the group consisting of: H; halo; unsubstituted or substituted C₁₋₆ alkyl; unsubstituted or substituted C₁₋₆ alkenyl; unsubstituted or substituted C₁₋₆alkynyl; C₁₋₆haloalkyl; CONR⁴R⁵; CONH(C₁₋₆alkyl)heterocyclyl; CONH(C₁₋₆alkyl)carbocyclyl; CONH(C₁₋₆alkyl)aryl; CONH(C₁₋₆alkyl)heteroaryl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkyl)carbocyclyl; C₁₋₆aralkyl; C₁₋₆heteroaralkyl; (C₁₋₆alkoxy)heterocyclyl; (C₁₋₆alkoxyl)carbocyclyl; (C₁₋₆alkoxyl)aryl; (C₁₋₆alkoxyl)heteroaryl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 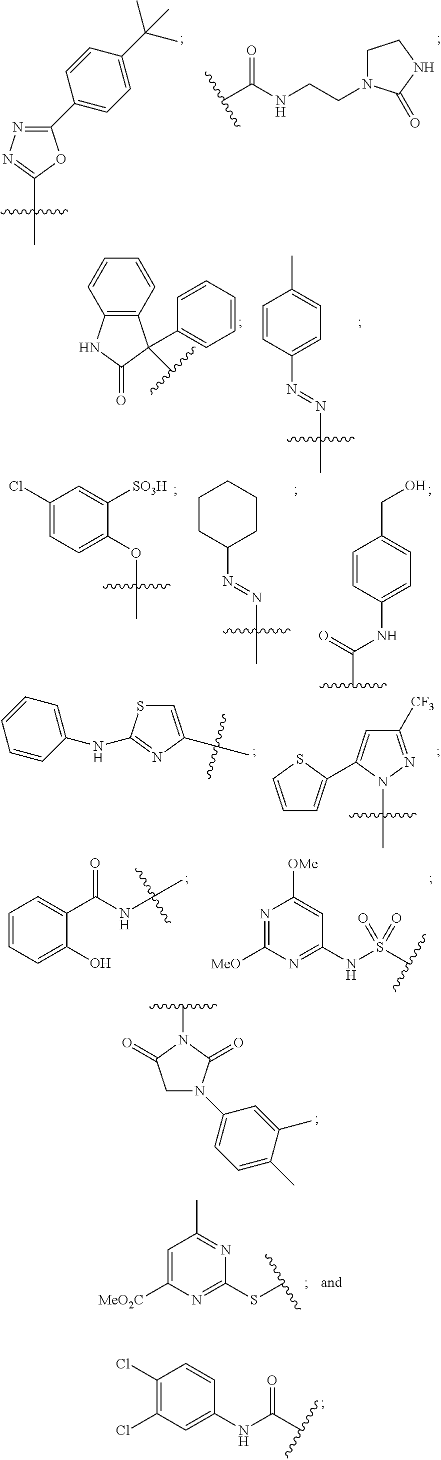
R^{1a}, R^{3a}, and R^{5a} are independently selected from the group consisting of: H; Cl; Br; I; unsubstituted or substituted C₁₋₆ alkyl; C₁₋₆haloalkyl; CONR⁴R⁵; CONH(C₁₋₆alkyl)heterocyclyl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkoxy)heterocyclyl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 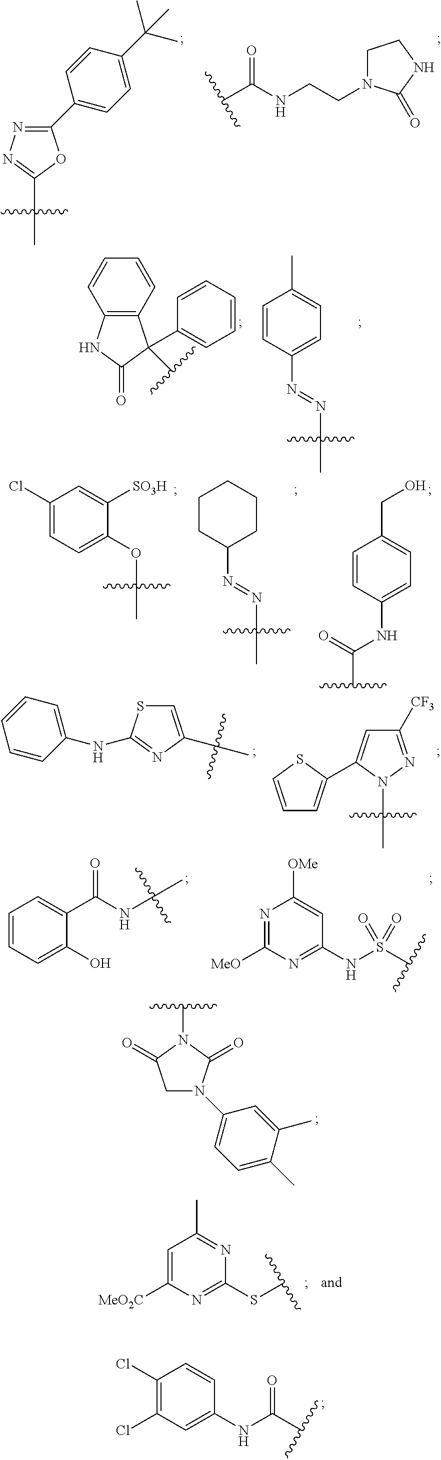
each R⁴ and R⁵ is independently selected from the group consisting of: H and unsubstituted or substituted C₁₋₆ alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 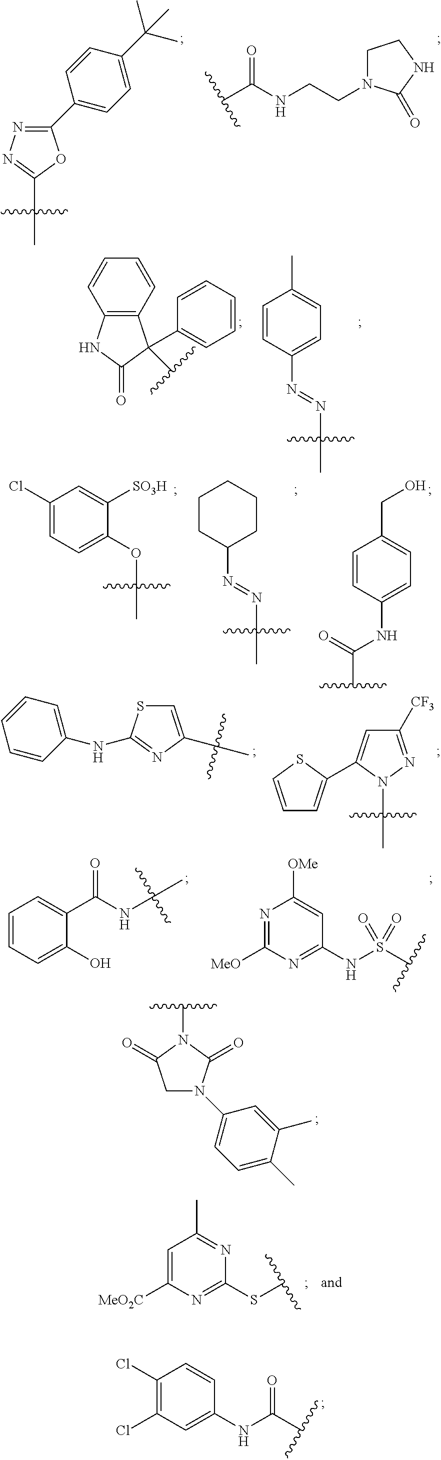
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

5. A compound of Formula (V): 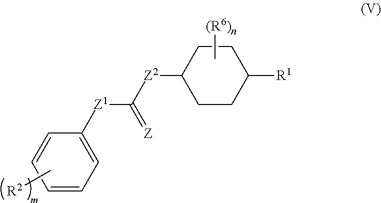
or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 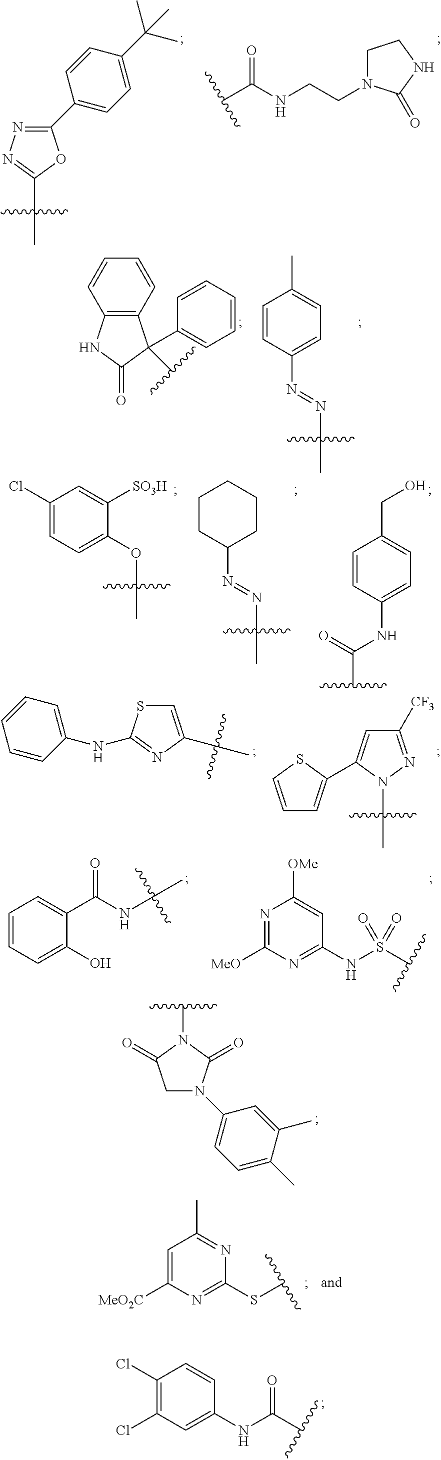
X is selected from the group consisting of: NR⁴, O, and S(O)ₚ;
m is an integer from 1 to 5;
n is an integer from 0 to 2;
p is an integer from 0 to 2;
W is [C(R⁵)₂]_{q};
q is an integer from 1 to 5;
R³ is selected from the group consisting of: unsubstituted or substituted heteroaryl; and 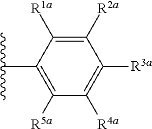
wherein
R^{2a}, R^{3a}, and R^{4a} are independently selected from the group consisting of: H; halo; unsubstituted or substituted C₁₋₆alkyl; unsubstituted or substituted C₁₋₆alkenyl; unsubstituted or substituted C₁₋₆alkynyl; C₁₋₆haloalkyl; CONR⁴R⁵; CONH(C₁₋₆alkyl)heterocyclyl; CONH(C₁₋₆alkyl)carbocyclyl; CONH(C₁₋₆alkyl)aryl; CONH(C₁₋₆alkyl)heteroaryl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkyl)carbocyclyl; C₁₋₆aralkyl; C₁₋₆heteroaralkyl; (C₁₋₆alkoxy)heterocyclyl; (C₁₋₆alkoxyl)carbocyclyl; (C₁₋₆alkoxyl)aryl; (C₁₋₆alkoxyl)heteroaryl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 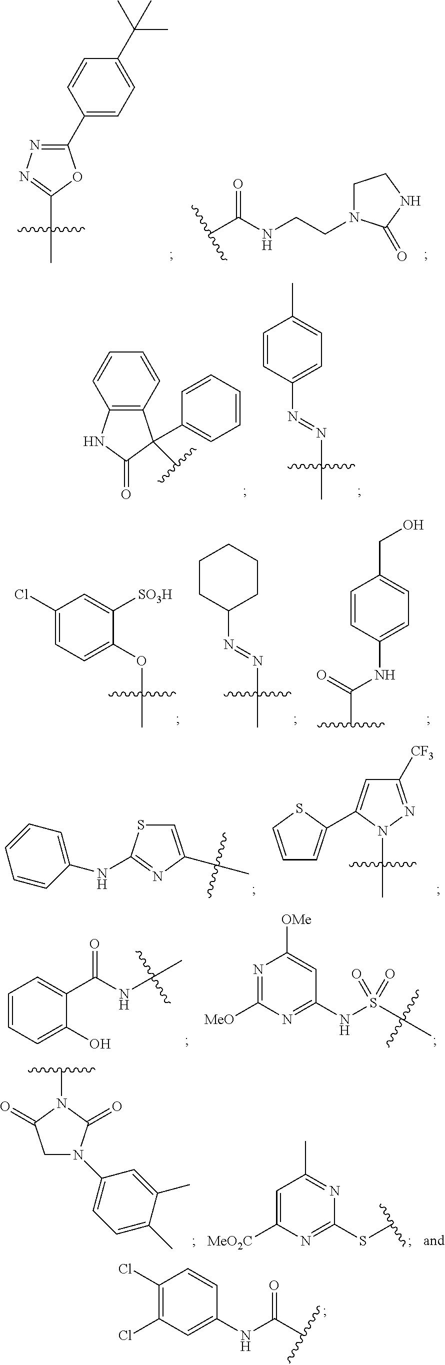
R^{1a} and R^{5a} are independently selected from the group consisting of: H; Cl; Br; I; unsubstituted or substituted C₁₋₆alkyl; C₁₋₆haloalkyl; CONR⁴R⁵; CONH(C₁₋₆alkyl)heterocyclyl; NR⁴R⁵; (C₁₋₆alkyl)NR⁴R⁵; (C₁₋₆alkoxy)NR⁴R⁵; (C₁₋₆alkyl)heterocyclyl; (C₁₋₆alkoxy)heterocyclyl; NR⁴COR⁵; COOR⁴; C₁₋₆haloalkoxy; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 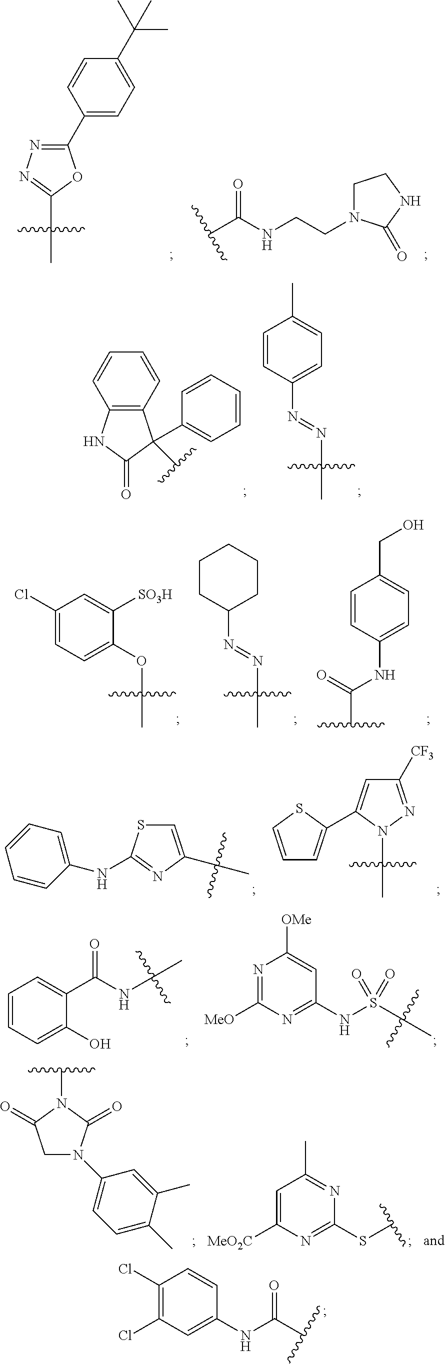
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 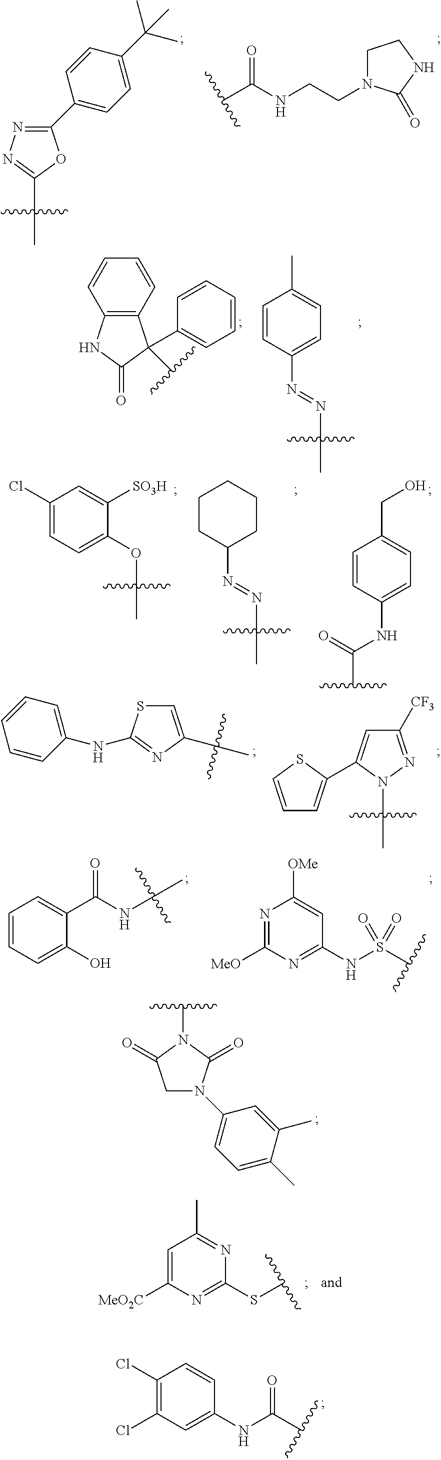
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

6. A compound of Formula (VI): 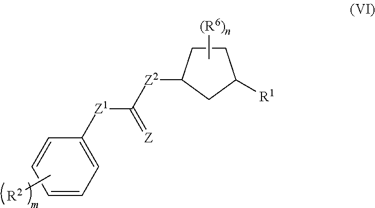
or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 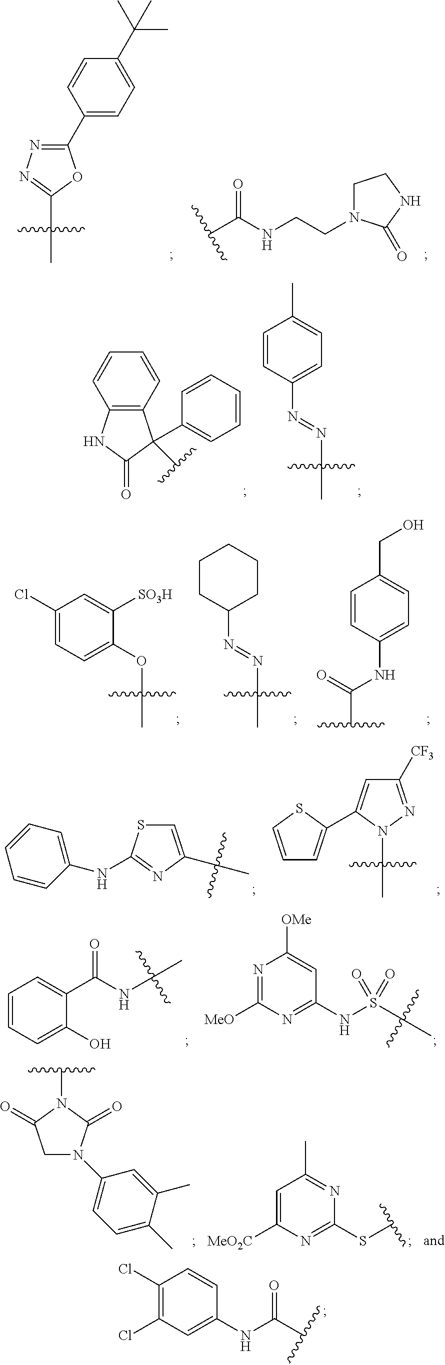
X is selected from the group consisting of: NR⁴, O, and S(O)ₚ;
m is an integer from 1 to 5;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 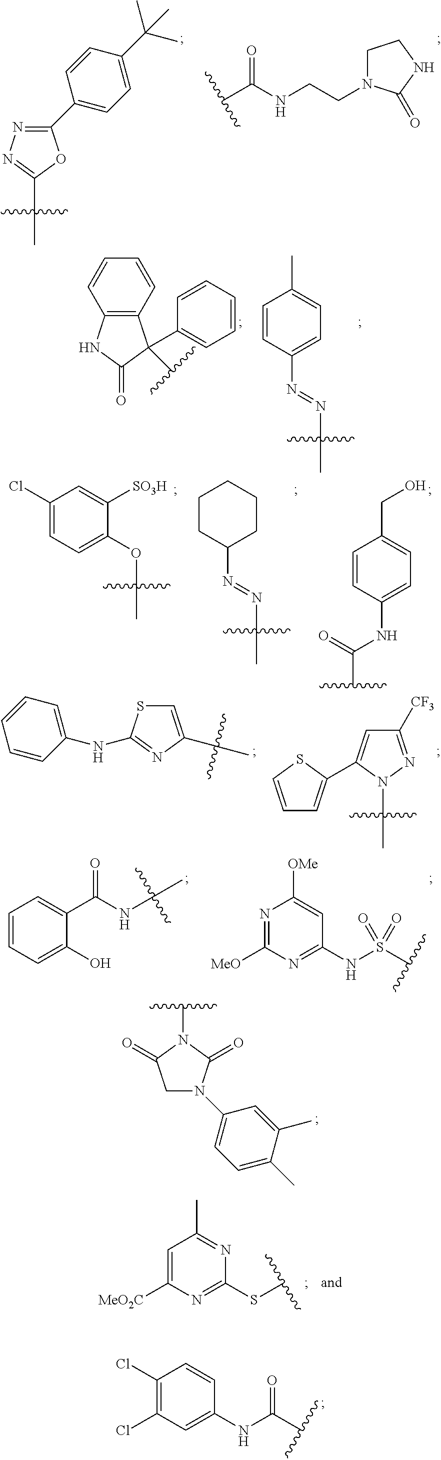
each R⁷ is independently selected from the group consisting of: H and unsubstituted or substituted C₁₋₆ alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

7. A compound of Formula (VII): 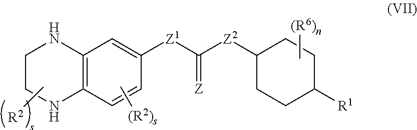
or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl;
unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 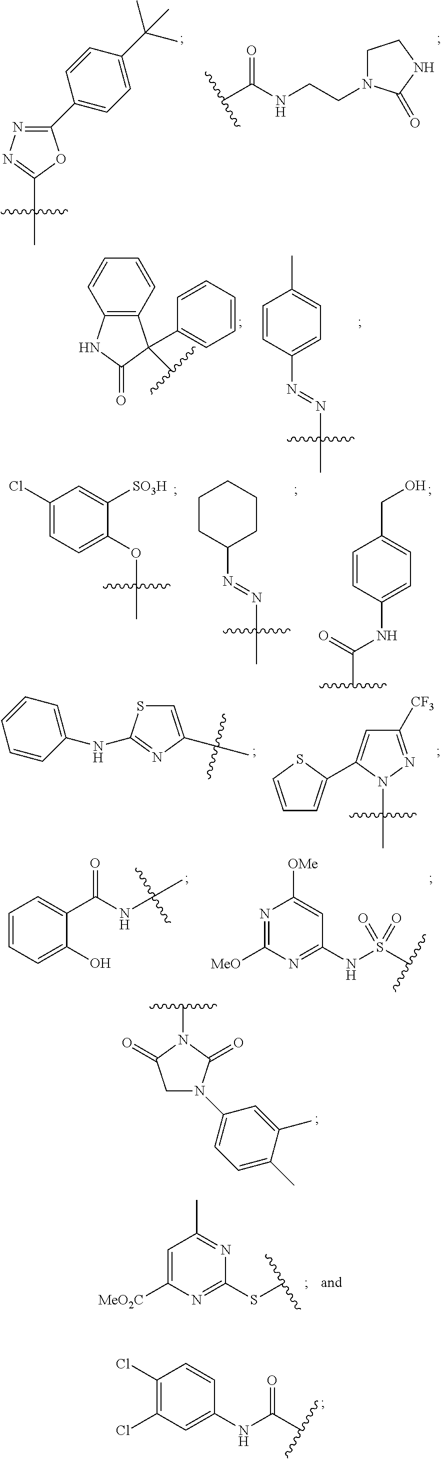
X is selected from the group consisting of: NR⁴, O, and S(O)ₚ;
each s is an integer from 0 to 2;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆ alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 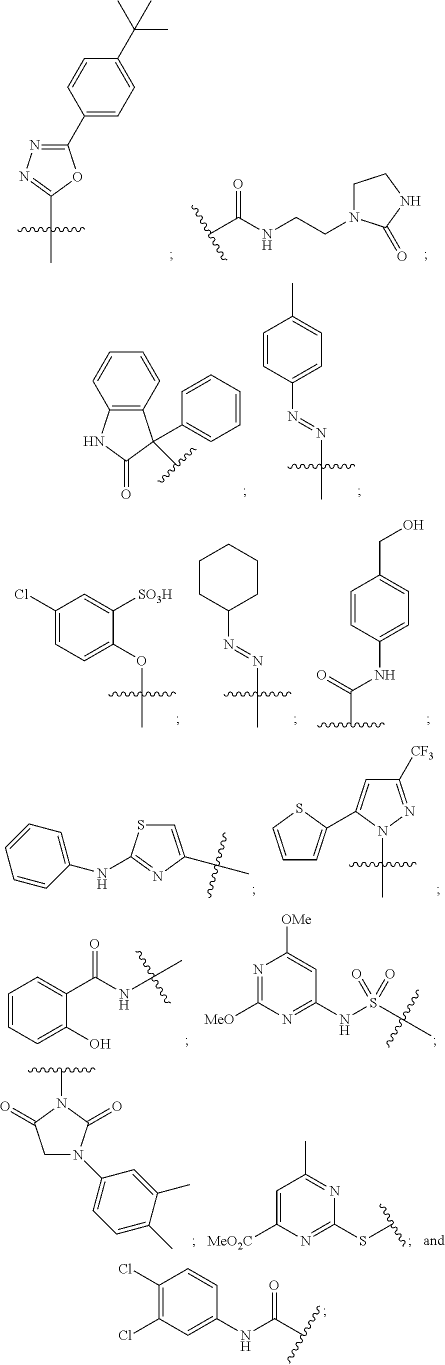
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

8. A compound of Formula (VIII): 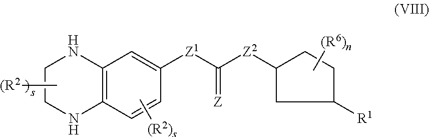
or a pharmaceutically acceptable salt thereof,
wherein:
Z, Z¹, and Z² are each independently selected from the group consisting of: NH, O, and S;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl;
unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl 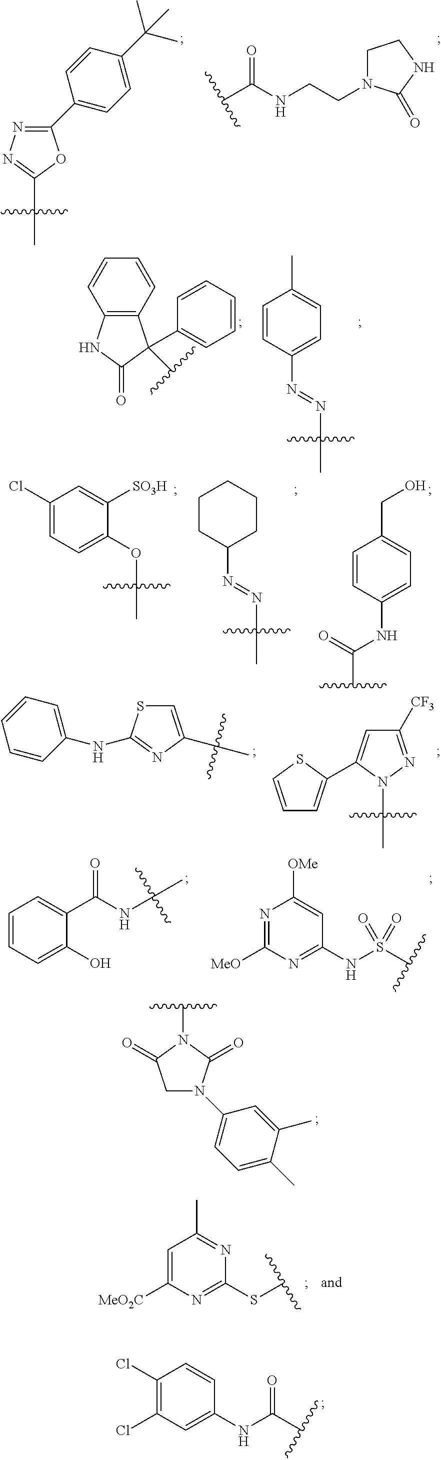
X is selected from the group consisting of: NR⁴, O, and S(O)ₚ;
each s is an integer from 0 to 2;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 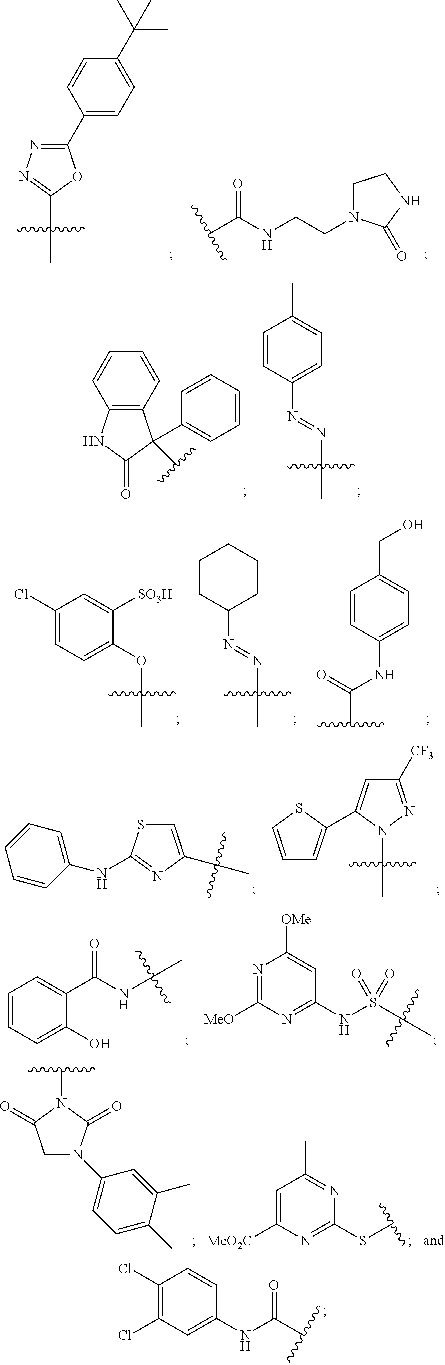
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

9. A compound of Formula (IX): 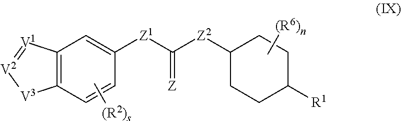
or a pharmaceutically acceptable salt thereof,
wherein:
Z, Z¹, and Z² are each independently selected from the group consisting of: NH, O, and S;
V¹, V², and V³ are each independently selected from the group consisting of: N, O, and S, such that the 5-membered ring is a heteroaryl ring;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 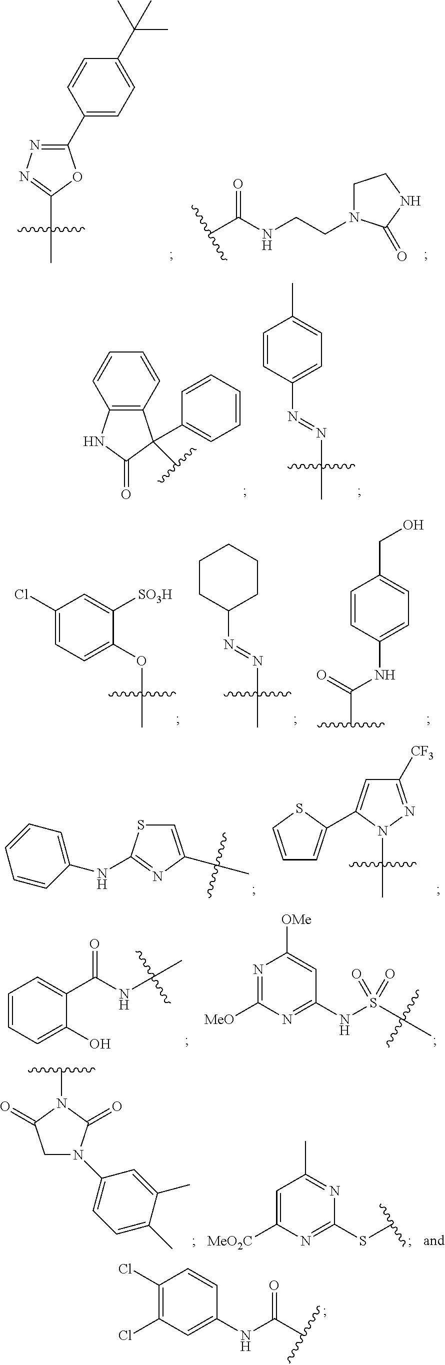
X is selected from the group consisting of: NR⁴, O, and S(O)ₚ;
each s is an integer from 0 to 2;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 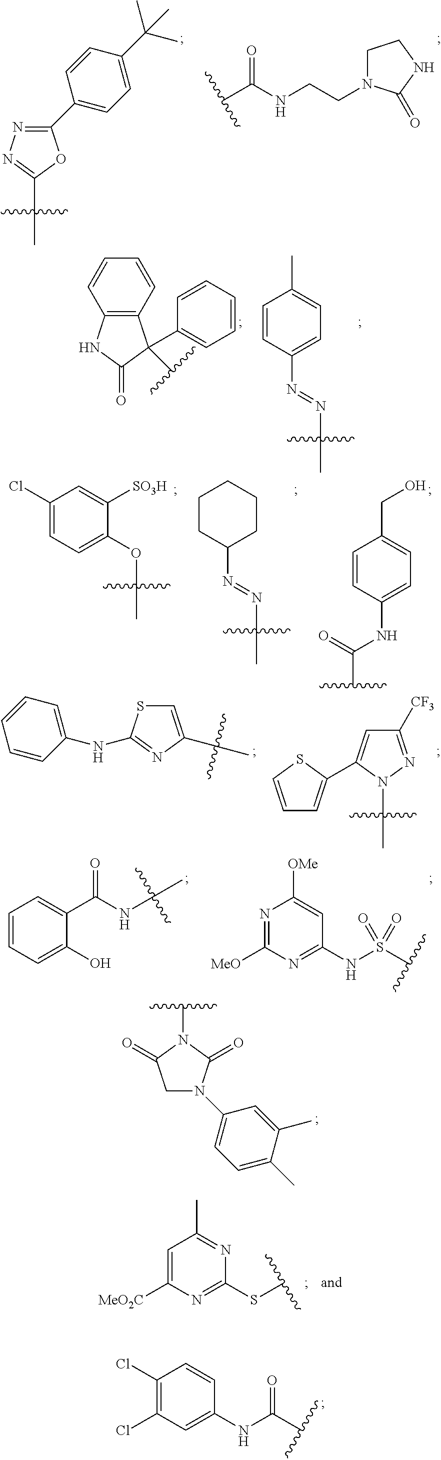
each R⁷ is independently selected from the group consisting of: H and unsubstituted or substituted C₁₋₆alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

10. A compound of Formula (X): 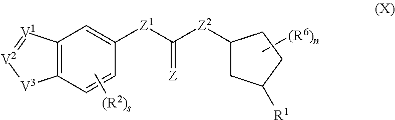
or a pharmaceutically acceptable salt thereof,
wherein:
Z, Z¹, and Z² are each independently selected from the group consisting of: NH, O, and S;
V¹, V², and V³ are each independently selected from the group consisting of: N, O, and S, such that the 5-membered ring is a heteroaryl ring;
R¹ is XWR³;
each R² is independently selected from the group consisting of: unsubstituted or substituted C₁₋₆alkyl; unsubstituted or substituted C₂₋₆alkenyl; unsubstituted or substituted C₂₋₆alkynyl; unsubstituted or substituted heteroaryl; unsubstituted or substituted heterocycle; C₁₋₆haloalkyl; C₁₋₆alkoxy; C₁₋₆haloalkoxy; halo; —CN; —SR⁴; —SO₂NR⁴; —COR⁴; —OCOR⁴; —CO₂R⁴; —CONHNR⁴R⁵; —OCONR⁴R⁵; —NO₂; —NR⁴R⁵; guanidine; —NR⁴COR⁵; —NR⁴SO₂R⁵; —CONR⁴R⁵; —OH; C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 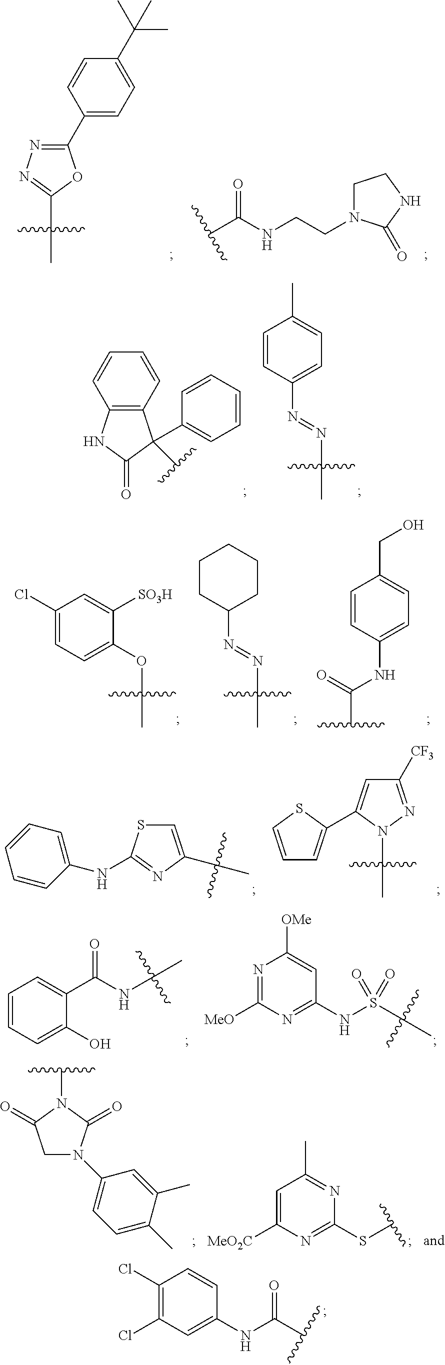
X is selected from the group consisting of: NR⁴, O, and S(O)ₚ;
each s is an integer from 0 to 2;
n is an integer from 0 to 2;
p is an integer from 0 to 2;
W is absent or [C(R⁵)₂]_{q};
q is an integer from 1 to 5;
R³ is selected from the group consisting of: unsubstituted or substituted aryl, and unsubstituted or substituted heteroaryl;
each R⁴ and R⁵ is independently selected from the group consisting of: H and unsubstituted or substituted C₁₋₆alkyl; and
each R⁶ is selected from the group consisting of: H, halo, unsubstituted or substituted C₁₋₆alkyl, unsubstituted or substituted C₂₋₆alkenyl, unsubstituted or substituted C₂₋₆alkynyl, (C₁₋₆alkoxy), —OH, —CONR⁴R⁵, —CONR⁷R⁸, —CN, —SR⁴, —SO₂NR⁴, —COR⁴, —CO₂R⁴, —CONHNR⁴R⁵, —OCONR⁴R⁵, —NO₂, —NR⁴R⁵, guanidine, —NR⁴COR⁵, (C₁₋₆alkoxy)NR⁴R⁵, unsubstituted or substituted carbocyclyl, unsubstituted or substituted heterocyclyl, unsubstituted or substituted aryl, unsubstituted or substituted heteroaryl, C₁₋₆alkylamino optionally substituted with a group consisting of: —OH, C₁₋₆alkoxy, —NR⁴R⁵, —COOH, substituted or unsubstituted C₁₋₆alkyl, —NR⁴SO₂R⁵, —CONR⁴R⁵, halo, aryl, heterocycle, and heteroaryl; (C₂₋₆alkenyl)-O—; C₁₋₆alkylsulfinyl; (aryl)-O—; (aryl)-(C═O)—; (aryl)NR⁴—; (aryl)SO₂NR⁴—; (heterocyclyl)-O—; (heterocyclyl)NR⁴—; (heterocyclyl)-(C═O)—; (heteroaryl)-O—; (heteroaryl)NR⁴—; (heteroaryl)-(C═O)—; (heteroaryl)SO₂NR⁴—; O—(CH₂)₂₋₄-heterocycle; O—(CH₂)₂₋₄—NR⁴R⁵; O—(CH₂)₂₋₄-heteroaryl; 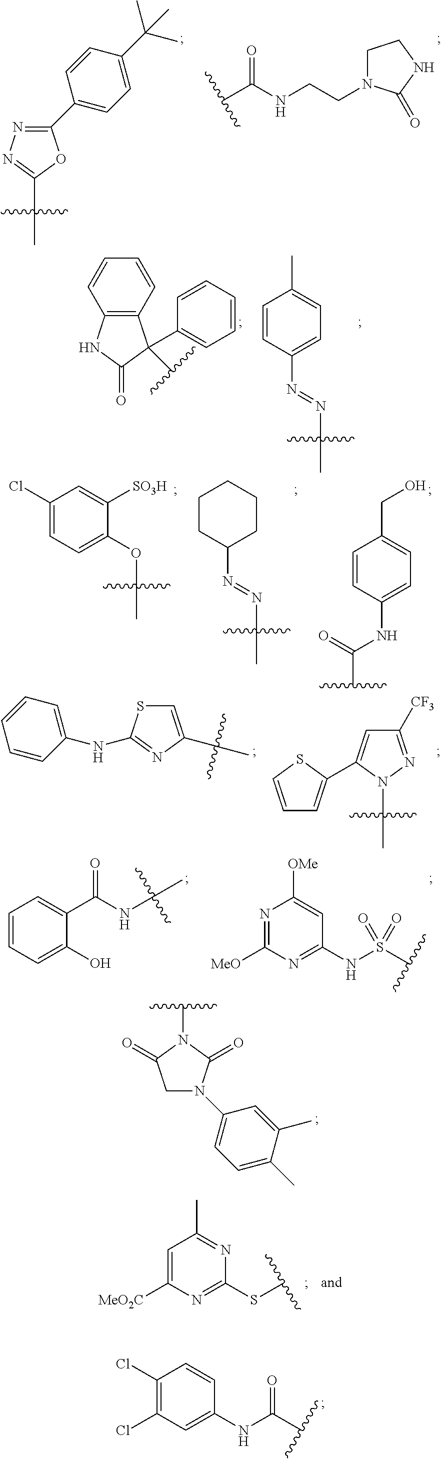
each R⁷ is independently selected from the group consisting of: H and unsubstituted or substituted C₁₋₆ alkyl; and
each R⁸ is independently selected from the group consisting of: H, unsubstituted or substituted C₁₋₆alkyl, carbocyclyl, (C₁₋₆alkyl)carbocyclyl, (C₁₋₆alkoxy)carbocyclyl, aryl, (C₁₋₆alkyl)aryl, (C₁₋₆alkoxy)aryl, heterocyclyl, (C₁₋₆alkyl)heterocyclyl, (C₁₋₆alkoxy)heterocyclyl, heteroaryl, (C₁₋₆alkyl)heteroaryl, and (C₁₋₆alkoxy)heteroaryl.

11.-35. (canceled)

36. A pharmaceutical composition comprising a pharmaceutically acceptable carrier or diluent and a compound of claim 1, or a pharmaceutically acceptable salt thereof.

37. A method for the treatment of a cancer, a hemolytic anemia not caused by an infectious agent in a patient, Wolcott-Rallison syndrome, a neurodegenerative disease, tuberous sclerosis complex, an autism spectrum disorder, a ribosomal defect disease, or a mental retardation disorder in a patient, the method comprising administering to the patient a therapeutically effective amount of a compound of claim 1, or a pharmaceutically acceptable salt thereof.

38.-53. (canceled)

54. A method of activating one or more eIF2α kinases in a cell, the method comprising contacting the cell with an effective amount of a compound of claim 1, or a pharmaceutically acceptable salt thereof.
